(19) United States
(12) Patent Application Publication
(10) Pub. No.: US 2016/0257961 A1
(43) Pub. Date: Sep. 8, 2016

(54) POLYNUCLEOTIDE CONSTRUCTS HAVING DISULFIDE GROUPS

(71) Applicants: Curt W. BRADSHAW, San Diego, CA (US); Laxman ELTEPU, Carlsbad, CA (US); Ayman KABAKIBI, San Diego, CA (US); Son LAM, San Diego, CA (US); Bin LIE, San Diego, CA (US); Dingguo LIE, San Diego, CA (US); Bryan R. MEADE, San Diego, CA (US); Sukumar SAKAMURI, San Diego, CA (US); SOLSTICE BIOLOGICS, LTD., Dubblin 2 (IE)

(72) Inventors: Curt W. BRADSHAW, San Diego, CA (US); Laxman ELTEPU, San Diego, CA (US); Ayman KABAKIBI, San Diego, CA (US); Son LAM, San Diego, CA (US); Bin LIU, San Diego, CA (US); Dingguo LIU, San Diego, CA (US); Bryan R. MEADE, San Diego, CA (US); Sukumar SAKAMURI, San Diego, CA (US)

(21) Appl. No.: 15/034,973

(22) PCT Filed: Nov. 6, 2014
(86) PCT No.: PCT/US14/64401

§ 371 Date: May 6, 2016

Related U.S. Application Data
(60) Provisional application No. 61/900,685, filed on Nov. 6, 2013.
(60) Provisional application No. 61/975,686, filed on Apr. 4, 2014.
(60) Provisional application No. 62/008,906, filed on Jun. 6, 2014.

(51) Int. Cl. C12N 15/113

(52) U.S. Cl. CPC C12N 15/1137; C12N 2310/14; C12N 2310/321; C12N 2310/311; C12N 2310/351

(57) ABSTRACT

The invention features polynucleotide constructs containing one or more components (i) containing a disulfide linkage, where each of the one or more components is attached to an internucleotide bridging group or a terminal group of the polynucleotide construct, and each of the one or more components (i) contains one or more bulky groups proximal to the disulfide group. The invention also features polynucleotide constructs containing one or more components (i) containing a disulfide linkage, where each of the one or more components (i) is attached to an internucleotide bridging group or a terminal group of the polynucleotide construct, and each of the one or more components (i) contains at least 4 atoms in a chain between the disulfide linkage and the phosphorus atom of the internucleotide bridging group or the terminal group; and where the chain does not contain a phosphate, an amide, an ester, or an alkenylene. The invention also features methods of delivering a polynucleotide to a cell using the polynucleotide constructs of the invention.

### FIELD OF THE INVENTION

This invention relates to compositions and methods for transfecting cells.

### BACKGROUND

Nucleic acid delivery to cells both in vitro and in vivo has been performed using various recombinant viral vectors, lipid delivery systems and electroporation. Such techniques have sought to treat various diseases and disorders by knocking-out gene expression, providing genetic constructs for gene therapy or to study various biological systems.

Polyanionic polymers such as polynucleotides do not readily diffuse across cell membranes. To overcome this problem for cultured cells, cationic lipids are typically combined with anionic polynucleotides to assist uptake. Unfortunately, this complex is generally toxic to cells, which means that both the exposure time and concentration of cationic lipid must be carefully controlled to insure transfection of viable cells.

The discovery of RNA interference (RNAi) as a cellular mechanism that selectively degrades mRNAs allows for both the targeted manipulation of cellular phenotypes in cell culture and the potential for development of directed therapeutics (Behlke, Mol. Ther. 13, 644-670, 2006; Xie et al., Drug Discov. Today 11, 67-73, 2006). However, because of their size and negative (anionic) charged nature, siRNAs are macromolecules with no ability to enter cells. Indeed, siRNAs are 25× in excess of Lipinski's “Rule of 5 s” for cellular delivery of membrane diffusible molecules that generally limits size to less than 500 Da.

Consequently, in the absence of a delivery vehicle or transfection agent, naked siRNAs do not enter cells, even at millimolar concentrations (Barquinero et al., Gene Ther. 11 Suppl 1, S3-9, 2004). Significant attention has been focused on the use of cationic lipids that both condense the siRNA and punch holes in the cellular membrane to solve the siRNA delivery problem. Although widely used, transfection reagents fail to achieve efficient delivery into many cell types, especially primary cells and hematopoietic cell lineages (T and B cells, macrophage). Moreover, lipofection reagents often result in varying degrees of cytotoxicity ranging from mild in tumor cells to high in primary cells.

### SUMMARY OF THE INVENTION

In one aspect, the invention provides a polynucleotide construct containing one or more components (i) containing a disulfide linkage, where each of the one or more components is attached to an internucleotide bridging group or a terminal group, e.g., a 3′ terminal group, of the polynucleotide construct, and each of the one or more components (i) contains one or more bulky groups proximal to the disulfide group. In specific embodiments, when the one or more components (i) includes an alkylene group linking the disulfide linkage to the terminal group, the number of atoms in the shortest chain between the terminal group and the disulfide linkage is 2, 3, 4, or 5; and/or the disulfide linkage of the one or more components (i) is not connected to the internucleotide bridging group by alkenylene.

In another aspect, the invention provides a polynucleotide construct containing one or more components (i) containing a disulfide linkage, where each of the one or more components (i) is attached to an internucleotide bridging group or a terminal group, e.g., 3′ terminal group, of the polynucleotide construct, and each of the one or more components (i) contains at least 4 atoms in a chain between the disulfide linkage and the phosphorus atom of the internucleotide bridging group or the terminal group;

where the chain does not contain a phosphate, an amide, an ester, or an alkenylene;

where, when the chain includes an alkylene group, the number of atoms between the terminal group and the disulfide group is 4 or 5.

In some embodiments, at least one of the one or more components (i) further includes one or more of a polypeptide, a carbohydrate, a neutral organic polymer, a positively charged polymer, a therapeutic agent, a targeting moiety, or an endosomal escape moiety.

In certain embodiments, at least one of the one or more components (i) includes a carbohydrate (e.g., N-acetyl galactosamine or mannose). In particular embodiments, at least one of the one or more components (i) includes a neutral organic polymer or a positively charged polymer. In other embodiments, the neutral organic polymer includes 1 to 200 alkylene oxide units (e.g., ethylene oxide). In yet other embodiments, at least one of the one or more components (i) includes a targeting moiety (e.g., a folate ligand). In still other embodiments, at least one of the one or more components (i) includes a polypeptide, e.g., a protein transduction domain. In certain embodiments, at least one of the one or more components (i) includes an endosomal escape moiety.

In other embodiments, the polynucleotide construct has 2 to 150 nucleotides, e.g., 5 to 50, 8 to 40, 10 to 32, 15 to 25, 18 to 25, or 20-25 nucleotides in a single strand.

In particular embodiments of any aspect, the disulfide linkage is not bonded to pyridyl (e.g., 2-pyridyl).

In some embodiments, each the one or more components (i), independently, contains a group having the structure of (R⁴)ᵣ-L-A¹-S—S-A²-A³-A⁴-,

where A¹ a bond or a linker including or being one or more optionally substituted N, O, S, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S (e.g., excluding pyridyl); optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ contains one or more of optionally substituted N, O, and S, none of the optionally substituted N, O, and S is directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₁₋₉ heterocyclylene having 1 to 4 heteroatoms selected from N, O, and S; optionally substituted C₆₋₁₄ arylene, optionally substituted C₁₋₉ heteroarylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or being one or more conjugating moieties; and

R⁴ is hydrogen, optionally substituted C₁₋₆ alkyl, a hydrophilic functional group, or a group including an auxiliary moiety selected from the group consisting of a small molecule, a polypeptide, a carbohydrate, a neutral organic polymer, a positively charged polymer, a therapeutic agent, a targeting moiety, an endosomal escape moiety, and combinations thereof;

where A⁴ is proximal to the internucleotide bridging group or the terminal group; and

where A¹ or A² contains one or more bulky groups proximal to —S—S—.

In certain embodiments, the one or more components (i) consists of a group having the structure of (R⁴)ᵣ-L-A¹-S—S-A²-A³-A⁴-.

In particular embodiments, a polynucleotide construct has the structure of Formula I:

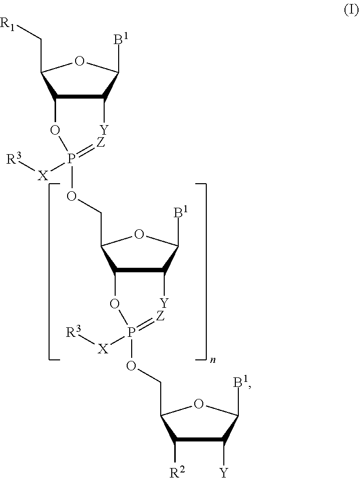

or a salt thereof,

where n is a number from 0 to 150;

each B¹ is independently a nucleobase;

each X is independently selected from the group consisting of O, S, and optionally substituted N;

each Y is independently selected from the group consisting of hydrogen, hydroxyl, halo, optionally substituted C₁₋₆ alkoxy, and a protected hydroxyl group;

each Z is independently O or S;

R¹ is selected from the group consisting of H, hydroxyl, optionally substituted C₁₋₆ alkoxy, a protected hydroxyl group, a monophosphate, a diphosphate, a triphosphate, a tetraphosphate, a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and any combination thereof, or R¹ is

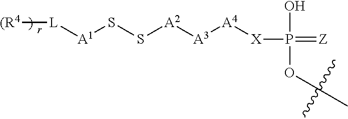

or a salt thereof;

R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an optionally substituted C₁₋₆ alkyl, an amino containing group, a biotin containing group, a digoxigenin containing group, a cholesterol containing group, a quencher containing group, a phosphothiol, a polypeptide, a carbohydrate, a neutral organic polymer, a positively charged polymer, a therapeutic agent, a targeting moiety, an endosomal escape moiety, and any combination thereof, or R² is

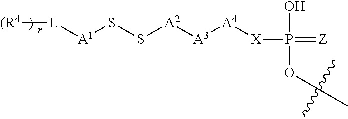

or a salt thereof; and

each R³ is independently absent, a hydrogen, optionally substituted C₁₋₆ alkyl, or a group having the structure of Formula II:

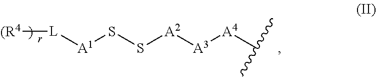

where A¹ is a bond or a linker including or being one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S (e.g., excluding pyridyl); optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ contains one or more of optionally substituted N, O, and S, the optionally substituted N, O, or S is not directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or being one or more conjugating moieties;

R⁴ is hydrogen, optionally substituted C₁₋₆ alkyl, a hydrophilic functional group, or a group including an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms in the shortest chain connecting —S—S— and X.

In certain embodiments, at least one R³ has the structure of formula (II).

In some embodiments, at least one of R¹ and R² is

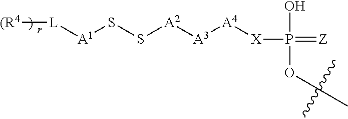

or a salt thereof.

In particular embodiments, when R¹ or R² is

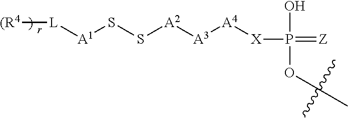

or a salt thereof in which A², A³, and A⁴ combine to form an alkylene group, the alkylene group is C₄₋₅ alkylene.

In other embodiments, when R¹ or R² is

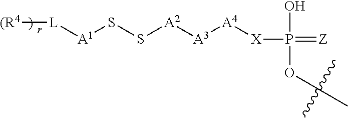

or a salt thereof, the group -A²-A³-A⁴-X— does not contain a phosphate, an amide, an ester, or an alkenylene.

In certain embodiments, each X is O. In particular embodiments, each Z is O.

In some embodiments, when a nucleoside is linked via its 3′-O—P—X— chain to R³ having the structure of formula (II), Y of the nucleoside is halo, optionally substituted C₁₋₆ alkoxy, or hydroxyl, e.g., Y is F or OMe.

In other embodiments, R⁴ is bound to L, to A¹, or to disulfide via a bond formed by a reaction selected from the group consisting of a pericyclic reaction; an alkylation or arylation of a hydroxyl, thiol, or amino moiety; and a reaction of a hydroxyl, thiol, or amino nucleophile with an electrophile. In yet other embodiments, the pericyclic reaction is a cycloaddition. In still other embodiments, the cycloaddition is Hüisgen cycloaddition.

In particular embodiments, R⁴ is bound to L, to A¹, or to the disulfide via an amide bond, a sulfonamide bond, a carboxylic ester, a thioester, optionally substituted C₆₋₁₄ aryl, optionally substituted C₁₋₉ heterocyclyl having 1 to 4 heteroatoms selected from N, O, and S; optionally substituted C₁₋₉ heteroaryl having 1 to 4 heteroatoms selected from N, O, and S; an imine; a hydrazone; an oxime; or a succinimide.

In certain embodiments, one or more of the hydrophilic functional groups and conjugating moieties are protected with protecting groups.

In some embodiments, L is formed through a condensation reaction with an aldehyde conjugating moiety to form an imine, enamine, oxime, or hydrazone bond.

In other embodiments, at most 90% of the disulfides are linked to one or more auxiliary moieties. In particular embodiments, at most 75% of the disulfides are linked to one or more auxiliary moieties. In certain embodiments, at most 50% of the disulfides are linked to one or more auxiliary moieties. In some embodiments, at most 25% of the disulfides are linked to one or more auxiliary moieties. In particular embodiments, at most 75% of the nucleotides in the polynucleotide construct are linked to the disulfide. In certain embodiments, at most 65% of the nucleotides in the polynucleotide construct are linked to the disulfide. In some embodiments, at most 55% of the nucleotides in the polynucleotide construct are linked to the disulfide. In particular embodiments, at most 45% of the nucleotides in the polynucleotide construct are linked to the disulfide.

In certain embodiments, the polynucleotide construct contains 1 to 100 groups formula (II). In other embodiments, the polynucleotide construct contains 2 to 50 groups of formula (II). In yet other embodiments, the polynucleotide construct contains 2 to 30 groups of formula (II). In still other embodiments, the polynucleotide construct contains 2 to 10 groups of formula (II). In further embodiments, the polynucleotide construct contains 5 to 50 nucleotides. In particular embodiments, the polynucleotide construct contains 8 to 40 nucleotides. In some embodiments, the polynucleotide construct contains 10 to 32 nucleotides.

In particular embodiments, at least one R⁴ includes or is a targeting moiety. In certain embodiments, at least one R⁴ includes or is a carbohydrate. In some embodiments, at least one R⁴ includes or is mannose. In other embodiments, at least one R⁴ includes or is N-acetyl galactosamine. In yet other embodiments, at least one R⁴ includes or is a folate ligand. In still other embodiments, at least one R⁴ includes or is a protein transduction domain. In certain other embodiments, at least one R⁴ includes or is an endosomal escape moiety. In particular embodiments, at least one R⁴ includes or is a prostate specific membrane antigen (PSMA).

In some embodiments, the ratio of R³ groups that are absent or H to R³ groups that have the structure of formula (II) is from 1:10 to 10:1, e.g., 1:5 to 5:1, 1:3 to 3:1, 1:2 to 2:1, or about 1:1.

In other embodiments, L includes or consists of 1 to 500 monomers, each of which is, independently, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; carbonyl; thiocarbonyl; imino; optionally substituted N; O; or S(O)ₘ, wherein m is 0, 1, or 2.

In particular embodiments, L contains or consists of one or more C₁₋₆ alkyleneoxy groups, e.g., ethyleneoxy. In certain embodiments, L contains fewer than 100 C₁₋₆ alkyleneoxy groups, e.g., ethyleneoxy. In still other embodiments, L includes or consists of polyethylene oxide, polypropylene oxide, poly(trimethylene oxide), polybutylene oxide, poly(tetramethylene oxide), or a diblock or triblock copolymer thereof. In particular embodiments, L includes or consists of polyethylene oxide.

In some embodiments, L contains or consists of one or more amino acid residues (e.g., Arg, Asn, Asp, Cys, Glu, Gin, His, Lys, Ser, Thr, Trp, or Tyr).

In other embodiments, L contains or is a group having the structure of formula (III):

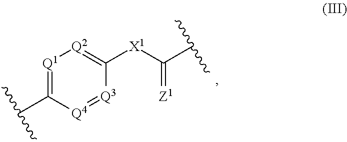

where each Q¹, Q², Q³, and Q⁴ is independently N or CR⁷;

X¹ is O or NR⁶;

Z¹ is O or S;

each R⁷ is independently selected from the group consisting of H; optionally substituted C₁₋₆ alkyl; optionally substituted C₂₋₆ alkenyl; optionally substituted C₂₋₆ alkynyl; halo; hydroxyl; —CHO; optionally substituted C₁₋₆ alkanoyl; carboxyl; cyano; nitro; optionally substituted amino; thiol; optionally substituted C₁₋₉ heterocyclyl having 1 to 4 heteroatoms selected from N, O, and S; optionally substituted C₁₋₉ heteroaryl having 1 to 4 heteroatoms selected from N, O, and S; optionally substituted C₆₋₁₄ aryl; optionally substituted C₃₋₈ cycloalkyl; and optionally substituted C₃₋₈ cycloalkenyl.

In particular embodiments, Q¹ is CR⁷; Q² is CR⁷; Q³ is CR⁷; Q⁴ is CR⁷; each R⁷ is independently H, optionally substituted C₁₋₆ alkyl, or halo (e.g., R⁷ is H); X¹ is CR⁷; and/or Z¹ is S.

In certain embodiments, L contains or is one or more groups having the structure of formula (IV):

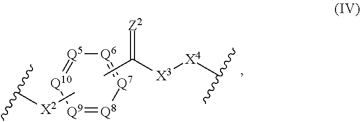

where each Q⁵, Q⁶, Q⁷, Q⁸, Q⁹, and Q¹⁰ is, independently, N, CR⁷, or C bonded to —X² or —C(Z²)X³X⁴, where no more than one of Q⁵, Q⁶, Q⁷, Q⁸, Q⁹, and Q¹⁰ is C bonded to —X², and no more than one of Q⁵, Q⁶, Q⁷, Q⁸, Q⁹, and Q¹⁰ is C bonded to —C(Z²)X³X⁴;

X² is optionally substituted C₁₋₆ cycloalkylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted diazaalkenylene; optionally substituted saturated diaza; unsaturated diaza; optionally substituted azacarbonyl; or oxacarbonyl;

X³ is a bond, O, NR⁷, or S;

X⁴ is absent, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O; optionally substituted C₁₋₉ heterocyclylene having 1 to 4 heteroatoms selected from N, O, and S; or optionally substituted (C₁₋₉ heterocyclyl)-C₁₋₄-alkylene having 1 to 4 heteroatoms selected from N, O, and S; and

Z² is O, S, or NR⁷; and

each R⁷ is independently selected from the group consisting of H, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁, heterocyclyl)-C₁₋₄-alkyl having 1 to 4 heteroatoms selected from N, O, and S; optionally substituted amino; and optionally substituted C₁₋₆ alkoxy; and

where the two of Q⁵, Q⁶, Q⁷, Q⁸, Q⁹, and Q¹⁰ linked to X² and —C(Z)X³X⁴ are not N.

In some embodiments, Q⁵ is N; Q⁶ is CR⁷; Q⁷ is C bonded to —C(Z²)X³X⁴; Q⁸ is CR⁷; Q⁹ is CR⁷; and/or Q¹⁰ is C bonded to X². Each R⁷ may be independently selected from the group consisting of H, halo, and optionally substituted C₁₋₆ alkyl (e.g., R⁷ is H). In other embodiments, X² is optionally substituted diazaalkenylene or optionally substituted saturated diaza. In yet other embodiments, X³ is NR⁷. In particular embodiments, X⁴ is absent. In certain embodiments, Z² is O.

In further embodiments, L includes or is one or more groups having the structure of formulas (VIa) and (VIb):

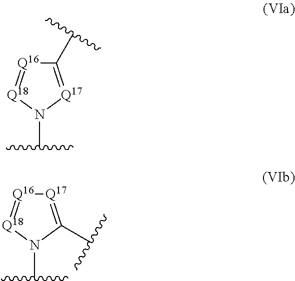

wherein each Q¹⁶, Q¹⁷, and Q¹⁸ is independently N or CR⁷;

each of R⁷ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In further embodiments, L includes or is one or more groups having the structure:

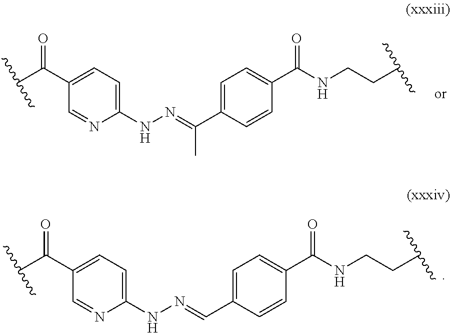 [SMILES: *CCNC(=O)c1ccc(/C(C)=N/Nc2ccc(C(*)=O)cn2)cc1 ; *CCNC(=O)c1ccc(/C=N/Nc2ccc(C(*)=O)cn2)cc1]

In other embodiments, L is a bond.

In some embodiments, A³ is selected from the group consisting of a bond, optionally substituted C₁₋₆ alkylene; optionally substituted C₆₋₁₄ arylene; O; optionally substituted N; and S.

In other embodiments, A³ is selected from the group consisting of a bond, optionally substituted C₁₋₆ alkylene; optionally substituted C₆₋₁₄ arylene; and O.

In certain embodiments, A⁴ is optionally substituted C₁₋₆ alkylene.

In particular embodiments, A includes or is a group having the structure:

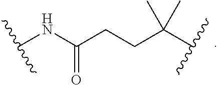 [SMILES: *NC(=O)CCC(*)(C)C]

In some embodiments, A¹ is a bond or includes or is one or more groups selected independently from the group consisting of optionally substituted C₁₋₆ alkylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S (e.g., exclude pyridyl); optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; optionally substituted (C₁₋₉ heterocyclyl)-C₁₋₄-alkylene having 1 to 4 heteroatoms selected from N, O, and S; optionally substituted N; and O.

In particular embodiments, A¹ is a bond or contains or is one or more groups selected independently from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In certain embodiments, A¹ is a bond or contains or is one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In other embodiments, A¹ is a bond or contains or is one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In particular embodiments, A¹ is a bond.

In certain embodiments, A² is optionally substituted C₁₋₆ alkylene, optionally substituted C₃₋₈ cycloalkylene; optionally substituted C₃₋₈ cycloalkenylene; optionally substituted C₆₋₁₄ arylene; or optionally substituted C₁₋₉ heteroarylene having 1 to 4 heteroatoms selected from N, O, and S.

In other embodiments, A² is optionally substituted C₁₋₆ alkylene, optionally substituted C₃₋₈ cycloalkylene; optionally substituted C₆₋₁₄ arylene; or optionally substituted C₁₋₉ heteroarylene having 1 to 4 heteroatoms selected from N, O, and S. In yet other embodiments, A² is optionally substituted optionally substituted C₆₋₁₄ arylene or optionally substituted C₁₋₉ heteroarylene having 1 to 4 heteroatoms selected from N, O, and S.

In still other embodiments, A² has a structure of formula (VI):

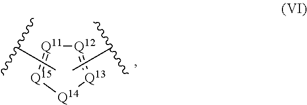

where

Q¹¹ is N, or C bonded to R¹⁰ or to the disulfide linkage;

Q¹² is N, or C bonded to R¹ or to A³;

Q¹³ is N or C bonded to R¹² or to A³;

Q¹⁴ is O, S, N bonded to R¹³ or to A³, or —C(R¹⁴ or bond to A³)=C(R¹⁵ or bond to A³)-;

Q¹⁵ is N, or C bonded to R¹⁶ or to the disulfide linkage;

each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁ 6 alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and

where one and only one of Q¹¹ and Q¹⁵ is bonded to the disulfide linkage, and one and only one of Q¹², Q¹³, and Q¹⁴ is bonded to A³.

In particular embodiments, Q¹¹ is C bonded to the disulfide linkage; Q¹² is C bonded to A³; Q¹³ is C bonded to R¹²; R¹² is H, halo, or C₁₋₆ alkyl; Q¹⁴ is O or —C(R¹⁴)═C(R¹⁵)—; R¹⁴ is H, halo, or C₁₋₆ alkyl; R¹⁵ is H, halo, or C₁₋₆ alkyl; Q¹⁵ is C bonded to R¹⁶; and/or R¹⁶ is H, halo, or C₁₋₆ alkyl.

In other embodiments, A³ has a structure of formula (VI):

wherein

Q¹¹ is N, or C bonded to R¹⁰ or to A²;

Q¹² is N, or C bonded to R¹¹ or to A⁴;

Q¹³ is N or C bonded to R¹² or to A⁴;

Q¹⁴ is O, S, N bonded to R¹³ or to A⁴, or —C(R¹⁴ or bond to A⁴)=C(R¹⁵ or bond to A⁴)-;

Q¹⁵ is N, or C bonded to R¹⁶ or to A²;

each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and

wherein

one and only one of Q¹¹ and Q¹⁵ is bonded to A², and

one and only one of Q¹², Q¹³, and Q¹⁴ is bonded to A⁴.

In certain embodiments, Q¹¹ is C bonded to A². In particular embodiments, Q¹² is C bonded to A⁴. In some embodiments, Q¹³ is C bonded to R¹². In other embodiments, R¹² is H, halo, or C₁₋₆ alkyl. In certain other embodiments, R¹⁴ is O. In yet other embodiments, Q¹⁴ is —C(R¹⁴)═C(R¹⁵)—. In still other embodiments, R¹⁴ is H, halo, or C₁₋₆ alkyl. In some embodiments, R¹⁵ is H, halo, or C₁₋₆ alkyl. In particular embodiments, Q¹⁵ is C bonded to R¹⁶. In certain embodiments, R¹⁶ is H, halo, or C₁₋₆ alkyl.

In some embodiments, when the carbon atom attached to the sulfur atom of —S—S-A²-A³-A⁴- is an alkylene carbon atom, the alkylene carbon atom is connected to at most one hydrogen atom, e.g., connected to no hydrogen atoms. In yet other embodiments, when the carbon atom attached to the sulfur atom of —S—S-A²-A³-A⁴- is an alkenylene carbon atom, the alkenylene carbon atom is not connected to a hydrogen atom. In still other embodiments, the carbon atom attached to the sulfur atom of —S—S-A²-A³-A⁴- is not an alkynylene carbon atom. In certain other embodiments, when the carbon atom attached to the sulfur atom of (R⁴)ᵣ-L-A¹-S—S— is an alkylene carbon atom, the carbon atom is connected to at most one hydrogen atom, e.g., connected to no hydrogen atoms.

In certain embodiments, A¹ and A², together with —S—S— to which they are attached, join to form an optionally substituted 5 to 16 membered ring, e.g., and optionally substituted 5 to 7 membered ring.

In particular embodiments, A¹, A², A³, and A⁴ or A², A³, and A⁴ and the disulfide linkage combine to form a group having the structure of any one of:

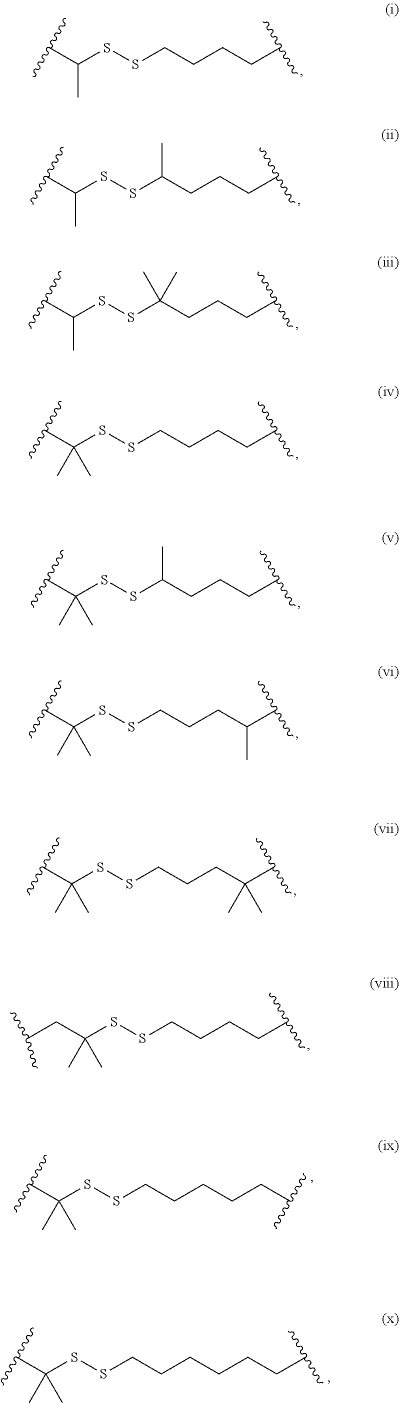

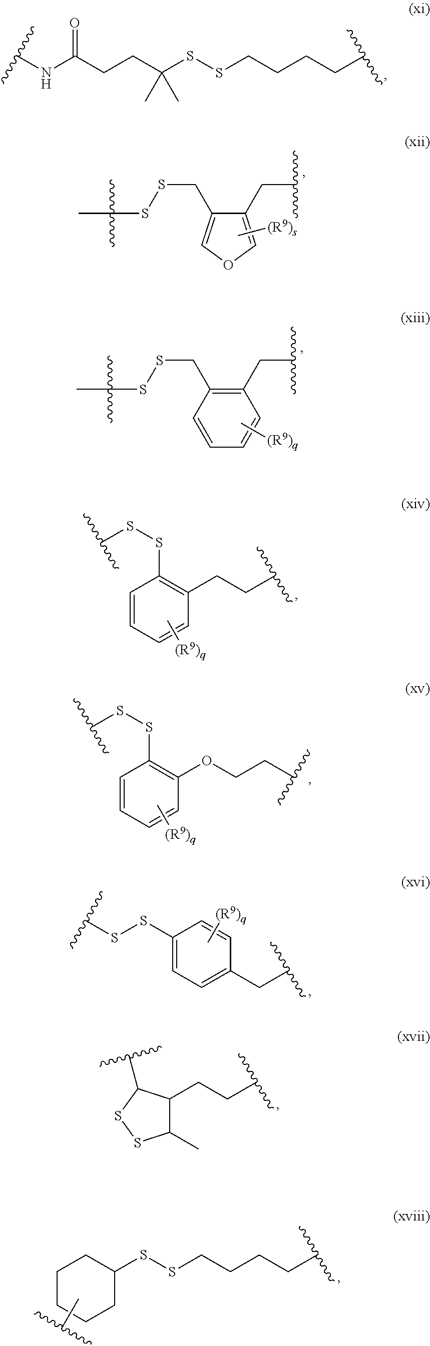

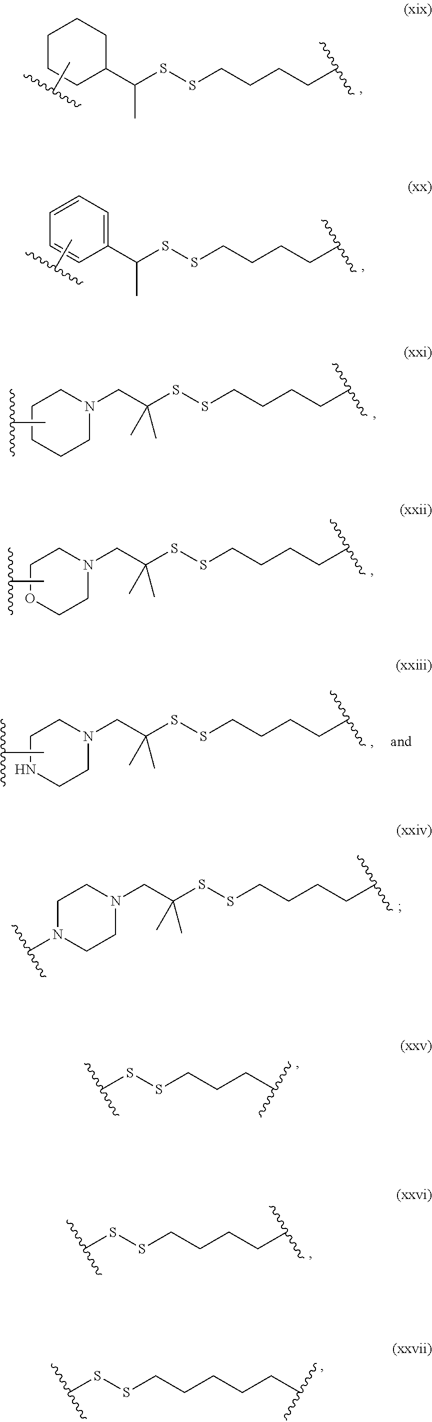

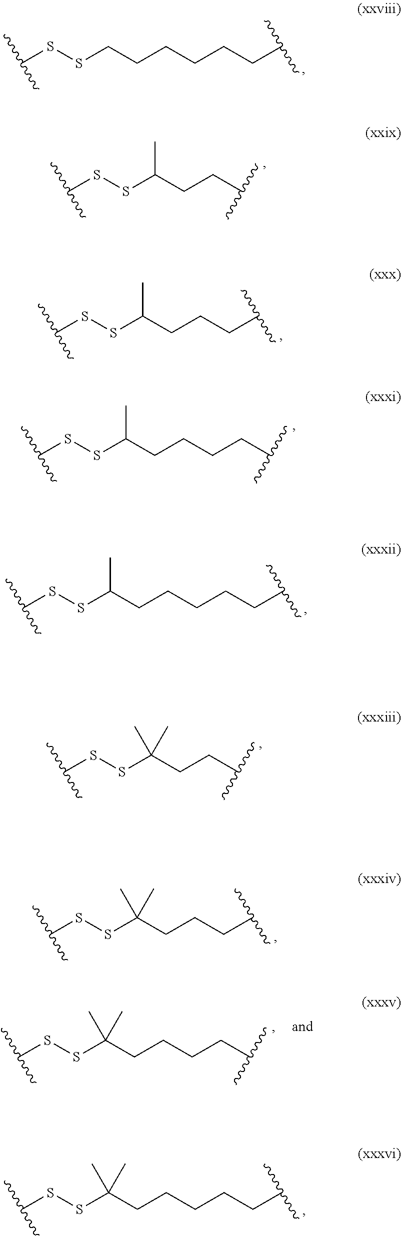

where,
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; optionally substituted amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In certain embodiments, R⁹ is halo or optionally substituted C₁₋₆ alkyl. In some embodiments, s is 0 or 1. In yet other embodiments, s is 0. In still other embodiments, q is 0, 1, or 2. In yet other embodiments, q is 0 or 1.

In certain embodiments, two adjacent R⁹ groups, together with the atoms to which each said R⁹ is attached combine to form C₂₋₅ heteroaryl optionally substituted with 1, 2, or 3 C₁₋₆ alkyl groups.

In certain embodiments, A², A³, A⁴, and —S—S— combine to form a structure:

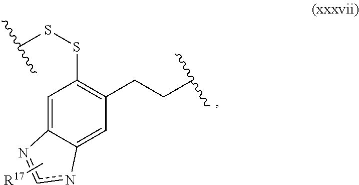

wherein the dotted lines represent one and only one double bond, and

R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In some embodiments, R¹⁷ is H or C₁₋₆ alkyl.

In particular embodiments, A², A³, A⁴, and the disulfide linkage combine to form a group having the structure of any one of:

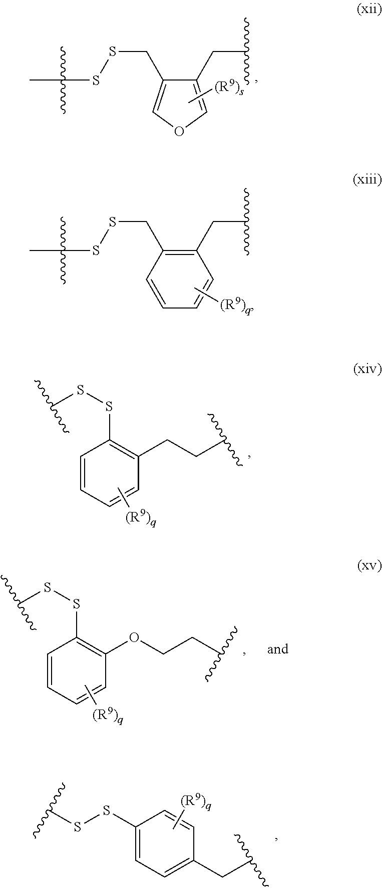

In particular embodiments, A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A and A², together with —S—S—, join to form an optionally substituted 5 to 16 membered ring. In certain embodiments, A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A¹ and A², together with —S—S—, join to form an optionally substituted 5 to 16 membered ring.

In some embodiments, R¹ is selected from the group consisting of H, hydroxyl, optionally substituted C₁₋₆ alkoxy, a protected hydroxyl group, a monophosphate, a diphosphate, a triphosphate, an optionally substituted C₁₋₆ alkyl, an amino containing group, a biotin containing group, a polypeptide, a carbohydrate, a neutral organic polymer, a positively charged polymer, a therapeutic agent, a targeting moiety, an endosomal escape moiety, and any combination thereof;

In certain embodiments, R² is selected from the group consisting of H, hydroxyl, optionally substituted C₁₋₆ alkoxy, a protected hydroxyl group, a monophosphate, a diphosphate, a triphosphate, an optionally substituted C₁₋₆ alkyl, an amino containing group, a biotin containing group, a polypeptide, a carbohydrate, a neutral organic polymer, a positively charged polymer, a therapeutic agent, a targeting moiety, an endosomal escape moiety, and any combination thereof

In particular embodiments, a polynucleotide construct includes one or more groups of Formula (V) attached to one or more internucleotide bridging groups or terminal nucleotide groups of the polynucleotide:

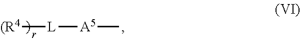

or a salt thereof,

where

each L is independently a bond or a conjugating group including or being one or more conjugating moieties;

each R⁴ is independently hydrogen, optionally substituted C₁₋₆ alkyl, a hydrophilic functional group, or a group including an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10; and

A⁵ is selected from the group consisting of:

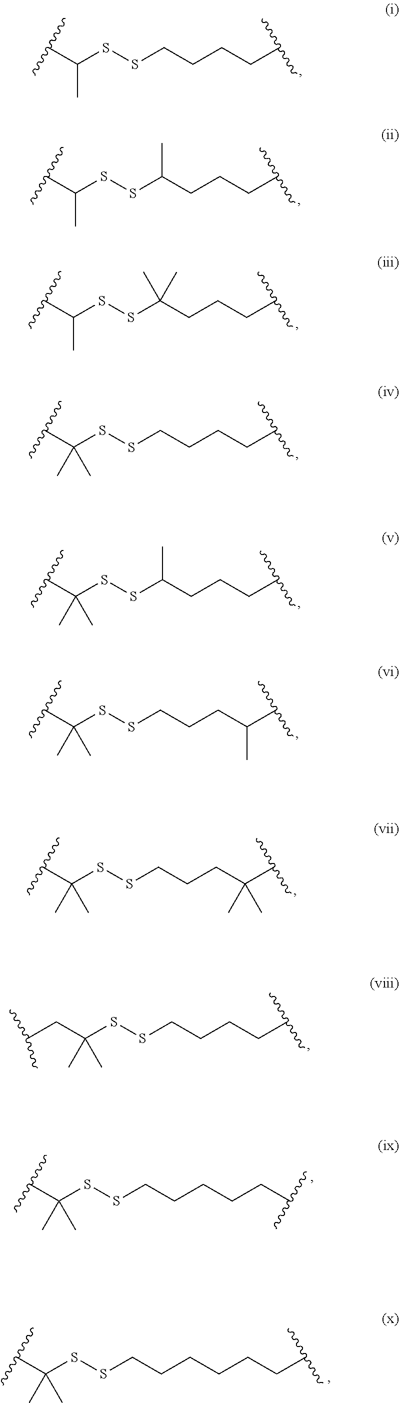

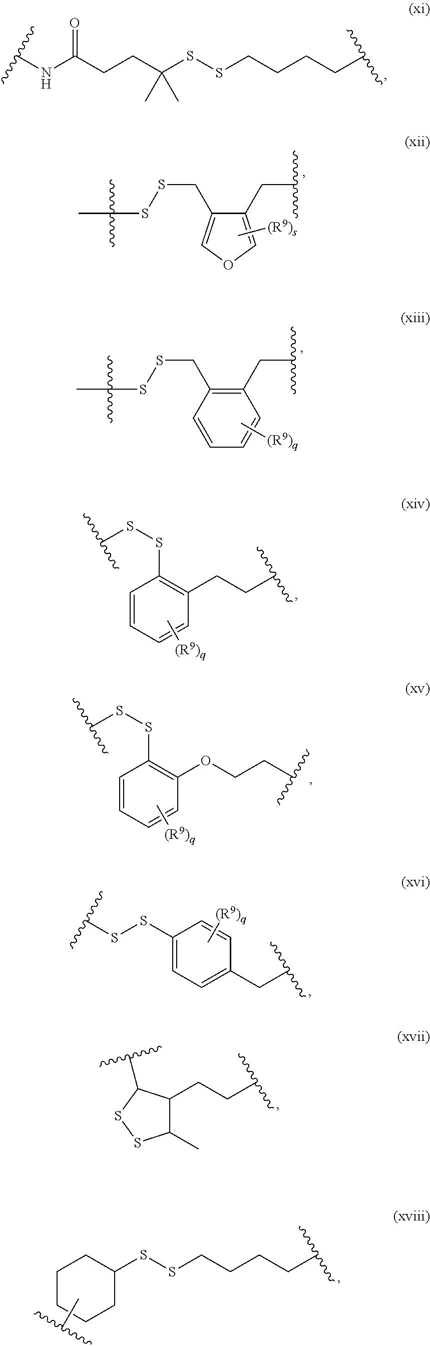

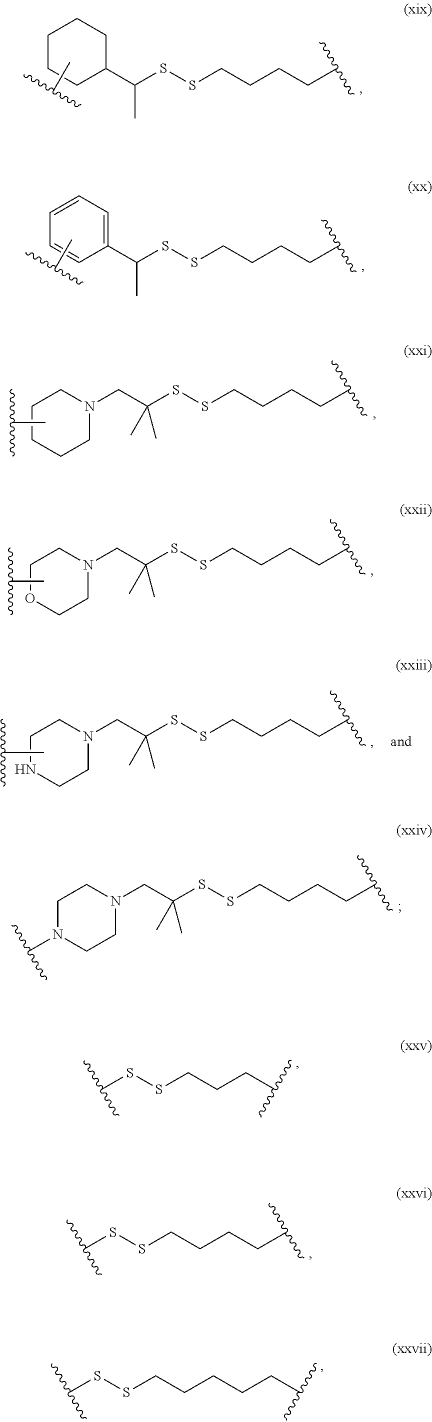

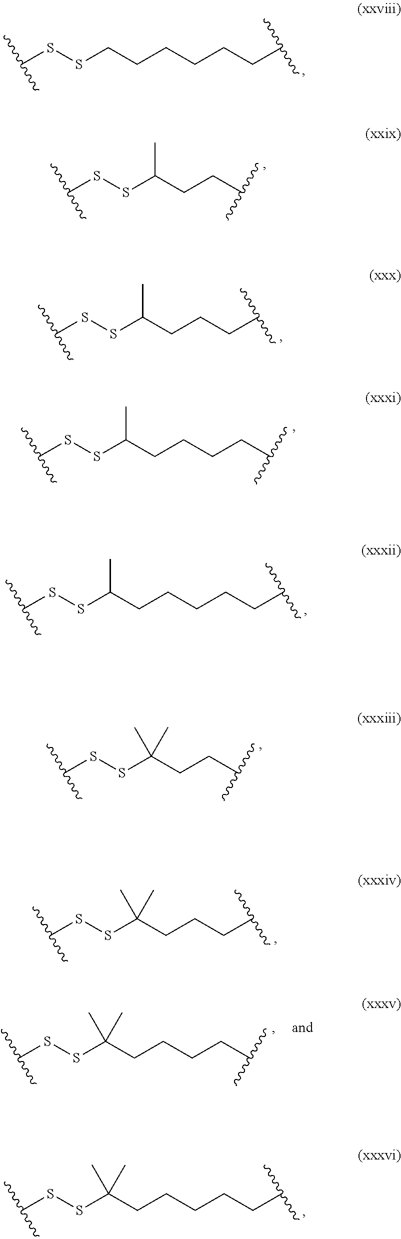

where, each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; optionally substituted amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (03-8 cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4;

s is 0, 1, or 2; and

where, when group of Formula (III) is attached at 5′ or 3′ end of the polynucleotide, A⁵ is not (i), (xviii), (xxv), (xxvi), (xxvii), or (xxviii).

In some embodiments, R⁹ is halo or optionally substituted C₁₋₆ alkyl. In particular embodiments, s is 0 or 1. In certain embodiments, s is 0. In other embodiments, q is 0, 1, or 2. In yet other embodiments, q is 0 or 1.

In certain embodiments, two adjacent R⁹ groups, together with the atoms to which each said R⁹ is attached, combine to form C₂₋₅ heteroaryl optionally substituted with 1, 2, or 3 C₁₋₆ alkyl groups.

In certain embodiments, A², A³, A⁴, and —S—S— combine to form a structure:

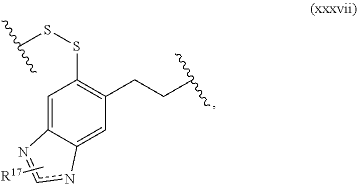

wherein the dotted lines represent one and only one double bond, and

R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In some embodiments, R¹⁷ is H or C₁₋₆ alkyl.

In particular embodiments, A⁵ has the structure of any one of:

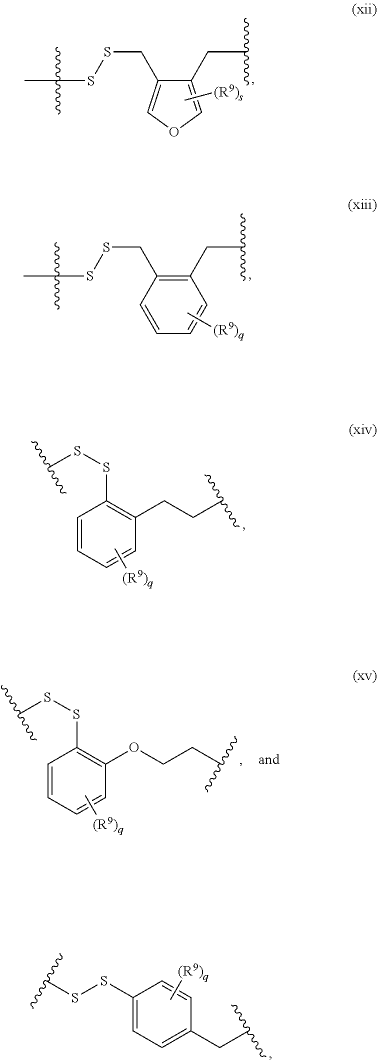

In yet another aspect, the invention provides a hybridized polynucleotide including any polynucleotide construct of the invention hybridized to a complementary polynucleotide, e.g., as siRNA.

In certain embodiments, the complementary polynucleotide contains one or more components (i), one or more groups of formula (II), or one or more groups of formula (III). In particular embodiments, no more than 75% of the total number of nucleotides have the component (i), a group of formula (II), or a group of formula (III). In some embodiments, the polynucleotide construct of the preceding aspects and the complementary nucleotide each have between 5 and 50 nucleotides. In particular embodiments, the polynucleotide construct of the preceding aspects and the complementary nucleotide each have between 10 and 32 nucleotides. In certain embodiments, the polynucleotide construct of the preceding aspects and the complementary nucleotide each have between 19 and 25 nucleotides. In other embodiments, the polynucleotide construct of the preceding aspects is the guide strand, and the complementary polynucleotide is the passenger strand. In certain embodiments, the passenger strand contains one or more phosphotriesters having a moiety that is not cleavable by an intracellular enzyme. In particular embodiments, the moiety that is not cleavable by the intracellular enzyme is optionally substituted C₁₋₆ alkyl.

In still another aspect, the invention provides a compound having a structure of Formula (VII):

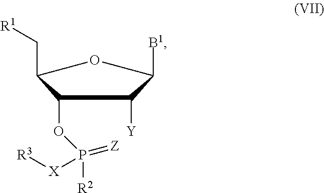

or a salt thereof,

where

B¹ is a nucleobase;

X is selected from the group consisting of O, S, and NR⁴;

Y is selected from the group consisting of hydrogen, hydroxyl, halo, optionally substituted C₁₋₆ alkoxy, and a protected hydroxyl group;

Z is absent, O, or S;

R¹ is selected from the group consisting of hydroxyl, optionally substituted C₁₋₆ alkoxy, a protected hydroxyl group, a monophosphate, a diphosphate, a triphosphate, a tetraphosphate, and a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and any combination thereof;

R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an optionally substituted amino, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and a combination thereof; and

R³ is a group having the structure of Formula (VIII):

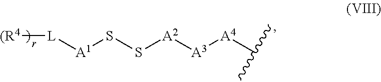

where A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is a bond or a conjugating group including or being one or more conjugating moieties;

R⁴ is absent, hydrogen, optionally substituted C₁₋₆ alkyl, a hydrophilic functional group, or a group including an auxiliary moiety selected from the group consisting of a small molecule, a polypeptide, a carbohydrate, a neutral organic polymer, a positively charged polymer, a therapeutic agent, a targeting moiety, an endosomal escape moiety, and a combination thereof;

r is an integer from 1 to 10;

where A², A³, and A⁴ combine to form a group having at least three atoms in the shortest chain connecting —S—S— and X.

In some embodiments, r is 1 to 7. In certain embodiments, each X is O. In particular embodiments, each Z is O. In other embodiments, Y is halo, optionally substituted C₁₋₆ alkoxy, or hydroxyl. In yet other embodiments, the Y is F. In still other embodiments, the Y is OMe.

In particular embodiments, R⁴ is bound to L, to A¹, or to disulfide via a bond formed by a reaction selected from the group consisting of a pericyclic reaction; an alkylation or arylation of a hydroxyl, thiol, or amino moiety; and a reaction of a hydroxyl, thiol, or amino nucleophile with an electrophile. In certain embodiments, R⁴ is bound to L, to A¹, or to disulfide via amide bond, a sulfonamide bond, a carboxylic ester, a thioester, an optionally substituted C₆₋₁₄ aryl or C₁₋₉ heteroaryl having 1 to 4 heteroatoms selected from N, O, and S; an imine; a hydrazone; an oxime; or a succinimide.

In certain embodiments, one or more of the hydrophilic functional groups and conjugating moieties are protected with protecting groups. In other embodiments, L is formed through a condensation reaction with an aldehyde conjugating moiety to form an imine, enamine or hydrazone bond. In yet other embodiments, at least one R⁴ is a targeting moiety. In still other embodiments, at least one R⁴ is a carbohydrate. In particular embodiments, at least one R⁴ is mannose. In some embodiments, at least one R⁴ is N-acetyl galactosamine. In certain embodiments, at least one R⁴ contains or is a folate ligand. In particular embodiments, at least one R⁴ contains at least one protein transduction domain. In some embodiments, at least one R⁴ is an endosomal escape moiety.

In certain embodiments, L includes or consists of 1 to 500 monomers, each independently being optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; carbonyl; thiocarbonyl; imino; optionally substituted N; O; or S. In some embodiments, L includes or consists of one or more C₁₋₆ alkyleneoxy groups. In particular embodiments, L includes or consists of less than 100 C₁₋₆ alkyleneoxy groups, e.g., ethyleneoxy. In certain embodiments, L includes or consists of less than 100 ethyleneoxy groups. In some embodiments, L includes or consists of one or more poly(alkylene oxide), e.g., polyethylene oxide, polypropylene oxide, poly(trimethylene oxide), polybutylene oxide, poly(tetramethylene oxide), and diblock or triblock co-polymers thereof.

In some embodiments, L includes or consists of one or more amino acid residues (e.g., at least one of the amino acid residues is selected from the group consisting of Arg, Asn, Asp, Cys, Glu, Gin, His, Lys, Ser, Thr, Trp, and Tyr).

In certain embodiments, L includes or is a group having the structure of formula (III):

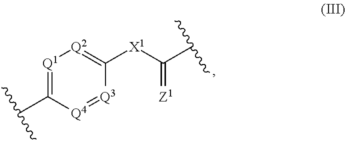

where each Q¹, Q², Q³, and Q⁴ is independently N or CR⁷;

X¹ is O or NR⁶;

Z¹ is O or S;

each R⁷ is independently selected from the group consisting of H; optionally substituted C₁₋₆ alkyl; optionally substituted C₂₋₆ alkenyl; optionally substituted C₂₋₆ alkynyl; halo; hydroxyl; —CHO; optionally substituted C₁₋₆ alkanoyl; carboxyl; cyano; nitro; optionally substituted amino; thiol; optionally substituted C₁₋₉ heterocyclyl having 1 to 4 heteroatoms selected from N, O, and S; optionally substituted C₁₋₉ heteroaryl having 1 to 4 heteroatoms selected from N, O, and S; optionally substituted C₆₋₁₄ aryl; optionally substituted C₃₋₈ cycloalkyl; and optionally substituted C₃₋₈ cycloalkenyl.

In some embodiments, Q¹ is CR⁷; Q² is CR⁷; Q³ is CR⁷; and/or Q⁴ is CR⁷. In certain embodiments, each R⁷ is independently H, optionally substituted C₁₋₆ alkyl, or halo. In particular embodiments, R⁷ is H. In some embodiments, X¹ is CR⁷. In certain embodiments, Z¹ is S.

In particular embodiments, L includes or consists of one or more groups having the structure of formula (IV):

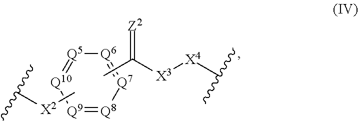

where each Q⁵, Q⁶, Q⁷, Q⁸, Q⁹, and Q¹⁰ is, independently, N, CR⁷, or C bonded to —X² or —C(Z²)X³X⁴, where no more than one of Q⁵, Q⁶, Q⁷, Q⁸, Q⁹, and Q¹⁰ is C bonded to —X², and no more than one of Q⁵, Q⁶, Q⁷, Q⁸, Q⁹, and Q¹⁰ is C bonded to —C(Z²)X³X⁴;

X² is optionally substituted C₁₋₆ cycloalkylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted diazaalkenylene; optionally substituted saturated diaza; unsaturated diaza; optionally substituted azacarbonyl; or oxacarbonyl;

X³ is a bond, O, NR⁷, or S;

X⁴ is absent, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O; optionally substituted C₁₋₉ heterocyclylene having 1 to 4 heteroatoms selected from N, O, and S; or optionally substituted (C₁₋₉ heterocyclyl)-C₁₋₄-alkylene having 1 to 4 heteroatoms selected from N, O, and S; and

Z² is O, S, or NR⁷; and

each R⁷ is independently selected from the group consisting of H, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; optionally substituted amino; and optionally substituted C₁₋₆ alkoxy; and

where the two of Q⁵, Q⁶, Q⁷, Q⁸, Q⁹, and Q¹⁰ linked to X² and —C(Z¹)X³X⁴ are not N.

In some embodiments, Q⁵ is N; Q⁶ is CR⁷; Q⁷ is C bonded to —C(Z²)X³X⁴; Q⁸ is CR⁷; Q⁹ is CR⁷; and Q¹⁰ is C bonded to X².

In other embodiments, each R⁷ is independently selected from the group consisting of H, halo, and optionally substituted C₁₋₆ alkyl. In yet other embodiments, R⁷ is H. X² is optionally substituted diazaalkenylene or optionally substituted saturated diaza. In certain other embodiments, X³ is NR⁷. In still other embodiments, X⁴ is absent. In particular embodiments, Z² is O.

In further embodiments, L includes or is one or more groups having the structure:

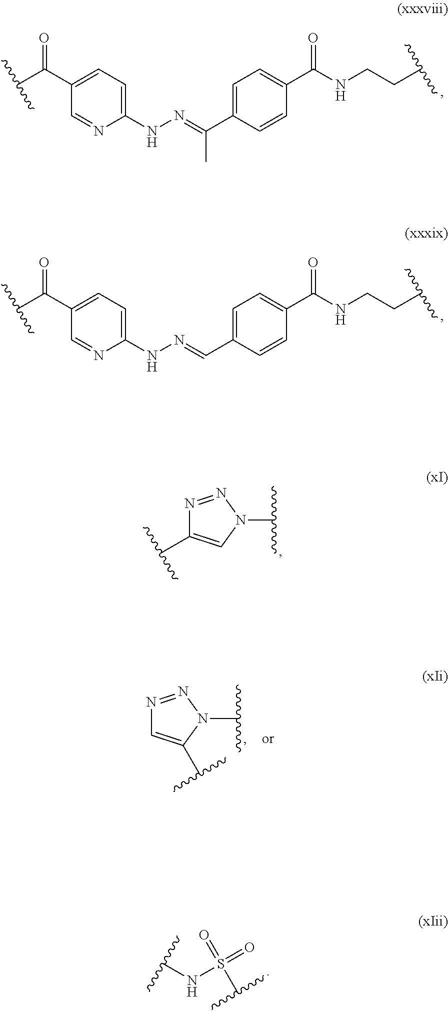 [SMILES: *CCNC(=O)c1ccc(/C(C)=N/Nc2ccc(C(*)=O)cn2)cc1 ; *CCNC(=O)c1ccc(/C=N/Nc2ccc(C(*)=O)cn2)cc1 ; *c1cn(*)nn1 ; *c1cnnn1* ; *NS(*)(=O)=O]

In particular embodiments, L is a bond.

In certain embodiments, A³ is selected from the group consisting of a bond, optionally substituted C₁₋₆ alkylene; optionally substituted C₆₋₁₄ arylene; O; optionally substituted N; and S. In some embodiments, A³ is selected from the group consisting of a bond, optionally substituted C₁₋₆ alkylene; optionally substituted C₆₋₁₄ arylene; and O.

In other embodiments, A⁴ is optionally substituted C₁₋₆ alkylene.

In yet other embodiments, A¹ includes or is a group having the structure:

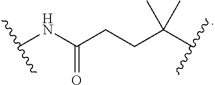 [SMILES: *NC(=O)CCC(*)(C)C]

In still embodiments, A¹ is a bond or includes or is one or more groups selected independently from the group consisting of optionally substituted C₁₋₆ alkylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; optionally substituted (C₁₋₉ heterocyclyl)-C₁₋₄-alkylene having 1 to 4 heteroatoms selected from N, O, and S; optionally substituted N; and O.

In particular embodiments, A¹ is a bond or includes or is one or more groups selected independently from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In certain embodiments, A¹ is a bond or includes or is one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In some embodiments, A¹ is a bond or includes or is one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

In certain embodiments, A¹ is a bond.

In particular embodiments, A² is optionally substituted C₁₋₆ alkylene, optionally substituted C₃₋₈ cycloalkylene; optionally substituted C₃₋₈ cycloalkenylene; optionally substituted C₆₋₁₄ arylene; or optionally substituted C₁₋₉ heteroarylene having 1 to 4 heteroatoms selected from N, O, and S.

In other embodiments, A² is optionally substituted C₁₋₆ alkylene, optionally substituted C₃₋₈ cycloalkylene; optionally substituted C₆₋₁₄ arylene; or optionally substituted C₁₋₉ heteroarylene having 1 to 4 heteroatoms selected from N, O, and S. In yet other embodiments, A² is optionally substituted optionally substituted C₆₋₁₄ arylene or optionally substituted C₁₋₉ heteroarylene having 1 to 4 heteroatoms selected from N, O, and S. In still other embodiments, A² has a structure of formula (VI):

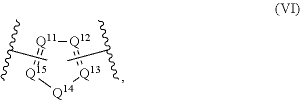

where

Q¹¹ is N, or C bonded to R¹⁰ or to the disulfide linkage;

Q¹² is N, or C bonded to R¹¹ or to A³;

Q¹³ is N or C bonded to R¹² or to A³;

Q¹⁴ is O, S, N bonded to R¹³ or to A³, or —C(R¹⁴ or bond to A³)=C(R¹⁵ or bond to A³)-;

Q¹⁵ is N, or C bonded to R¹⁶ or to the disulfide linkage;

each of R¹⁰, R¹¹, R²¹, R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and where one and only one of Q¹¹ and Q¹⁵ is bonded to the disulfide linkage, and one and only one of Q¹², Q¹³, and Q¹⁴ is bonded to A³.

In some embodiments, Q¹³ is C bonded to A³. In other embodiments, Q¹¹ is C bonded to R¹⁰. In other embodiments Q¹² is C bonded to R¹¹. In other embodiments Q¹⁴ is —C(R¹⁴)═C(R¹⁵)—. In other embodiments Q¹⁵ is bonded to the disulfide linkage.

In certain embodiments, Q¹¹ is C bonded to the disulfide linkage; Q¹² is C bonded to A³; and/or Q¹³ is C bonded to R¹². R¹² may be H, halo, or C₁₋₆ alkyl. In particular embodiments, Q¹⁴ is O. In some embodiments, Q¹⁴ is —C(R¹⁴)═C(R¹⁵)—. In other embodiments, R¹⁴ is H, halo, or C₁₋₆ alkyl. In yet other embodiments, R¹⁵ is H, halo, or C₁₋₆ alkyl. In still other embodiments, Q¹⁵ is C bonded to R¹⁶ (e.g., R¹⁶ is H, halo, or C₁₋₆ alkyl).

In other embodiments, A³ has a structure of formula (VI):

wherein

Q¹¹ is N, or C bonded to R¹⁰ or to A²;

Q¹² is N, or C bonded to R¹¹ or to A⁴;

Q¹³ is N or C bonded to R² or to A⁴;

Q¹⁴ is O, S, N bonded to R¹³ or to A⁴, or —C(R¹⁴ or bond to A⁴)=C(R¹⁵ or bond to A⁴)-;

Q¹⁵ is N, or C bonded to R¹⁶ or to A²;

each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and

wherein

one and only one of Q¹¹ and Q¹⁵ is bonded to A², and

one and only one of Q¹², Q¹³, and Q¹⁴ is bonded to A⁴.

In certain embodiments, Q¹¹ is C bonded to A². In particular embodiments, Q¹² is C bonded to A⁴. In some embodiments, Q¹³ is C bonded to R¹². In other embodiments, R¹² is H, halo, or C₁₋₆ alkyl. In certain other embodiments, R¹⁴ is O. In yet other embodiments, Q¹⁴ is —C(R¹⁴)═C(R¹⁵)—. In still other embodiments, R¹⁴ is H, halo, or C₁₋₆ alkyl. In some embodiments, R¹⁵ is H, halo, or C₁₋₆ alkyl. In particular embodiments, Q¹⁵ is C bonded to R¹⁶. In certain embodiments, R¹⁶ is H, halo, or C₁₋₆ alkyl.

In some embodiments, when the carbon atom attached to the sulfur atom of —S—S-A²-A³-A⁴- is an alkylene carbon atom, the alkylene carbon atom is connected to at most one hydrogen atom, e.g., not connected to a hydrogen atom. In yet other embodiments, when the carbon atom attached to the sulfur atom of —S—S-A²-A³-A⁴- is an alkenylene carbon atom, the alkenylene carbon atom is not connected to a hydrogen atom. In still other embodiments, the carbon atom attached to the sulfur atom of —S—S-A²-A³-A⁴- is not an alkynylene carbon atom. In certain other embodiments, when the carbon atom attached to the sulfur atom of (R⁴)ᵣ-L-A¹-S—S— is an alkylene carbon atom, the carbon atom is connected to at most one hydrogen atom, e.g., not connected to a hydrogen atom.

In particular embodiments, A¹ and A², together with —S—S— to which they are attached, join to form an optionally substituted 5 to 16 membered ring, e.g., an optionally substituted 5 to 7 membered ring.

In yet other embodiments, A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A¹ and A², together with —S—S—, join to form an optionally substituted 5 to 16 membered ring.

In some embodiments, -A¹-S—S-A²-A³-A⁴- or —S—S-A²-A³-A⁴- is:

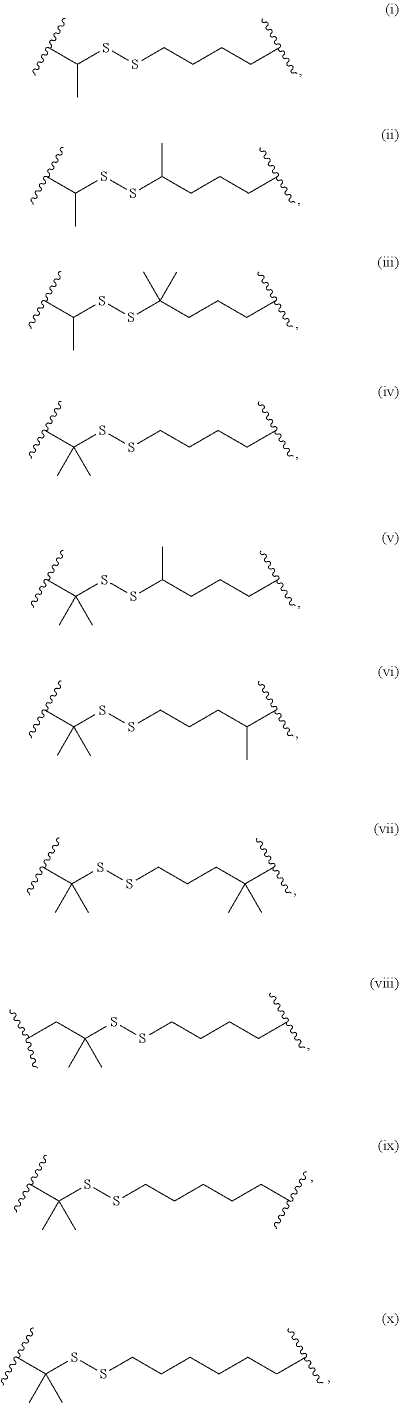

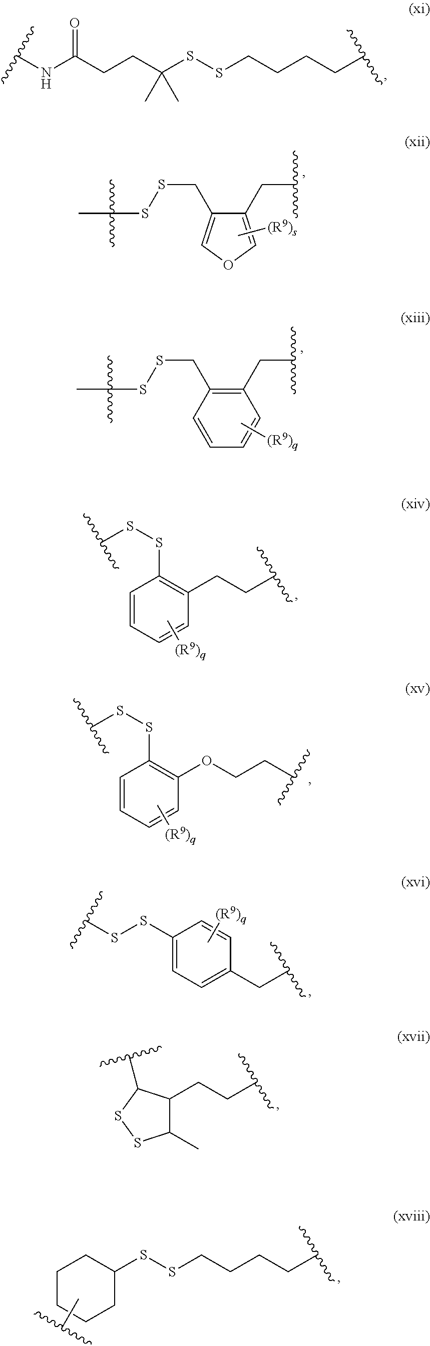

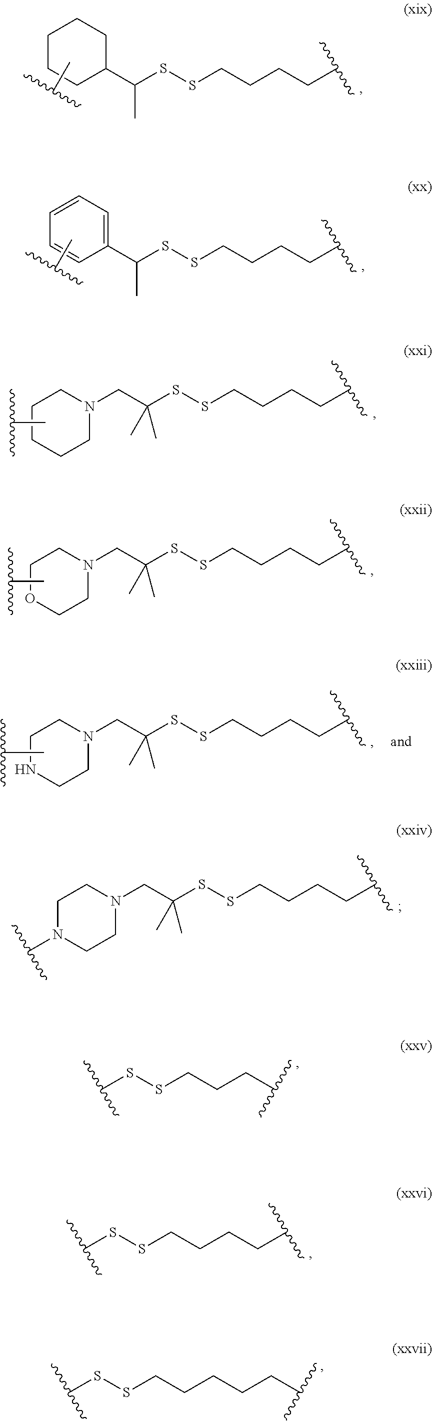

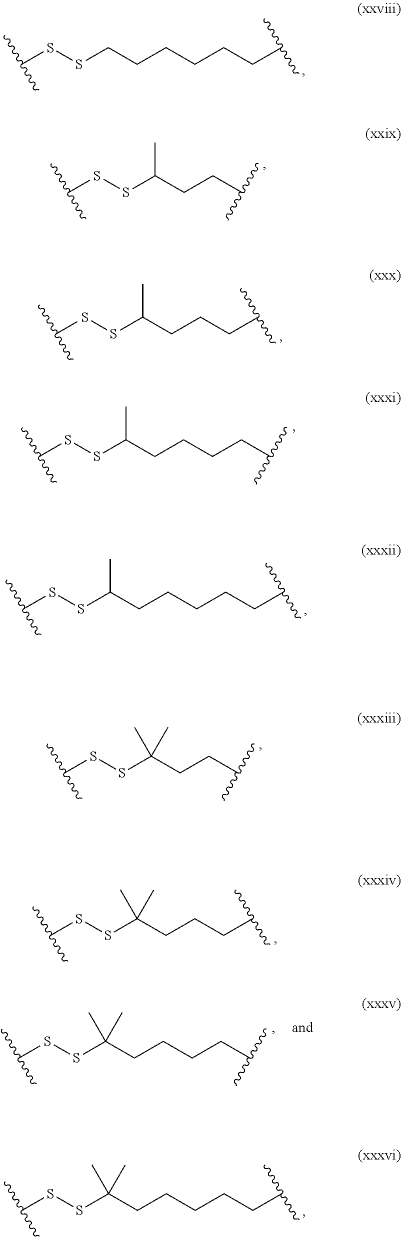

where,

each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; optionally substituted amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In certain embodiments, R⁹ is halo or optionally substituted C₁₋₆ alkyl. In other embodiments, s is 0 or 1. In yet other embodiments, s is 0. In still other embodiments, q is 0, 1, or 2. In certain other embodiments, q is 0 or 1.

In certain embodiments, two adjacent R⁹ groups, together with the atoms to which each said R⁹ is attached, combine to form C₂₋₅ heteroaryl optionally substituted with 1, 2, or 3 C₁₋₆ alkyl groups.

In certain embodiments, A2, A3, A4, and —S—S— combine to form a structure:

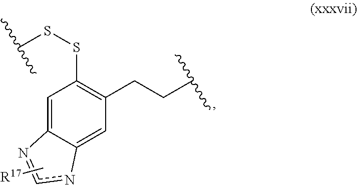

wherein the dotted lines represent one and only one double bond, and

R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In some embodiments, R¹⁷ is H or C₁₋₆ alkyl.

In particular embodiments, —S—S-A²-A³-A⁴- have the structure of any one of:

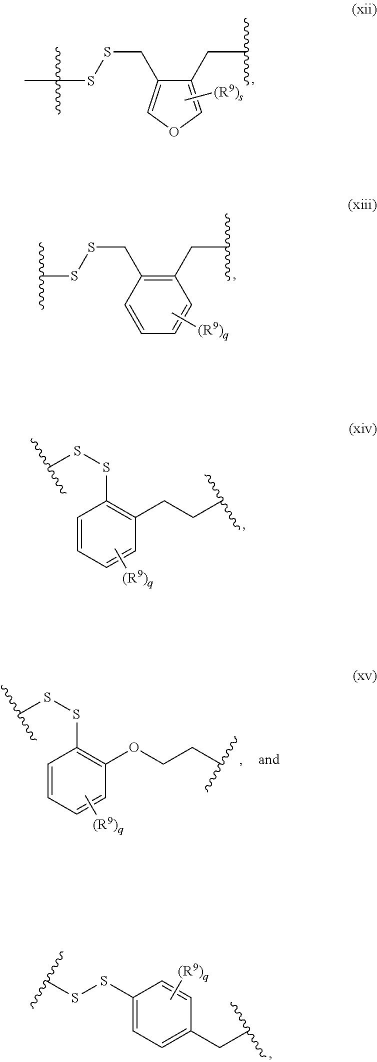

In another aspect, the invention provides a method of delivering a polynucleotide construct to a cell including contacting the cell with the polynucleotide construct of any of the embodiment the preceding aspects or the hybridized polynucleotide of any embodiment of the preceding aspects.

In another aspect, the invention provides method of delivering a polynucleotide construct to a cell. The method involves contacting the cell with the polynucleotide construct of the invention or the hybridized polynucleotide of the invention.

In certain embodiments of any aspect of the invention, none of the component (i), R⁴, L, and A¹ contains a guanidinyl group. In another aspect, for any of the above, the disulfide linkage or —S—S— groups may be replaced with a thioester or —C(O)S— or —C(S)S— group.

### DEFINITIONS

The term “about,” as used herein, represents a value that is ±10% of the recited value.

The term “activated carbonyl,” as used herein, represents a functional group having the formula of —C(O)R^{A} where R^{A} is a halogen, optionally substituted C₁₋₆ alkoxy, optionally substituted C₆₋₁₀ aryloxy, optionally substituted C₂₋₉ heteroaryloxy (e.g., —OBt), optionally substituted C₂-C₉ heterocyclyloxy (e.g., —OSu), optionally substituted pyridinium (e.g., 4-dimethylaminopyridinium), or —N(OMe)Me.

The term “activated phosphorus center,” as used herein, represents a trivalent phosphorus (III) or a pentavalent phosphorus (V) center, in which at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “activated silicon center,” as used herein, represents a tetrasubstituted silicon center, in which at least one of the substituents is a halogen, optionally substituted C₁₋₆ alkoxy, optionally substituted amino.

The term “activated sulfur center,” as used herein, represents a tetravalent sulfur wherein at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “alkanoyl,” as used herein, represents a hydrogen or an alkyl group (e.g., a haloalkyl group) that is attached to the parent molecular group through a carbonyl group and is exemplified by formyl (i.e., a carboxyaldehyde group), acetyl, propionyl, butyryl, isobutyryl, and the like. Exemplary unsubstituted alkanoyl groups include from 1 to 7 carbons. In some embodiments, the alkyl group is further substituted with 1, 2, 3, or 4 substituents as described herein.

The term “(C_{x1-y1} aryl)-C_{x2-y2}-alkyl,” as used herein, represents an aryl group of x1 to y1 carbon atoms attached to the parent molecular group through an alkylene group of x2 to y2 carbon atoms. Exemplary unsubstituted (C_{x1-y1} aryl)-C_{x2-y2}-alkyl groups are from 7 to 16 carbons. In some embodiments, the alkylene and the aryl each can be further substituted with 1, 2, 3, or 4 substituent groups as defined herein for the respective groups. Other groups followed by “alkylene” are defined in the same manner, where “alkylene” refers to a C₁₋₆ alkylene, unless otherwise noted, and the attached chemical structure is as defined herein.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted with 1, 2, 3, or 4 substituent groups selected, independently, from the group consisting of aryl, cycloalkyl, heterocyclyl (e.g., heteroaryl), as defined herein, and the substituent groups described for alkyl. In addition, when an alkenyl group is present in a bioreversible group of the invention it may be substituted with a thioester or disulfide group that is bound to a conjugating moiety, a hydrophilic functional group, or an auxiliary moiety as defined herein.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. The alkenylene group may be unsubstituted or substituted (e.g., optionally substituted alkenylene) as described for alkenyl groups.

The term “alkoxy,” as used herein, represents a chemical substituent of formula —OR, where R is a C₁₋₆ alkyl group, unless otherwise specified. In some embodiments, the alkyl group can be further substituted with 1, 2, 3, or 4 substituent groups as defined herein.

The term “alkyl,” as used herein, refers to an acyclic straight or branched chain saturated hydrocarbon group having from 1 to 12 carbons, unless otherwise specified. Alkyl groups are exemplified by methyl; ethyl; n- and iso-propyl; n-, sec-, iso- and tert-butyl; neopentyl, and the like, and may be optionally substituted with one, two, three, or, in the case of alkyl groups of two carbons or more, four substituents independently selected from the group consisting of: (1) alkoxy; (2) alkylsulfinyl; (3) amino; (4) arylalkoxy; (5) (arylalkyl)aza; (6) azido; (7) halo; (8) (heterocyclyl)oxy; (9) (heterocyclyl)aza; (10) hydroxy; (11) nitro; (12) oxo; (13) aryloxy; (14) sulfide; (15) thioalkoxy; (16) thiol; (17) —CO₂R^{A}, where R^{A} is selected from the group consisting of (a) alkyl, (b) aryl, (c) hydrogen, and (d) arylalkyl; (18) —C(O)NR^{B}R^{C}, where each of R^{B} and R^{C} is, independently, selected from the group consisting of (a) hydrogen, (b) alkyl, (c) aryl, and (d) aryl-alkylene; (19) —SO₂R^{D}, where R^{D} is selected from the group consisting of (a) alkyl, (b) aryl, and (c) aryl-alkylene; (20) —SO₂NR^{E}R^{F}, where each of R^{E} and R^{F} is, independently, selected from the group consisting of (a) hydrogen, (b) alkyl, (c) aryl and (d) arylalkyl; (21) silyl; (22) cyano; and (23) —S(O)R^{H} where R^{H} is selected from the group consisting of (a) hydrogen, (b) alkyl, (c) aryl, and (d) arylalkyl. In some embodiments, each of these groups can be further substituted as described herein. In certain embodiments, the alkyl carbon atom bonding to the parent molecular group is not oxo-substituted.

The term “alkylene,” as used herein, refers to a saturated divalent, trivalent, or tetravalent hydrocarbon group derived from a straight or branched chain saturated hydrocarbon by the removal of at least two hydrogen atoms. Alkylene can be trivalent only if bonded to one aza group that is not an optional substituent; alkylene can be trivalent or tetravalent only if bonded to two aza groups that are not optional substituents. The valency of alkylene defined herein does not include the optional substituents. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. The term “C_{x-y} alkylene” represents alkylene groups having between x and y carbons. Exemplary values for x are 1, 2, 3, 4, 5, and 6, and exemplary values for y are 2, 3, 4, 5, 6, 7, 8, 9, 10, 11, and 12. In some embodiments, the alkylene can be further substituted with 1, 2, 3, or 4 substituent groups as defined herein for an alkyl group. Similarly, the suffix “ene” designates a divalent radical of the corresponding monovalent radical as defined herein. For example, alkenylene, alkynylene, arylene, aryl alkylene, cycloalkylene, cycloalkyl alkylene, cycloalkenylene, heteroarylene, heteroaryl alkylene, heterocyclylene, and heterocyclyl alkylene are divalent forms of alkenyl, alkynyl, aryl, aryl alkyl, cycloalkyl, cycloalkyl alkyl cycloalkenyl, heteroaryl, heteroaryl alkyl, heterocyclyl, and heterocyclyl alkyl. For aryl alkylene, cycloalkyl alkylene, heteroaryl alkylene, and heterocyclyl alkylene, the two valences in the group may be located in the acyclic portion only or one in the cyclic portion and one in the acyclic portion. In addition, when an alkyl or alkylene, alkenyl or alkenylene, or alkynyl or alkynylene group is present in a group bonded to an internucleotide bridging group or to a terminal phosphorus-containing moiety bonded to a nucleoside, it may be substituted with an ester, thioester, or disulfide group that is bound to a conjugating moiety, a hydrophilic functional group, or an auxiliary moiety as defined herein. For example, the alkylene group of an aryl-C₁-alkylene or a heterocyclyl-C₁-alkylene can be further substituted with an oxo group to afford the respective aryloyl and (heterocyclyl)oyl substituent group.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. Alkynyl groups may be optionally substituted with 1, 2, 3, or 4 substituent groups that are selected, independently, from aryl, alkenyl, cycloalkyl, heterocyclyl (e.g., heteroaryl), as defined herein, and the substituent groups described for alkyl.

The term “alkynylene,” as used herein, refers to a straight-chain or branched-chain divalent substituent including one or two carbon-carbon triple bonds and containing only C and H when unsubstituted. Non-limiting examples of the alkenylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. The alkynylene group may be unsubstituted or substituted (e.g., optionally substituted alkynylene) as described for alkynyl groups.

The term “amino,” as used herein, represents —N(R^{N1})₂ or —N(R^{N1})C(NR^{N1})N(R^{N1})₂ wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, aryl-alkyl, cycloalkyl, cycloalkylalkyl, heterocyclyl (e.g., heteroaryl), heterocyclylalkyl (e.g., heteroarylalkyl), or two R^{N1} combine to form a heterocyclyl, and wherein each R^{N2} is, independently, H, alkyl, or aryl. In one embodiment, amino is —NH₂, or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl. Each R^{N1} group may be independently unsubstituted or substituted as described herein. In addition, when an amino group is present in a bioreversible group of the invention it may be substituted with an ester, thioester, or disulfide group that is bound to a conjugating moiety, a hydrophilic functional group, or an auxiliary moiety as defined herein.

The term “antibody,” as used herein, is used in the broadest sense and specifically covers, for example, single monoclonal antibodies, antibody compositions with polyepitopic specificity, single chain antibodies, and fragments of antibodies (e.g., antigen binding fragment or Fc region). “Antibody” as used herein includes intact immunoglobulin or antibody molecules, polyclonal antibodies, multispecific antibodies (i.e., bispecific antibodies formed from at least two intact antibodies) and immunoglobulin fragments (such as Fab, F(ab′)₂, or Fv), so long as they recognize antigens and/or exhibit any of the desired agonistic or antagonistic properties described herein. Antibodies or fragments may be humanized, human, or chimeric.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four, or five substituents independently selected from the group consisting of: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, or thioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)_{q}CO₂R^{A}, where q is an integer from zero to four, and R^{A} is selected from the group consisting of (a) alkyl, (b) aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b) alkyl, (c) aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein. In addition, when an aryl group is present in a bioreversible group of the invention it may be substituted with an ester, thioester, or disulfide group that is bound to a conjugating moiety, a hydrophilic functional group, or an auxiliary moiety as defined herein.

The term “aryl alkyl,” as used herein, represents an alkyl group substituted with an aryl group. The aryl and alkyl portions may be substituted as the individual groups as described herein.

The term “auxiliary moiety” refers to any moiety, including, but not limited to, a small molecule, a polypeptide, a carbohydrate, a neutral organic polymer, a positively charged polymer, a therapeutic agent, a targeting moiety, an endosomal escape moiety, and any combination thereof, which can be conjugated to a nucleotide construct disclosed herein. Generally, but not always the case, an “auxiliary moiety” is linked or attached to a nucleotide construct disclosed herein by forming one or more covalent bonds to one or more conjugating groups present on a bioreversible group. However, in alternative embodiments an “auxiliary moiety” may be linked or attached to a nucleotide construct disclosed herein by forming one or more covalent bonds to any portion of the nucleotide construct in addition to conjugating groups present on a bioreversible group, such as to the 2′, 3′, or 5′ positions of a nucleotide sugar molecule, or on any portion of a nucleobase. Although the name for a particular auxiliary moiety may imply a free molecule, it will be understood that such a free molecule is attached to a nucleotide construct. One skilled in the art will readily understand appropriate points of attachment of a particular auxiliary moiety to a nucleotide construct.

The term “aza,” as used herein, represents a divalent —N(R^{N1})— group or a trivalent —N═ group. The aza group may be unsubstituted, where R^{N1} is H or absent, or substituted, where R^{N1} is as defined for “amino.” Aza may also be referred to as “N,” e.g., “optionally substituted N.” Two aza groups may be connected to form “diaza.”

The term “azido,” as used herein, represents an N₃ group.

The term “bioreversible group,” as used herein, represents a moiety comprising a functional group that can be actively cleaved intracellularly, e.g., via the action of one or more intracellular enzymes (e.g., an intracellular reductase) or passively cleaved intracellularly, such as by exposing the group to the intracellular environment or a condition present in the cell (e.g., pH, reductive or oxidative environment, or reaction with intracellular species, such as glutathione). Exemplary bioreversible groups comprise disulfides.

The term “bulky group,” as used herein, represents any substituent or a group of substituents as defined herein, in which the radical of the bulky group bears one hydrogen atom or fewer if the radical is sp³-hybridized carbon, bears no hydrogen atoms if the radical is sp²-hybridized carbon. The radical is not sp-hybridized carbon. The bulky group bonds to another group only through a carbon atom. For example, the statements “bulky group bonded to the disulfide linkage,” “bulky group attached to the disulfide linkage,” and “bulky group linked to the disulfide linkage” indicate that the bulky group is bonded to the disulfide linkage through a carbon radical.

The term “carbene” as used herein, represents a functional group that is a divalent carbon species having six valence electrons and the structure ═C: or —C(R^{B}): wherein R^{B} is selected from H, optionally substituted C₁₋₁₂ alkyl, optionally substituted C₆₋₁₄ aryl, optionally substituted (C₆₋₁₄ aryl)-C₁₋₁₂-alkylene, or optionally substituted carbonyl; and C is a carbon with two electrons that are not part of a covalent bond. The two electrons may be paired (e.g., singlet carbene) or unpaired (e.g., triplet carbene).

The term “carbocyclic,” as used herein, represents an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbohydrate,” as used herein, represents a compound which comprises one or more monosaccharide units having at least 5 carbon atoms (which may be linear, branched or cyclic) with an oxygen, nitrogen or sulfur atom bonded to each carbon atom. The term “carbohydrate” therefore encompasses monosaccharides, disaccharides, trisaccharides, tetrasaccharides, oligosaccharides, and polysaccharides. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅₋₆ sugars; di- and trisaccharides include sugars having two or three monosaccharide units (e.g., C₅₋₆ sugars).

The term “carbonyl,” as used herein, represents a C(O) group. Examples of functional groups which comprise a “carbonyl” include esters, ketones, aldehydes, anhydrides, acyl chlorides, amides, carboxylic acids, and carboxylates.

The term “component of a coupling reaction,” as used herein, represents a molecular species capable of participating in a coupling reaction. Components of coupling reactions include hydridosilanes, alkenes, and alkynes.

The term “component of a cycloaddition reaction,” as used herein, represents a molecular species capable of participating in a cycloaddition reaction. In cycloaddition reactions in which bond formation involves [4n+2] π electrons where n is 1, one component will provide 2π electrons, and another component will provide 4 π electrons. Representative components of cycloaddition reactions that provide 2π electrons include alkenes and alkynes. Representative components of cycloaddition reactions that provide 4π electrons include 1,3-dienes, α, β-unsaturated carbonyls, and azides.

The term “conjugating group,” as used herein, represents a divalent or higher valency group containing one or more conjugating moieties. The conjugating group links one or more auxiliary moieties to a bioreversible group (e.g., a group containing a disulfide moiety).

The term “conjugating moiety,” as used herein, represents a functional group that is capable of forming one or more covalent bonds to another group (e.g., a functional group that is a nucleophile, electrophile, a component in a cycloaddition reaction, or a component in a coupling reaction) under appropriate conditions. The term also refers to the residue of a conjugation reaction, e.g., amide group. Examples of such groups are provided herein.

The term “coupling reaction,” as used herein, represents a reaction of two components in which one component includes a nonpolar σ bond such as Si—H or C—H and the second component includes a π bond such as an alkene or an alkyne that results in either the net addition of the a bond across the π bond to form C—H, Si—C, or C—C bonds or the formation of a single covalent bond between the two components. One coupling reaction is the addition of Si—H across an alkene (also known as hydrosilylation). Other coupling reactions include Stille coupling, Suzuki coupling, Sonogashira coupling, Hiyama coupling, and the Heck reaction. Catalysts may be used to promote the coupling reaction. Typical catalysts are those which include Fe(II), Cu(I), Ni(0), Ni(II), Pd(0), Pd(II), Pd(IV), Pt(0), Pt(II), or Pt(IV).

The term “cycloaddition reaction” as used herein, represents reaction of two components in which [4n+2] π electrons are involved in bond formation when there is either no activation, activation by a chemical catalyst, or activation using thermal energy, and n is 1, 2, or 3. A cycloaddition reaction is also a reaction of two components in which [4n]π electrons are involved, there is photochemical activation, and n is 1, 2, or 3. Desirably, [4n+2] π electrons are involved in bond formation, and n=1. Representative cycloaddition reactions include the reaction of an alkene with a 1,3-diene (Diels-Alder reaction), the reaction of an alkene with an α,β-unsaturated carbonyl (hetero Diels-Alder reaction), and the reaction of an alkyne with an azide (Hüisgen cycloaddition).

The term “cycloalkenyl,” as used herein, refers to a non-aromatic carbocyclic group having from three to ten carbons (e.g., a C₃-C₁₀ cycloalkylene), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenylene,” as used herein, refers to a divalent carbocyclic non-aromatic group having from three to ten carbons (e.g., C₃-C₁₀ cycloalkenylene), unless otherwise specified. Non-limiting examples of the cycloalkenylene include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl; cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as described for cycloalkyl.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted as defined herein (e.g., optionally substituted cycloalkyl). The cycloalkyl groups of this disclosure can be optionally substituted with: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, or thioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)_{q}CO₂R^{A}, where q is an integer from zero to four, and R^{A} is selected from the group consisting of (a) alkyl, (b) aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b) alkyl, (c) aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein.

The term “cycloalkyl alkyl,” as used herein, represents an alkyl group substituted with a cycloalkyl group. The cycloalkyl and alkyl portions may be substituted as the individual groups as described herein.

The term “electrophile” or “electrophilic group,” as used herein, represents a functional group that is attracted to electron rich centers and is capable of accepting pairs of electrons from one or more nucleophiles so as to form one or more covalent bonds. Electrophiles include, but are not limited to, cations; polarized neutral molecules; nitrenes; nitrene precursors such as azides; carbenes; carbene precursors; activated silicon centers; activated carbonyls; alkyl halides; alkyl pseudohalides; epoxides; electron-deficient aryls; activated phosphorus centers; and activated sulfur centers. Typically encountered electrophiles include cations such as H⁺ and NO⁺, polarized neutral molecules, such as HCl, alkyl halides, acyl halides, carbonyl containing compounds, such as aldehydes, and atoms which are connected to good leaving groups, such as mesylates, triflates, and tosylates.

The term “endosomal escape moiety,” as used herein, represents a moiety which enhances the release of endosomal contents or allows for the escape of a molecule from an internal cellular compartment such as an endosome.

The term “halo,” as used herein, represents a halogen selected from bromine, chlorine, iodine, and fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls. In some embodiments, the haloalkyl group can be further substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. In one embodiment, the heteroaryl is substituted with 1, 2, 3, or 4 substituents groups as defined for a heterocyclyl group.

The term “heteroaryl alkyl,” as used herein, represents an alkyl group substituted with a heteroaryl group. The heteroaryl and alkyl portions may be substituted as the individual groups as described herein.

The term “heterocyclyl,” as used herein, represents a 5-, 6- or 7-membered ring, unless otherwise specified, containing one, two, three, or four heteroatoms independently selected from the group comprising nitrogen, oxygen, and sulfur. The 5-membered ring has zero to two double bonds, and the 6- and 7-membered rings have zero to three double bonds. Certain heterocyclyl groups include from 2 to 9 carbon atoms. Other such groups may include up to 12 carbon atoms. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, purinyl, thiadiazolyl (e.g., 1,3,4-thiadiazole), tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Heterocyclic groups also include groups of the formula

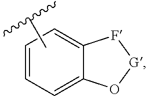

where

F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocyclyl groups mentioned herein may be optionally substituted with one, two, three, four or five substituents independently selected from the group consisting of: (1) alkanoyl (e.g., formyl, acetyl, and the like); (2) alkyl (e.g., alkoxyalkylene, alkylsulfinylalkylene, aminoalkylene, azidoalkylene, acylalkylene, haloalkylene (e.g., perfluoroalkyl), hydroxyalkylene, nitroalkylene, or thioalkoxyalkylene); (3) alkenyl; (4) alkynyl; (5) alkoxy (e.g., perfluoroalkoxy); (6) alkylsulfinyl; (7) aryl; (8) amino; (9) aryl-alkylene; (10) azido; (11) cycloalkyl; (12) cycloalkyl-alkylene; (13) cycloalkenyl; (14) cycloalkenyl-alkylene; (15) halo; (16) heterocyclyl (e.g., heteroaryl); (17) (heterocyclyl)oxy; (18) (heterocyclyl)aza; (19) hydroxy; (20) oxo; (21) nitro; (22) sulfide; (23) thioalkoxy; (24) —(CH₂)_{q}CO₂R^{A}, where q is an integer from zero to four, and R^{A} is selected from the group consisting of (a) alkyl, (b) aryl, (c) hydrogen, and (d) aryl-alkylene; (25) —(CH₂)_{q}CONR^{B}R^{C}, where q is an integer from zero to four and where R^{B} and R^{C} are independently selected from the group consisting of (a) hydrogen, (b) alkyl, (c) aryl, and (d) aryl-alkylene; (26) —(CH₂)_{q}SO₂R^{D}, where q is an integer from zero to four and where R^{D} is selected from the group consisting of (a) alkyl, (b) aryl, and (c) aryl-alkylene; (27) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) aryl-alkylene; (28) thiol; (29) aryloxy; (30) cycloalkoxy; (31) arylalkoxy; (31) heterocyclyl-alkylene (e.g., heteroaryl-alkylene); (32) silyl; (33) cyano; and (34) —S(O)R^{H} where R^{H} is selected from the group consisting of (a) hydrogen, (b) alkyl, (c) aryl, and (d) aryl-alkylene. In some embodiments, each of these groups can be further substituted as described herein. For example, the alkylene group of an aryl-C₁-alkylene or a heterocyclyl-C₁-alkylene can be further substituted with an oxo group to afford the respective aryloyl and (heterocyclyl)oyl substituent group. In addition, when a heterocyclyl group is present in a bioreversible group of the invention it may be substituted with an ester, thioester, or disulfide group that is bound to a conjugating moiety, a hydrophilic functional group, or an auxiliary moiety as defined herein.

The term “heterocyclyl alkyl,” as used herein, represents an alkyl group substituted with a heterocyclyl group. The heterocyclyl and alkyl portions may be substituted as the individual groups as described herein.

The term “hydrophilic functional group,” as used herein, represents a moiety that confers an affinity to water and increases the solubility of an alkyl moiety in water. Hydrophilic functional groups can be ionic or non-ionic and include moieties that are positively charged, negatively charged, and/or can engage in hydrogen-bonding interactions. Exemplary hydrophilic functional groups include hydroxy, amino, carboxyl, carbonyl, thiol, phosphates (e.g., a mono-, di-, or tri-phosphate), polyalkylene oxides (e.g., polyethylene glycols), and heterocyclyls.

The terms “hydroxyl” and “hydroxy,” as used interchangeably herein, represent an —OH group.

The term “imine,” as used herein, represents a group having a double bond between carbon and nitrogen, which can be represented as “C═N.” In a particular embodiment, where a proton is a to the imine functional group, the imine may also be in the form of the tautomeric enamine. A type of imine bond is the hydrazone bond, where the nitrogen of the imine bond is covalently attached to a trivalent nitrogen (e.g., C═N—N(R)₂). In some embodiments, each R can be, independently, H, OH, optionally substituted C₁₋₆ alkoxy, or optionally substituted C₁₋₆ alkyl.

The term “nitrene,” as used herein, represents a monovalent nitrogen species having six valence electrons and the structure ═N: or —NR^{A}: where R^{A} is selected from optionally substituted C₁₋₁₂ alkyl, optionally substituted C₆₋₁₂ aryl, optionally substituted (C₆₋₁₂ aryl)-C₁₋₁₂-alkylene, or optionally substituted carbonyl; and N is a nitrogen with four valence electrons, at least two of which are paired. The two remaining electrons may be paired (i.e., singlet nitrene) or unpaired (i.e., triplet nitrene).

The term “nitro,” as used herein, represents an —NO₂ group.

A “non-naturally occurring amino acid” is an amino acid not naturally produced or found in a mammal.

By “nonpolar σ bond” is meant a covalent bond between two elements having electronegativity values, as measured according to the Pauling scale, that differ by less than or equal to 1.0 units. Non-limiting examples of nonpolar 6 bonds include C—C, C—H, Si—H, Si—C, C—Cl, C—Br, C—I, C—B, and C—Sn bonds.

The term “nucleobase,” as used herein, represents a nitrogen-containing heterocyclic ring found at the 1′ position of the sugar moiety of a nucleotide or nucleoside. Nucleobases can be unmodified or modified. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethylcytosine, 5-formylcytosine, 5-carboxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289 302, (Crooke et al., ed., CRC Press, 1993). Certain nucleobases are particularly useful for increasing the binding affinity of the polymeric compounds of the invention, including 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications 1993, CRC Press, Boca Raton, pages 276-278). These may be combined, in particular embodiments, with 2′-O-methoxyethyl sugar modifications. United States patents that teach the preparation of certain of these modified nucleobases as well as other modified nucleobases include, but are not limited to, the above noted U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; and 5,681,941. For the purposes of this disclosure, “modified nucleobases,” as used herein, further represents nucleobases, natural or nonnatural, which comprise one or more protecting groups as described herein.

The terms “nucleophile,” as used herein, represent an optionally substituted functional group that engages in the formation of a covalent bond by donating electrons from electron pairs or π bonds. Nucleophiles may be selected from alkenes, alkynes, aryl, heteroaryl, hydrazine groups, hydroxy groups, phenoxy groups, amino groups, alkylamino groups, anilido groups, thio groups, and thiophenoxy groups.

The term “nucleoside,” as used herein, represents a nucleobase-sugar combination. The term “nucleotide,” as used herein, refers to a nucleoside that further includes an internucleotide bridging group or a terminal nucleotide group, such as a phosphate group, covalently linked to the sugar portion of the nucleoside. For those nucleosides that include a pentafuranosyl sugar, the internucleotide bridging group or terminal group, e.g., phosphate group, can be linked to either the 2′, 3′ or 5′ hydroxyl moiety of the sugar. The sugar may or may not be a naturally occurring sugar, e.g., ribose or deoxyribose, and it may be a modified form of a naturally occurring sugar, e.g., 2′ modified ribose. Exemplary modified sugars include 2-position sugar modifications, in which the 2-OH is replaced by a group such as an H, OR, R, halo (e.g., F), SH, SR, NH₂, NHR, NR₂, or CN, wherein R is an alkyl moiety. Modified sugars also include, e.g., non-ribose sugars, such as mannose, arabinose, glucopyranose, galactopyranose, 4-thioribose, and other sugars, heterocycles, or carbocycles. Nucleotides also include locked nucleic acids (LNA), peptide nucleic acids, glycerol nucleic acids, morpholino nucleic acids, and threose nucleic acids.

The term “polynucleotide” as used herein, represents two or more nucleotides and/or nucleosides covalently bound together by an internucleotide bridging group. Polynucleotides may be linear or circular. Moreover, for the purposes of this disclosure, the term “polynucleotide” is in reference to both oligonucleotides and longer sequences, and to mixtures of nucleotides, e.g., mixtures of DNA and RNA or mixtures of RNA and 2′ modified RNA. The term “polynucleotide” encompasses polynucleotides that comprise one or more strands, unless stated otherwise.

In other embodiments, the natural sugar phosphorodiester backbone can be replaced with a protein nucleotide (PNA) backbone having repeating N-(2-aminoethyl)-glycine units linked by peptide bonds. Other types of modifications for polynucleotides designed to be more resistant to nuclease degradation are described U.S. Pat. Nos. 6,900,540 and 6,900,301, incorporated herein by reference.

The term “internucleotide bridging group,” as used herein, represents a group which covalently links nucleotides and/or nucleosides together. A “terminal nucleotide” group is located at the 5′, 3′, or 2′ end of a nucleotide. A terminal nucleotide group may or may not be capable of being connected to other nucleosides or nucleotides. Exemplary internucleotide bridging groups and terminal nucleotide groups include phosphate, thiophosphate, phosphonate (e.g., methyl phosphonate), phosphoramidate, boranophosphate, amide, methylene methylimino, formacetal, thioformacetal, sulfonyl, guanidine, and methylthiourea. Others are known in the art, see, e.g., Current Medicinal Chemistry, 2001, Vol. 8, No. 10, 1157. It will be understood that an internucleotide bridging group is bound to two nucleosides, and a terminal nucleotide group is bound to a single nucleoside, e.g., at the 3′ or 5′ end.

The terms “oxa” and “oxy,” as used interchangeably herein, represents a divalent oxygen atom that is connected to two groups (e.g., the structure of oxy may be shown as —O—).

The term “oxo,” as used herein, represents a divalent oxygen atom that is connected to one group (e.g., the structure of oxo may be shown as ═O).

The term “polypeptide,” as used herein, represents two or more amino acid residues linked by peptide bonds. Moreover, for purposes of this disclosure, the term “polypeptide” and the term “protein” are used interchangeably herein in all contexts, unless provided for otherwise, e.g., naturally occurring or engineered proteins. A variety of polypeptides may be used within the scope of the methods and compositions provided herein. In a certain embodiment, polypeptides include antibodies or fragments of antibodies containing an antigen-binding site. Polypeptides made synthetically may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine.

The term “Ph,” as used herein, represents phenyl.

The terms “photolytic activation” or “photolysis,” as used herein, represent the promotion or initiation of a chemical reaction by irradiation of the reaction with light. The wavelengths of light suitable for photolytic activation range between 200-500 nm and include wavelengths that range from 200-260 nm and 300-460 nm. Other useful ranges include 200-230 nm, 200-250 nm, 200-275 nm, 200-300 nm, 200-330 nm, 200-350 nm, 200-375 nm, 200-400 nm, 200-430 nm, 200-450 nm, 200-475 nm, 300-330 nm, 300-350 nm, 300-375 nm, 300-400 nm, 300-430 nm, 300-450 nm, 300-475 nm, and 300-500 nm.

The term “protecting group,” as used herein, represents a group intended to protect a functional group (e.g., a hydroxyl, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkylene ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkylene groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “sterically hindered,” as used herein, describes a chemical group having half-life of at least 24 hours in the presence of an intermolecular or an intramolecular nucleophile or electrophile.

The term “subject,” as used herein, represents a human or non-human animal (e.g., a mammal).

The term “sulfide” as used herein, represents a divalent —S— or ═S group.

The term “targeting moiety,” as used herein, represents any moiety that specifically binds or reactively associates or complexes with a receptor or other receptive moiety associated with a given target cell population.

The term “therapeutically effective dose,” as used herein, represents the quantity of an siRNA, or polynucleotide according to the invention necessary to ameliorate, treat, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in vivo administration of the pharmaceutical composition, and animal models may be used to determine effective dosages for treatment of particular disorders.

The term “thiocarbonyl,” as used herein, represents a C(S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thiol,” as used herein, represents an —SH group.

The term “disorder,” as used herein, is intended to be generally synonymous, and is used interchangeably with, the terms “disease,” “syndrome,” and “condition” (as in a medical condition), in that all reflect an abnormal condition presented by a subject, or one of its parts, that impairs normal functioning, and is typically manifested by distinguishing signs and symptoms.

The term “treating” as used in reference to a disorder in a subject, is intended to refer to reducing at least one symptom of the disorder by administrating a therapeutic (e.g., a nucleotide construct of the invention) to the subject.

As used herein and in the appended claims, the singular forms “a,” “and,” and “the” include plural referents unless the context clearly dictates otherwise. Thus, for example, reference to “a targeting moiety” includes a plurality of such targeting moieties, and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. The publications cited within this disclosure are incorporated in-full for all that they disclose. But, for purposes of this disclosure, any term which is presented in the publications or in the art which is identical to any term expressly defined in this disclosure, the term's definition presented in this disclosure will control in all respects.

### BRIEF DESCRIPTION OF THE DRAWINGS

The application file contains at least one drawing executed in color. Copies of this patent or patent application with color drawings will be provided by the Office upon request and payment of the necessary fee.

FIG. 1A shows a siRNA of the invention containing two strands, where one of the strands contains disulfide linkages of the invention.

FIG. 1B shows a siRNA of the invention containing two strands, where both strands contain disulfide linkages of the invention.

FIG. 2 shows a representative polynucleotide construct of the invention and the RP-HPLC trace for the same polynucleotide.

FIG. 3 shows a mass spectrum of crude mixture of polynucleotide of the invention, the structure of which is shown in FIG. 2.

FIG. 4 shows a mass spectrum of purified polynucleotide of the invention, the structure of which is shown in FIG. 2.

FIG. 5A shows the structure of single-strand RNA constructs of the invention having one or three ADS conjugation sites.

FIG. 5B shows a photograph of the gel analysis of the single-strand RNA constructs of the invention. The structure of the constructs is described in FIGS. 6A, 6B, and 8.

FIG. 5C shows a photograph of the gel analysis of the single-strand RNA constructs of the invention. The structure of the constructs is described in FIGS. 6A, 6B, and 7A.

FIG. 5D shows a photograph of the gel analysis of the single-strand RNA constructs of the invention. The structure of the constructs is described in FIGS. 6A, 6B, and 7B.

FIG. 6A shows the general structure of representative siRNA constructs of the invention.

FIG. 6B shows the ADS conjugation group that is incorporated in the siRNA constructs shown in FIG. 6A.

FIG. 7A shows a structure of a representative targeting moiety (Folate) linked to a representative conjugating moiety.

FIG. 7B shows a structure of a representative targeting moiety (GalNAc) linked to a representative conjugating moiety.

FIG. 8 shows a structure of a representative targeting moiety (Mannose) linked to a representative conjugating moiety.

FIG. 9A shows dose curves for siRNA conjugate of the invention ((Folate)₃-siRNN-Cy3) binding to KB cell.

FIG. 9B shows a graph determining dissociation constants (K_{d}) for siRNA conjugates of the invention ((Folate)₃-siRNN-Cy3 or (Folate)₁-siRNN-Cy3) and KB cells.

FIG. 10A shows dose curves for siRNA conjugate of the invention ((GalNAc)₉-siRNN-Cy3) binding to HepG2 cells.

FIG. 10B shows a graph determining dissociation constants (K_{d}) for siRNA conjugates of the invention ((GalNAc)₉-siRNN-Cy3 or (GalNAc)₃-siRNN-Cy3) and HepG2 cells.

FIG. 11A shows dose curves for siRNA conjugate of the invention (Mannose)₁₈-siRNN-Cy3 binding to primary peritoneal macrophages.

FIG. 11B shows a graph determining dissociation constants (K_{d}) for siRNA conjugates of the invention ((Mannose)₁₈-siRNN-Cy3 or (Mannose)₆-siRNN-Cy3) and primary peritoneal macrophages.

FIG. 12 is an image of NFκB-RE-Luc mice 4 hours after intraperitoneal administration of tumor necrosis factor-α (TNF-α). Comparison is provided to negative controls. The mice treated with siRNA of the invention exhibit diminished levels of Luciferase compared to the negative control mouse.

FIGS. 13 and 14 are graphs showing efficacy of exemplary siRNA compounds listed in Table 4 n inhibiting ApoB gene expression in vitro in primary mouse hepatocytes from C57/Bl6 mouse. The determined IC₅₀ values are provided in tables under each graph.

FIGS. 15A and 15B are graphs showing efficacy of exemplary siRNA compounds listed in Table 4 in inhibiting ApoB gene expression in vivo in C57Bl6 mice. FIG. 15A is a graph demonstrating dose response function at 72 hours measured by liver ApoB gene expression normalized to 32 microglobulin (B2M) gene expression in vivo versus administration of a vehicle only. FIG. 15B is a graph demonstrating time course of liver ApoB gene expression in vivo 96, 72, 48, and 24 hours following administration of siRNA (SB0097, see Table 4) normalized to B2M gene expression in vivo versus administration of vehicle only.

FIGS. 16 and 17 are images of general structures encompassed by the present invention.

FIGS. 18A and 18B show results from mouse primary bone marrow cell experiments. FIG. 18A shows the normalized amount of mannose receptor expression in macrophages over time. FIG. 18B shows a graph of GAPDH mRNA normalized to B2M after treatment with 48 hour treatment with exemplary siRNA compounds listed in Table 4.

### DETAILED DESCRIPTION

The ability to deliver certain bioactive agents to the interior of cells is problematic due to the selective permeability of the cell plasma membrane. The plasma membrane of the cell forms a barrier that restricts the intracellular uptake of molecules to those which are sufficiently non-polar and smaller than approximately 500 daltons in size. Previous efforts to enhance the cellular internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques can result in poor cellular uptake and intracellular sequestration into the endocytic pathway. Due to their anionic charge and large size of about 14,000 Daltons, delivery of siRNA is a formidable challenge in mammals, including humans. However, cationically charged peptides and proteins have led to advancements in polynucleotide delivery. For example, linking peptide transduction domains (PTDs) to a nucleic acid has provided some advancement in polynucleotide delivery. nucleotide construct

The invention provides nucleotide constructs comprising one or more bioreversible groups (e.g., disulfides). Sterically-hindered disulfides are particularly advantageous. Disulfides bonded to at least one bulky group exhibit greater stability during the nucleotide construct synthesis compared to disulfides that are not bonded to at least one bulky group, as the latter may react with a phosphorus (III) atom of the nucleotide construct to cleave the disulfide bond.

The invention demonstrates that relatively large moieties, e.g., a polypeptide, a carbohydrate, a neutral organic polymer, a positively charged polymer, a therapeutic agent, a targeting moiety, an endosomal escape moiety, or combination thereof, may be linked to bioreversible groups connected to internucleotide bridging groups, without affecting the ability of the bioreversible group to be cleaved intracellularly. The invention also provides for nucleotide constructs comprising bioreversible groups that have hydrophobic or hydrophilic functional groups, and/or conjugating moieties, wherein these conjugating moieties allow for attachment of a polypeptide, a small molecule, a carbohydrate, a neutral organic polymer, a positively charged polymer, a therapeutic agent, a targeting moiety, an endosomal escape moiety, or any combination thereof to an internucleotide bridging group or a terminal nucleotide group. The invention further provides for a nucleotide construct that comprises one or more bioreversible groups comprising one or more hydrophobic or hydrophilic functional groups, and/or one or more conjugating groups having one or more conjugating moieties that allow for the attachment of an auxiliary moiety, e.g., a polypeptide, a small molecule, a carbohydrate, a neutral organic polymer, a positively charged polymer, a therapeutic agent, a targeting moiety, an endosomal escape moiety, or any combination thereof, to the nucleotide construct. In a certain embodiment, the nucleotide constructs disclosed herein contain a certain number of bioreversible groups reducing the overall negative charge of the constructs, thereby allowing for or facilitating the uptake of the constructs by a cell. The nucleotide constructs described herein can allow for or facilitate the intracellular transport of a polynucleotide itself or a polynucleotide linked to an attached auxiliary moiety, e.g., a small molecule, peptide, polypeptide, carbohydrate, neutral organic polymer, positively charged polymer, therapeutic agent, targeting moiety, endosomal escape moiety, or combination thereof. The action of intracellular enzymes (e.g., intracellular protein disulfide isomerase, thioredoxin, or thioesterases) or exposure to the intracellular environment can result in the cleavage of the disulfide or thioester linkage, thereby releasing the auxiliary moiety and/or unmasking the polynucleotide. The unmasked polynucleotide can then, e.g., initiate an antisense or RNAi-mediated response. Further, the nucleotide constructs of the invention also allow for or facilitate the intracellular delivery of a polynucleotide or a polynucleotide linked through a disulfide or a thioester linkage to an attached auxiliary moiety, e.g., a small molecule, peptide, polypeptide, carbohydrate, neutral organic polymer, positively charged polymer, therapeutic agent, targeting moiety, endosomal escape moiety, or combination thereof, without the need for carriers, such as liposomes, or cationic lipids. Preferably, the linkage between the auxiliary moiety and the polynucleotide includes a disulfide linkage. Each of the features is further described herein.

The invention provides methods and compositions to facilitate and improve the cellular uptake of polynucleotides by reducing or neutralizing the charge associated with anionically charged polynucleotides, and optionally adding further functionality to the molecule, e.g., cationic peptides, targeting moiety, and/or endosomal escape moiety. In particular embodiments, the compositions of the invention may promote uptake of a polynucleotide by generating nucleotide constructs that have a cationic charge.

The invention provides compositions and methods for the delivery of sequence specific polynucleotides useful for selectively treating human disorders and for promoting research. The compositions and methods of the invention effectively deliver polynucleotides, including siRNAs, RNA, and DNA to subjects and to cells, without the drawbacks of current nucleic acid delivery methods. The invention provides compositions and methods which overcome size and charge limitations that make RNAi constructs difficult to deliver into cells or make the constructs undeliverable. By reversibly neutralizing the anionic charge of nucleic acids (e.g., dsRNA), a nucleotide construct comprising a bioreversible group according to the invention can deliver nucleic acids into a cell in vitro and in vivo.

The invention provides nucleotide constructs comprising a charge neutralizing moiety (e.g., a component (i) or a group of formula (II) used as a protecting group for an internucleotide or a terminal group). The construct can further include auxiliary moieties useful in cellular transfection and cellular modulation. Such auxiliary moieties can include a small molecule, peptide, a polypeptide, a carbohydrate, a neutral organic polymer, a positively charged polymer, a therapeutic agent, a targeting moiety, an endosomal escape moiety, or any combination thereof.

The invention provides compositions and methods for the delivery of nucleotide constructs comprising one or more targeting moieties for targeted delivery to specific cells (e.g., cells having asialoglycoprotein receptors on their surface (e.g., hepatocytes), tumor cells (e.g., tumor cells having folate receptors on their surface), cells bearing mannose receptor (e.g., macrophages, dendritic cells, and skin cells (e.g., fibroblasts or keratinocytes))). Non-limiting examples of mannose receptor superfamily include MR, Endo180, PLA2R, MGL, and DEC205. Targeted delivery of the nucleotide constructs of the invention may involve receptor mediated internalization. In some embodiments, targeting moieties may include mannose, N-acetyl galactosamine (GalNAc), or a folate ligand.

As demonstrated herein, the addition of one or more removable (e.g., reversibly attached) charge neutralizing moieties to a nucleic acid can facilitate cell transfection. Any nucleic acid, regardless of sequence composition, can be modified. Accordingly, the invention is not limited to any particular sequence (i.e., any particular siRNA, dsRNA, DNA or the like).

The invention provides nucleotide constructs having, in some embodiments, one or more bioreversible moieties that contribute to chemical and biophysical properties that enhance cellular membrane penetration and resistance to exo- and endonuclease degradation. The invention further provides reagents for the synthesis of the nucleotide constructs disclosed herein, e.g., phosphoramidite reagents. Moreover, these bioreversible groups are stable during the synthetic processes.

In cells, the bioreversible moieties can be removed by the action of enzymes (e.g., enzymes having thioreductase activity (e.g., protein disulfide isomerase or thioredoxin)) or by exposure to the intracellular conditions (e.g., an oxidizing or reducing environment) or reactants (e.g., glutathione or other free thiol) to yield biologically active polynucleotide compounds that are capable of hybridizing to and/or having an affinity for specific endogenous nucleic acids.

The bioreversible moieties can be used with antisense polynucleotides of synthetic DNA or RNA or mixed molecules of complementary sequences to a target sequence belonging to a gene or to an mRNA whose expression they are specifically designed to block or down-regulate. These inhibitory polynucleotides may be directed against a target mRNA sequence or, alternatively against a target DNA sequence, and hybridize to the nucleic acid to which they are complementary thereby inhibiting transcription or translation. Accordingly, the nucleotide constructs disclosed herein can effectively block or down-regulate gene expression.

The nucleotide constructs of the invention may also be directed against certain bicatenary DNA regions (homopurine/homopyrimidine sequences or sequences rich in purines/pyrimidines) and thus form triple helices. The formation of a triple helix, at a particular sequence, can block the interaction of protein factors which regulate or otherwise control gene expression and/or may facilitate irreversible damage to be introduced to a specific nucleic acid site if the resulting polynucleotide is made to possess a reactive functional group.

Polynucleotides

The invention provides nucleotide constructs that contain polynucleotides (“polynucleotide constructs”) having one or more charge neutralizing groups (e.g., a component (i), a group of formula (II), or a group of formula (IIA)) attached to an internucleotide bridging group or terminal nucleotide group (5′- or 3′-terminal group). The one or more charge neutralizing groups can contain a bioreversible group, such as a disulfide or a thioester linkage. Preferably, the one or more charge neutralizing groups include a disulfide linkage. The one or more charge neutralizing groups can contain one or more auxiliary moieties linked to the internucleotide bridging group or terminal nucleotide group through a bioreversible group (e.g., a disulfide or a thioester linkage; preferably, a disulfide linkage). Examples of such auxiliary moieties include a small molecule, a conjugating moiety, a hydrophilic functional group, a polypeptide, a carbohydrate, a neutral organic polymer, a positively charged polymer, a therapeutic agent, a targeting moiety, an endosomal escape moiety, and any combination thereof. The bioreversible group may be able to undergo a separate reaction, e.g., intramolecularly, to leave an unmodified internucleotide bridging group or terminal nucleotide group. While various sugars and backbones can be employed, as described in the definition of nucleotide provided herein, the polynucleotide will typically employ a ribose, deoxyribose, or LNA sugar and phosphate or thiophosphate internucleotide bridging groups. Mixtures of these sugars and bridging groups in a single polynucleotide are also contemplated.

The polynucleotides constructs described herein feature bioreversible groups that can be selectively cleaved intracellularly (e.g., by exposure to the passive environment, action of enzymes, or other reactants) thereby facilitating the intracellular delivery of polynucleotides to cells. Exemplary bioreversible groups include disulfide linkages.

For example, the polynucleotide constructs described herein can include disulfide linkages that can be cleaved by intracellular enzymes having thioreductase activity. Upon entry into a cell, these disulfide linkages (e.g., those contained between A¹ group and A² group of formula (II)) can be selectively cleaved by enzymes in order to unmask the nucleic acid. Disulfide linkages described herein can also provide a useful handle by which to functionalize the nucleic acid with one or more auxiliary moieties (e.g., one or more targeting moieties) and other conjugates, or with groups that will modify the physicochemical properties of the nucleic acid (e.g., hydrophilic groups such as hydroxy (—OH) groups). The strategy can be readily generalized to a number of structurally and functionally diverse nucleic acids in order to allow for targeted cellular delivery without the use of separate delivery agents.

The polynucleotide constructs described herein can include, e.g., 1-40 independent bioreversible groups. For example, the polynucleotide constructs disclosed herein can include between 1-30, 1-25, 1-20, 2-15, 2-10, or 1-5 independent bioreversible groups. In particular embodiments, no more than 75% of the constituent nucleotides include a bioreversible group (e.g., no more than 50%, 55%, 60%, 65%, 70%, or 75% include a bioreversible group). In another embodiment, up to 90% of nucleotides within a polynucleotide construct of the invention can have a bioreversible group. In yet another embodiment, no more than half of the bioreversible groups will include hydrophobic termini, e.g., alkyl groups (e.g., when (R⁴)ᵣ-L-A¹ combine to form a hydrophobic group). The polynucleotide constructs disclosed herein can feature any combination of bioreversible groups, e.g., that include a conjugating moiety, a hydrophilic functional group, a polypeptide, a small molecule, a carbohydrate, a neutral organic polymer, a positively charged polymer, a therapeutic agent, a targeting moiety, an endosomal escape moiety, or any combination thereof. The polynucleotide construct will generally be up to 150 nucleotides in length. In some embodiments, the polynucleotide construct consists of 5-100, 5-75, 5-50, 5-25, 8-40, 10-32, 15-25, or 20-25 nucleotides in length.

In certain embodiments, the polynucleotide construct contains one or more components (i), each of the components contains, independently, a polypeptide, a carbohydrate, a neutral organic polymer, a positively charged polymer, a therapeutic agent, a targeting moiety, or an endosomal escape moiety; where each of the components (i) includes a linker to an internucleotide bridging group of the polynucleotide construct, the linker containing a disulfide or a thioester (preferably, a disulfide) and one or more bulky groups proximal to the disulfide group and rendering the disulfide group sterically hindered.

In particular embodiments, the locations of bioreversible groups within a polynucleotide construct are selected so as to improve the stability of the resulting construct (e.g., to increase half life of the polynucleotide construct in the absence of the reagents (e.g., an oxidizing or reducing environment) responsible for cleaving the disulfide linkage). In particular, for double stranded polynucleotides, the location of the bioreversible groups will be such that a stable at mammalian physiological temperature double-stranded molecule is formed.

In other embodiments, the nature of each bioreversible group can be selected so as to generate favorable solubility and delivery properties. Such variations can include modulating the linker length, e.g., between the internucleotide bridging group or terminal nucleotide group and the disulfide group and/or between the disulfide group and any conjugating moiety, hydrophilic functional group, or auxiliary moiety. Reductions in solubility caused by hydrophobic bioreversible groups can be offset, in part, by the use of one or more hydrophilic bioreversible groups elsewhere in the polynucleotide. In a particular embodiment, the sugar on the 3′ end of an internucleotide bridging group having a bioreversible group does not include a 2′ OH group, e.g., includes a 2′ F or OMe group instead.

For example, some of the polynucleotide constructs described herein can have a structure according to Formula I,

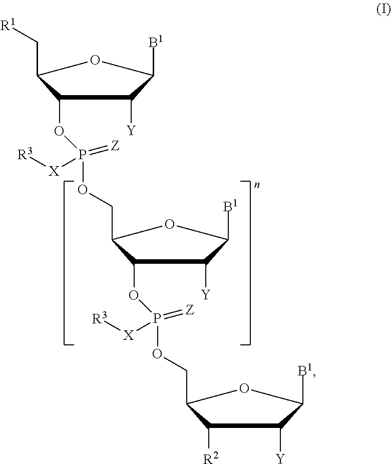

or a salt thereof,

wherein n is a number from 0 to 150;
each B¹ is independently a nucleobase;
each X is independently selected from the group consisting of O, S, and optionally substituted N;

each Y is independently selected from the group consisting of hydrogen, hydroxyl, halo, optionally substituted C₁₋₆ alkoxy, and a protected hydroxyl group;

each Z is independently O or S;

R¹ is selected from the group consisting of H, hydroxyl, optionally substituted C₁₋₆ alkoxy, a protected hydroxyl group, a monophosphate, a diphosphate, a triphosphate, a tetraphosphate, a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and any combination thereof, or R¹ is

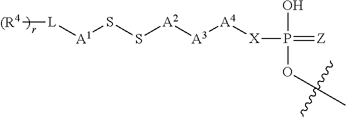

or a salt thereof;

R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an optionally substituted C₁₋₆ alkyl, an amino containing group, a biotin containing group, a digoxigenin containing group, a cholesterol containing group, a quencher containing group, a phosphothiol, a polypeptide, a carbohydrate, a neutral organic polymer, a positively charged polymer, a therapeutic agent, a targeting moiety, an endosomal escape moiety, and any combination thereof, or R² is

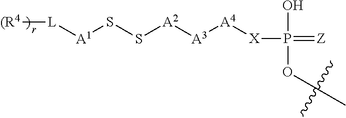

or a salt thereof; and

each R³ is independently absent, a hydrogen, optionally substituted C₁₋₆ alkyl, or a group having the structure of Formula II:

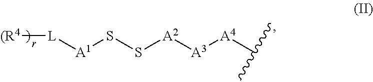

wherein A¹ is a bond or a linker containing or being one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ includes one or more of optionally substituted N, O, and S, said optionally substituted N, O, or S is not directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or consisting of one or more conjugating moieties;

R⁴ is hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

wherein A², A³, and A⁴ combine to form a group having at least three atoms in the shortest chain connecting —S—S— and X; and

wherein at least one R³ has the structure of formula (II).

The disulfide linkage in the polynucleotide and nucleotides of the invention may be replaced by another bioreversible group, e.g., a thioester moiety. For example, the group of formula (II), (IIa), (VIII), or (VIIIa) may be replaced with the group of formula (IIb):

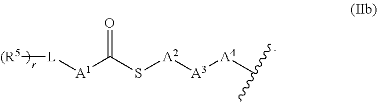

One of skill in the art would be able to adapt the synthetic methods described herein to prepare such polynucleotides and nucleotides. Thus, the thioester-containing groups are considered to be within the scope of the present invention.

Certain embodiments of formula (I) include those in which X and Z are both O. In some embodiments, polynucleotide constructs disclosed herein largely comprise the structure of formula (I) but the depicted internucleotide bridging group of formula (I) is replaced with another internucleotide bridging group (e.g., modified polynucleotide backbones) described herein. In alternate embodiments, polynucleotide constructs disclosed herein largely contain the structure of formula (I) but the depicted group R¹ and/or R² of formula (I) is replaced with a terminal nucleotide group having group R³. Polynucleotide constructs disclosed herein may have modified polynucleotide backbones. Examples of modified polynucleotide backbones include, for example, phosphorothioates, chiral phosphorothioates, phosphorodithioates, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Representative United States patents that teach the preparation of the above phosphorus-containing linkages include U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference. Nucleotide constructs disclosed herein having modified polynucleotide backbones that do not include a phosphorus atom therein may have backbones that are formed by short chain alkyl or cycloalkyl internucleotide bridging groups, mixed heteroatom and alkyl or cycloalkyl internucleotide bridging groups, or one or more short chain heteroatomic or heterocyclic internucleotide bridging groups. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above polynucleotides include U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Exemplary -A¹-S—S-A²-A³-A⁴- or —S—S-A²-A³-A⁴- groups are as follows:

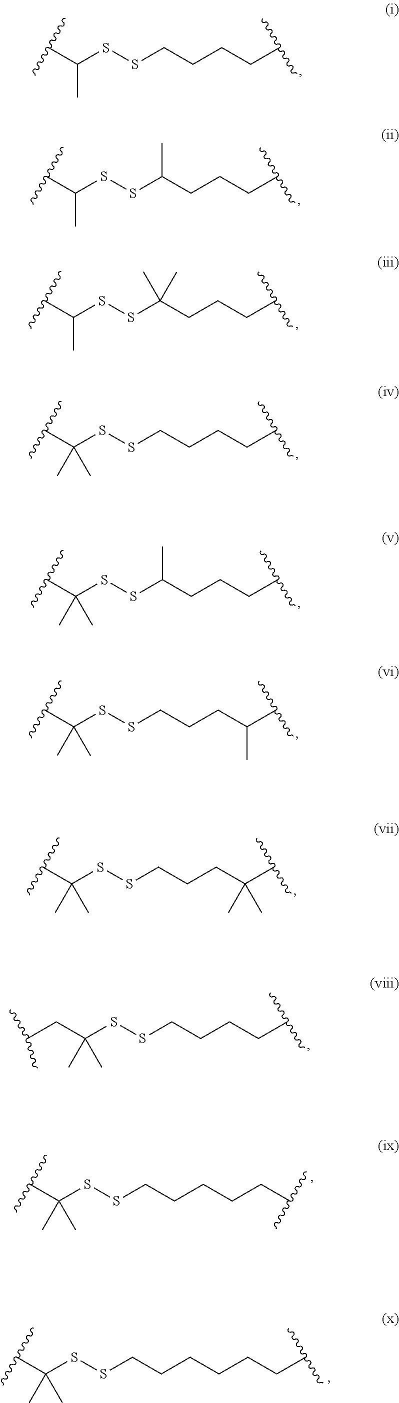

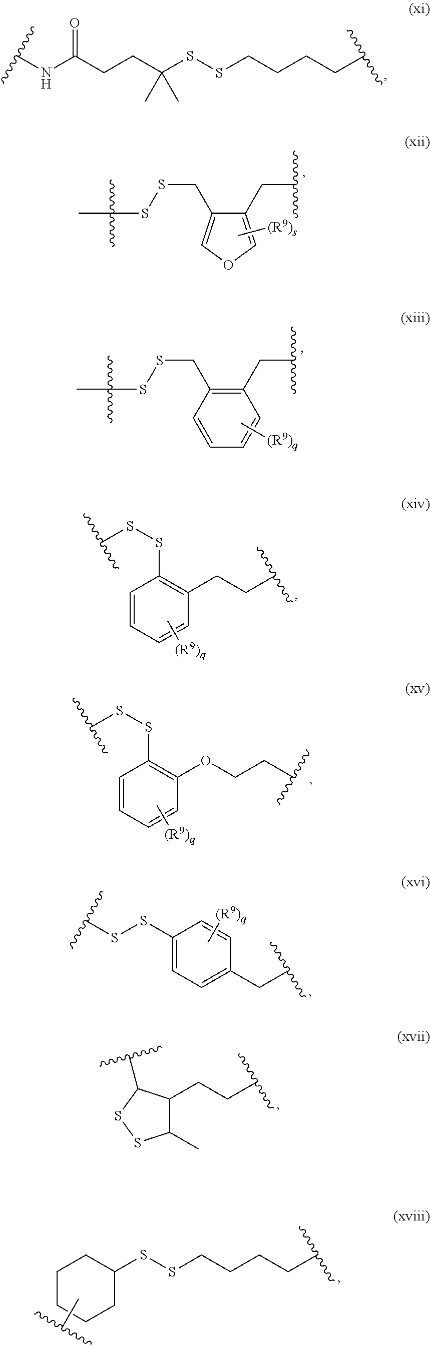

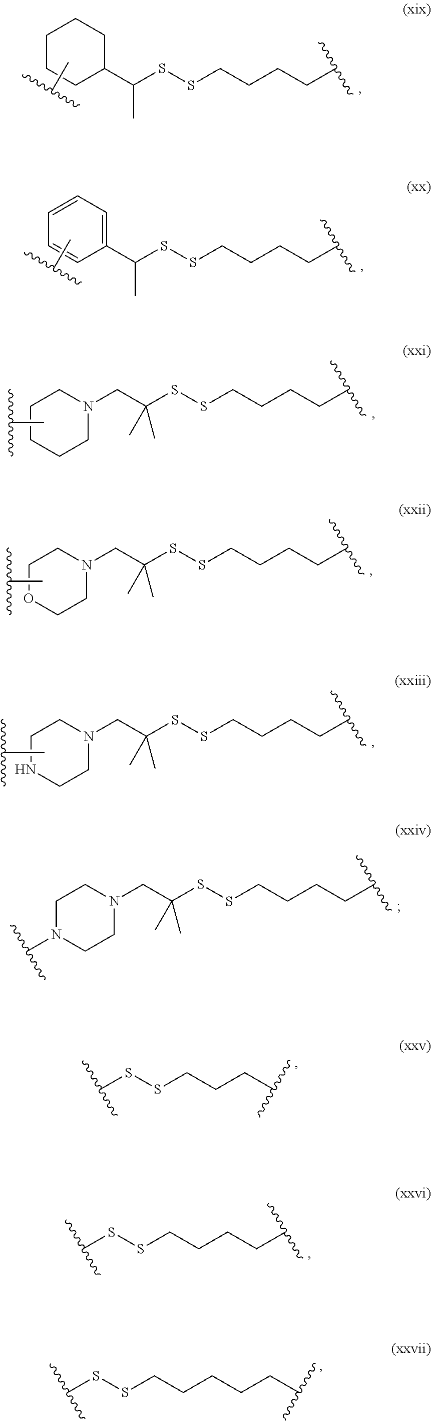

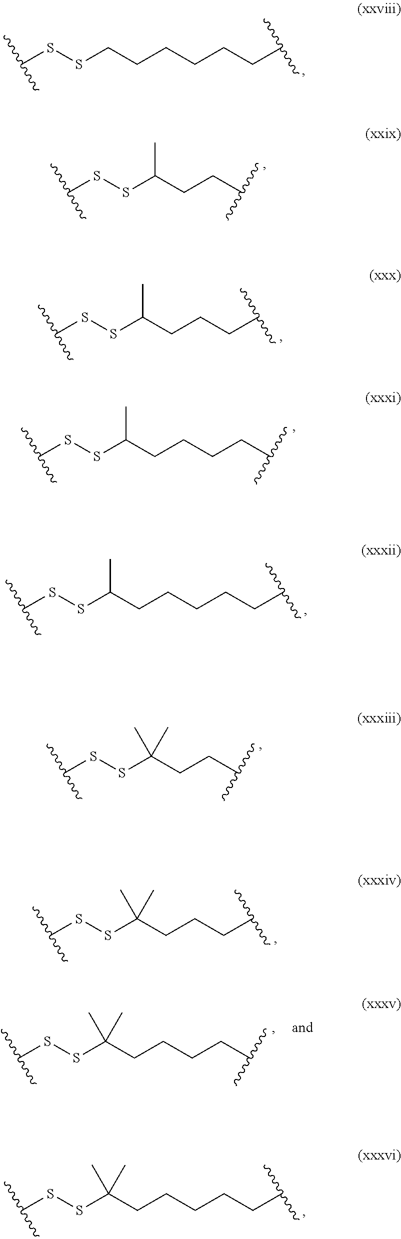

where

each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from nitrogen, oxygen, and sulfur; optionally substituted (C₁₋₉ heteroaryl)-C₁₋₄-alkyl having 1 to 4 heteroatoms selected from nitrogen, oxygen; optionally substituted C₁₋₉ heterocyclyl having 1 to 4 heteroatoms selected from nitrogen, oxygen, and sulfur; optionally substituted (C₁₋₉ heterocyclyl)-C₁₋₄-alkyl having 1 to 4 heteroatoms selected from nitrogen, oxygen, and sulfur; optionally substituted amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

The invention further provides methods for manufacturing the polynucleotide constructs of the invention. Methods for the preparation of nucleotides and polynucleotides are known in the art. For example, the practice of phosphoramidite chemistry to prepare polynucleotides is known from the published work of Caruthers and Beaucage and others. See, e.g., U.S. Pat. Nos. 4,458,066; 4,500,707; 5,132,418; 4,415,732; 4,668,777; 4,973,679; 5,278,302, 5,153,319; 5,218,103; 5,268,464; 5,000,307; 5,319,079; 4,659,774; 4,672,110; 4,517,338; 4,725,677; and RE34,069, each of which is herein incorporated by reference, describe methods of polynucleotide synthesis. Additionally, the practice of phosphoramidite chemistry has been systematically reviewed by Beaucage et al., Tetrahedron, 48: 2223-2311, 1992; and Beaucage et al., Tetrahedron, 49:6123-6194, 1993, as well as references referred to therein, all of which are herein incorporated by reference.

Nucleic acid synthesizers are commercially available, and their use is generally understood by persons of ordinary skill in the art as being effective in generating nearly any polynucleotide of reasonable length which may be desired.

In practicing phosphoramidite chemistry, useful 5′OH sugar blocking groups are trityl, monomethoxytrityl, dimethoxytrityl and trimethoxytrityl, especially dimethoxytrityl (DMTr). In practicing phosphoramidite chemistry, useful phosphite activating groups are dialkyl substituted nitrogen groups and nitrogen heterocycles. One approach includes the use of the di-isopropylamino activating group.

Polynucleotides can be synthesized by a Mermade-6 solid phase automated polynucleotide synthesizer or any commonly available automated polynucleotide synthesizer. Triester, phosphoramidite, or hydrogen phosphonate coupling chemistries (described in, for example, M. Caruthers, Oligonucleotides: Antisense Inhibitors of Gene Expression, pp. 7-24, J. S. Cohen, ed. (CRC Press, Inc. Boca Raton, Fla., 1989); Oligonucleotide synthesis, a practical approach, Ed. M. J. Gait, IRL Press, 1984; and Oligonucleotides and Analogues, A Practical Approach, Ed. F. Eckstein, IRL Press, 1991) are employed by these synthesizers to provide the desired polynucleotides. The Beaucage reagent, as described in, for example, Journal of American Chemical Society, 112:1253-1255, 1990, or elemental sulfur, as described in Beaucage et al., Tetrahedron Letters 22:1859-1862, 1981, is used with phosphoramidite or hydrogen phosphonate chemistries to provide substituted phosphorothioate polynucleotides.

For example, the reagents containing the protecting groups recited herein can be used in numerous applications where protection is desired. Such applications include, but are not limited to, both solid phase and solution phase, polynucleotide synthesis and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. For use with phosphoramidite chemistry, various phosphoramidite reagents are commercially available, including 2′-deoxy phosphoramidites, 2′-O-methyl phosphoramidites and 2′-O-hydroxyl phosphoramidites. Any other means for such synthesis may also be employed. The actual synthesis of the polynucleotides is well within the talents of those skilled in the art. It is also well known to use similar techniques to prepare other polynucleotides such as the phosphorothioates, methyl phosphonates and alkylated derivatives. It is also well known to use similar techniques and commercially available modified phosphoramidites and controlled-pore glass (CPG) products such as biotin, Cy3, fluorescein, acridine or psoralen-modified phosphoramidites and/or CPG (available from Glen Research, Sterling Va.) to synthesize fluorescently labeled, biotinylated or other conjugated polynucleotides.

In particular embodiments, a method of manufacturing a polynucleotide construct of the invention involves the use of one or more nucleotide constructs having Formula (Ia):

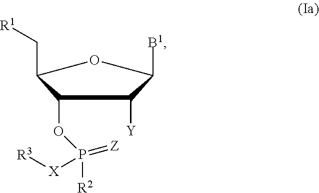

or a salt thereof,

B¹ is a nucleobase;

X is O, S, or optionally substituted N;

Y is a hydrogen, hydroxyl, halo, optionally substituted C₁₋₆ alkoxy, or a protected hydroxyl group;

Z is absent;

R¹ is protected hydroxyl (e.g., 4,4′-dimethoxytrityl group (DMT));

R² is —N(R³)R⁴ or —N(C₁₋₆ alkyl)₂ (e.g., —N(iPr)₂); and

R³ is a group having the structure of Formula (IIa):

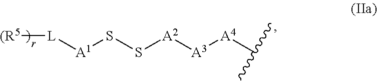

where A¹ is a bond or a linker containing or consisting of one or more of optionally substituted N, O, S, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₆ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from nitrogen, oxygen, and sulfur; optionally substituted (C₁₋₉ heteroaryl)-C₁₋₄-alkylene having 1 to 4 heteroatoms selected from nitrogen, oxygen; optionally substituted C₁₋₉ heterocyclylene having 1 to 4 heteroatoms selected from nitrogen, oxygen, and sulfur; and optionally substituted (C₁₋₉ heterocyclyl)-C₁₋₄-alkylene having 1 to 4 heteroatoms selected from nitrogen, oxygen, and sulfur, provided that when A¹ comprises one or more of optionally substituted amino, O, and S, none of said optionally substituted amino, O, and S is directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from nitrogen, oxygen, and sulfur; and optionally substituted C₁₋₉ heterocyclylene having 1 to 4 heteroatoms selected from nitrogen, oxygen, and sulfur; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from nitrogen, oxygen, and sulfur; optionally substituted C₁₋₉ heterocyclylene having 1 to 4 heteroatoms selected from nitrogen, oxygen, and sulfur; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from nitrogen, oxygen, and sulfur;

L is a bond or a conjugating group including or consisting of one or more conjugating moieties;

R⁵ is hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms in the shortest chain connecting —S—S— and X; and

each R⁴ and R⁶ is independently selected from the group consisting of hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₁₋₉ heterocyclyl having 1 to 4 heteroatoms selected from nitrogen, oxygen, and sulfur; and optionally substituted C₃₋₁₀ (heterocycle)oyl having 1 to 4 heteroatoms selected from nitrogen, oxygen, and sulfur.

The invention further provides methods to process a polynucleotide construct synthesized by using a method of manufacture disclosed herein. For example, post synthesis of the polynucleotide construct, if a nucleobase contains one or more protecting groups, the protecting groups may be removed; and/or for any -L-A¹-S—S-A²-A³-A⁴- containing a hydrophilic functional group or conjugating moiety that is protected by a protecting group, then the protecting group may be removed.

Additionally, post synthesis of the polynucleotide construct, a group containing one or more of a small molecule, a polypeptide, a carbohydrate, a neutral organic polymer, a positively charged polymer, a therapeutic agent, a targeting moiety, and an endosomal escape moiety can be linked to one or more conjugating moieties of one or more bioreversible groups.

Nucleotides

The invention further provides compounds containing a single nucleotide (“compound of the invention”). Thus, the invention features a compound that has a structure according to Formula (VII):

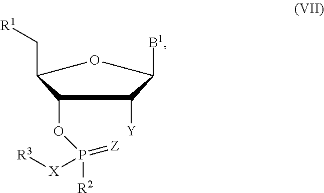

or a salt thereof,

where

B¹ is a nucleobase;

X is O, S, or NR⁴;

Y is hydrogen, hydroxyl, halo, optionally substituted C₁₋₆ alkoxy, or a protected hydroxyl group;

Z is absent, O, or S;

R¹ is hydroxyl, optionally substituted C₁₋₆ alkoxy, a protected hydroxyl group, a monophosphate, a diphosphate, a triphosphate, a tetraphosphate, and a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, or any combination thereof;

R² is H, hydroxyl, optionally substituted C₁₋₆ alkoxy, a protected hydroxyl group, a monophosphate, a diphosphate, a triphosphate, a tetraphosphate, a pentaphosphate, and an optionally substituted amino, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, or any combination thereof; and

R³ is a group having the structure of Formula (VIII):

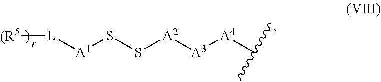

where

A¹ is a bond or a linker including or consisting of one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ comprises one or more of optionally substituted N, O, and S, said optionally substituted N, O, or S is not directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or consisting of one or more conjugating moieties;

R⁵ is absent, hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, or any combination thereof, where the hydrophilic functional group is optionally protected with a protecting group;

r is an integer from 1 to 10;

wherein A², A³, and A⁴ combine to form a group having at least three atoms in the shortest chain connecting —S—S-A¹-R⁵ and —X—; and

each R⁴ and R⁶ is independently selected from the group consisting of hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₁₋₉ heterocyclyl having 1 to 4 heteroatoms selected from nitrogen, oxygen, and sulfur; and optionally substituted C₆₋₁₀ (heterocycle)oyl having 1 to 4 heteroatoms selected from nitrogen, oxygen, and sulfur.

Other embodiments of the compound of formula (VII) include the following: Z is absent;

A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₄-alkylene; optionally substituted C₁₋₉ heteroarylene having 1 to 4 heteroatoms selected from nitrogen, oxygen, and sulfur; optionally substituted (C₁₋₉ heteroaryl)-C₁₋₄-alkylene having 1 to 4 heteroatoms selected from nitrogen, oxygen; optionally substituted C₁₋₉ heterocyclylene having 1 to 4 heteroatoms selected from nitrogen, oxygen, and sulfur; and optionally substituted (C₁₋₉ heterocyclyl)-C₁₋₄-alkylene having 1 to 4 heteroatoms selected from nitrogen, oxygen, and sulfur; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from nitrogen, oxygen, and sulfur; and optionally substituted C₁₋₉ heterocyclylene having 1 to 4 heteroatoms selected from nitrogen, oxygen, and sulfur; or A¹ and A², together with —S—S—, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from nitrogen, oxygen, and sulfur; optionally substituted C₁₋₉ heterocyclylene having 1 to 4 heteroatoms selected from nitrogen, oxygen, and sulfur; O; NR⁶; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from nitrogen, oxygen, and sulfur;

L is a bond or a conjugating group including or consisting of one or more conjugating moieties;

R⁵ is absent, hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms in the shortest chain connecting —S—S— and X; and

each R⁴ is independently hydrogen; optionally substituted C₁₋₆ alkyl; optionally substituted C₂₋₇ alkanoyl; hydroxyl; optionally substituted C₁₋₆ alkoxy; optionally substituted C₃, cycloalkyl; optionally substituted C₃₋₈ cycloalkenyl; optionally substituted C₆₋₁₄ aryl; optionally substituted C₆₋₁₅ aryloyl; optionally substituted C₂₋₉ heterocyclyl having 1 to 4 heteroatoms selected from nitrogen, oxygen, and sulfur; or optionally substituted C₃₋₁₀ (heterocycle)oyl having 1 to 4 heteroatoms selected from nitrogen, oxygen, and sulfur.

In yet other embodiments of the compound of formula (VII) -A¹-S—S-A²-A³-A⁴- or —S—S-A²-A³-A⁴- group is one of the following:

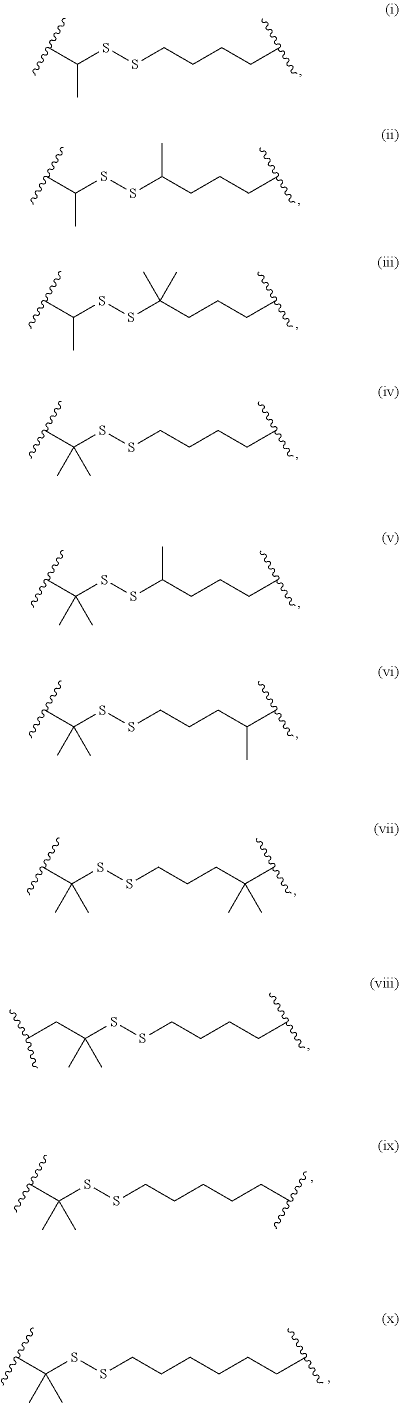

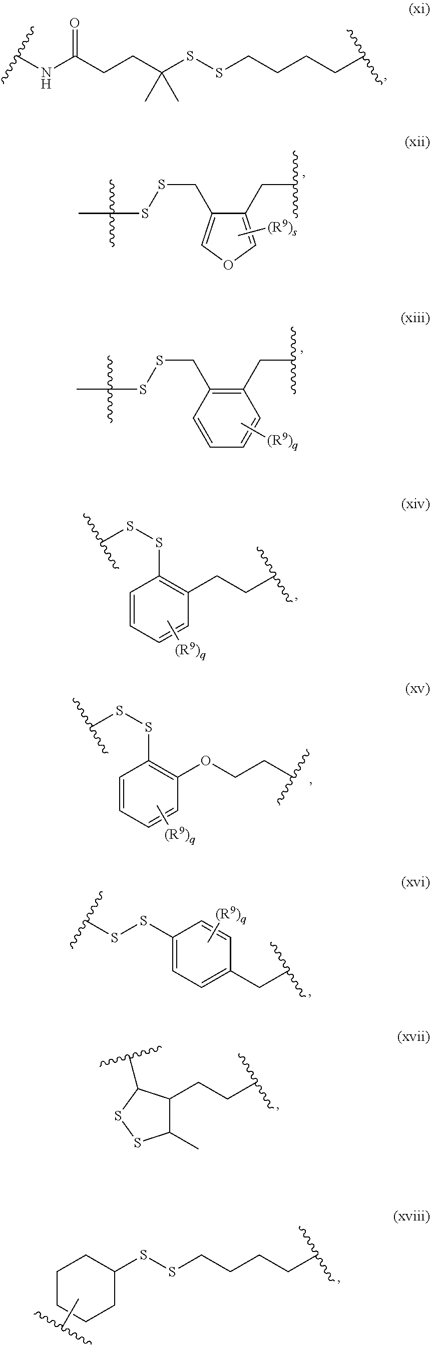

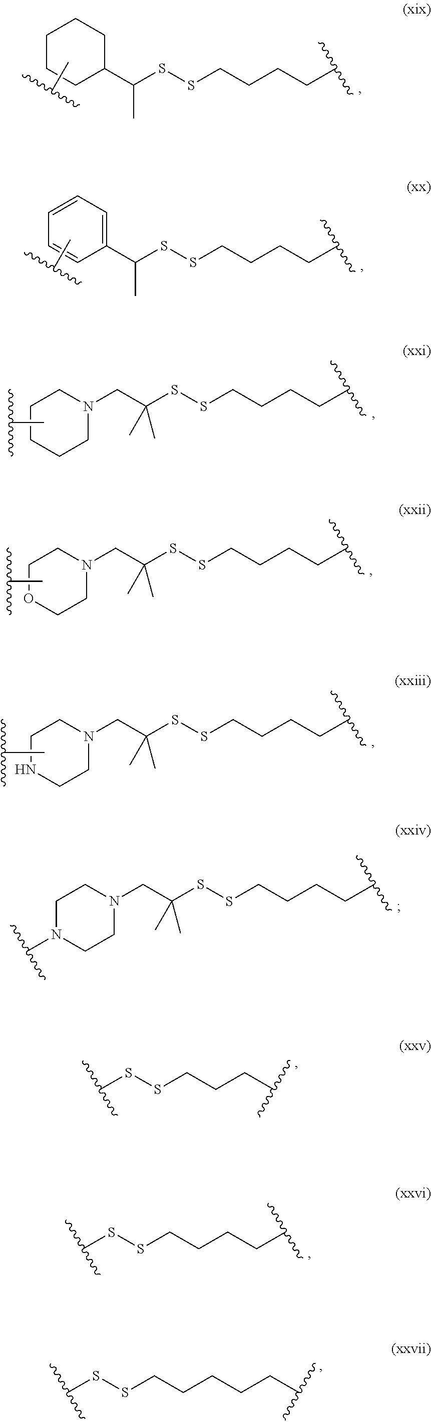

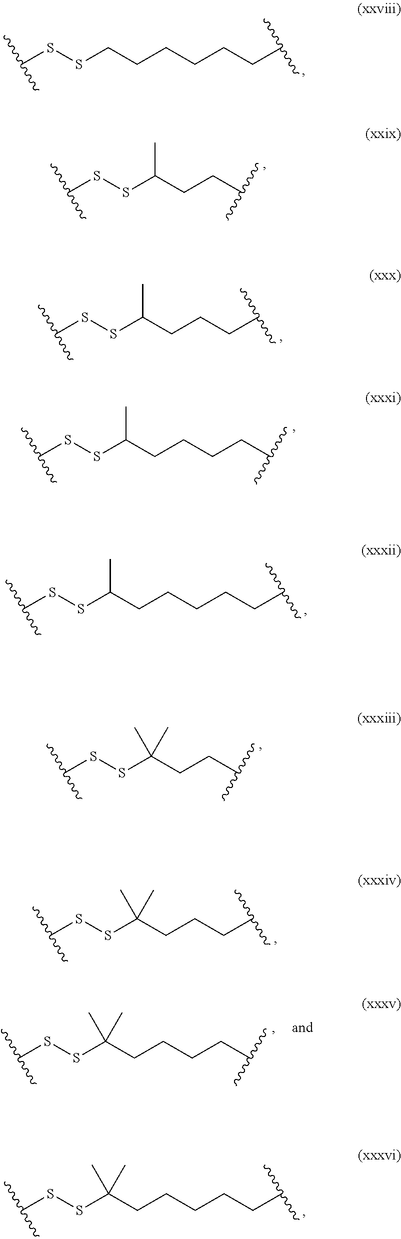

where

each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from nitrogen, oxygen, and sulfur; optionally substituted (C₁₋₉ heteroaryl)-C₁₋₄-alkyl having 1 to 4 heteroatoms selected from nitrogen, oxygen; optionally substituted C₁₋₉ heterocyclyl having 1 to 4 heteroatoms selected from nitrogen, oxygen, and sulfur; optionally substituted (C₁₋₉ heterocyclyl)-C₁₋₄-alkyl having 1 to 4 heteroatoms selected from nitrogen, oxygen, and sulfur; optionally substituted amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In particular embodiments, the auxiliary moiety can be attached to the group containing a disulfide linkage by forming one or more covalent bonds to a conjugating moiety found in the conjugating group.

Conjugates

Nucleotide constructs of the invention may contain one or more conjugating groups having one or more conjugating moieties. The conjugating moieties can in turn be used to attach various other auxiliary moieties, e.g., a small molecule, a polypeptide, a carbohydrate, a neutral organic polymer, a positively charged polymer, a therapeutic agent, a targeting moiety, an endosomal escape moiety, or combination thereof, to the nucleotide construct. In a certain embodiment, more than one type of conjugating moiety is present in a nucleotide construct, thereby allowing the selective and/or sequential coupling of auxiliary moieties to the nucleotide construct. The location of attachment in a polynucleotide construct is determined by the use of the appropriate nucleotide construct in the synthesis of the polymer. A nucleotide construct containing one more conjugating moieties will react, under appropriate conditions, with one or more corresponding conjugating moieties on auxiliary moieties. The auxiliary moiety may intrinsically possess the conjugating moiety, e.g., terminal or lysine amine groups and thiol groups in peptides or polypeptides, or it may be modified to include a small linking group to introduce the conjugating moiety. Introduction of such linking groups is well known in the art. It will be understood that an auxiliary moiety attached to a nucleotide construct of the invention includes any necessary linking group.

Diverse bond-forming methods can be used to conjugate the auxiliary moiety to the nucleotide constructs described herein. Exemplary reactions include: Hüisgen cycloaddition between an azide and an alkyne to form a triazole; the Diels-Alder reaction between a dienophile and a diene/hetero-diene; bond formation via other pericyclic reactions such as the ene reaction; amide or thioamide bond formation; sulfonamide bond formation; alcohol or phenol alkylation (e.g., with diazo compounds), condensation reactions to form oxime, hydrazone, or semicarbazide group, conjugate addition reactions by nucleophiles (e.g., amines and thiols), disulfide bond formation, and nucleophilic substitution at a carboxylic functionality (e.g., by an amine, thiol, or hydroxyl nucleophile). Other exemplary methods of bond formation are described herein and known in the art.

Nucleophile/Electrophile Reactions

Nucleophiles and electrophiles can engage in bond forming reactions selected from, without limitation, insertion by an electrophile into a C—H bond, insertion by an electrophile into an O—H bond, insertion by an electrophile into an N—H bond, addition of the electrophile across an alkene, addition of the electrophile across an alkyne, addition to electrophilic carbonyl centers, substitution at electrophilic carbonyl centers, addition to ketenes, nucleophilic addition to isocyanates, nucleophilic addition to isothiocyanates, nucleophilic substitution at activated silicon centers, nucleophilic displacement of an alkyl halide, nucleophilic displacement at an alkyl pseudohalide, nucleophilic addition/elimination at an activated carbonyl, 1,4-conjugate addition of a nucleophile to an α, β-unsaturated carbonyl, nucleophilic ring opening of an epoxide, nucleophilic aromatic substitution of an electron deficient aromatic compound, a nucleophilic addition to activated phosphorus centers, nucleophilic substitution at activated phosphorous centers, nucleophilic addition to activated sulfur centers, and nucleophilic substitution at activated sulfur centers.

A nucleophilic conjugating moiety may be selected from optionally substituted alkenes, optionally substituted alkynes, optionally substituted aryl, optionally substituted heterocyclyl, hydroxyl groups, amino groups, alkylamino groups, anilido groups, and thio groups.

An electrophilic conjugating moiety may be selected from nitrenes, nitrene precursors such as azides, carbenes, carbene precursors, activated silicon centers, activated carbonyls, anhydrides, isocyanates, thioisocyanates, succinimidyl esters, sulfosuccinimidyl esters, maleimides, alkyl halides, alkyl pseudohalides, epoxides, episulfides, aziridines, electron-deficient aryls, activated phosphorus centers, and activated sulfur centers.

For example, conjugation can occur via a condensation reaction to form a linkage that is a hydrazone bond.

Conjugation via the formation of an amide bond can be mediated by activation of a carboxyl-based conjugating moiety and subsequent reaction with a primary amine-based conjugating moiety. Activating agents can be various carbodiimides like: EDC (1-Ethyl-3-(3-dimethylaminopropyl)carbodiimide hydrochloride), EDAC (1-ethyl-3(3-dimethylaminopropyl)carbodiimide hydrochloride), DCC (dicyclohexyl carbodiimide), CMC (1-Cyclohexyl-3-(2-morpholinoethyl) carbodiimide), DIC (diisopropyl carbodiimide) or Woodward's reagent K (N-ethyl-3-phenylisoxazolium-3′-sulfonate). Reaction of an activated NHS-Ester-based conjugating moiety with a primary amine-based conjugating moiety also results in formation of an amide bond.

The nucleotide construct may contain a carbonyl-based conjugating moiety. Conjugation via the formation of a secondary amine can be achieved by reacting an amine-based conjugating moiety with an aldehyde-based conjugating moiety, followed by reducing with a hydride donor like sodium cyanoborohydride. Aldehyde-based conjugating moieties can be introduced for instance by oxidation of sugar moieties or by reaction with SFB (succinimidyl-p-formyl benzoate) or SFPA (succinimidyl-p-formylphenoxyacetate).

Ether formation can also be used to conjugate auxiliary moieties to the nucleotide constructs of the invention. Conjugation via ether linkages can be mediated by reaction of an epoxide-based conjugating moiety with a hydroxy-based conjugating moiety.

Thiols can also be used as conjugating moieties. For example, conjugation via the formation of disulfide bonds can be accomplished by pyridyldisulfide mediated thiol-disulfide exchange. Introduction of sulfhydryl-based conjugating moieties is mediated for instance by Traut's Reagent (2-iminothiolane) SATA (N-succinimidyl S-acetylthioacetate, SATP (succinimidyl acetylthiopropionate), SPDP (N-succinimidyl 3-(2-pyridyldithio)propionate, SMPT (succinimidyloxycarbonyl-α-methyl-α-(2-pyridyldithio)toluene), N-acetylhomocysteinethiolactone, SAMSA (S-acetylmercaptosuccinic anhydride), AMBH (2-Acedamido-4-mercaptobuturic acid hydrazide), and cystamine (2,2′-dithiobis(ethylamine).

Conjugation via the formation of thioether linkages can be performed by reacting a sulfhydryl based conjugating moieties with maleimide- or iodoacetyl-based conjugating moieties or by reacting with epoxide-based conjugating moieties. Maleimide-based conjugating moieties can be introduced by SMCC (succinimidyl-4-(N-maleimidomethyl)cyclohexane-1-carboxylate), sulfo-SMCC (sulfosuccinimidyl 4-(N-maleidomethyl)-cyclohexane-1-carboxylate), MBS (m-Maleimidobenzoyl-N-hydroxysuccinimide ester), sulfo-MBS (m-Maleimidobenzoyl-N-sulfohydroxy succinimide ester), SMPB (Succinimidyl-4-(p-maleidophenyl)butyrate), sulfo-SMPB (sulfosuccinimidyl 4-(p-maleimidophenyl)butyrate), GMBS (N-α-maleimidobuturyl-oxysuccinimide ester), sulfo GMBS (N-α-maleimidobuturyl-oxysulfosuccinimide ester).

Thiol-based conjugating moieties can also react with iodoacetyl-based conjugating moieties. Iodoacetyl-based conjugating moieties can be inserted with SIAB (N-succinimidyl(4-iodoacetyl)aminobenzoate, sulfo SIAB (sulfo-succinimidyl(4-iodoacetyl)-aminobenzoate), SIAX (succinimidyl6-[(iodoacetyl-amino]hexanoate), SIAXX (succinimidyl6-[6-(((iodoacetyl)amino)-hexanoyl)amino]hexanoate), SIAC (succinimidyl 4-(((iodoacetyl)amino)methyl)-cyclohexane-1-carboxylate), SIACX (succinimidyl 6-((((4-(iodoacetyl)amino)methyl)-cyclohexane-1-carbonyl)amino) hexanoate), and NPIA (p-nitrophenyl iodoacetate).

Conjugation via the formation of a carbamate linkage can be performed by reaction of a hydroxy-based conjugating moiety with CDI (N,N′-carbonyldiimidazole) or DSC (N,N′-disuccinimidyl carbonate) or N-hydroxysuccinimidylchloroformate and subsequent reaction with an amine-based conjugating moiety.

Photolytic and Thermolytic Conjugation

Alternatively, the conjugating moiety can employ photolytic or thermolytic activation in order to form the desired covalent bond. Conjugating moieties that include azide functionality are one example. Thus, conjugation can also be achieved by the introduction of a photoreactive conjugating moiety. Photoreactive conjugating moieties are aryl azides, halogenated aryl azides, benzophenones certain diazo compounds and diazirine derivatives. They react with amino-based conjugating moieties or with conjugating moieties that have activated hydrogen bonds.

The azide-based conjugating moieties are UV labile and, upon photolysis, can lead to the formation of nitrene electrophiles that can react with nucleophilic conjugating moieties such as aryl-based conjugating moieties or alkenyl-based conjugating moieties. Alternatively, the heating of these azide compounds can also result in nitrene formation.

Cycloaddition Reactions

Cycloaddition reactions can be used to form the desired covalent bond. Representative cycloaddition reactions include, but are not limited to, the reaction of an alkene-based conjugating moiety with a 1,3-diene-based conjugating moiety (Diels-Alder reaction), the reaction of an alkene-based conjugating moiety with an α,β-unsaturated carbonyl-based conjugating moiety (hetero Diels-Alder reaction), and the reaction of an alkyne-based conjugating moiety with an azide-based conjugating moiety (Hüisgen cycloaddition). Selected, non-limiting examples of conjugating moieties that comprise reactants for cycloaddition reactions are: alkenes, alkynes, 1,3-dienes, α,β-unsaturated carbonyls, and azides. For example, the Hüisgen cycloaddition between azides and alkynes has been used for the functionalization of diverse biological entities.

Coupling Reactions

Conjugating moieties also include, but are not limited to, reactants for hydrosilylation, olefin cross-metathesis, conjugate addition, Stille coupling, Suzuki coupling, Sonogashira coupling, Hiyama coupling, and Heck reaction. Conjugation moieties for these reactions include hydridosilanes, alkenes (e.g., activated alkenes, such as enones or enoates), alkynes, aryl halides, aryl pseudohalides (e.g., triflates or nonaflates), alkyl halides, and alkyl pseudohalides (e.g., triflates, nonaflates, and phosphates). Catalysts for cross-coupling reactions are well-known in the art. Such catalysts may be organometallic complexes or metal salts (e.g., Pd(0), Pd(II), Pt(O), Pt(II), Pt(IV), Cu(I), or Ru(II)). Additives, such as ligands (e.g., PPh₃, PCy₃, BINAP, dppe, dppf, SIMes, or SIPr) and metal salts (e.g., LiCl), may be added to facilitate cross-coupling reactions.

Auxiliary Moieties for Conjugation

Various auxiliary moieties can be conjugated to the nucleotide constructs of the invention (e.g., siRNA), and the auxiliary moieties can have any number of biological or chemical effects. Biological effects include, but are not limited to, inducing intracellularization, binding to a cell surface, targeting a specific cells type, allowing endosomal escape, altering the half-life of the polynucleotide in vivo, and providing a therapeutic effect. Chemical effects include, but are not limited to, changing the solubility, charge, size, and reactivity.

Small Molecules

Small molecule-based auxiliary moieties (e.g., organic compounds having molecular weights of 1000 Da or less) can be conjugated to nucleotide constructs of the invention. Examples of such small molecules include, but are not limited to, substituted or unsubstituted alkanes, alkenes, or alkynes, e.g., hydroxy-substituted, NH₂-substituted, mono-, di-, or trialkyl amino substituted, guanidino substituted, heterocyclyl substituted, and protected versions thereof. Other small molecules include steroids (e.g., cholesterol), other lipids, bile, and amino acids. A small molecule may be added to a polynucleotide to provide neutral or positive charge or to alter the hydrophilicity or hydrophobicity of the polynucleotide.

Polypeptides

A polypeptide (including a fusion polypeptide) refers to a polymer in which the monomers are amino acid residues which are joined together through amide bonds. When the amino acids are alpha-amino acids, either the L-optical isomer or the D-optical isomer can be used. A polypeptide encompasses an amino acid sequence and includes modified sequences such as glycoproteins, retro-inverso polypeptides, D-amino acid and the like. A polypeptide includes naturally occurring proteins, as well as those which are recombinantly or synthetically synthesized. A polypeptide may comprise more than one domain have a function that can be attributed to the particular fragment or portion of a polypeptide. A domain, for example, includes a portion of a polypeptide which exhibits at least one useful epitope or functional domain. Two or more domains may be functionally linked such that each domain retains its function yet comprises a single peptide or polypeptide (e.g., a fusion polypeptide). For example, a functional fragment of a PTD includes a fragment which retains transduction activity. Biologically functional fragments, for example, can vary in size from a fragment as small as an epitope capable of binding an antibody molecule, to a large polypeptide capable of participating in the characteristic induction or programming of phenotypic changes within a cell.

In some embodiments, retro-inverso polypeptides are used. “Retro-inverso” means an amino-carboxy inversion as well as enantiomeric change in one or more amino acids (i.e., levorotatory (L) to dextrorotatory (D)). A polypeptide of the invention encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995). The overall structural features of a retro-inverso polypeptide are similar to those of the parent L-polypeptide. The two molecules, however, are roughly mirror images because they share inherently chiral secondary structure elements. Main-chain peptidomimetics based on peptide-bond reversal and inversion of chirality represent important structural alterations for peptides and proteins, and are highly significant for biotechnology. Antigenicity and immunogenicity can be achieved by metabolically stable antigens such as all-D- and retro-inverso-isomers of natural antigenic peptides and polypeptide. Several PTD-derived peptidomimetics are provided herein.

Polypeptides and fragments can have the same or substantially the same amino acid sequence as the naturally derived polypeptide or domain. “Substantially identical” means that an amino acid sequence is largely, but not entirely, the same, but retains a functional activity of the sequence to which it is related. An example of a functional activity is that the fragment is capable of transduction, or capable of binding to an RNA. For example, fragments of full length TAT are described herein that have transduction activity. In general two peptides, polypeptides or domains are “substantially identical” if their sequences are at least 85%, 90%, 95%, 98% or 99% identical, or if there are conservative variations in the sequence. A computer program, such as the BLAST program (Altschul et al., 1990) can be used to compare sequence identity.

A polypeptide of the invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the backbone, the amino acid side-chains and the amino or carboxyl termini. It will be appreciated that the same type of modification may be present in the same or varying degrees at several sites in a given peptide or polypeptide. Also, a given polypeptide may contain many types of modifications. A polypeptide may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann N.Y. Acad Sci 663:48-62 (1992)).

A polypeptide domain or a fusion polypeptide of the invention can be synthesized by commonly used methods such as those that include t-BOC or FMOC protection of alpha-amino groups. Both methods involve stepwise synthesis in which a single amino acid is added at each step starting from the C-terminus of the peptide or polypeptide (See, Coligan, et al., Current Protocols in Immunology, Wiley Interscience, 1991, Unit 9). Polypeptides of the invention can also be synthesized by the well known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp. 27-62, using a copoly(styrene-divinylbenzene) containing 0.1-1.0 mMol amines/g polymer. On completion of chemical synthesis, the polypeptides can be deprotected and cleaved from the polymer by treatment with liquid HF-10% anisole for about ¼-1 hours at 0° C. After evaporation of the reagents, the polypeptides are extracted from the polymer with a 1% acetic acid solution, which is then lyophilized to yield the crude material. The polypeptides can be purified by such techniques as gel filtration on Sephadex G-15 using 5% acetic acid as a solvent. Lyophilization of appropriate fractions of the column eluate yield homogeneous peptide or polypeptide, which can then be characterized by standard techniques such as amino acid analysis, thin layer chromatography, high performance liquid chromatography, ultraviolet absorption spectroscopy, molar rotation, or measuring solubility. If desired, the polypeptides can be quantified by the solid phase Edman degradation.

Carbohydrates

Carbohydrate-based auxiliary moieties that can be attached to the nucleotide constructs of the invention include monosaccharides, disaccharides, and polysaccharides. Examples include allose, altrose, arabinose, cladinose, erythrose, erythrulose, fructose, D-fucitol, L-fucitol, fucosamine, fucose, fuculose, galactosamine, D-galactosaminitol, N-acetyl-galactosamine, galactose, glucosamine, N-acetyl-glucosamine, glucosaminitol, glucose, glucose-6-phosphate gulose glyceraldehyde, L-glycero-D-mannos-heprose, glycerol, glycerone, gulose idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tararic acid, threose, xylose and xylulose. A monosaccharide can be in D- or L-configuration. A monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), a imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides, such as galactosamine, glucosamine, mannosamine, fucosmine, quinavosamine, neuraminic acid, muramic acid, lactosediamine, acosamine, bacillosamine, daunosamine, desosamine, forosamine, garosamine, kanosamine, kanosamine, mycaminose, myosamine, persosamine, pneumosamine, purpurosamine, rhodosmine. It is understood that the monosaccharide and the like can be further substituted. Di- and polysaccharides include abequose, acrabose, amicetose, amylopectin, amylose, apiose, arcanose, ascarylose, ascorbic acid, boivinose, cellobiose, cellotriose, cellulose, chacotriose, chalcose, chitin, colitose, cyclodextrin, cymarose, dextrin, 2-deoxyribose, 2-deoxyglucose diginose, digitalose, digitoxose, evalose, evemitrose, fructooligosaccharide, galto-oligosaccharide, gentianose, genitiobiose, glucan, gluicogen, glycogen, hamamelose, heparin, inulin, isolevoglucosenone, isomaltose, isomaltotriose, isopanose, kojibiose, lactose, lactosamine, lactosediamine, laminarabiose, levoglucosan, levoglucosenone, β-maltose, maltriose, mannan-oligosaccharide, manninotriose, melezitose, melibiose, muramic acid, mycarose, mycinose, neuraminic acid, migerose, nojirimycon, noviose, oleandrose, panose, paratose, planteose, primeverose, raffinose, rhodone, rutinose, oleandrose, panose, paratose, planteose, primeverose, raffinose, rhodinose, rutinose, sarmentose, sedoheptulose, sedoheptulosan, solatriose, sophorose, stachyose, streptose, sucrose, α,α-trehalose, trahalosamine, turanose, tyvelose, xylobiose, umbelliferose and the like.

Polymers

The nucleotide constructs described herein can also comprise covalently attached neutral or charged (e.g., cationic) polymer-based auxiliary moieties. Examples of positively charged polymers include poly(ethylene imine) (PEI), spermine, spermidine, and poly(amidoamine) (PAMAM). Neutral polymers include poly(C₁₋₆ alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(acrylic acid), poly(ethyloxazoline), poly(alkylacrylates), poly(acrylamide), poly(N-alkylacrylam ides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, poly(2-ethylacrylic acid), N-isopropylacrylamide polymers, polyphosphazine and poly(N,N-dialkylacrylamides). Exemplary polymer auxiliary moieties may have molecular weights of less than 100, 300, 500, 1000, or 5000. Other polymers are known in the art.

Therapeutic Agents

Therapeutic agents, which include diagnostic/imaging agents, can be covalently attached as auxiliary moieties to the nucleotide constructs of the invention or can be administered as a co-therapy as described herein. They can be naturally occurring compounds, synthetic organic compounds, or inorganic compounds. Exemplary therapeutic agents include, but are not limited to, antibiotics, antiproliferative agents, rapamycin macrolides, analgesics, anesthetics, antiangiogenic agents, vasoactive agents, anticoagulants, immunomodulators, cytotoxic agents, antiviral agents, antithrombotic drugs, antibodies, neurotransmitters, psychoactive drugs, and combinations thereof. Additional examples of therapeutic agents include, but are not limited to, cell cycle control agents; agents which inhibit cyclin protein production; cytokines, including, but not limited to, Interleukins 1 through 13 and tumor necrosis factors; anticoagulants, anti-platelet agents; TNF receptor domains and the like. Typically the therapeutic agent is neutral or positively charged. In certain instances, where the therapeutic agent is negatively charged, an additional charge neutralization moiety (e.g., a cationic peptide) can be used.

A therapeutic moiety can be linked as an auxiliary moiety to a nucleotide construct disclosed herein to allow for diagnostic assay/imaging. Examples of such moieties include, but are not limited to, detectable labels, such as an isotope, a radioimaging agent, a marker, a tracer, a fluorescent label (e.g., rhodamine), and a reporter molecule (e.g., biotin).

Exemplary diagnostic agents include, but are not limited to, imaging agents, such as those that are used in positron emission tomography (PET), computer assisted tomography (CAT), single photon emission computerized tomography, X-ray, fluoroscopy, and magnetic resonance imaging (MRI). Suitable materials for use as contrast agents in MRI include, but are not limited to, gadolinium chelates, as well as iron, magnesium, manganese, copper, and chromium chelates. Examples of materials useful for CAT and X-rays include, but are not limited to, iodine based materials.

Examples of radioimaging agents emitting radiation (detectable radio-labels) that may be suitable are exemplified by indium-111, technitium-99, or low dose iodine-131. Detectable labels, or markers, for use in conjunction with or attached to the nucleotide constructs of the invention as auxiliary moieties may be a radiolabel, a fluorescent label, a nuclear magnetic resonance active label, a luminescent label, a chromophore label, a positron emitting isotope for PET scanner, a chemiluminescence label, or an enzymatic label. Fluorescent labels include, but are not limited to, green fluorescent protein (GFP), fluorescein, and rhodamine. The label may be for example a medical isotope, such as for example and without limitation, technetium-99, iodine-123 and -131, thallium-201, gallium-67, fluorine-18, indium-111, etc.

Other therapeutic agents known in the art can likewise be used in conjunction with, or attached to the nucleotide constructs of the invention as auxiliary moieties.

Targeting Moieties

The invention provides for one or more targeting moieties which can be attached to a nucleotide construct disclosed herein as an auxiliary moiety, for example as a targeting auxiliary moiety. A targeting moiety (e.g., extracellular targeting moiety) is selected based on its ability to target constructs of the invention to a desired or selected cell population that expresses the corresponding binding partner (e.g., either the corresponding receptor or ligand) for the selected targeting moiety. For example, a construct of the invention could be targeted to cells expressing epidermal growth factor receptor (EGFR) by selected epidermal growth factor (EGF) as the targeting moiety. Alternatively, the targeting moiety (e.g., intracellular targeting moiety) can target constructs of the invention to a desired site within the cell (e.g., endoplasmic reticulum, Golgi apparatus, nucleus, or mitochondria). Non-limiting examples of the intracellular targeting moieties include compounds P38 and P39 of Table 3 and peptide fragments thereof (i.e., MKWVTFISLLFLFFSSAYS and MIRTLLLSTLVAGALS, respectively).

A polynucleotide construct of the invention, thus, may include one or more targeting moieties selected from the group consisting of intracellular targeting moieties, extracellular targeting moieties, and combinations thereof. Thus, the inclusion of one or more extracellular targeting moieties (e.g., each extracellular targeting moiety independently selected from the group consisting of folate, mannose, galactosamine (e.g., N-acetyl galactosamine), and prostate specific membrane antigen) and one or more intracellular targeting moiety (e.g., a moiety targeting endoplasmic reticulum, Golgi apparatus, nucleus, or mitochondria) in the polynucleotide construct of the invention can facilitate the delivery of the polynucleotides to a specific site within the specific cell population.

Some of the extracellular targeting moieties of the invention are described herein. In one embodiment, the targeting moiety is a receptor binding domain. In another embodiment, the targeting moiety is or specifically binds to a protein selected from the group comprising insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressing cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof. In further embodiments, the targeting moiety is erythroblastic leukemia viral oncogene homolog (ErbB) receptor (e.g., ErbB1 receptor; ErbB2 receptor; ErbB3 receptor; and ErbB4 receptor). In other embodiments, a targeting moiety may selectively bind to asialoglycoprotein receptor, a manno receptor, or a folate receptor. In particular embodiments, the targeting moiety contains one or more N-acetyl galactosamines (GalNAc), mannoses, or a folate ligand. In certain embodiments, the folate ligand has the structure:

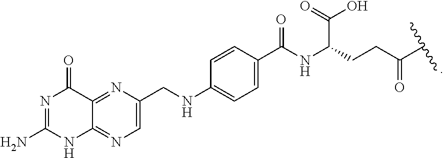 [SMILES: *C(=O)CC[C@H](NC(=O)c1ccc(NCc2cnc3[nH]c(N)nc(=O)c3n2)cc1)C(=O)O]

The targeting moiety can also be selected from bombesin, gastrin, gastrin-releasing peptide, tumor growth factors (TGF), such as TGF-α and TGF-β, and vaccinia virus growth factor (VVGF). Non-peptidyl ligands can also be used as the targeting moiety and may include, for example, steroids, carbohydrates, vitamins, and lectins. The targeting moiety may also be selected from a polypeptide, such as somatostatin (e.g., a somatostatin having the core sequence cyclo[Cys-Phe-D-Trp-Lys-Thr-Cys], and in which, for example, the C-terminus of the somatostatin analog is: Thr-NH₂), a somatostatin analog (e.g., octreotide and lanreotide), bombesin, a bombesin analog, or an antibody, such as a monoclonal antibody.

Other peptides or polypeptides for use as a targeting auxiliary moiety in nucleotide constructs of the invention can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, peptides from complementarity determining regions of an antitumor antibody, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK)), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

Immunoreactive ligands for use as a targeting moiety in nucleotide constructs of the invention include an antigen-recognizing immunoglobulin (also referred to as “antibody”), or antigen-recognizing fragment thereof. As used herein, “immunoglobulin” refers to any recognized class or subclass of immunoglobulins such as IgG, IgA, IgM, IgD, or IgE. Typical are those immunoglobulins which fall within the IgG class of immunoglobulins. The immunoglobulin can be derived from any species. Typically, however, the immunoglobulin is of human, murine, or rabbit origin. In addition, the immunoglobulin may be polyclonal or monoclonal, but is typically monoclonal.

Targeting moieties of the invention may include an antigen-recognizing immunoglobulin fragment. Such immunoglobulin fragments may include, for example, the Fab′, F(ab′)₂, Fᵥ or Fab fragments, single-domain antibody, ScFv, or other antigen-recognizing immunoglobulin fragments. Fc fragments may also be employed as targeting moieties. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art. See Parham, J. Immunology, 131, 2895, 1983; Lamoyi et al., J. Immunological Methods, 56, 235, 1983.

Targeting moieties of the invention include those targeting moieties which are known in the art but have not been provided as a particular example in this disclosure.

Endosomal Escape

The invention provides for one or more endosomal escape moieties which can be attached to a nucleotide construct disclosed herein as an auxiliary moiety, for example, as an endosomal escape auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; peptides or polypeptides such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Exemplary endosomal escape moieties are provided in Table 3 in Example 1.

Delivery Domain

The invention provides for one or more delivery domain moieties which can be attached to a nucleotide construct disclosed herein as an auxiliary moiety, for example as an delivery domain auxiliary moiety. A delivery domain is a moiety that induces transport of a polynucleotide of the invention into a cell, by any mechanism. Typically, nucleotide constructs of the invention will be internalized by macropinocytosis, phagocytosis, or endocytosis (e.g., clathrin-mediated endocytosis, caveolae-mediated endocytosis, and lipid-raft dependent endocytosis), see, e.g., Chem. Soc. Rev., 2011, 40, 233-245. Delivery domains may include peptides or polypeptides (e.g., peptide transduction domains), carbohydrates (hyaluronic acid), and positively charged polymers (poly(ethylene imine), as described herein.

Cellular delivery can be accomplished by macromolecule fusion of “cargo” biological agents (in this case the polynucleotide) to a cationic Peptide Transduction Domain (PTD; also termed Cell Penetrating Peptide (CPP)) such as TAT or (Arg₈) (Snyder and Dowdy, 2005, Expert Opin. Drug Deliv. 2, 43-51). PTDs can be used to deliver a wide variety of macromolecular cargo, including the polynucleotides described herein (Schwarze et al., 1999, Science 285, 1569-1572; Eguchi et al., 2001, J. Biol. Chem. 276, 26204-26210; and Koppelhus et al., 2002, Antisense Nucleic Acid Drug Dev. 12, 51-63). Cationic PTDs enter cells by macropinocytosis, a specialized form of fluid phase uptake that all cells perform.

Biophysical studies on model vesicles suggest that cargo escape from macropinosome vesicles into the cytoplasm, thus requiring a pH decrease (Magzoub et al., 2005, Biochemistry 44, 14890-14897). The cationic charge of the PTDs is essential for the molecules to traverse the cell membrane. Not surprisingly, conjugation of cationic PTDs (6-8 positive charges) to anionic siRNAs (˜40 negative charges) results in charge neutralization and inactivation of the PTD with no siRNA entering the cells (Turner et al., Blood Cells Mol. Dis., 38(1):1-7, 2007). However, chemical conjugation of cationic PTDs to a nucleotide construct described herein (e.g., anionic RNA or DNA) still results in the nucleotide construct being able to be taken up by cells, and therefore the novel and nonobvious nucleotide constructs disclosed herein do not suffer from any charge neutralization deleterious artifacts seen with other similar methods. Further, cleavage of these PTDs intracellularly allows the polynucleotide to be irreversibly delivered to the targeted cell.

The discovery of several proteins which could efficiently pass through the plasma membrane of eukaryotic cells has led to the identification of a class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Exemplary PTD sequences are provided in Table 1. The invention further provides for one or more of the PTDs listed in Table 1 or other PTDs known in the art (see, e.g., Joliot et al., Nature Cell Biology, 6(3):189-196, 2004) to be conjugated to the nucleotide constructs disclosed herein as auxiliary moieties. Strategies for conjugation include the use of a bifunctional linker that includes a functional group that can be cleaved by the action of an intracellular enzyme.

TABLE 1

| PTD | Sequence | SEQ ID NO. |
|---|---|---|
| TAT | RKKRRQRRR | SEQ ID NO.: 1 |
| Penetratin | RQIKIWFQNRRMKWKK | SEQ ID NO.: 2 |
| Buforin II | TRSSRAGLQFPVGRVHRLLRK | SEQ ID NO.: 3 |
| Transportan | GWTLNSAGYLLGKINKALAAL | SEQ ID NO.: 4 |
| | AKKIL | |
| MAP (model | KLALKLALKALKAALKLA | SEQ ID NO.: 5 |
| amphipathic | | |
| peptide) | | |
| K-FGF | AAVALLPAVLLALLAP | SEQ ID NO.: 6 |
| Ku70 | VPMLK-PMLKE | SEQ ID NO.: 7 |
| Prion | MANLGYWLLALFVTMWTDVGL | SEQ ID NO.: 8 |
| | CKKRPKP | |
| pVEC | LLIILRRRIRKQAHAHSK | SEQ ID NO.: 9 |
| Pep-1 | KETWWETWWTEWSQPKKKRKV | SEQ ID NO.: 10 |
| SynB1 | RGGRLSYSRRRFSTSTGR | SEQ ID NO.: 11 |
| Pep-7 | SDLWEMMMVSLACQY | SEQ ID NO.: 12 |
| (phage | | |
| display) | | |
| HN-1 | TSPLNIHNGQKL | SEQ ID NO.: 13 |
| (phage | | |
| display) | | |

Exemplary auxiliary moieties which comprise TAT peptides that can be conjugated to any of the nucleotide constructs described herein are provided in Table 2.

TABLE 2
Sequence (N′ to C′)

PEG-(PTD)
GG-(PTD)-PEG-(PTD)
PEG-(PTD)-PEG-(PTD)
GG-(PTD)-PEG-PEG-PEG-(PTD)
PEG-(PTD)-PEG-PEG-PEG-(PTD)
GG-(PTD)-PEG-(PTD)-PEG-(PTD)
GG-(PTD)-PEG-PEG-PEG-(PTD)-PEG-PEG-PEG-(PTD)

PEG = a poly(ethyleneglycol) linker having six repeat units

In a particular embodiment, the auxiliary moieties described in Table 2 include a covalent bond to Z′ at the N′ terminus, where Z′ is the residue of conjugation of 6-hydrazinonicotinic acid (HyNic) or an amino group of a polypeptide R^{z} to an aldehyde.

Further exemplary cationic PTD (CPP) sequences are provided in Table 3 in Example 1.

Thus, PTDs that can be conjugated to a nucleotide construct of the invention include, but are not limited to, AntHD, TAT, VP22, cationic prion protein domains, and functional fragments thereof. Not only can these peptides pass through the plasma membrane, but the attachment of other peptide or polypeptides, such as the enzyme β-galactosidase, are sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type, and these proteins can efficiently transduce ˜100% of cells in culture with no apparent toxicity (Nagahara et al., Nat. Med. 4:1449-52, 1998). In addition to full-length proteins, peptide transduction domains have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is considerable flexibility in particle size in this process.

In a particular embodiment, the invention therefore provides methods and compositions that combine the use of PTDs, such as TAT and poly-Arg, with a nucleotide construct disclosed herein to facilitate the targeted uptake of the construct into and/or release within targeted cells. Nucleotide constructs disclosed herein therefore provide methods whereby a therapeutic or diagnostic agent which is linked as an auxiliary moiety can be targeted to be delivered in certain cells by the nucleotide constructs further comprising one or more PTDs linked as auxiliary moieties.

The nucleotide construct of the invention can be an siRNA or other inhibitory nucleic acid sequence that itself provides a therapeutic or diagnostic benefit. However, in some instances it may be desirable to attach additional auxiliary moieties as therapeutics or to promote uptake. In the case of PTDs, the PTDs serve as additional charge modifying moieties to promote uptake of the nucleotide construct by neutralizing the charge on the nucleotide construct or typically providing a slight net cationic charge to the nucleotide construct. It will be further understood, that the nucleotide construct may include other auxiliary moieties such as, but not limited to, targeting moieties, biologically active molecules, therapeutics, small molecules (e.g., cytotoxics), and the like. In such instances the nucleotide construct having such auxiliary moieties may be neutrally charged or cationically charged depending upon the auxiliary moieties size and charge. In instances where the auxiliary moieties are anionically charged the addition of cationically charged peptides (e.g., PTDs) can further neutralize the charge or improve the net cationic charge of the construct.

In general, the delivery domain that is linked to a nucleotide construct disclosed herein can be nearly any synthetic or naturally-occurring amino acid sequence which assists in the intracellular delivery of a nucleic construct disclosed herein into targeted cells. For example, transfection can be achieved in accordance with the invention by use of a peptide transduction domain, such as an HIV TAT protein or fragment thereof, that is covalently linked to a conjugating moiety of a nucleotide construct of the invention. Alternatively, the peptide transduction domain can comprise the Antennapedia homeodomain or the HSV VP22 sequence, the N-terminal fragment of a prion protein or suitable transducing fragments thereof such as those known in the art.

The type and size of the PTD will be guided by several parameters including the extent of transfection desired. Typically the PTD will be capable of transfecting at least about 20%, 25%, 50%, 75%, 80% or 90%, 95%, 98% and up to, and including, about 100% of the cells. Transfection efficiency, typically expressed as the percentage of transfected cells, can be determined by several conventional methods.

PTDs will manifest cell entry and exit rates (sometimes referred to as k₁ and k₂, respectively) that favor at least picomolar amounts of a nucleotide construct disclosed herein into a targeted cell. The entry and exit rates of the PTD and any cargo can be readily determined or at least approximated by standard kinetic analysis using detectably-labeled fusion molecules. Typically, the ratio of the entry rate to the exit rate will be in the range of between about 5 to about 100 up to about 1000.

In one embodiment, a PTD useful in the methods and compositions of the invention comprises a polypeptide featuring substantial alpha-helicity. It has been discovered that transfection is optimized when the PTD exhibits significant alpha-helicity. In another embodiment, the PTD comprises a sequence containing basic amino acid residues that are substantially aligned along at least one face of the peptide or polypeptide. A PTD domain useful in the invention may be a naturally occurring peptide or polypeptide or a synthetic peptide or polypeptide.

In another embodiment, the PTD comprises an amino acid sequence comprising a strong alpha helical structure with arginine (Arg) residues down the helical cylinder.

In yet another embodiment, the PTD domain comprises a polypeptide represented by the following general formula: B_{P1}-X_{P1}-X_{P2}-X_{P3}-B_{P2}-X_{P4}-X_{P5}-B_{P3} (SEQ ID NO:14) wherein B_{P1}, B_{P2}, and B_{P3} are each independently a basic amino acid, the same or different; and X_{P1}, X_{P2}, X_{P3}, X_{P4}, and X_{P5} are each independently an alpha-helix enhancing amino acid, the same or different.

In another embodiment, the PTD domain is represented by the following general formula: B_{P1}-X_{P1}-X_{P2}-B_{P2}-B_{P3}-X_{P3}-X_{P4}-B_{P4} (SEQ ID NO:15) wherein B_{P1}, B_{P2}, B_{P3}, and B_{P4} are each independently a basic amino acid, the same or different; and X_{P1}, X_{P2}, X_{P3}, and X_{P4} are each independently an alpha-helix enhancing amino acid the same or different.

Additionally, PTD domains comprise basic residues, e.g., lysine (Lys) or arginine (Arg), and further can include at least one proline (Pro) residue sufficient to introduce “kinks” into the domain. Examples of such domains include the transduction domains of prions. For example, such a polypeptide comprises KKRPKPG (SEQ ID NO:16).

In one embodiment, the domain is a polypeptide represented by the following sequence: X_{P}-X_{P}-R-X_{P}-(P/X_{P})-(B_{P}/X_{P})-B_{P}-(P/X_{P})-X_{P}-B_{P}-(B_{P}/X_{P}) (SEQ ID NO:17), wherein X is any alpha helical promoting residue such as alanine; P/X_{P} is either proline or X_{P} as previously defined; B_{P} is a basic amino acid residue, e.g., arginine (Arg) or lysine (Lys); R is arginine (Arg) and B_{P}/X_{P} is either B_{P} or X_{P} as defined above.

In another embodiment the PTD is cationic and consists of between 7 and 10 amino acids and has the formula KX_{P1}RX_{P2}X_{P1} (SEQ ID NO:18), wherein X_{P1} is R or K and X_{P2} is any amino acid. An example of such a polypeptide comprises RKKRRQRRR (SEQ ID NO:1). In another example, the PTD is a cationic peptide sequence having 5-10 arginine (and/or lysine) residues over 5-15 amino acids.

Additional delivery domains in accord with this disclosure include a TAT fragment that comprises at least amino acids 49 to 56 of TAT up to about the full-length TAT sequence (see, e.g., SEQ ID NO:1). A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment that impede alpha helix formation or stability. In a more specific embodiment, the TAT fragment will include at least one amino acid substitution with an alpha-helix enhancing amino acid. Typically the TAT fragment will be made by standard peptide synthesis techniques although recombinant DNA approaches may be used in some cases. In one embodiment, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence are substantially aligned along at least one face of that sequence.

Additional transduction proteins (PTDs) that can be used in the compositions and methods of the invention include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence.

Also included are chimeric PTD domains. Such chimeric PTDs include parts of at least two different transducing proteins. For example, chimeric PTDs can be formed by fusing two different TAT fragments, e.g., one from HIV-1 and the other from HIV-2 or one from a prion protein and one from HIV.

A PTD can be linked as an auxiliary moiety to a nucleotide construct of the invention using phosphoramidate or phosphotriester linkers at an internucleotide bridging group or at the 3′ or 5′ ends. For example, a siRNA construct comprising a 3′-amino group with a 3-carbon linker may be utilized for linking the siRNA construct to a PTD. The siRNA construct may be conjugated to the PTD via a heterobifunctional cross linker.

The PTD can be attached as an auxiliary moiety to a nucleotide construct via a bioreversible group, whereby the bioreversible group can be cleaved intracellularly, e.g., by an intracellular enzyme (e.g., protein disulfide isomerase, thioredoxin, or a thioesterase) and thereby release the polynucleotide.

For example, in addition to the PTD being conjugated between the 5′ and 3′ ends, a PTD can be conjugated directly to a polynucleotide (e.g., an RNA or DNA) comprising a nucleotide construct disclosed herein, at the 5′ and/or 3′ end via a free thiol group. For example, a PTD can be linked to the polynucleotide by a disulfide linkage. This approach can be applied to any polynucleotide length and will allow for delivery of the polynucleotide (e.g., siRNA) into cells. The polynucleotide can also include, for example, one or more delivery domains and/or a protecting group that contains a basic group. Once inside the cell the polynucleotide reverts to an unprotected polynucleotide based on the intracellular conditions, e.g., reducing environment, by hydrolysis or other enzymatic activity (e.g., protein disulfide isomerase, thioredoxin, or thioesterase activity).

Peptide linkers that can be used in the constructs and methods of the invention will typically comprise up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the nucleic acid. For example, the peptide linker sequence can be positioned between the peptide transduction domain and the nucleic acid domain, e.g., to provide molecular flexibility. The length of the linker moiety is chosen to optimize the biological activity of the peptide or polypeptide comprising, for example, a PTD domain fusion construct and can be determined empirically without undue experimentation. Examples of linker moieties are -Gly-Gly-, GGGGS (SEQ ID NO:19), (GGGGS)_{N} (SEQ ID NO:20), GKSSGSGSESKS (SEQ ID NO:21), GSTSGSGKSSEGKG (SEQ ID NO:22), GSTSGSGKSSEGSGSTKG (SEQ ID NO:23), GSTSGSGKPGSGEGSTKG (SEQ ID NO:24), or EGKSSGSGSESKEF (SEQ ID NO:25). Peptide or polypeptide linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide or polypeptide linkers are those described in U.S. Pat. Nos. 4,751,180 and 4,935,233, which are hereby incorporated by reference.

### Pharmaceutical Compositions

Delivery of a nucleotide construct of the invention can be achieved by contacting a cell with the construct using a variety of methods known to those of skill in the art. In particular embodiments, a nucleotide construct of the invention is formulated with various carriers, dispersion agents and the like, as are described more fully elsewhere herein.

A pharmaceutical composition according to the invention can be prepared to include a nucleotide construct disclosed herein, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington: The Science and Practice of Pharmacy, 21^{st} Ed., Gennaro, Ed., Lippencott Williams & Wilkins (2005), and The United States Pharmacopeia: The National Formulary (USP 36 NF31), published in 2013; the contents of which are hereby incorporated by reference. The pH and exact concentration of the various components of the pharmaceutical composition are adjusted according to routine skills in the art. See Goodman and Gilman's, The Pharmacological Basis for Therapeutics.

The pharmaceutical compositions according to the invention may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, transdermal application, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a pharmaceutically acceptable carrier is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the invention are related to the characteristics of the pharmaceutical composition and the particular therapeutic effect to be achieve. The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

For topical formulations, the base composition can be prepared with any solvent system, such as those Generally Regarded as Safe (GRAS) by the U.S. Food & Drug Administration (FDA). GRAS solvent systems include many short chain hydrocarbons, such as butane, propane, n-butane, or a mixture thereof, as the delivery vehicle, which are approved by the FDA for topical use. The topical compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805, U.S. Pat. No. 4,980,378, U.S. Pat. No. 5,082,866, U.S. Pat. No. 6,118,020 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

Topical compositions can be provided in any useful form. For example, the compositions of the invention may be formulated as solutions, emulsions (including microemulsions), suspensions, creams, foams, lotions, gels, powders, balm, or other typical solid, semi-solid, or liquid compositions used for application to the skin or other tissues where the compositions may be used. Such compositions may contain other ingredients typically used in such products, such as colorants, fragrances, thickeners, antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetration enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals.

In some formulations, the composition is formulated for ocular application. For example, a pharmaceutical formulation for ocular application can include a polynucleotide construct as described herein in an amount that is, e.g., up to 99% by weight mixed with a physiologically acceptable ophthalmic carrier medium such as water, buffer, saline, glycine, hyaluronic acid, mannitol, and the like. For ophthalmic delivery, a polynucleotide construct as described herein may be combined with ophthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the polynucleotide construct in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the invention to improve the retention of the compound.

Topical compositions can be delivered to the surface of the eye, e.g., one to four times per day, or on an extended delivery schedule such as daily, weekly, bi-weekly, monthly, or longer, according to the routine discretion of a skilled clinician. The pH of the formulation can range from about pH 4-9, or about pH 4.5 to pH 7.4.

For nucleotide constructs of the invention, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

While the nucleotide constructs described herein may not require the use of a carrier for delivery to the target cell, the use of carriers may be advantageous in some embodiments. Thus, for delivery to the target cell, the nucleotide construct of the invention can non-covalently bind a carrier to form a complex. The carrier can be used to alter biodistribution after delivery, to enhance uptake, to increase half-life or stability of the polynucleotide (e.g., improve nuclease resistance), and/or to increase targeting to a particular cell or tissue type.

Exemplary carriers include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition of the invention in combination with a nucleotide construct of the invention.

### Intracellular Activity of Nucleotide Constructs

The invention provides compositions and methods for delivering nucleotide constructs disclosed herein (e.g., RNA, DNA, nucleic acids comprising modified bases, other anionic nucleic acids, and the like). The invention therefore provides methods and compositions useful for delivery of non-coding nucleotide constructs that exert a regulating effect on gene or protein expression.

Polynucleotide constructs of the invention may be single stranded or double stranded. When double stranded, one or both strands may include one or more bioreversible groups. When the polynucleotide acts as siRNA, the passenger strand may include a group that is irreversibly bound to an internucleotide bridging group, e.g., a C₁₋₆ alkyl phosphotriester. Typically, such a group is located after the first or second nucleotide from the 3′ end. The irreversible group prevents the passenger strand from acting as a guide strand and thereby prevents or reduces possible off-target effects.

RNA interference (RNAi) is the process whereby messenger RNA (mRNA) is degraded by small interfering RNA (siRNA) derived from double-stranded RNA (dsRNA) containing an identical or very similar nucleotide sequence to that of a target gene to be silenced. This process prevents the production of a protein encoded by the targeted gene through post-transcriptional, pre-translational manipulation. Accordingly, silencing of dominant disease genes or other target genes can be accomplished.

In vivo RNAi proceeds by a process in which the dsRNA is cleaved into short interfering RNAs (siRNAs) by an enzyme called Dicer, a dsRNA endoribonuclease, (Bernstein et al., 2001; Hamilton & Baulcombe, 1999, Science 286: 950; Meister and Tuschl, 2004, Nature 431, 343-9), thus producing multiple molecules from the original single dsRNA. siRNAs are loaded into the multimeric RNAi Silencing Complex (RISC) resulting in both catalytic activation and mRNA target specificity (Hannon and Rossi, Nature 431, 371-378, 2004; Novina and Sharp, Nature 430, 161-164, 2004). During siRNA loading into RISC, the antisense or guide strand is separated from the siRNA and remains docked in Argonaute-2 (Ago2), the RISC catalytic subunit (Leuschner et al., EMBO Rep. 7, 314-320, 2006). Certain cellular compartments, such as endoplasmic reticulum (ER), Golgi apparatus, ER-Golgi intermediate compartment (ERGIC), P-bodies, and early endosomes are enriched in Ago2. mRNAs exported from the nucleus into the cytoplasm are thought to pass through activated RISCs prior to ribosomal arrival, thereby allowing for directed, post-transcriptional, pre-translational regulation of gene expression. In theory, each and every cellular mRNA can be regulated by induction of a selective RNAi response.

The ability of 21-23 bp siRNAs to efficiently induce an RNAi response in mammalian cells is now routine (Sontheimer, Nat. Rev. Mol. Cell. Biol. 6, 127-138, 2005). The IC₅₀ for siRNAs is in the 10-100 pM range, significantly below the best drugs with IC₅₀ values in the 1-10 nM range. Consequently, due to its exquisite selectivity, RNAi has become a corner-stone for directed manipulation of cellular phenotypes, mapping genetic pathways, discovering and validating therapeutic targets, and has significant therapeutic potential.

Aspects of RNAi include (1) dsRNA, rather than single-stranded antisense RNA, is the interfering agent; (2) the process is highly specific and is remarkably potent (only a few dsRNA molecules per cell are required for effective interference); (3) the interfering activity (and presumably the dsRNA) can cause interference in cells and tissues far removed from the site of introduction. However, effective delivery of dsRNA is difficult. For example, a 21 bp dsRNA with a molecular weight of 13,860 Daltons cannot traverse the cell membrane to enter the cytoplasm, due to (1) the size and (2) the extremely negative (acidic) charge of the RNA. The methods and compositions provided by the invention enable the delivery of nucleotide constructs, such as dsRNA, into a cell through charge neutralization and improved uptake.

dsRNA comprising siRNA sequences that are complementary to a nucleotide sequence of the target gene can be prepared in any number of methods. Methods and techniques for identifying siRNA sequences are known in the art. The siRNA nucleotide sequence can be obtained from the siRNA Selection Program, Whitehead Institute for Biomedical Research, Massachusetts Institute of Technology, Cambridge, Mass. (currently available at http:[//]jura.wi.mit.edu/bioc/siRNAext/; note that brackets have been added to remove hyperlinks) after supplying the Accession Number or GI number from the National Center for Biotechnology Information website (available on the World Wide Web at ncbi.nlm.nih.gov). Alternatively, dsRNA containing appropriate siRNA sequences can be ascertained using the strategy of Miyagishi and Taira (2003). Commercially available RNAi designer algorithms also exist (http:[//]rnaidesigner.invitrogen.com/rnaiexpress/). Preparation of RNA to order is commercially available.

Nucleotide constructs of the invention may also act as miRNA to induce cleavage of mRNA. Alternatively, nucleotide constructs of the invention may act as antisense agents to bind to mRNA, either to induce cleavage by RNase or to sterically block translation.

Exemplary methods by which the nucleotide constructs of the invention can be transported into a cell are described herein.

### Therapeutic Methods

Various diseases and disorders can be treated using nucleotide constructs of the invention. For example, growth of tumor cells can be inhibited, suppressed, or destroyed upon delivery of an anti-tumor siRNA. For example, an anti-tumor siRNA can be an siRNA targeted to a gene encoding a polypeptide that promotes angiogenesis. Various angiogenic proteins associated with tumor growth are known in the art. The nucleotide constructs described herein can therefore be used in the treatment of diseases such as anti-proliferative disorders (e.g., cancer), virus infections, and genetic diseases. Other diseases that may be treated using polynucleotides on the invention are in ocular disorders such as age-related macular degeneration (e.g., as described in U.S. Pat. No. 7,879,813 and U.S. 2009/0012030) and topical disorders such as psoriasis.

The compositions containing an effective amount can be administered for prophylactic or therapeutic treatments. In prophylactic applications, compositions can be administered to a subject with a clinically determined predisposition or increased susceptibility to cancer, or any disease described herein. Compositions of the invention can be administered to the subject (e.g., a human) in an amount sufficient to delay, reduce, or prevent the onset of clinical disease. In therapeutic applications, compositions are administered to a subject (e.g., a human) already suffering from disease (e.g., cancer, such as leukemia or a myelodysplastic syndrome) in an amount sufficient to cure or at least partially arrest the symptoms of the condition and its complications.

Amounts effective for this use may depend on the severity of the disease or condition and the weight and general state of the subject, but generally range from about 0.05 μg to about 1000 μg (e.g., 0.5-100 μg) of an equivalent amount of the agent per dose per subject. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6 hours, 8-12 hours 14-16 hours, 18-24 hours, every 2-4 days, every 1-2 weeks, and once a month). Alternatively, continuous intravenous infusions sufficient to maintain therapeutically effective concentrations in the blood are contemplated.

The therapeutically effective amount of one or more agents present within the compositions of the invention and used in the methods of this disclosure applied to mammals (e.g., humans) can be determined by the ordinarily-skilled artisan with consideration of individual differences in age, weight, and the condition of the mammal. Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

One or more nucleotide constructs of the invention may be used in combination with either conventional methods of treatment or therapy or may be used separately from conventional methods of treatment or therapy.

When one or more nucleotide constructs of the invention are administered in combination therapies with other agents, they may be administered sequentially or concurrently to an individual. Alternatively, pharmaceutical compositions according to the invention may comprise a combination of a nucleotide construct of the invention in association with a pharmaceutically acceptable excipient, as described herein, and another therapeutic or prophylactic agent known in the art.

The following examples are meant to illustrate the invention. They are not meant to limit the invention in any way.

### EXAMPLES

### Example 1

### Synthesis and Purification of the Nucleotides and Polynucleotides of the Invention

### General Synthesis Procedure

The polynucleotide constructs of the invention can be prepared according to the generalized and specific methods and schemes described herein. For example, starting materials containing thiols underwent a reaction with 2,2′-dipyridyl disulfide affording the corresponding pyridyl disulfide compounds (e.g., see Scheme 1), which were then subjected to a reaction with nucleoside phosphordiamidites to generate nucleotide constructs of the invention (e.g., see Scheme 1). These nucleotide constructs were then used in standard oligonucleotide synthesis protocols to form polynucleotide constructs. These polynucleotide constructs were then deprotected and purified using HPLC.

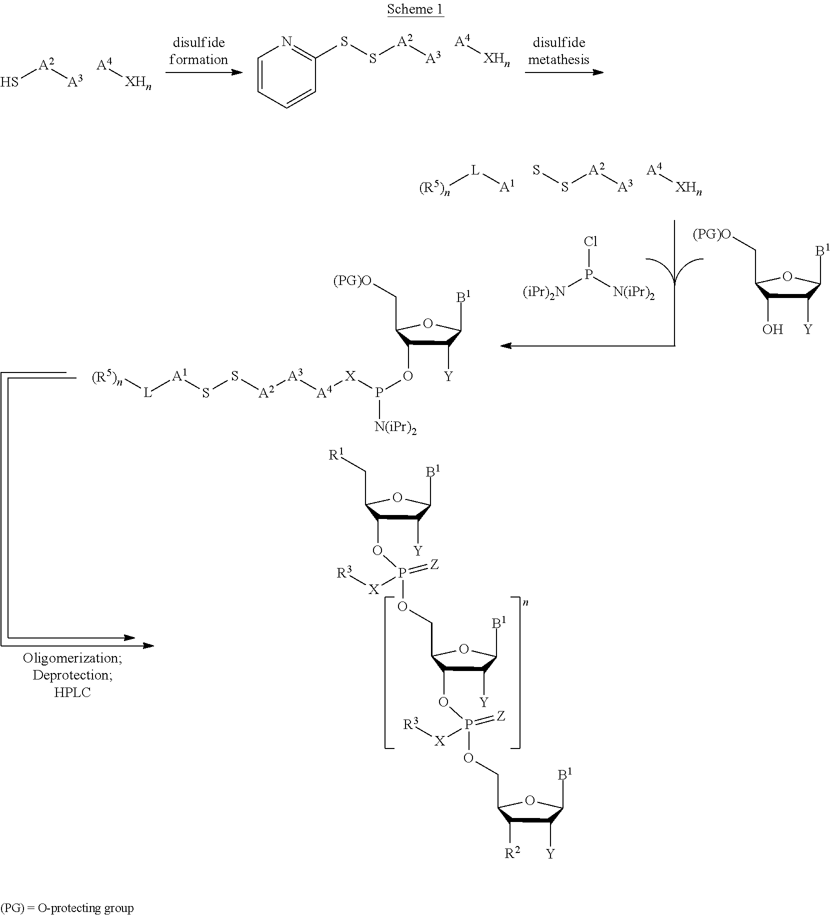

### Specific Syntheses of the Nucleotides of the Invention

Exemplary syntheses of nucleotides of the invention are described below.

### Precursors

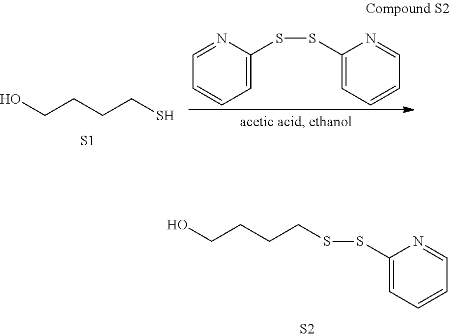

To a solution of 4-Mecaptol-butanol (10.0 g, 94 mmol) and dithiopyridine (25.0 g, 113 mmol) in 400 mL of ethanol was added 7.0 mL of acetic acid. The reaction mixture was stirred for 1 hour at room temperature before being condensed under vacuum. 500 mL of ethyl acetate was added to the crude product and the solution was washed sequentially by aqueous 1N NaOH solution (200 mL) and brine (200 mL), and then dried over anhydrous Na₂SO₄. The solvent was evaporated in vacuo and the crude mixture was purified by silica gel column chromatography using ethyl acetate/hexane solvent system (0-40% gradient on Combi Flash Rf Instrument) to give 12.8 g (64%) of product S2 as colorless oil. ¹H NMR (500 MHz): δ8.45 (d, J 4.5 Hz, 1H), 7.70 (d, J 8.0 Hz, 1H), 7.62 (m, 1H), 7.06 (m, 1H), 3.65 (t, J 6.0 Hz, 2H), 2.83 (t, J 7.0 Hz, 2H), 1.80 (m, 2H), 1.70 (br s, 1H), 1.65 (m, 2H).

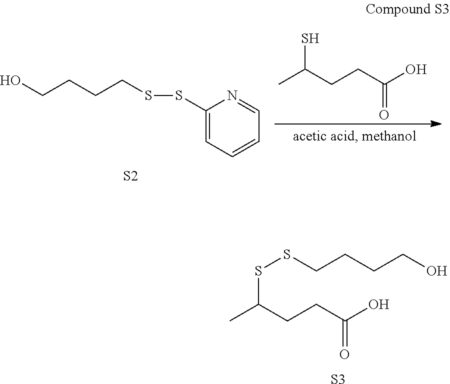

To a solution of S2 (1.3 g, 6.0 mmol) and 4-sulfanylpentanoic acid (0.67 g, 5.0 mmol) in 30 mL of methanol was added 30 μL of acetic acid. The reaction mixture was stirred for 16 hours at room temperature before being condensed in vacuo. The crude mixture was purified by silica gel column chromatography using ethyl acetate/hexane/2% acetic acid solvent system (0-70% gradient on Combi Flash Rf Instrument) to give 1.13 g (95%) of product S3 as colorless oil. ¹H NMR (500 MHz): δ4.95 (br s, 1H), 3.68 (t, J 6.0 Hz, 2H), 2.88 (m, 1H), 2.71 (t, J 7.0 Hz, 2H), 2.50 (m, 2H), 1.98 (m, 1H), 1.18 (m, 1H), 1.75 (m, 2H), 1.65 (m, 2H), 1.32 (d, J 7.0 Hz, 3H).

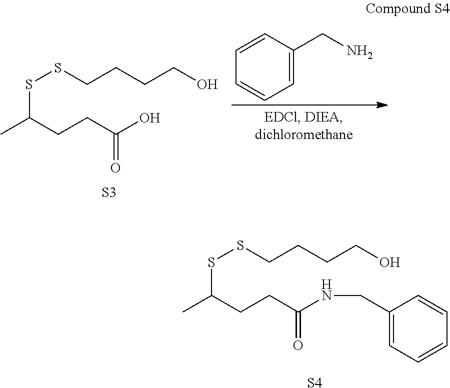

To a solution of S3 (1.13 g, 5.0 mmol), benzylamine (0.84 mL, 7.7 mmol) and 3.6 mL of N, N-diisopropylethylamine (DIEA) in 25.0 mL of dichloromethane was added 1-ethyl-3-(3-dimethylaminopropyl) carbodiimide (EDCl, 1.5 g, 7.7 mmol). The reaction mixture was stirred for 2 hours at room temperature before being condensed in vacuo. The crude mixture was purified by silica gel column chromatography using ethyl acetate/hexane solvent system (0-100% gradient on Combi Flash Rf Instrument) to give 1.17 g (70%) of product S4 as colorless oil. ¹H NMR (500 MHz): δ7.22-7.31 (m, 5H), 6.55 (br s, 1H, 4.35 (d, J 5.5 Hz, 2H), 4.20 (br s, 1H), 3.55 (m, 2H), 2.80 (m, 1H), 2.60 (t, J 7.5 Hz, 2H), 2.25 (t, J 7.5 Hz, 2H), 1.85 (m, 1H), 1.75 (m, 1H), 1.65 (m, 2H), 1.55 (m, 2H), 1.25 (d, J 6.5 Hz, 3H).

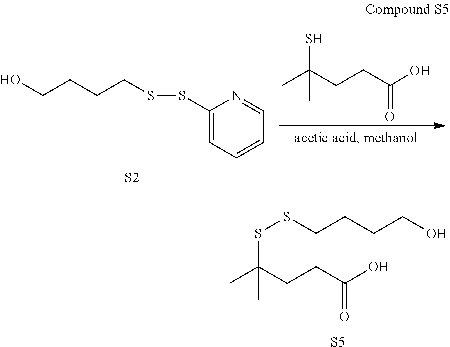

To a solution of S2 (1.82 g, 8.4 mmol) and 4-sulfanyl-4-methylpentanoic acid (1.04 g, 7.0 mmol) in 45.0 mL of methanol was added 35 μL of acetic acid. The reaction mixture was stirred for 16 hours at room temperature before being condensed in vacuo. The crude mixture was purified by silica gel column chromatography using ethyl acetate/hexane/2% acetic acid solvent system (0-70% gradient on Combi Flash Rf Instrument) to give 0.82 g (50%) of product S5 as colorless oil. ¹H NMR (500 MHz): δ7.25 (br s, 1H), 3.63 (t, J 6.0 Hz, 2H), 2.69 (m, 2H), 2.40 (m, 2H), 1.83 (m, 2H), 1.70 (m, 2H), 1.62 (m, 2H), 1.25 (s, 6H).

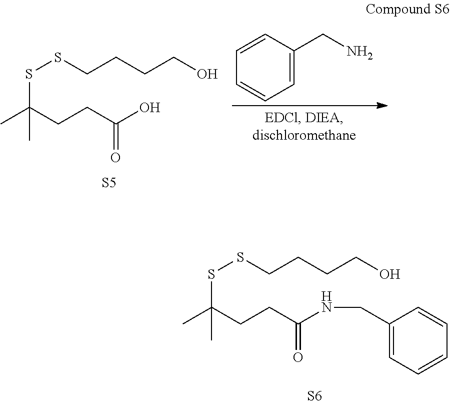

To a solution of S5 (0.82 g, 3.25 mmol), benzylamine (0.53 mL, 4.88 mmol) and 2.3 mL of N, N-diisopropylethylamine (DIEA) in 20.0 mL of dichloromethane was added 1-ethyl-3-(3-dimethylaminopropyl) carbodiimide (EDCl, 0.94 g, 4.88 mmol). The reaction mixture was stirred for 2 hours at room temperature before being condensed in vacuo. The crude mixture was purified by silica gel column chromatography using ethyl acetate/hexane solvent system (0-100% gradient on Combi Flash Rf Instrument) to give 0.80 g (73%) of product S6 as colorless oil. ¹H NMR (500 MHz): δ7.22-7.40 (m, 5H), 6.30 (br s, 1H), 4.37 (d, J=6.0 Hz, 2H), 3.60 (m, 2H), 2.80 (m, 1H), 2.68 (m, 2H), 2.25 (m, 2H), 1.85 (m, 2H), 1.75 (m, 1H), 1.65 (m, 2H), 1.55 (m, 2H), 1.25 (s, 6H).

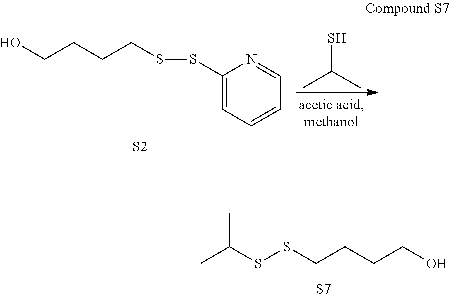

To a solution of S2 (1.0 g, 4.6 mmol) and 2-propanethiol (0.52 mL, 5.5 mmol) in 20.0 mL of methanol was added 15 μL of acetic acid. The reaction mixture was stirred for 16 hours at room temperature before being condensed under vacuum. The crude mixture was diluted with 100 mL of ethyl acetate and washed sequentially by aqueous 1N NaOH solution (200 mL) and brine (200 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.40 g (40%) of product S7 as colorless oil. ¹H NMR (500 MHz): δ3.63 (t, J 6.5 Hz, 2H), 2.89 (m, 1H), 2.70 (t, J 7.0 Hz, 2H), 1.80 (s, 1H), 1.75 (m, 2H), 1.65 (m, 1H), 1.27 (d, J 7.0 Hz, 6H).

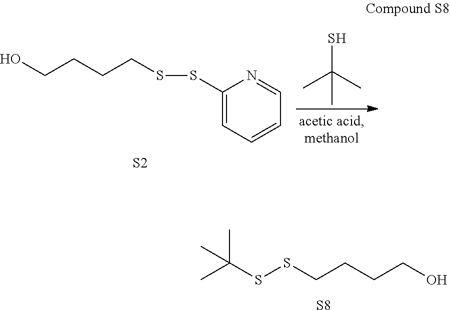

To a solution of S2 (6.0 g, 27.7 mmol) and 2-methyl-2-propanethiol (2.5 g, 27.7 mmol) in 100 mL of methanol was added 100 μL of acetic acid. The reaction mixture was stirred for 16 hours at room temperature before being condensed under vacuum. The crude mixture was diluted with 400 mL of ethyl acetate and washed sequentially by aqueous 1N NaOH solution (200 mL) and brine (200 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 3.0 g (60%) of product S8 as colorless oil. ¹H NMR (500 MHz): δ3.65 (m, 2H), 2.75 (t, J 7.5 Hz, 2H), 1.75 (m, 2H), 1.65 (m, 2H), 1.30 (s, 9H).

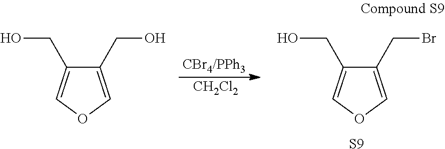

To a solution of 3,4-dishydroxymethylfuran (1.0 g, 7.8 mmol), triphenylphosphine (2.3 g, 8.6 mmol) in 25.0 mL of dichloromethane was added carbon tetrabromide (2.85 g, 8.6 mmol). The reaction mixture was stirred for 16 hours at room temperature before being condensed in vacuo. The crude mixture was purified by silica gel column chromatography using ethyl acetate/hexane solvent system (0-35% gradient on Combi Flash Rf Instrument) to give 1.09 g (74%) of product S9 as colorless oil which was quickly dissolved in methanol for next reaction. ¹H NMR (500 MHz): δ7.50 (s, 1H), 7.40 (s, 1H), 4.65 (s, 2H), 4.46 (s, 2H).

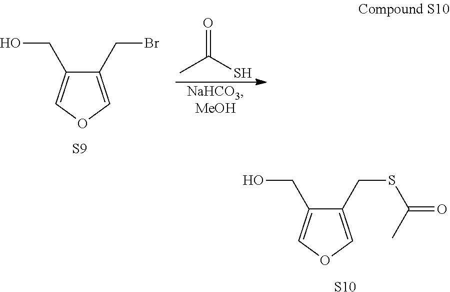

To a solution of S9 (1.09 g, 5.7 mmol) and thioacetic acid (0.52 g, 6.8 mmol) in 10.0 mL of methanol was added NaHCO₃ (0.58 g, 6.8 mmol) portion wise. The reaction mixture was stirred for 2 hours at room temperature before being neutralized to pH 7 with 1N HCl solution and the volatiles were evaporated in vacuo. The residue was diluted with 200 mL of ethyl acetate and washed sequentially by saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 0.80 g (75%) of product S10 as colorless oil. ¹H NMR (500 MHz): δ7.37 (s, 1H), 7.35 (s, 1H), 4.53 (d, J 5.5 Hz, 2H), 4.00 (s, 2H), 2.34 (s, 3H), 1.88 (t, J 5.5 Hz, 1H).

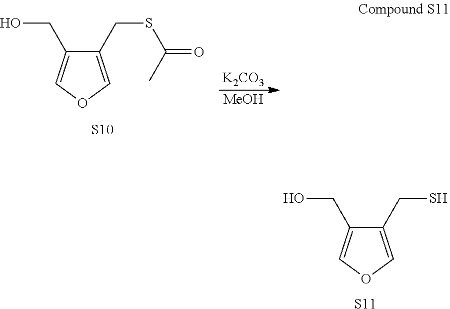

To a solution of S10 (0.60 g, 3.2 mmol) in 15.0 mL of methanol was added K₂CO₃ (0.53 g, 3.86 mmol) portion wise under Argon atmosphere. The reaction mixture was stirred for 30 minutes at room temperature before being neutralized to pH 7 with 1N HCl solution and the volatiles were evaporated in vacuo. The residue was diluted with 100 mL of ethyl acetate and washed sequentially by saturated NaHCO₃ solution (30 mL) and brine (30 mL), then dried over anhydrous Na₂SO₄. The solvent was evaporated in vacuo and the crude mixture directly used in the next reaction.

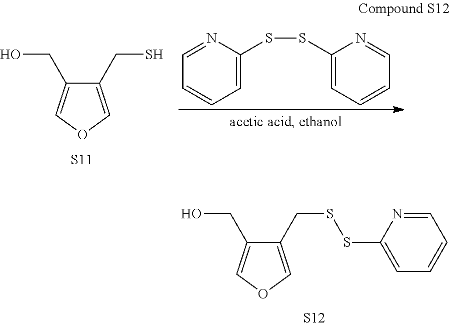

To a solution of crude S11 (0.46 g, 3.2 mmol) and dithiopyridine (0.85 g, 3.8 mmol) in 12.0 mL of ethanol was added 200 μL of acetic acid. The reaction mixture was stirred for 45 minutes at room temperature before being condensed in vacuo. The crude mixture was purified by silica gel column chromatography using ethyl acetate/hexane solvent system (0-30% gradient on Combi Flash Rf Instrument) to give 0.40 g (50% yield) of product S12 as colorless oil. ¹H NMR (500 MHz): δ8.46 (d, J 5.0 Hz, 1H), 7.56 (m, 1H), 7.40 (d, J 8.0 Hz, 1H), 7.32 (s, 2H), 7.09 (m, 1H), 4.65 (s, 2H), 3.97 (s, 2H), 1.60 (br s, 1H).

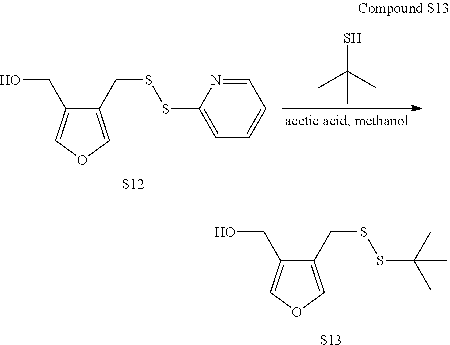

To a solution of S12 (0.39 g, 1.5 mmol) and tert-butyl mercaptan (0.21 mL, 1.8 mmol) in 20.0 mL of methanol was added 50 μL of acetic acid. The reaction mixture was stirred for 40 hours at room temperature before being condensed in vacuo. The crude mixture was purified by silica gel column chromatography using ethyl acetate/hexane solvent system (0-50% gradient on Combi Flash Rf Instrument) to give 0.33 g (95%) of product S13 as colorless oil. ¹H NMR (500 MHz): δ7.40 (s, 1H), 7.37 (s, 1H), 4.60 (s, 2H), 3.82 (s, 2H), 1.84 (br s, 1H), 1.34 (s, 9H).

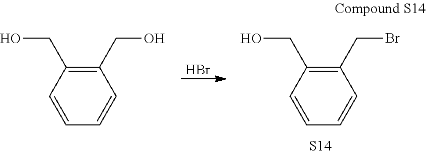

To a solution of 48% hydrobromic acid (15.0 mL) was added 1,2-benzenedimethanol (4.0 g, 29.0 mmol) and the reaction mixture was stirred for 2 hours at room temperature. 1N NaOH aqueous solution was added to the reaction mixture to neutralize the solution to pH 7. The resulting mixture was diluted with ethyl acetate (200 mL), washed sequentially by saturated NaHCO₃ solution (20 mL) and brine (20 mL), and then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 2.6 g (45%) of product S14 as white solid. ¹H NMR (500 MHz): δ7.30-7.45 (m, 4H), 4.85 (s, 2H), 4.64 (s, 2H), 1.81 (br s, 1H).

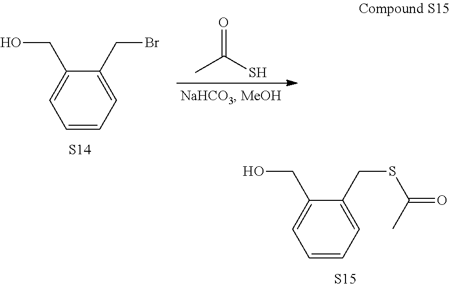

To a solution of S14 (1.0 g, 5.0 mmol) and Thioacetic acid (0.46 g, 6.0 mmol) in 10.0 mL of methanol was added NaHCO₃ (0.50 g, 6.0 mmol) portion wise. The reaction mixture was stirred for 2 hours at room temperature before being neutralized to pH 7 with 1N HCl solution and the volatiles were evaporated in vacuo. The residue was diluted with 200 mL of ethyl acetate and washed sequentially by saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 0.97 g (99%) of product S15 as colorless oil. ¹H NMR (500 MHz): δ7.40 (m, 2H), 7.25 (m, 2H), 4.73 (d, J 5.5 Hz, 2H), 4.24 (s, 2H), 2.34 (s, 3H), 2.05 (t, J 5.5 Hz, 1H).

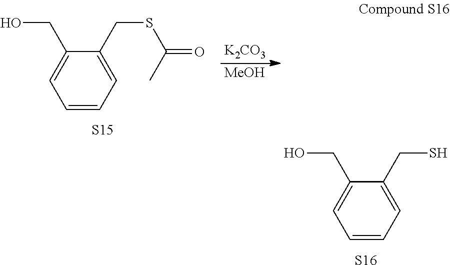

To a solution of S15 (0.75 g, 3.8 mmol) in 20.0 mL of methanol was added K₂CO₃ (0.64 g, 4.6 mmol) portion wise under Argon atmosphere. The reaction mixture was stirred for 30 minutes at room temperature before being neutralized to pH 7 with 1N HCl solution and the volatiles were evaporated in vacuo. The residue was diluted with 100 mL of ethyl acetate and washed sequentially by saturated NaHCO₃ solution (30 mL) and brine (30 mL), then dried over anhydrous Na₂SO₄. The solvent was evaporated in vacuo and the crude product directly used in the next reaction.

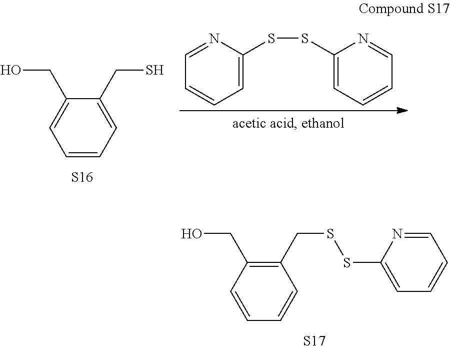

To a solution of crude S16 (0.52 g, 3.4 mmol) and Dithiopyridine (0.89 mg, 4.05 mmol) in 15.0 mL of ethanol was added 0.30 mL of acetic acid. The reaction mixture was stirred for 30 minutes at room temperature before being condensed in vacuo. The crude mixture was purified by silica gel column chromatography using ethyl acetate/hexane solvent system (0-30% gradient on Combi Flash Rf Instrument) to give 0.52 g (50%) of product S17 as colorless oil. ¹H NMR (500 MHz): δ8.42 (d, J 5.0 Hz, 1H), 7.25-7.51 (m, 7H), 4.83 (s, 2H), 4.19 (s, 2H), 3.85 (br s, 1H).

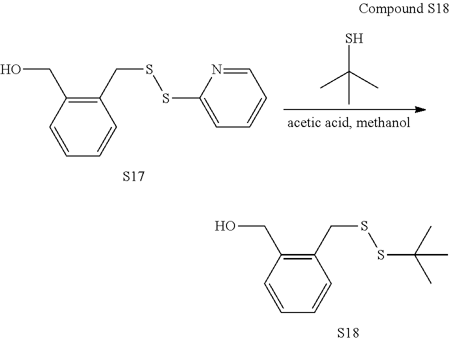

To a solution of S17 (0.42 g, 1.6 mmol) and tert-butyl mercaptan (0.21 mL, 1.9 mmol) in 20.0 mL of methanol was added 50 μL of acetic acid. The reaction mixture was stirred for 48 hours at room temperature before being condensed in vacuo. The crude mixture was purified by silica gel column chromatography using ethyl acetate/hexane solvent system (0-50% gradient on Combi Flash Rf Instrument) to give 0.32 g (94% yield) of product S18 as colorless oil. ¹H NMR (500 MHz): δ7.40 (m, 1H), 7.26-7.30 (m, 3H), 4.80 (d, 2H, J 4.0 Hz), 4.06 (s, 2H), 1.95 (br s, 1H), 1.35 (s, 9H).

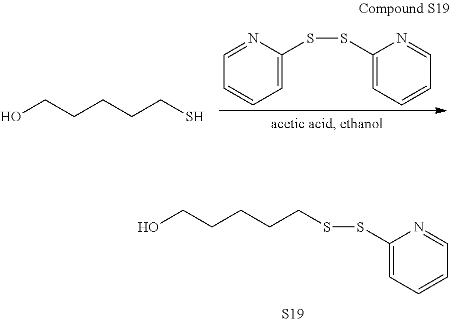

To a solution of 5-Mecaptobutanol (0.85 g, 7.1 mmol) and Dithiopyridine (1.87 g, 8.5 mmol) in 25.0 mL of ethanol was added 0.2 mL of acetic acid. The reaction mixture was stirred for 1 hour at room temperature before being condensed under vacuum. 50.0 mL of ethyl acetate was added to the crude product and the solution was washed sequentially by 1N NaOH aqueous solution (50 mL) and brine (30 mL), and then dried over anhydrous Na₂SO₄. The solvent was evaporated in vacuo and the crude mixture was purified by silica gel column chromatography using ethyl acetate/hexane solvent system (0-40% gradient on Combi Flash Rf Instrument) to give 1.21 g (75%) of product S19 as colorless oil. ¹H NMR (500 MHz): δ8.45 (d, J 5.0 Hz, 1H), 7.71 (d, J 8.0 Hz, 1H), 7.63 (m, 1H), 7.07 (m, 1H), 3.62 (t, J 6.5 Hz, 2H), 2.81 (t, J 7.5 Hz, 2H), 1.73 (m, 2H), 1.56 (m, 2H), 1.48 (m, 2H).

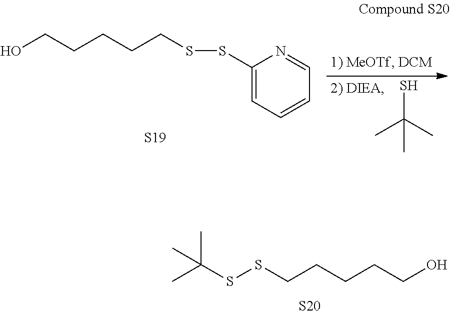

To a solution of S19 (1.2 g, 5.3 mmol) in 20.0 mL of dichloromethane was added Methyl trifluoromethanesulfonate (0.87 g, 5.3 mmol). The reaction mixture was stirred for 15 minutes at room temperature followed by addition of 2-Methyl-2-propanethiol (1.2 mL, 10.6 mmol) and DIEA (2.7 mL, 15.9 mmol). The reaction mixture was stirred for another 1 hour before being condensed under vacuum. The crude mixture was purified by silica gel column chromatography using ethyl acetate/hexane solvent system (0-30% gradient on Combi Flash Rf Instrument) to give 0.67 g (61%) of product S20 as colorless oil. ¹H NMR (500 MHz): δ3.65 (t, J 6.5 Hz, 2H), 2.70 (t, J 7.0 Hz, 2H), 1.67 (m, 2H), 1.57 (m, 2H), 1.45 (m, 2H), 1.32 (s, 9H).

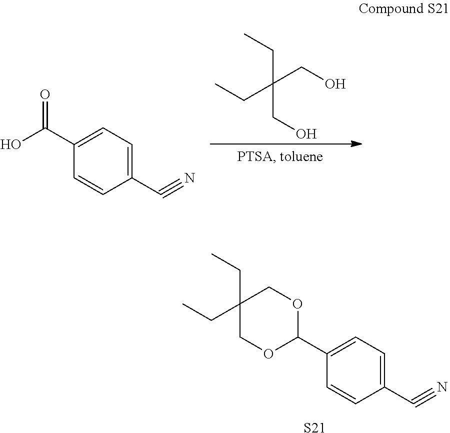

The suspension of 4-cyanobenzaldehyde (5.0 g, 38.1 mmol), 2,2-diethyl-1,3-propanediol (5.5 g, 41.9 mmol) and p-Toluenesulfonic acid monohydrate (0.21 g, 1.14 mmol) in 250 mL of toluene was refluxed with Dean-Stark apparatus for 16 hours. The reaction mixture was cooled to room temperature and the volatile was removed under reduced pressure. The crude mixture was diluted with 300 mL of ethyl acetate and washed sequentially by saturated NaHCO₃ solution (30 mL) and brine (30 mL), then dried over anhydrous Na₂SO₄. The solvent was evaporated in vacuo and the crude mixture was purified by silica gel column chromatography using ethyl acetate/hexane solvent system (0-20% gradient on Combi Flash Rf Instrument) to give 8.7 g (94%) of product S21 as white solid. ¹H NMR (500 MHz): δ7.66 (d, J 6.5 Hz, 2H), 7.61 (d, J 8.5 Hz, 2H), 5.4 (s, 1H), 3.97 (d, J 11.5 Hz, 2H), 3.61 (d, J 12.0 Hz, 2H), 1.79 (q, J 7.5 Hz, 2H), 1.15 (q, J 7.5 Hz, 2H), 0.89 (t, J 7.5 Hz m, 3H), 0.82 (t, J 7.5 Hz m, 3H).

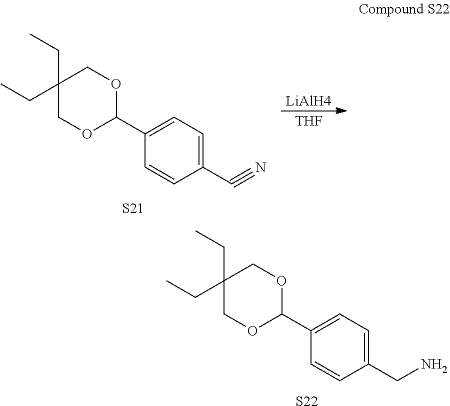 [SMILES: CCC1(CC)COC(c2ccc(C#N)cc2)OC1 ; "LiAlH4" ; "S21" ; CCC1(CC)COC(c2ccc(CN)cc2)OC1 ; "S22" ; "THF"]

The suspension of lithium aluminum hydride (0.94 g, 24.6 mmol) in THF was cooled to 0° C., to which was added drop wise a solution of S21 (2.0 g, 8.2 mmol) in 25.0 mL of THF under Argon atmosphere. The reaction mixture was warmed to room temperature and further stirred for 3 hours. The suspension was cooled to 0° C. by ice bath, quenched with saturated Na₂SO₄ solution and filtered through a pad of celite. The filtrate was condensed under reduced pressure. The crude mixture was diluted with 100 mL of ethyl acetate and washed sequentially by saturated NaHCO₃ solution (20 mL) and brine (20 mL), then dried over anhydrous Na₂SO₄. The solvent was evaporated in vacuo to get crude intermediate S22 as colorless oil, which was used in the next reaction without further purification.

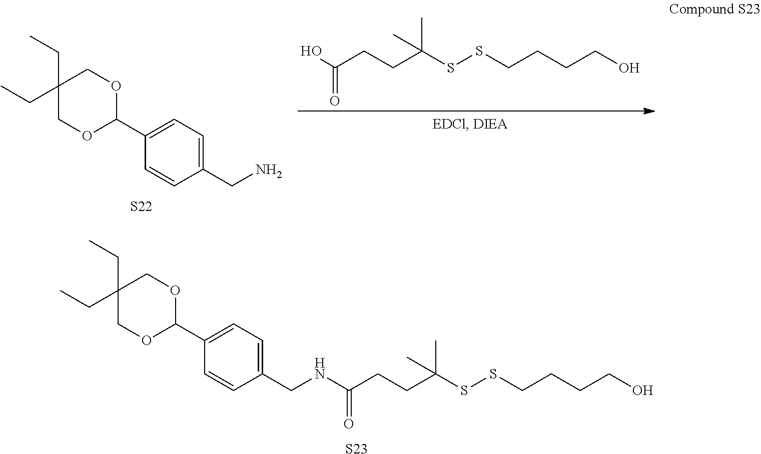 [SMILES: CCC1(CC)COC(c2ccc(CN)cc2)OC1 ; CC(C)(CCC(=O)O)SSCCCCO ; "S22" ; CCC1(CC)COC(c2ccc(CNC(=O)CCC(C)(C)SSCCCCO)cc2)OC1 ; "S23"]

To a solution of S5 (2.8 g, 11.0 mmol), EDCl (2.5 g, 13.0 mmol) and DIEA (7.6 mL, 44.0 mmol) in 25.0 mL of dichloromethane was added a solution of S22 (2.84 g, 11.0 mmol) in 10.0 mL of dichloromethane. The reaction mixture was stirred for 16 hours at room temperature before being condensed under reduced pressure. The crude mixture was purified by silica gel column chromatography using ethyl acetate/hexane solvent system (0-40% gradient on Combi Flash Rf Instrument) to give 2.5 g (47%) of product S23 as colorless oil. ¹H NMR (500 MHz): δ7.45 (d, J 8.0 Hz, 2H), 7.26 (d, J 8.0 Hz, 2H), 5.85 (br s, 1H), 5.37 (s, 1H), 5.29 (s, 2H), 4.41 (d, J 5.5 Hz, 2H), 3.93 (d, J 11.5 Hz, 2H), 3.60 (m, 4H), 2.69 (t, J 7.5 Hz, 2H), 2.29 (m, 2H), 1.93 (m, 2H), 1.80 (q, J 7.5 Hz, 2H), 1.75 (m, 2H), 1.60 (m, 2H), 1.28 (s, 6H), 1.13 (q, J 7.5 Hz, 2H), 0.89 (t, J 7.5 Hz, 3H), 0.81 (t, J 7.5 Hz, 3H).

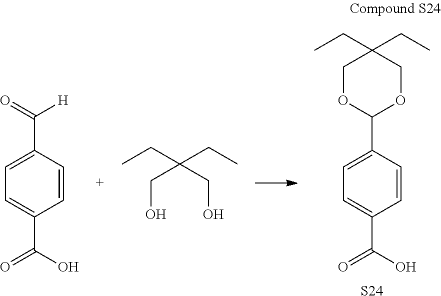

To a suspension of 4-Formyl benzoic acid (15.01 g, 100 mmol) and 2,2-diethyl-1,3-propanediol (14.54 g, 110 mmol) in toluene (250 mL) was added p-toluenesulfonic acid monohydrate (0.57 g, 3.0 mmol). The mixture was refluxed overnight with a Dean stark apparatus. The reaction mixture was cooled to room temp to form large amount of precipitates. The solid was filtered, heated with 100 mL of ethyl acetate and cooled to collect the precipitate, and dried under high vacuum to give 20 g of the title compound S24. The filtrate was washed with water and brine, dried over anhydrous Na₂SO₄ and evaporated to give a white solid, which was recrystallized from ethyl acetate to give another 1.5 g of S24 (total 21.5 g, 81%). ¹H NMR (500 MHz, CDCl3): δ8.12 (2H, d, J 8.5 Hz), 7.61 (2H, d, J 8.5 Hz), 5.45 (1H, s), 3.98 (2H, d, J 11.5 Hz), 3.62 (2H, d, J 11.5 Hz), 1.83 (2H, q, J 7.5 Hz), 1.16 (2H, q, J 7.5 Hz), 0.90 (3H, t, J 7.5 Hz), 0.83 (3H, t, J 7.5 Hz).

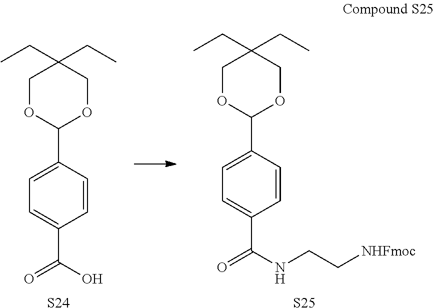

To a solution of S24 (1.32 g, 5.0 mmol) and mono-Fmoc ethylenediamine HCl salt (1.75 g, 5.5 mmol) in dimethylformamide (15.0 mL) were added HATU (2.28 g, 6.0 mmol) and N, N-diisopropylethylamine (4.35 mL, 25.0 mmol). The resulting mixture was stirred for 30 min and the volatiles removed under high vacuum to give a brown solid. The solid was washed with ethyl acetate three times to afford 1.95 g (74%) of pure compound S25 as a white solid. ¹H NMR (500 MHz, CDCl3): δ7.78 (2H, d, J 8.0 Hz), 7.74 (2H, d, J 7.5 Hz), 7.55 (2H, d, J 7.5 Hz), 7.53 (2H, d, J 8.0 Hz), 7.37 (2H, t, J 7.5 Hz), 7.26 (2H, t, J 7.5 Hz), 7.07 (1H, br s), 5.47 (1H, br s), 5.38 (1H, s), 4.40 (2H, d, J 6.5 Hz), 4.16 (1H, t, J 6.5 Hz), 3.95 (2H, d, 11.5 Hz), 3.58 (2H, d, J 11.5 Hz), 3.55-3.50 (2H, m), 3.43-3.35 (2H, m), 1.81 (2H, q, J 7.5 Hz), 1.14 (2H, q, J 7.5 Hz), 0.89 (3H, t, J 7.5 Hz), 0.82 (3H, t, J 7.5 Hz)

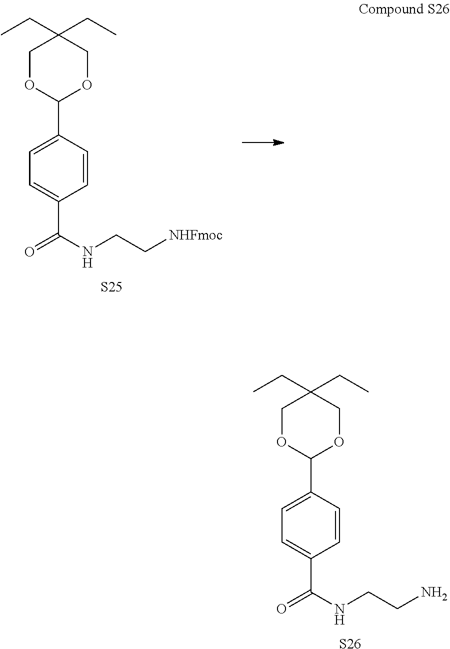

To a solution of compound S25 (1.95 g, 3.68 mmol) in dimethylformamide (15 mL) was added 3 mL of piperidine and the mixture was stirred for 30 min. The mixture was washed with hexane (20 mL×2) and the dimethylformamide layer was evaporated under high vacuum to give crude compound S26, which was used in the next reaction without further purification.

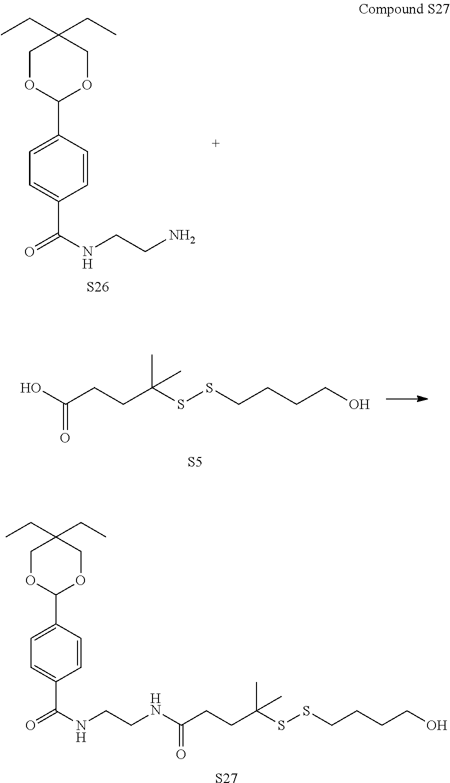 [SMILES: CCC1(CC)COC(c2ccc(C(=O)NCCN)cc2)OC1 ; "S26" ; CC(C)(CCC(=O)O)SSCCCCO ; "S5" ; CCC1(CC)COC(c2ccc(C(=O)NCCNC(=O)CCC(C)(C)SSCCCCO)cc2)OC1 ; "S27"]

To a mixture of compound S26 and S5 (0.87 g, 3.45 mmol) in dimethylformamide (10 mL) were added HATU (1.68 g, 4.4 mmol) and N,N-diisopropylethylamine (1.2 mL, 6.9 mmol). The mixture was stirred for 1 hour and the volatiles were removed under high vacuum to give a brown solid. The solid was washed with ethyl acetate several times and dried under high vacuum to afford 0.95 g (51%) of the title compound S27 as a white solid. ¹H NMR (500 MHz, CDCl3): δ7.81 (2H, d, J 8.5 Hz), 7.57 (2H, d, J 8.5 Hz), 7.19 (1H, br s), 6.42 (1H, br s), 5.42 (1H, s), 3.96 (2H, d, J 11.0 Hz), 3.64-3.55 (6H, m), 3.53-3.47 (2H, m), 2.66 (2H, t, J 7.5 Hz), 2.31-2.26 (2H, m), 2.05 (1H, br s), 1.90-1.85 (2H, m), 1.82 (2H, q, J 7.5 Hz), 1.75-1.66 (2H, m), 1.63-1.55 (2H, m), 1.25 (6H, s), 1.15 (2H, q, J 7.5 Hz), 0.89 (3H, t, J 7.5 Hz), 0.82 (3H, t, J 7.5 Hz).

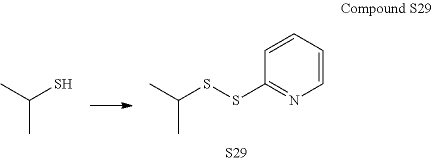

To a solution of Isopropylthiol (7.6 g, 100 mmol) in ethanol (300 mL) were added dithiodipyridine (24.2 g, 110 mmol) and acetic acid (7.0 mL). The mixture was stirred overnight, and then evaporated to give a residue, which was dissolved in 200 mL of ethyl acetate. The solution was washed with 1N NaOH (50 mL×3) and brine. The organic layer was dried over anhydrous Na₂SO₄, filtered and evaporated to give a residue, which was subjected to flash silica gel column purification on an ISCO companion (ethyl acetate/hexane=5%-20%) to give 14.4 g (77%) of the title compound S29 as a colorless oil. ¹H NMR (500 MHz, CDCl₃): δ8.44 (1H, d, J 5.0 Hz), 7.75 (1H, d, J 8.0 Hz), 7.63 (1H, td, J 8.0, 1.5 Hz), 7.06 (1H, m), 3.13 (1H, m), 1.33 (6H, d, J 7.0 Hz).

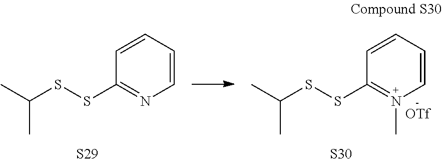

To a solution of compound S29 (1.86 g, 10.0 mmol) in dichloromethane (5.0 mL) was added MeOTf (1.64 g, 10.0 mmol). The mixture was stirred for 15 min and washed with hexane (10 mL×2). The dichloromethane layer was evaporated to give the crude salt as yellow oil (S30), which was used directly in the next reaction.

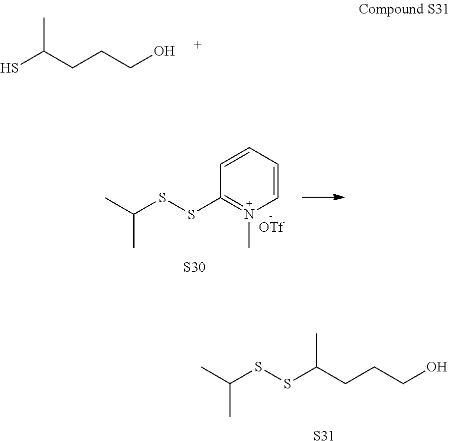

To a solution of 4-mercapto-4-methylbutan-1-ol (0.36 g, 3.0 mmol) in dichloromethane was added the crude S30 (1.26 g, 3.6 mmol) and N, N-diisopropylethylamine (1.0 mL). The mixture was stirred for 10 min, volatiles were removed under vacuum to give a residue, which was subjected to flash silica gel column purification on an ISCO companion instrument (ethyl acetate/hexane=5%-40%) to give 0.50 g (85%) of the title compound S31 as a colorless oil. ¹H NMR (500 MHz, CDCl₃): δ3.67 (2H, t, J 6.5 Hz), 2.96 (1H, J 6.5 Hz), 2.83 (1H, m), 1.77-1.67 (3H, m), 1.63-1.55 (1H, m), 1.32 (3H, d, J 6.5 Hz), 1.30 (6H, d, J 6.5 Hz).

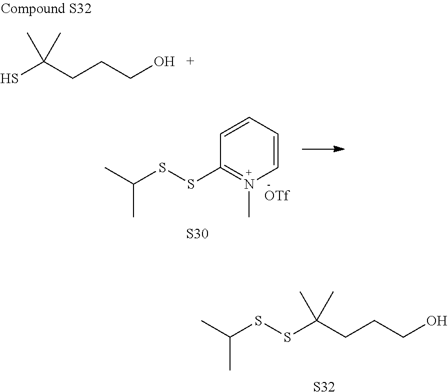

To a solution of 4-mercapto-4-methylpentan-1-ol (0.19 g, 1.39 mmol) in dichloromethane was added the crude S30 (0.58 g, 1.66 mmol) and N,N-diisopropylethylamine (1.0 mL). The mixture was stirred for 10 min, volatiles were removed under vacuum to give a residue, which was subjected to flash silica gel column purification on an ISCO companion (ethyl acetate/hexane=5%-40%) to give 0.26 g (89%) of the title compound S32 as a colorless oil. ¹H NMR (500 MHz, CDCl₃): δ3.66 (2H, t, J 5.5 Hz), 2.94 (1H, J 6.5 Hz), 1.72-1.60 (4H, m), 1.29 (6H, s), 1.29 (6H, d, J 6.5 Hz).

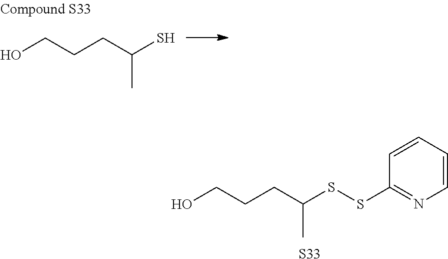

To a solution of 4-mercapto-4-methylbutan-1-ol (0.18 g, 1.5 mmol) in methanol (5.0 mL) were added dithiodipyridine (0.35 g, 1.6 mmol) and acetic acid (30 μL). The mixture was stirred for 30 min, then evaporated to give a residue, which was subjected to flash silica gel column purification on an ISCO companion (ethyl acetate/hexane=15%-70%) to give 0.27 g (78%) of the title compound S33 as a colorless oil. ¹H NMR (500 MHz, CDCl₃): δ8.84 (1H, d, J 5.0 Hz), 7.73 (1H, d, J 8.0 Hz), 7.63 (1H, td, J 8.0, 1.5 Hz), 7.07 (1H, m), 3.64 (2H, t, J 6.5 Hz), 2.99 (1H, m), 1.82-1.60 (4H, m), 1.34 (3H, d, J 7.0 Hz).

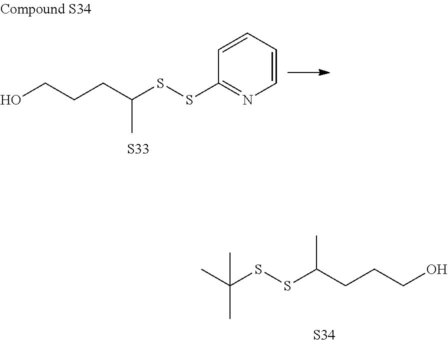

To a solution of compound S33 (0.27 g, 1.15 mmol) in dichloromethane (5.0 mL) was added MeOTf (0.19 g, 1.15 mmol). The mixture was stirred for 15 min, and then 2-methyl-2-propanethiol (0.21 g, 2.3 mmol) and N, N-Diisopropylethylamine (1.0 mL) were added. The resulting mixture was stirred for another 30 min. Evaporation of the mixture afforded a residue, which was subjected to flash silica gel column purification on an ISCO companion (ethyl acetate/hexane=5%-40%) to give 0.19 g (79%) of the title compound S34 as a colorless oil. ¹H NMR (500 MHz, CDCl₃): δ3.67 (2H, t, J 6.5 Hz), 2.84 (1H, m), 1.75-1.65 (3H, m), 1.62-1.55 (1H, m), 1.32 (9H, s), 1.31 (3H, d, J 7.0 Hz).

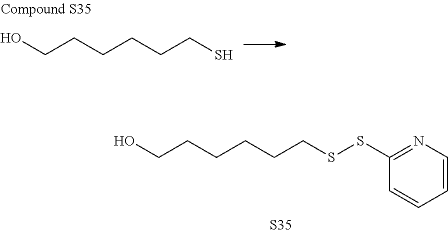 [SMILES: OCCCCCCS ; OCCCCCCSSc1ccccn1 ; "S35"]

To a solution of 6-mercapto-1-hexanol (2.68 g, 20.0 mmol) in methanol (50.0 mL) were added dithiodipyridine (6.6 g, 30.0 mmol) and acetic acid (1.0 mL). The mixture was stirred for 30 min, then evaporated to give a residue, which was subjected to flash silica gel column purification on an ISCO companion (ethyl acetate/hexane=15%-70%) to give 4.37 g (90%) of the title compound S35 as a colorless oil. ¹H NMR (500 MHz, CDCl₃): δ8.46 (1H, d, J 4.5 Hz), 7.72 (1H, d, J 8.0 Hz), 7.64 (1H, td, J 8.0, 1.5 Hz), 7.07 (1H, m), 3.63 (2H, t, J 6.5 Hz), 2.80 (2H, t, J 7.0 Hz), 1.72 (2H, p, J 7.5 Hz), 1.60-1.53 (2H, m), 1.47-1.40 (2H, m), 1.39-1.34 (2H, m).

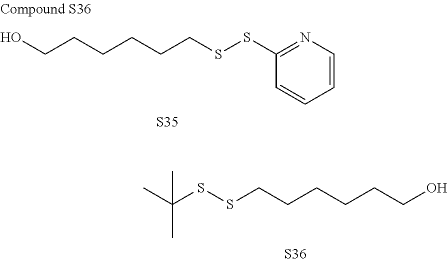

To a solution of compound S35 (1.0 g, 4.1 mmol) in dichloromethane (15.0 mL) was added MeOTf (0.67 g, 4.1 mmol). The mixture was stirred for 15 min, and then 2-methyl-2-propanethiol (0.9 mL, 8.2 mmol) and N, N-diisopropylethylamine (2.0 mL) were added. The resulting mixture was stirred for another 30 min. Evaporation of the mixture afforded a residue, which was subjected to flash silica gel column purification on an ISCO companion (ethyl acetate/hexane=5%-60%) to give 0.61 g (67%) of the title compound S36 as a colorless oil. ¹H NMR (500 MHz, CDCl₃): δ3.65 (2H, t, J 6.5 Hz), 2.70 (2H, t, J 7.0 Hz), 1.70-1.64 (2H, m), 1.62-1.55 (2H, m), 1.45-1.35 (4H, m), 1.33 (9H, s).

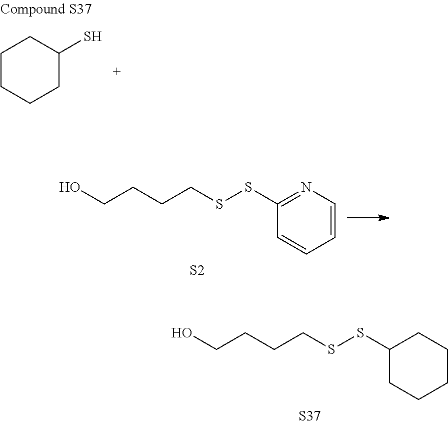

To a solution of compound S2 (0.43 g, 2.0 mmol) in dichloromethane (10.0 mL) was added MeOTf (0.33 g, 2.0 mmol). The mixture was stirred for 15 min, and then cyclohexanethiol (0.23 g, 2.0 mmol) and N, N-diisopropylethylamine (1.0 mL) were added. The resulting mixture was stirred for another 30 min. Evaporation of the mixture afforded a residue, which was subjected to flash silica gel column purification on an ISCO companion (ethyl acetate/hexane=5%-60%) to give 0.36 g (81%) of the title compound S37 as a colorless oil. ¹H NMR (500 MHz, CDCl₃): δ3.67 (2H, t, J 6.5 Hz), 2.74-2.68 (1H, m), 2.71 (1H, t, J 7.0 Hz), 2.05-2.00 (2H, m), 1.81-1.74 (4H, m), 1.71-1.65 (2H, m), 1.65-1.58 (1H, m), 1.40-1.20 (6H, m).

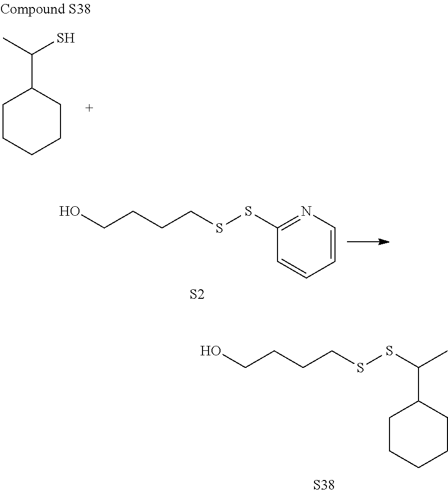

To a solution of compound S2 (0.65 g, 3.0 mmol) in dichloromethane (12.0 mL) was added MeOTf (0.49 g, 3.0 mmol). The mixture was stirred for 15 min, and then 1-cyclohexylethane-1-thiol (0.42 g, 3.6 mmol) and N, N-diisopropylethylamine (1.0 mL) were added. The resulting mixture was stirred for another 30 min. Evaporation of the mixture afforded a residue, which was subjected to flash silica gel column purification on an ISCO companion (ethyl acetate/hexane=5%-60%) to give 0.58 g (78%) of the title compound S38 as a colorless oil. ¹H NMR (500 MHz, CDCl₃): δ3.68 (2H, t, J 6.5 Hz), 2.75-2.65 (1H, m), 2.70 (2H, t, J 7.0 Hz), 1.82-1.72 (6H, m), 1.70-1.63 (3H, m), 1.58-1.52 (1H, m), 1.28 (3H, d, J 7.0 Hz), 1.30-1.05 (5H, m).

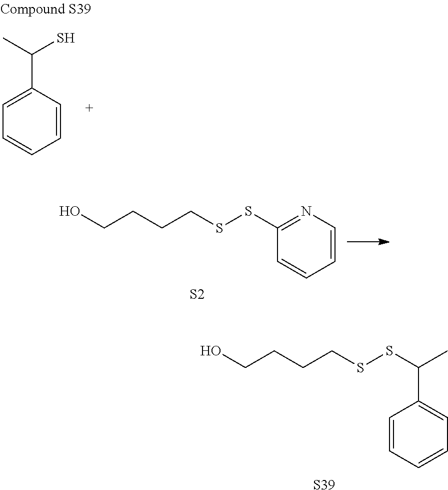

To a solution of compound S2 (0.43 g, 2.0 mmol) in methanol (5.0 mL) were added benzylethane-1-thiol (0.28 g, 2.0 mmol) and acetic acid (30 μL). The resulting mixture was stirred overnight. Evaporation of the mixture afforded a residue, which was subjected to flash silica gel column purification on an ISCO companion (ethyl acetate/hexane=5%-60%) to give 0.24 g (50%) of the title compound S39 as a colorless oil. ¹H NMR (500 MHz, CDCl₃): δ7.38-7.30 (4H, m), 7.27-7.23 (1H, m), 3.59 (2H, t, J 6.5 Hz), 2.30 (2H, t, J 7.0 Hz), 1.67 (3H, d, J 7.0 Hz), 1.62-1.51 (4H, m).

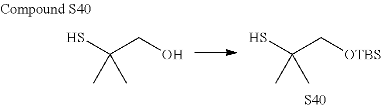

To a solution of 2-mercapto-2-methylpropan-1-ol (0.50 g, 4.7 mmol) in dichloromethane (15.0 mL) were added TBDMSCl (0.75 g, 4.9 mmol) and imidazole (0.48 g, 7.1 mmol) at 0° C. and stirred for 30 min forming large amount of white precipitates. The white solid was filtered off and washed with 10 mL of dichloromethane. The filtrate was evaporated to give a residue, which was subjected to flash silica gel column purification on an ISCO companion (ethyl acetate/hexane=0%-30%) to give 0.66 g (64%) of the title compound S40 as a colorless oil. ¹H NMR (500 MHz, CDCl₃): δ3.47 (2H, s), 1.32 (6H, s), 0.92 (9H, s), 0.07 (6H, s).

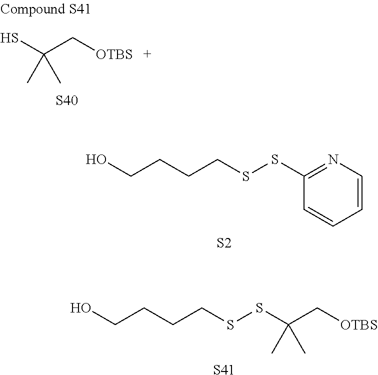

To a solution of compound S2 (0.78 g, 3.6 mmol) in dichloromethane (12.0 mL) was added MeOTf (0.59 g, 3.6 mmol). The mixture was stirred for 15 min, and then S40 (0.66 g, 3.0 mmol) and N,N-diisopropylethylamine (1.0 mL) were added. The resulting mixture was stirred for another 30 min. Evaporation of the mixture afforded a residue, which was subjected to flash silica gel column purification on an ISCO companion (ethyl acetate/hexane=5%-60%) to give 0.80 g (82%) of the title compound S41 as a colorless oil. ¹H NMR (500 MHz, CDCl₃): δ3.58 (2H, t, J 6.5 Hz), 3.41 (2H, s), 2.62 (2H, t, J 7.0 Hz), 1.70-1.63 (2H, m), 1.62-1.55 (2H, m), 1.17 (6H, s), 0.81 (9H, s), 0.03 (6H, s).

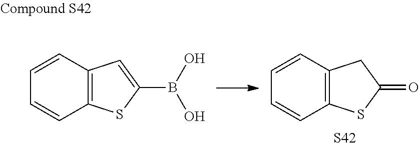 [SMILES: OB(O)c1cc2ccccc2s1 ; O=C1Cc2ccccc2S1 ; "S42"]

To a solution of thianaphthene-2-boronic acid (3.09 g, 17.0 mmol) in EtOH (30.0 mL) was added hydrogen peroxide (30%, 5.6 fmL) dropwise. After stirring overnight, the reaction mixture was carefully concentrated under reduced pressure, diluted with water (100 mL), and extracted with ethyl acetate (70 mL×3). The combined organic layer was dried over anhydrous sodium sulfate and concentrated in vacuo to afford a residue, which was subjected to flash silica gel column purification on a ISCO companion (ethyl acetate/hexane=0%-20%) to give 2.17 g (85%) of the title compound S42 as a colorless oil. ¹H NMR (500 MHz, CDCl₃): δ7.34 (1H, dd, J 8.0 Hz), 7.31-7.28 (2H, m), 7.22 (1H, td, J 8.0, 1.0 Hz), 3.98 (2H, s).

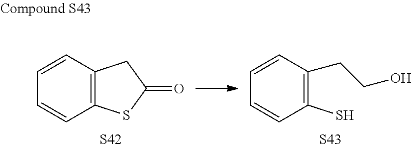 [SMILES: O=C1Cc2ccccc2S1 ; OCCc1ccccc1S ; "S42" ; "S43"]

To a solution of LiAlH₄ (1.1 g, 28.8 mmol) in THF (40.0 mL) was added a solution of compound S42 (2.16 g, 14.4 mmol) in THF. The mixture was stirred overnight and the reaction mixture was quenched with water (20 mL) carefully while cooling to 0° C., followed by addition of 50 mL of 1N HCl. The phases were separated, and the aqueous layer was extracted with ethyl acetate (2×50 mL). The combined organic layers were washed with brine, dried over anhydrous sodium sulfate and concentrated in vacuo to afford a residue, which was subjected to flash silica gel column purification on a ISCO companion (ethyl acetate/hexane=10%-50%) to give 0.69 g (31%) of the title compound S43 as a colorless oil. ¹H NMR (500 MHz, CDCl₃): δ7.31 (1H, dd, J 7.5, 1.5 Hz), 7.20 (1H, dd, J 7.5, 1.5 Hz), 7.16-7.08 (2H, m), 3.91 (2H, t, J 6.5 Hz), 3.41 (1H, s), 2.98 (1H, J 6.5 Hz).

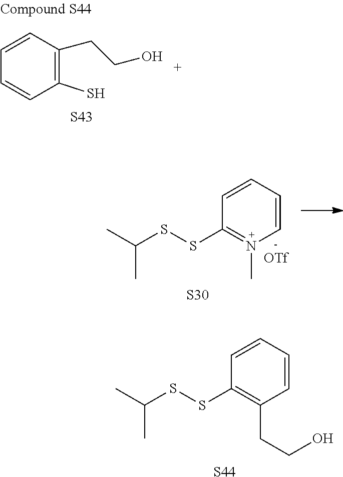

To a solution of compound S43 (0.23 g, 1.5 mmol) in dichloromethane (5.0 mL) was added the disulfide pyridinium salt S30 (0.70 g, 2.0 mmol) and N,N-diisopropylethylamine (1.0 mL). The mixture was stirred for 10 min and the volatiles were removed under vacuum to give a residue, which was subjected to flash silica gel column purification on a ISCO companion (ethyl acetate/hexane=5%-50%) to give 0.29 g (85%) of the title compound S44 as a colorless oil. ¹H NMR (500 MHz, CDCl₃): δ7.79 (1H, d, J 8.0 Hz), 7.27-7.23 (1H, m), 7.21-7.18 (2H, m), 3.91 (2H, t, J 6.5 Hz), 3.10 (2H, t, J 6.5 Hz), 3.07-3.03 (1H, m), 1.30 (6H, d, J 7.0 Hz).

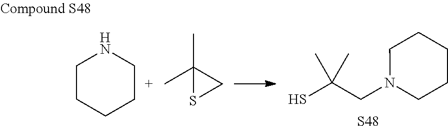

The mixture of Isobutylene Sulfide (0.88 g, 10.0 mmol) and piperidine (0.84 mL, 8.5 mmol) was heated to 80° C. and stirred for 4 hours. Evaporation of the volatiles afforded the crude product S48, which was used directly in next step without purification.

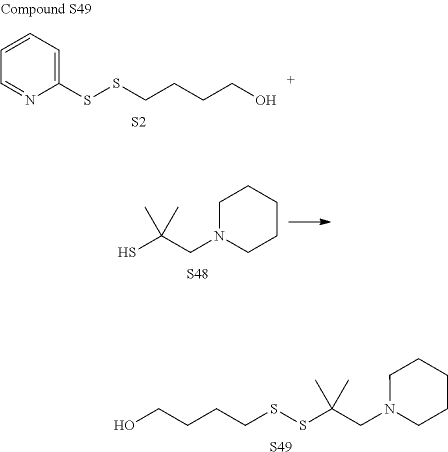

To a solution of compound S2 (0.65 g, 3.0 mmol) in dichloromethane (12.0 mL) was added MeOTf (0.49 g, 3.0 mmol). The mixture was stirred for 15 min, and then the crude S48 (0.49 g, 3.0 mmol) and diisopropylethylamine (1.0 mL) were added. The resulting mixture was stirred for another 30 min. Evaporation of the mixture afforded a residue, which was subjected to flash silica gel column purification on an ISCO companion (ethyl acetate/hexane=5%-60%) to give 0.50 g (52% for two steps) of the title compound S49 as a colorless oil. ¹H NMR (500 MHz, CDCl₃): δ3.69 (2H, m), 2.72 (2H, t, J 7.0 Hz), 2.49 (4H, m), 2.37 (2H, s), 1.80-1.70 (2H, m), 1.70-1.62 (2H, m), 1.55-1.47 (4H, m), 1.40-1.34 (2H, m), 1.27 (6H, s).

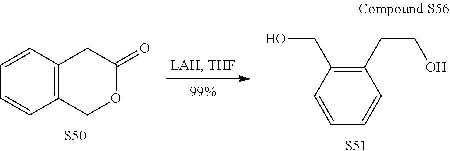 [SMILES: O=C1Cc2ccccc2CO1 ; OCCc1ccccc1CO ; "S50" ; "S51"]

The suspension of lithium aluminum hydride (1.03 g, 27.0 mmol) in THF was cooled to 0° C., to which was added drop wise a solution of 3-Isochromanone S50 (2.0 g, 13.5 mmol) in 25 mL of THF under Argon atmosphere. The reaction mixture was warmed up to room temperature and further stirred for 3 hours. The suspension was cooled to 0° C. again by ice bath, quenched with saturated Na₂SO₄ solution and filtered through a pad of celite. The filtrate was condensed under reduced pressure. The crude mixture was diluted with 100 mL of ethyl acetate and washed sequentially by saturated NaHCO₃ solution (20.0 mL) and brine (20.0 mL), then dried over anhydrous Na₂SO₄. The solvent was evaporated in vacuo to get intermediate S51 as a colorless oil (2.01 g, 99% yield), which directly went to the next step. ¹H NMR (500 MHz): δ7.34-7.22 (m, 4H), 4.65 (s, 2H), 3.89 (t, J 6.0 Hz, 2H), 2.96 (t, J 6.0 Hz, 2H)

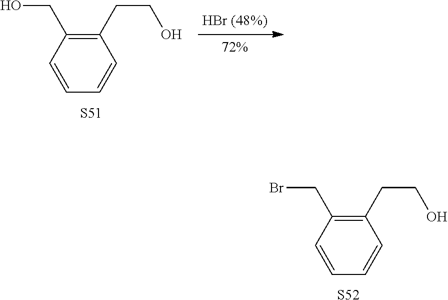 [SMILES: OCCc1ccccc1CO ; "S51" ; OCCc1ccccc1CBr ; "S52"]

To intermediate S51 (4.0 g, 26.5 mmol) was added a solution of 48% hydrobromic acid (20.0 mL) drop wise. The reaction mixture was stirred for 3 hours at room temperature before being poured into ice water. The resulting mixture was extracted with ethyl ether (200 mL), washed sequentially by saturated NaHCO₃ solution (20.0 mL) and brine (20.0 mL), and then dried over anhydrous Na₂SO₄. The solvent was evaporated in vacuo to get intermediate S52 as a light yellow oil (4.2 g, 72% yield), which directly went to the next step.

¹H NMR (500 MHz): δ7.37-7.15 (m, 4H), 4.59 (s, 2H), 3.94 (t, J 6.5 Hz, 2H), 3.03 (t, J 6.5 Hz, 2H)

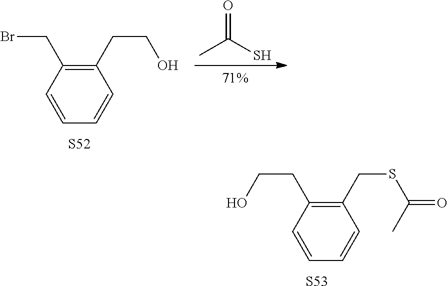

To a solution of S52 (5.5 g, 25.6 mmol) and thioacetic acid (2.24 g, 30.7 mmol) in 50.0 mL of methanol was added NaHCO₃ (2.58 g, 30.7 mmol) portion wise. The reaction mixture was stirred for 2 hours at room temperature before neutralized to pH 7 with 1 N HCl solution and the volatiles evaporated in vacuo. The residue was diluted with 300 mL of ethyl acetate and washed with brine (50.0 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give product S53 as a light yellow oil (3.8 g, 71% yield).

¹H NMR (500 MHz): δ7.30-7.18 (m, 4H), 4.20 (s, 2H), 3.87 (t, J 7.0 Hz, 2H), 2.92 (t, J 7.0 Hz, 2H), 2.34 (s, 3H)

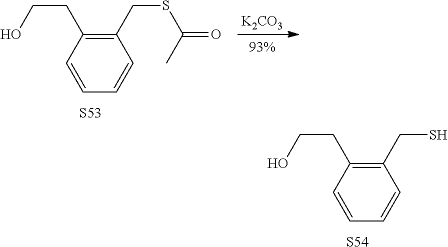

To a solution of S53 (3.8 g, 18.1 mmol) in 50 mL of methanol was added K₂CO₃ (3.0 g, 21.7 mmol) portion wise under argon atmosphere. The reaction mixture was stirred for 30 minutes at room temperature before neutralized to pH 7 with 1 N HCl solution and the volatile evaporated in vacuo. The residue was diluted with 200 mL of ethyl acetate and washed with brine (50.0 mL), then dried over anhydrous Na₂SO₄. The solvent was evaporated in vacuo to give crude product S54 as light yellow oil (2.8 g, 93% yield), which directly went to the next step reaction.

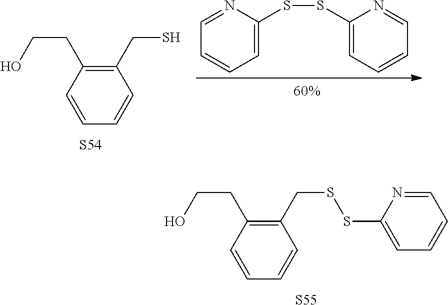

To a solution of crude S54 (2.8 g, 16.7 mmol) and dithiopyridine (4.4 g, 20.0 mmol) in 50.0 mL of ethanol was added 1.0 mL of acetic acid. The reaction mixture was stirred for 3 hours at room temperature before being condensed in vacuo. The crude mixture was purified by silica gel column chromatography using ethyl acetate/hexane solvent system (0-50% gradient on Combi Flash Rf Instrument) to give product S55 as colorless oil (2.5 g, 60% yield).

¹H NMR (500 MHz): δ8.43 (d, J 4.5 Hz, 1H), 7.58-7.55 (m, 2H), 7.26-7.07 (m, 5H), 4.14 (s, 2H), 3.96 (t, J 6.5 Hz, 2H), 3.04 (t, J 6.5 Hz, 2H)

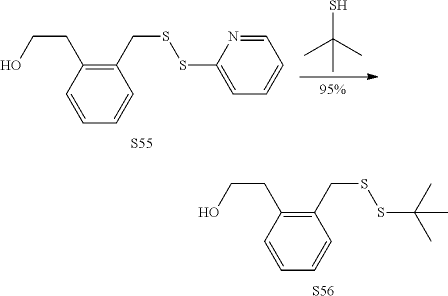

To a solution of S55 (1.14 g, 4.1 mmol) and tert-butyl mercaptan (560 μL, 4.9 mmol) in 25 mL of methanol was added 100 μL of acetic acid. The reaction mixture was stirred for 48 hours at room temperature before being condensed in vacuo. The crude mixture was purified by silica gel column chromatography using ethyl acetate/hexane solvent system (0-50% gradient on Combi Flash Rf Instrument) to give product S56 as colorless oil (0.90 g, 97% yield, 0.14 g of S55 was recovered).

¹H NMR (500 MHz): δ7.29-7.20 (m, 4H), 4.03 (s, 2H), 3.92 (t, J 6.5 Hz, 2H), 3.01 (t, J 6.5 Hz, 2H), 1.36 (s, 9H)

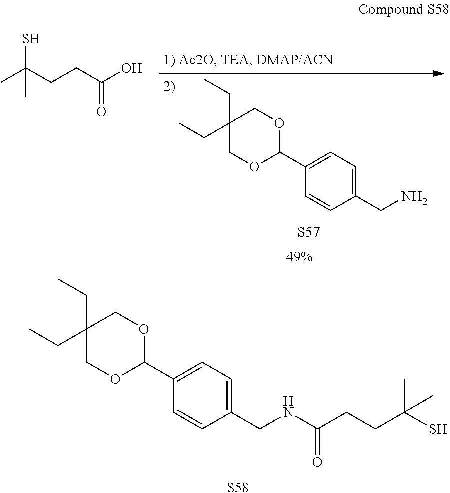

To a solution of 4-Sulfanyl-4-methylpentanoic acid (5.0 g, 33.7 mmol) and acetic anhydride (3.5 mL, 37.1 mmol) in 30.0 mL of acetonitrile under argon atmosphere was added triethylamine (9.4 mL, 67.4 mmol) and catalytic amount of DMAP. The reaction mixture was stirred at room temperature for 30 min before intermediate S57 (12.6 g, 50.55 mmol) in 15.0 mL of acetonitrile was added. The reaction mixture was stirred at room temperature overnight before being condensed in vacuo. The crude mixture was purified by silica gel column chromatography using ethyl acetate/hexane solvent system (0-50% gradient on Combi Flash Rf Instrument) to give product S58 as light yellow oil (6.2 g, 49% yield). ¹H NMR (500 MHz): δ7.32 (d, J 8.5 Hz, 2H), 7.26 (d, J 8.5 Hz, 2H), 5.7 (brs, 1H), 5.37 (s, 1H), 4.41 (d, J 5.5 Hz, 2H), 3.94 (d, J 11.5 Hz, 2H), 3.58 (d, J 11.5 Hz, 2H), 2.37 (m, 2H), 1.93 (m, 2H), 1.81 (q, J 7.5 Hz, 2H), 1.38 (s, 6H), 1.13 (q, J 8.0 Hz, 2H), 0.89 (t, J 7.5 Hz, 3H), 0.81 (t, J 8.0 Hz, 3H), 1.83 (m, 2H), 1.70 (m, 2H), 1.62 (m, 2H), 1.25 (s, 6H)

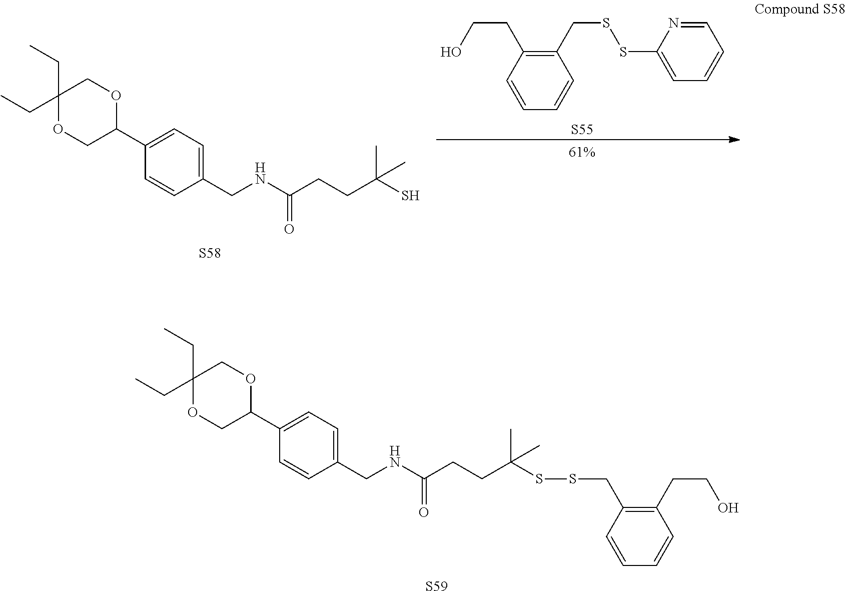 [SMILES: "S55" ; CCC1(CC)COC(c2ccc(CNC(=O)CCC(C)(C)S)cc2)CO1 ; OCCc1ccccc1CSSc1ccccn1 ; "S58" ; CCC1(CC)COC(c2ccc(CNC(=O)CCC(C)(C)SSCc3ccccc3CCO)cc2)CO1 ; "S59"]

To a solution of S55 (0.50 g, 1.8 mmol) and S58 (0.68 g, 1.8 mmol) in 10.0 mL of methanol was added 100 μL of acetic acid. The reaction mixture was stirred for 16 hours at room temperature before being condensed in vacuo. The crude mixture was purified by silica gel column chromatography using ethyl acetate/hexane solvent system (0-50% gradient on Combi Flash Rf Instrument) to give product S59 as light yellow oil (0.60 g, 61% yield). ESI MS for C₃₀H₄₃NO₄S₂ calculated 545. observed [M+H]⁺ 546. ¹H NMR (500 MHz): δ7.44 (d, J 8.0 Hz, 2H), 7.30-7.18 (m, 6H), 5.78 (brs, 1H), 5.36 (s, 1H), 4.41 (d, J 5.5 Hz, 2H), 4.07 (s, 2H), 3.93 (d, J 11.5 Hz, 2H), 3.81 (brs, 2H), 3.58 (d, J 11.5 Hz, 2H), 3.02 (t, J 7.5 Hz, 2H), 2.86 (brs, 1H), 2.34 (m, 2H), 2.05 (m, 2H), 1.81 (q, J 7.5 Hz, 2H), 1.30 (s, 6H), 1.13 (q, J 8.0 Hz, 2H), 0.89 (t, J 8.0 Hz, 3H), 0.81 (t, J 7.5 Hz, 3H)

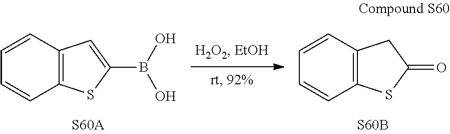 [SMILES: OB(O)c1cc2ccccc2s1 ; O=C1Cc2ccccc2S1 ; "S60B"]

To a solution of compound S60A (30.0 g, 168.5 mmol) in EtOH (120 mL) was added 30% Hydrogen peroxide (50 mL) dropwise over 45 min (caution: exothermic). Reaction mixture became turbid with white precipitate. TLC showed completion of reaction at 3 hr, and the reaction mixture was diluted with water (300 mL), carefully extracted with dichloromethane (200 mL×3). The combined organic layer was dried over anhydrous sodium sulfate and concentrated in vacuo to afford crude product. This was purified by flash silica gel column (220 g) using ISCO companion (ethyl acetate/hexane, 0-20% over 15 column volumes) to give 23.5 g (92%) of compound S60B as a light yellow oil which became solid on standing at room temperature. ¹H NMR (500 MHz, CDCl₃): δ7.34 (1H, dd, J 8.0 Hz), 7.31-7.28 (2H, m), 7.22 (1H, td, J 8.0, 1.0 Hz), 3.98 (2H, s)

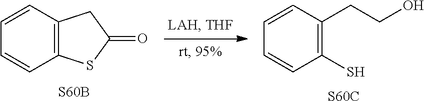 [SMILES: "S60B" ; "S60C" ; O=C1Cc2ccccc2S1 ; OCCc1ccccc1S]

To an ice cold solution of LiAlH₄ (7.4 g, 200.0 mmol) in diethyl ether (200 mL) was added dropwise a solution of compound S60B (15.0 g, 100.0 mmol) in diethyl ether over 1 hr (caution: gas evolution and exothermic). The reaction mixture was allowed to reach room temperature and stirring continued overnight. TLC showed completion of reaction, which was carefully quenched with aq. Sodium sulfate until gas evolution stopped to give a white precipitate. To this 100 m L of 10% H₂SO₄ was carefully added and the organic layer separated. Aqueous layer extracted with 3×75 mL ether and the combined organic layers washed with water, brine, dried over sodium sulfate and evaporated to give compound S60C (14.6 g, 95%) as colorless oil which was used in the next reaction without further purification. ¹H NMR (500 MHz, CDCl₃): δ7.31 (1H, dd, J 7.5, 1.5 Hz), 7.20 (1H, dd, J 7.5, 1.5 Hz), 7.16-7.08 (2H, m), 3.91 (2H, t, J 6.5 Hz), 3.41 (1H, s), 2.98 (1H, J 6.5 Hz)

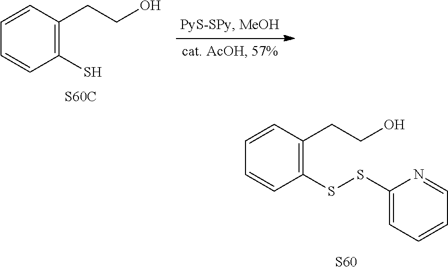 [SMILES: "S60C" ; "S60" ; OCCc1ccccc1S ; OCCc1ccccc1SSc1ccccn1]

To a solution of dithiodipyridine (52.0 g, 236.3 mmol) and acetic acid (3.0 mL) in methanol (200 mL) at room temperature was added a solution of compound S60C (14.6 g, 94.5 mmol) in methanol (50 mL) and stirred overnight. Volatiles removed and to the residue 100 mL diethyl ether was added and the separated solids were filtered, washed with diethyl ether (3×50 mL). The combined ether washings evaporated to give crude product which on flash silica gel column purification using ISCO companion (ethyl acetate/hexane, 0-50%) gave 14.1 g (57%) of compound S60. ¹H NMR (500 MHz, CDCl₃): δ8.48 (1H, d, J 5.0 Hz), 7.65-7.60 (3H, m), 7.25-7.18 (3H, m), 7.13-7.10 (1H, m), 3.96 (2H, t, J 6.5 Hz), 3.17 (1H, t, J 6.5 Hz)

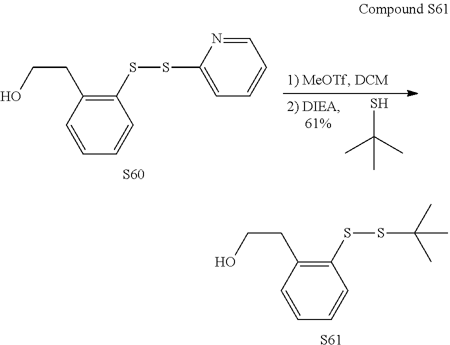

To a solution of compound S60 (4.5 g, 17.0 mmol) in 30.0 mL of dichloromethane was added MeOTf drop wise at room temperature. The reaction mixture was stirred for 10 minutes before tert-butyl mercaptan (1.9 mL, 17.0 mmol) and DIEA (6.0 mL, 34.0 mmol) were added. The reaction mixture was stirred for another 30 min at room temperature before being condensed in vacuo. The crude mixture was purified by silica gel column chromatography using ethyl acetate/hexane solvent system (0-30% gradient on Combi Flash Rf Instrument) to give product S61 as colorless oil (2.5 g, 61% yield).

¹H NMR (500 MHz): δ7.84 (d, J 5.0 Hz, 1H), 7.25-7.13 (m, 3H), 3.92 (t, J 7.0 Hz, 2H), 3.12 (t, J 7.0 Hz, 2H), 1.30 (s, 9H)

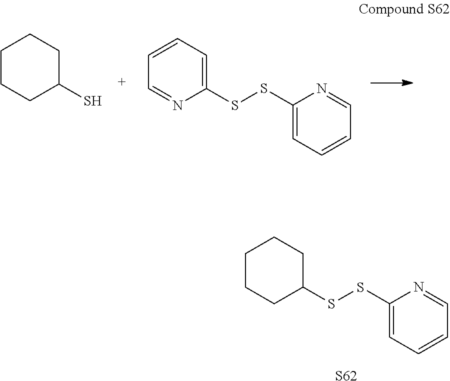 [SMILES: SC1CCCCC1 ; c1ccc(SSc2ccccn2)nc1 ; c1ccc(SSC2CCCCC2)nc1 ; "S62"]

Compound S62 was prepared according to the procedure described for compound S55 using AcOH activator as reported previously. ¹H NMR (500 MHz, CDCl₃): δ8.45 (1H, s), 7.78 (1H, d, J 8.0 Hz), 7.64 (1H, t, J 8.0 Hz), 7.09-7.04 (1H, m), 2.90-2.80 (1H, m), 2.06-1.98 (2H, m), 1.80-1.73 (2H, m), 1.63-1.56 (1H, m), 1.45-1.35 (2H, m), 1.33-1.18 (3H, m)

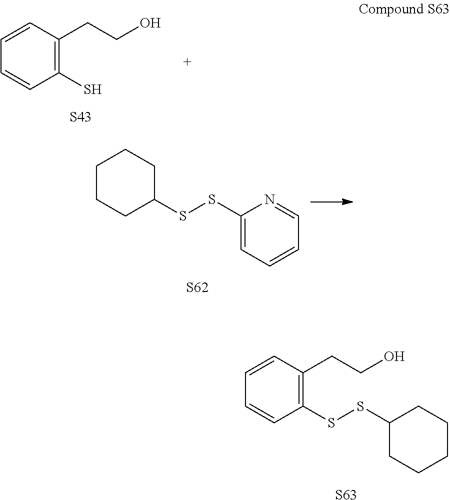 [SMILES: OCCc1ccccc1S ; "S43" ; c1ccc(SSC2CCCCC2)nc1 ; "S62" ; OCCc1ccccc1SSC1CCCCC1 ; "S63"]

Compound S63 was prepared according to the procedure described for compound S41 using MeOTf activator as reported previously. ¹H NMR (500 MHz, CDCl₃): δ7.80 (1H, d, J=8.0 Hz), 7.30-7.23 (1H, m), 7.21-7.17 (2H, m), 3.90 (2H, t, J 6.5 Hz), 3.09 (2H, t, J 6.5 Hz), 2.82-2.70 (1H, m), 2.06-1.98 (2H, m), 1.80-1.72 (2H, m), 1.63-1.55 (1H, m), 1.41-1.18 (5H, m)

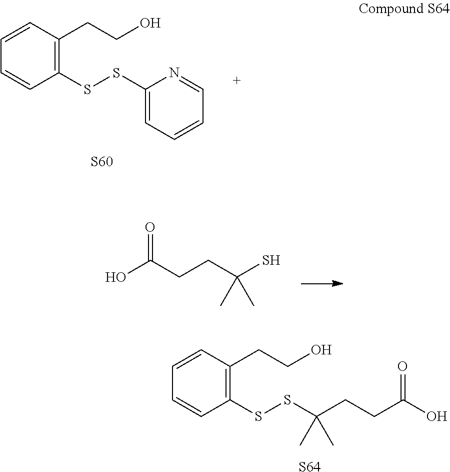

Compound S64 was prepared according to the procedure described for compound S41 using MeOTf activator as reported previously. ¹H NMR (500 MHz, CDCl₃): δ7.81 (1H, d, J 8.0 Hz), 7.26-7.21 (1H, m), 7.19-7.13 (2H, m), 3.93 (2H, t, J 6.5 Hz), 3.13 (2H, t, J 6.5 Hz), 2.38-2.34 (2H, m), 1.90-1.86 (2H, m), 1.27 (1H, s)

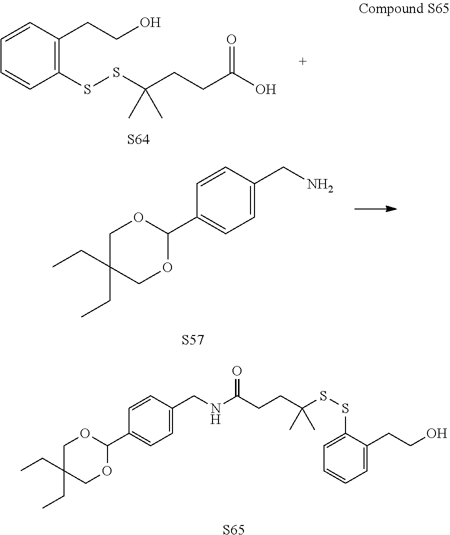 [SMILES: CC(C)(CCC(=O)O)SSc1ccccc1CCO ; "S64" ; CCC1(CC)COC(c2ccc(CN)cc2)OC1 ; "S57" ; CCC1(CC)COC(c2ccc(CNC(=O)CCC(C)(C)SSc3ccccc3CCO)cc2)OC1 ; "S65"]

To a mixture of compound S57 (1.13 g, 4.54 mmol) and S64 (1.24 g, 4.13 mmol) in DMF (12 mL) were added HCTU (2.56 g, 6.20 mmol) and N, N-Diisopropylethylamine (1.76 mL, 10.3 mmol). The mixture was stirred for 1 hour and the volatiles were removed under high vacuum to give a residue, which was subjected to flash silica gel column purification on a ISCO companion (ethyl acetate/hexane, 10-70%) to give 1.28 g (58%) of the title compound S65 as a colorless oil. ¹H NMR (500 MHz, CDCl₃): δ7.81 (1H, d, J 8.0 Hz), 7.47 (2H, d, J 8.0 Hz), 7.21-7.10 (3H, m), 7.07 (1H, t, J 7.5 Hz), 7.01 (1H, d, J 7.5 Hz), 5.40 (1H, s), 4.92 (1H, s, br), 4.24 (2H, d, J 5.5 Hz), 3.96 (2H, d, J 11.5 Hz), 3.73 (2H, t, J 6.5 Hz), 3.61 (2H, d, J 11.5 Hz), 2.97 (2H, t, J 6.5 Hz), 2.10-2.02 (2H, m), 1.84 (2H, q, J 7.5 Hz), 1.81-1.76 (2H, m), 1.29 (6H, s), 1.15 (2H, q, J 7.5 Hz), 0.90 (3H, t, J 7.5 Hz), 0.82 (3H, t, J 7.5 Hz)

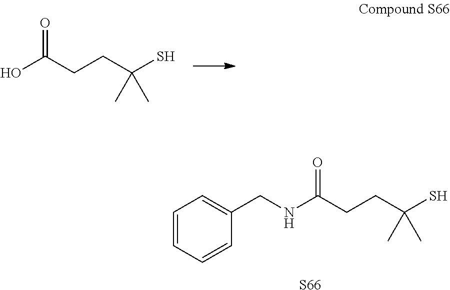

To a mixture of 2-Methyl-2-mercaptopentanoic acid (0.74 g, 5.0 mmol) and acetic anhydride (0.52 mL, 5.5 mmol) in acetonitrile (10.0 mL) were added triethylamine (1.39 mL, 10.0 mmol) and DMAP (5 mg). The mixture was stirred for 1 hour, and then benzylamine (1.37 mL, 12.5 mmol) was added to the mixture and stirring continued for overnight. The volatiles were removed under vacuum to give a residue, which was subjected to flash silica gel column purification on an ISCO companion (ethyl acetate/hexane, 10-70%) to give 0.70 g (59%) of the title compound S66 as colorless oil. ¹H NMR (500 MHz, CDCl₃): δ7.36-7.32 (2H, m), 7.30-7.26 (3H, m), 5.73 (1H, s), 4.45 (2H, d, J 6.0 Hz), 2.43-2.38 (2H, m), 1.98-1.94 (2H, m), 1.39 (6H, s)

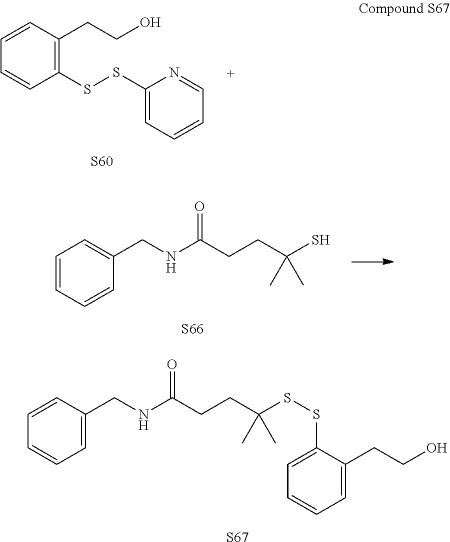 [SMILES: OCCc1ccccc1SSc1ccccn1 ; "S60" ; CC(C)(S)CCC(=O)NCc1ccccc1 ; "S66" ; CC(C)(CCC(=O)NCc1ccccc1)SSc1ccccc1CCO ; "S67"]

Compound S67 was prepared according to the procedure described for compound S41 using MeOTf activator as reported previously. ¹H NMR (500 MHz, CDCl₃): δ7.81 (1H, d, J 8.0 Hz), 7.37-7.26 (3H, m), 7.21-7.15 (3H, m), 7.08-7.02 (2H, m), 5.14 (1H, s, br), 4.28 (2H, d, J 5.5 Hz), 3.89 (2H, t, J 6.5 Hz), 3.08 (2H, t, J 6.5 Hz), 2.12-2.05 (2H, m), 1.87-1.82 (2H, m), 1.29 (6H, s)

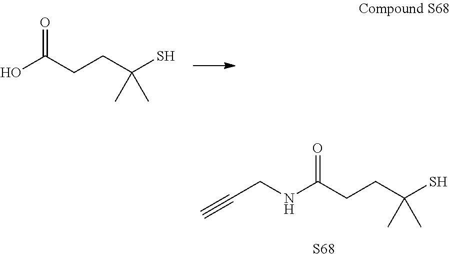

To a mixture of 2-Methyl-2-mercaptopentanoic acid (0.74 g, 5.0 mmol) and acetic anhydride (0.52 mL, 5.5 mmol) in acetonitrile (10.0 mL) were added triethylamine (1.39 mL, 10.0 mmol) and DMAP (5 mg). The mixture was stirred for 1 hour, and then propargylamine (0.69 g, 12.5 mmol) was added to the mixture and stirring continued for overnight. The volatiles were removed under vacuum to give a residue, which was subjected to flash silica gel column purification on an ISCO companion (ethyl acetate/hexane, 5-55%) to give 0.72 g (59%) of the title compound S68 as a white solid. ¹H NMR (500 MHz, CDCl₃): δ5.66 (1H, s), 4.06 (2H, dd, J 5.0, 2.5 Hz), 2.41-2.37 (2H, m), 2.23 (1H, t, J 2.5 Hz), 1.95-1.91 (2H, m), 1.39 (6H, s)

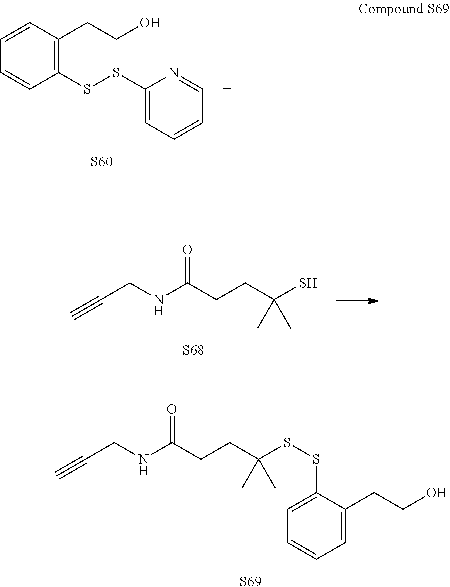 [SMILES: OCCc1ccccc1SSc1ccccn1 ; "S60" ; C#CCNC(=O)CCC(C)(C)S ; "S68" ; C#CCNC(=O)CCC(C)(C)SSc1ccccc1CCO ; "S69"]

Compound S69 was prepared according to the procedure described for compound S41 using MeOTf activator as reported previously. ¹H NMR (500 MHz, CDCl₃): δ7.83 (1H, d, J 8.0 Hz), 7.30-7.16 (3H, m), 5.05 (1H, s), 3.95 (2H, t, J 6.5 Hz), 3.88 (2H, dd, J 5.5, 2.5 Hz), 3.15 (2H, t, J 6.5 Hz), 2.23 (1H, t, J 2.5 Hz), 2.10-2.04 (2H, m), 1.83-1.79 (2H, m), 1.28 (6H, s)

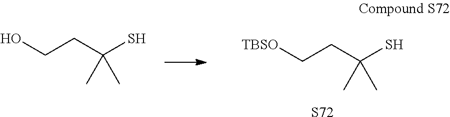

To a solution of 2-Mercapto-2-Methylbutan-1-ol (1.2 g, 10 mmol) in dichloromethane (25.0 mL) were added TBDMSCl (1.58 g, 10.5 mmol) and imidazole (1.02 g, 15 mmol) at 0° C. and stirred for 30 mins forming large amount of white precipitates. The white solid was filtered off and washed with 30.0 mL of dichloromethane. The filtrate was evaporated to give a residue, which was subjected to flash silica gel column purification on an ISCO companion (ethyl acetate/hexane 0-30%) to give 1.63 g (71%) of the title compound S72 as colorless oil. ¹H NMR (500 MHz, CDCl₃): δ7.83 (1H, d, J 8.0 Hz), 7.30-7.16 (3H, m), 5.05 (1H, s), 3.95 (2H, t, J 6.5 Hz), 3.88 (2H, dd, J 5.5, 2.5 Hz), 3.15 (2H, t, J 6.5 Hz), 2.23 (1H, t, J 2.5 Hz), 2.10-2.04 (2H, m), 1.83-1.79 (2H, m), 1.28 (6H, s)

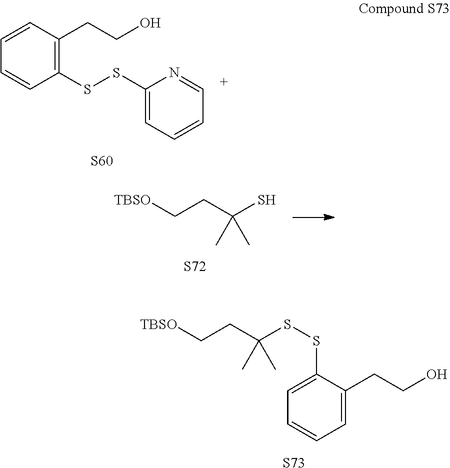 [SMILES: OCCc1ccccc1SSc1ccccn1 ; "S60" ; CC(C)(S)CCO[Si](C)(C)C(C)(C)C ; "S72" ; CC(C)(CCO[Si](C)(C)C(C)(C)C)SSc1ccccc1CCO ; "S73"]

Compound S73 was prepared according to the procedure described for compound S41 using MeOTf activator as reported previously. ¹H NMR (500 MHz, CDCl₃): δ7.83 (1H, d, J 8.0 Hz), 7.30-7.12 (3H, m), 3.91 (2H, t, J 6.5 Hz), 3.68 (2H, t, J 7.0 Hz), 3.12 (2H, t, J 6.5 Hz), 1.83 (1H, t, J 6.5 Hz), 1.28 (6H, s), 0.87 (9H, s), 0.03 (6H, s)

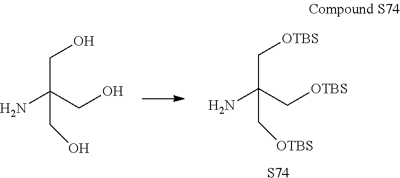 [SMILES: NC(CO)(CO)CO ; CC(C)(C)[Si](C)(C)OCC(N)(CO[Si](C)(C)C(C)(C)C)CO[Si](C)(C)C(C)(C)C ; "S74"]

To a solution of TBDMSCl (6.7 g, 44.6 mmol) and imidazole (6.3 g, 92.9 mmol) in DMF (5.0 mL) was added trihydroxy amine (1.5 g, 12.4 mmol) and stirred for 1 hour. The mixture was diluted with water (15.0 mL), and extracted with dichloromethane (3×15.0 mL). The combined organic layers were dried over anhydrous sodium sulfate and the filtrate was evaporated in vacuum to afford a residue, which was subjected to flash silica gel column purification on a ISCO companion (ethyl acetate/hexane, 0-20%) to give 4.0 g (70%) of S74 as colorless oil. ¹H NMR (500 MHz, CDCl₃): δ3.48 (6H, s), 0.89 (27H, s), 0.04 (18H, s)

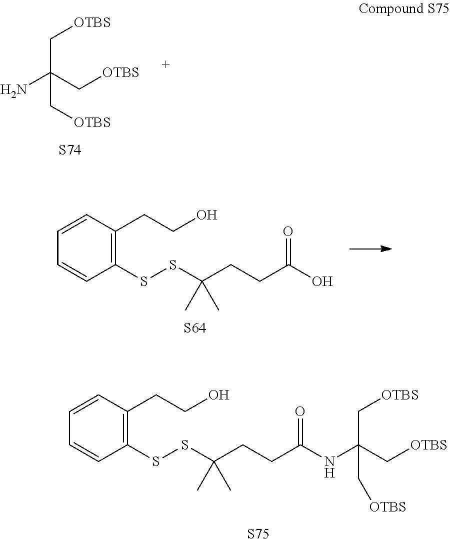 [SMILES: CC(C)(C)[Si](C)(C)OCC(N)(CO[Si](C)(C)C(C)(C)C)CO[Si](C)(C)C(C)(C)C ; "S74" ; CC(C)(CCC(=O)O)SSc1ccccc1CCO ; "S64" ; CC(C)(CCC(=O)NC(CO[Si](C)(C)C(C)(C)C)(CO[Si](C)(C)C(C)(C)C)CO[Si](C)(C)C(C)(C)C)SSc1ccccc1CCO ; "S75"]

To a mixture of compound S64 (0.6 g, 2.0 mmol) and S74 (1.16 g, 2.5 mmol) in DMF (10.0 mL) were added HATU (1.14 g, 3.0 mmol) and N, N-Diisopropylethylamine (0.85 mL, 5 mmol). The mixture was stirred for 1 hour and the volatiles were removed under high vacuum to give a residue, which was subjected to flash silica gel column purification on ISCO companion (ethyl acetate/hexane, 10-40%) to give 0.60 g (40%) of compound S75 as colorless oil. ¹H NMR (500 MHz, CDCl₃): δ7.81 (1H, d, J 8.0 Hz), 7.26-7.12 (3H, m), 5.45 (1H, s), 3.92 (2H, t, J 6.5 Hz), 3.80 (6H, s), 3.11 (2H, t, J 6.5 Hz), 2.14-2.10 (2H, m), 1.90-1.86 (2H, m), 1.23 (6H, s), 0.90 (27H, s), 0.04 (18H, s)

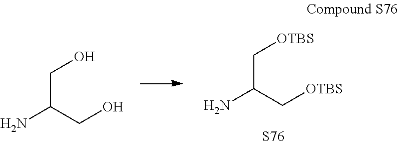 [SMILES: NC(CO)CO ; CC(C)(C)[Si](C)(C)OCC(N)CO[Si](C)(C)C(C)(C)C ; "S76"]

To a solution of TBDMSCl (7.2 g, 48 mmol), N, N-Diisopropylethylamine (5.0 mL, 29 mmol) and DMAP (50 mg) in dichloromethane (50.0 mL) was added 2-Amino-1,3-propan-diol (2.0 g, 22 mmol) and the mixture was stirred overnight. Volatiles were removed under high vacuum to give a residue, which was subjected to flash silica gel column purification on ISCO companion (ethyl acetate/hexane, 50-100% containing 2% triethylamine) to give 1.2 g (17%) of compound S76 as colorless oil. ¹H NMR (500 MHz, CDCl₃): δ3.70 (2H, dd, J 10.0, 5.5 Hz), 3.63 (2H, dd, J 10.0, 5.5 Hz), 3.04 (1H, m), 0.90 (18H, s), 0.07 (12H, s)

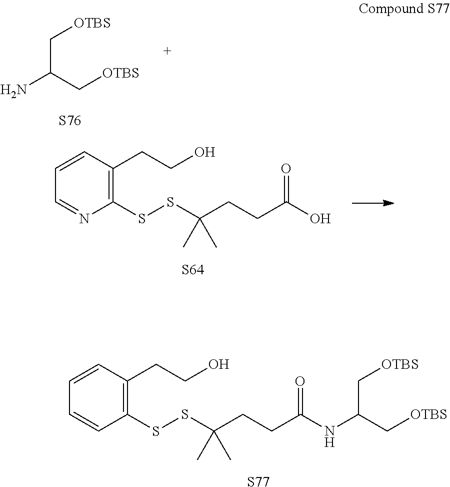 [SMILES: CC(C)(C)[Si](C)(C)OCC(N)CO[Si](C)(C)C(C)(C)C ; "S76" ; CC(C)(CCC(=O)O)SSc1ncccc1CCO ; "S64" ; CC(C)(CCC(=O)NC(CO[Si](C)(C)C(C)(C)C)CO[Si](C)(C)C(C)(C)C)SSc1ccccc1CCO ; "S77"]

To a mixture of compound S64 (0.77 g, 2.56 mmol) and S76 (0.82 g, 2.56 mmol) in DMF (10.0 mL) were added HATU (1.17 g, 3.07 mmol) and N, N-Diisopropylethylamine (0.87 mL, 5.12 mmol). The mixture was stirred for 1 hour and the volatiles were removed under high vacuum to give a residue, which was subjected to flash silica gel column purification on ISCO companion (ethyl acetate/hexane, 10%-40%) to give 0.52 g (34%) of the title compound S77 as colorless oil. ¹H NMR (500 MHz, CDCl₃): δ7.81 (1H, d, J 7.5 Hz), 7.26-7.12 (3H, m), 5.59 (1H, d, J 8.5 Hz), 3.94 (2H, t, J 6.5 Hz), 3.92-3.82 (1H, m), 3.68 (2H, dd, J 13.5, 4.5 Hz), 3.50 (2H, dd, J 9.5, 6.5 Hz), 3.12 (2H, t, J 6.5 Hz), 2.16-2.10 (2H, m), 1.92-1.84 (2H, m), 1.26 (6H, s), 0.90 (18H, s), 0.07 (12H, s)

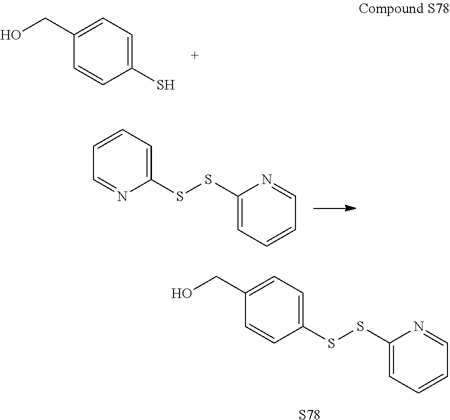

Compound S78 was prepared according to the procedure described for compound S55 using AcOH activator as reported previously. ¹H NMR (500 MHz, CDCl₃): δ8.47 (1H, d, J 4.5 Hz), 7.70-7.60 (2H, m), 7.52 (2H, d, J 8.5 Hz), 7.31 (2H, d, J 8.5 Hz), 7.10 (1H, t, J 6.0 Hz), 4.67 (2H, s)

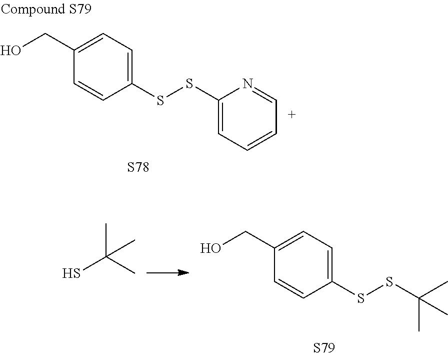

Compound S79 was prepared according to the procedure described for compound S41 using MeOTf activator as reported previously. ¹H NMR (500 MHz, CDCl₃): δ7.55 (2H, d, J 8.0 Hz), 7.29 (2H, d, J 8.0 Hz), 4.67 (2H, s), 1.31 (9H, s)

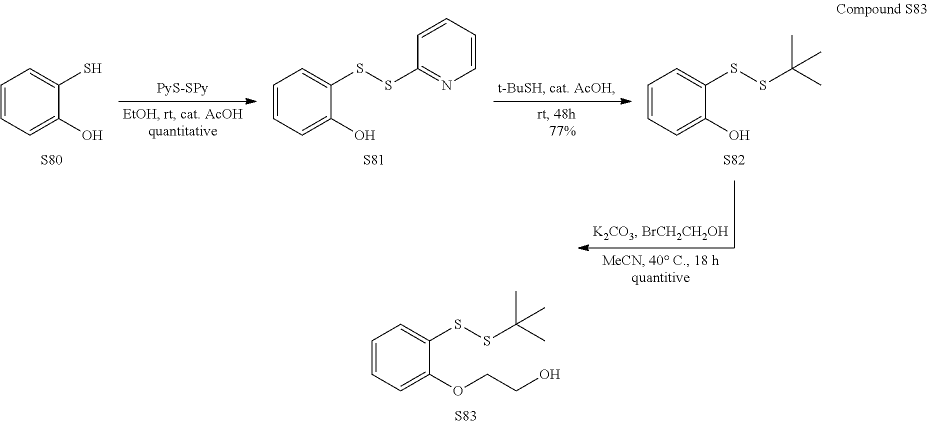

Compound S83 was prepared according to the procedure outlined in the above scheme.

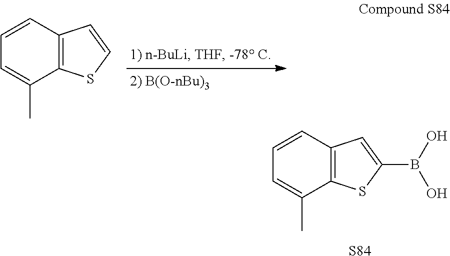

7-Methylbenzo[b]thiophene (0.74 g, 5 mmol) was dissolved in ether under argon and the solution cooled to 0°. n-Butyl lithium (2.0 ml of 2.5M in hexane, 5 mmol) was added whilst maintaining the temperature at 0-5°. The mixture was stirred at 0° for 10 minutes, then for 45 minutes at room temperature. It was cooled to 0° and tributyl borate (1.47 ml, 5.5 mmol) was added dropwise. After stirring for 1 hour at 0°, the mixture was warmed to room temperature and allowed to stand overnight, quenched with 1M hydrochloric acid. The aqueous phase was extracted with ether and the ether layer was extracted with aqueous sodium hydroxide (1M). The basic aqueous layer was acidified with concentrated hydrochloric acid to pH 2 and extracted with ether (2×50 mL). The combined organic layers were dried over anhydrous Na₂SO₄. The solvent was evaporated in vacuo to give the crude S84 (0.80 g) as a white solid.

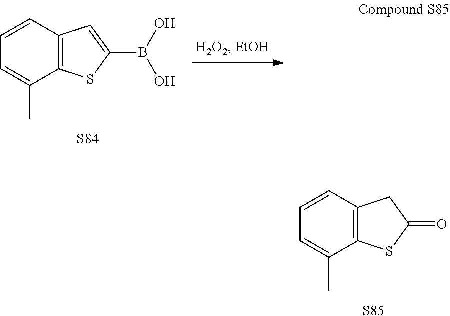

To a solution of crude S84 (0.80 g, 4.2 mmol) in EtOH (10.0 mL) was added hydrogen peroxide (30%, 1.4 mL) dropwise. After stirring overnight, the reaction mixture was carefully concentrated under reduced pressure, diluted with water (30 mL), and extracted with ethyl acetate (20 mL×3). The combined organic layer was dried over anhydrous sodium sulfate and concentrated in vacuo to afford a residue, which was subjected to flash silica gel column purification on a ISCO companion (ethyl acetate/hexane, 0-20%) to give 0.51 g (74%) of compound S85 as a colorless oil. ¹H NMR (500 MHz, CDCl₃): δ7.13 (3H, s), 4.00 (2H, s), 2.31 (3H, s)

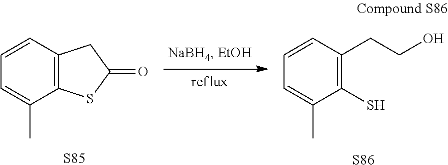

To a solution of S85 (0.51 g, 3.1 mmol) in EtOH (5 mL) was added NaBH₄ (0.59 g, 15.5 mmol) in one portion and the mixture was refluxed for another 15 mins, then cooled to room temperature. Volatiles evaporated to give a slurry white solid, which was dissolved in water and acidified to pH 2 with 1 M HCl. The mixture was extracted with dichloromethane (3×20 mL) and the combined organic layers were washed with brine, dried over anhydrous sodium sulfate and concentrated in vacuo to afford the crude compound S86 as colorless oil. ¹H NMR (500 MHz, CDCl₃): δ7.11-7.04 (3H, m), 3.92 (2H, t, J 6.5 Hz), 3.30 (1H, s), 3.05 (2H, t, J 6.5 Hz), 2.39 (3H, s)

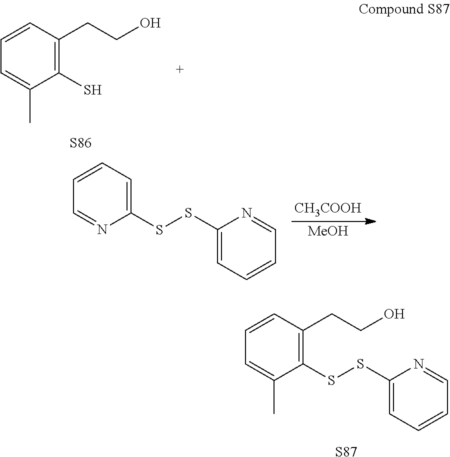

To a solution of dithiodipyridine (1.7 g, 7.8 mmol) and acetic acid (0.03 mL) in MeOH (10 mL) was added the crude S86 in MeOH (5 mL). The reaction mixture was stirred for 30 mins, then evaporated to give a yellow residue, which was subjected to flash silica gel column purification on a ISCO companion (ethyl acetate/hexane, 0-40%) to give 0.38 g (44%) of compound S87 as colorless oil. ¹H NMR (500 MHz, CDCl₃): δ8.49 (1H, d, J 5.0 Hz), 7.64-7.58 (2H, m), 7.19 (1H, t, J 7.0 Hz), 7.13 (2H, t, J 6.5 Hz), 3.83 (2H, t, J 7.0 Hz), 3.26 (2H, t, J 6.5 Hz), 2.55 (3H, s)

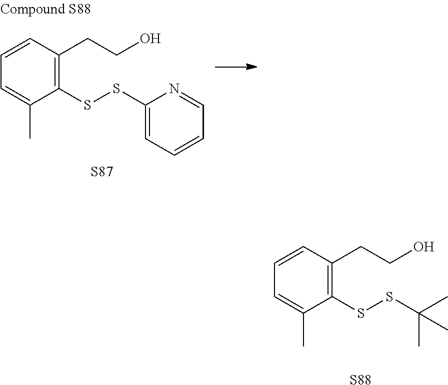 [SMILES: Cc1cccc(CCO)c1SSc1ccccn1 ; "S87" ; Cc1cccc(CCO)c1SSC(C)(C)C ; "S88"]

To a solution of compound S87 (0.57 g, 2.0 mmol) in 10.0 mL of dichloromethane was added MeOTf (0.36 g, 2.0 mmol) at room temperature. The reaction mixture stirred for 10 minutes before tert-butylmercaptan (0.23 mL, 2.2 mmol) and diisopropylethylamine (0.5 mL) were added. The reaction mixture stirred for another 30 min at room temperature before being condensed in vacuo. The crude mixture purified using flash silica gel column purification on ISCO companion (ethyl acetate/hexane, 0-50%) to give compound S88 as colorless oil (0.46 g, 87%). ¹H NMR (500 MHz): δ7.17 (1H, t, J 7.0 Hz), 7.11 (m, 2H), 3.89 (2H, t, J 7.0 Hz), 3.34 (2H, t, J 7.0 Hz), 2.64 (3H, s), 1.27 (s, 9H)

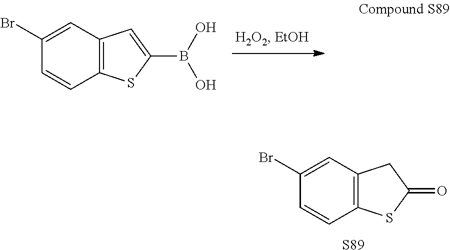 [SMILES: OB(O)c1cc2cc(Br)ccc2s1 ; O=C1Cc2cc(Br)ccc2S1 ; "S89"]

To a solution of 5-bromobenzo[b]thiophene-2-boronic acid (1.0 g, 3.90 mmol) in EtOH (12.0 mL) was added hydrogen peroxide (30%, 1.5 mL) dropwise. After stirring overnight, the reaction mixture was carefully concentrated under reduced pressure, diluted with water (30 mL), and extracted with ethyl acetate (20 mL×3). The combined organic layer was dried over anhydrous sodium sulfate and concentrated in vacuo to afford a residue, which was subjected to flash silica gel column purification on a ISCO companion (ethyl acetate/hexane, 0-20%) to give 0.64 g (72%) of compound S89 as colorless oil.

¹H NMR (500 MHz, CDCl₃): δ7.44 (1H, s), 7.43 (1H, d, J 8.0 Hz), 7.21 (1H, d, J 8.0 Hz), 3.96 (2H, s)

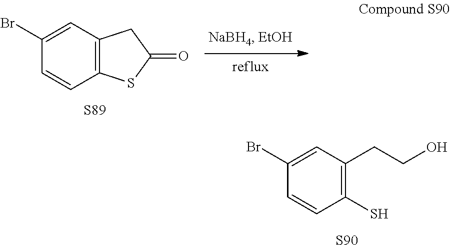 [SMILES: O=C1Cc2cc(Br)ccc2S1 ; "S89" ; OCCc1cc(Br)ccc1S ; "S90"]

To a solution of S89 (0.64 g, 2.8 mmol) in EtOH (10 mL) was added NaBH₄ (0.53 g, 13.9 mmol) in one portion while refluxing. The reaction mixture was refluxed for another 15 mins, cooled to room temperature, volatiles evaporated to give a slurry white solid which was dissolved in water and the solution was acidified to pH 2 with 1 M HCl. The water layer was extracted with dichloromethane (3×20 mL), the combined organic layers were washed with brine and dried over anhydrous sodium sulfate, concentrated in vacuo to afford the crude compound S90 as a white solid. ¹H NMR (500 MHz, CDCl₃): δ7.37 (1H, s), 7.23 (1H, d, J 8.0 Hz), 7.18 (1H, d, J 8.0 Hz), 3.90 (2H, t, J 6.5 Hz), 3.42 (1H, s), 2.94 (2H, t, J 6.5 Hz)

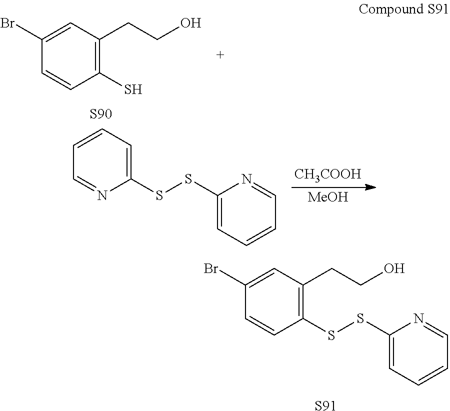

To a solution of dithiodipyridine (1.84 g, 8.34 mmol) and acetic acid (0.03 mL) in MeOH (10 mL) was added the crude S90 in MeOH (5 mL) and the mixture was stirred for 30 min then evaporated to give a yellow residue, This was subjected to flash silica gel column purification on a ISCO companion (ethyl acetate/hexane, 0-40%) to give 0.50 g (53% for two steps) of the compound S91 as colorless oil.

¹H NMR (500 MHz, CDCl₃): δ8.47 (1H, d, J 5.0 Hz), 7.64-7.58 (3H, m), 7.31-7.26 (2H, m), 7.13 (1H, m), 3.95 (2H, t, J 6.5 Hz), 3.12 (2H, t, J 6.5 Hz)

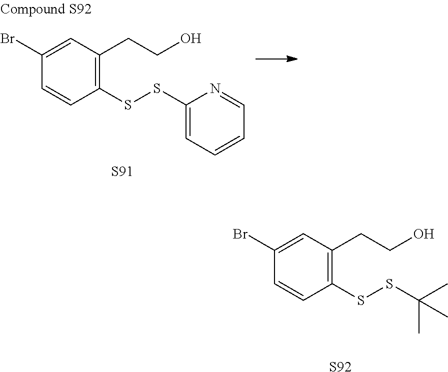 [SMILES: OCCc1cc(Br)ccc1SSc1ccccn1 ; "S91" ; CC(C)(C)SSc1ccc(Br)cc1CCO ; "S92"]

To a solution of compound S91 (0.50 g, 1.47 mmol) in 10.0 mL of dichloromethane was added MeOTf (0.24 g, 1.47 mmol) at room temperature. The reaction mixture stirred for 10 minutes before tert-butylmercaptan (0.18 mL, 1.62 mmol) and diisopropylethylamine (0.5 mL) were added. The reaction mixture stirred for another 30 min at room temperature and condensed in vacuo. The crude mixture purified using flash silica gel column purification on ISCO companion (ethyl acetate/hexane solvent, 0-50%) to give compound S92 as colorless oil (0.37 g, 78%). ¹H NMR (500 MHz): δ7.72 (2H, d, J 8.5 Hz), 7.34 (2H, m), 3.91 (2H, t, J 7.0 Hz), 3.07 (2H, t, J 7.0 Hz), 1.29 (s, 9H)

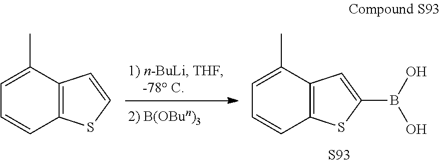

4-Methylbenzothiophene (1.0 g, 6.75 mmol) was dissolved in ether under argon and the solution cooled to 0° C. n-Butyllithium (2.7 mL of 2.5M in hexane, 6.75 mmol) was added while maintaining the temperature at 0-5° C. The mixture was stirred at 0° C. for 10 minutes, then 45 minutes at room temperature; cooled again to 0° C., and tributyl borate (1.99 mL, 7.43 mmol) was added drop wise. Reaction mixture stirred for 1 hour at 0° C., then warmed to room temperature and allowed to stand overnight followed by quenching with 1M hydrochloric acid. The aqueous phase extracted with ether (2×30 mL), and the combined organic layers were washed with aqueous sodium hydroxide (1M). The aqueous basic layer acidified with concentrated hydrochloric acid to pH 2 and extracted with ether (2×30 mL). The combined organic layers were dried over anhydrous Na₂SO₄. The solvent was evaporated in vacuo to give the crude S93 (1.05 g, 81%) as a white solid, which was used directly in next step without further purification.

¹H NMR (500 MHz, CD₃OD): δ7.93 (1H, s), 7.70 (1H, d, J 8.0 Hz), 7.25 (1H, t, J 7.0 Hz), 7.13 (1H, d, J 7.0 Hz), 7.04 (1H, d, J 7.0 Hz), 2.62 (3H, s)

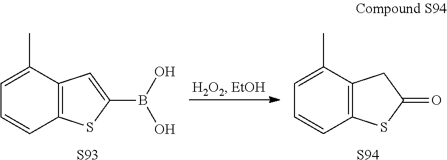

To a solution of crude S93 (1.05 g, 5.5 mmol) in EtOH (10.0 mL) was added hydrogen peroxide (30%, 1.0 mL) drop wise. After stirring overnight, the reaction mixture was carefully concentrated under reduced pressure, diluted with water (30 mL), and extracted with ethyl acetate (3×20 mL). The combined organic layers were dried over anhydrous sodium sulfate and concentrated in vacuo to afford a residue, which was subjected to flash silica gel column purification on a ISCO companion (ethyl acetate/hexane=5-15%) to give 0.80 g (89%) of the title compound S94 as a colorless oil. ¹H NMR (500 MHz, CDCl₃): δ7.23-7.17 (2H, m), 7.04 (1H, d, J 7.0 Hz), 3.85 (2H, s), 2.28 (3H, s).

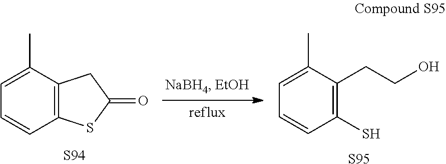

To a solution of S94 (0.69 g, 4.2 mmol) in EtOH (25 mL) was added NaBH₄ (0.79 g, 21 mmol) in one portion while refluxing. The mixture was refluxed for another 15 min and then cooled to room temperature. The mixture was evaporated to give a slurry white solid, which was dissolved in water. The mixture was acidified to pH 2 with 1 M HCl. The mixture was extracted with dichloromethane (3×20 mL). The combined organic layers were washed with brine and dried over anhydrous sodium sulfate and concentrated in vacuo to afford a residue, which was subjected to flash silica gel column purification on a ISCO companion (ethyl acetate/hexane=0-40%) to give 0.67 g (95%) of the title compound S95 as a colorless oil.

¹H NMR (500 MHz, CDCl₃): δ7.16 (1H, m), 7.00-6.96 (2H, m), 3.86 (2H, t, J 7.0 Hz), 3.44 (1H, s), 3.06 (2H, t, J 7.0 Hz), 2.35 (3H, s)

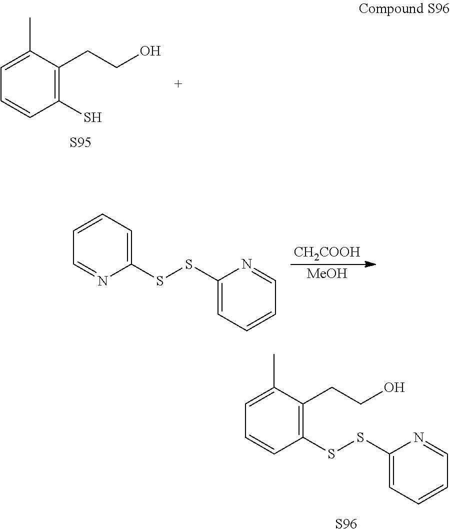

To a solution of dithiodipyridine (2.64 g, 12.0 mmol) and acetic acid (0.1 mL) in MeOH (60 mL) was added the solution of S95 (0.66 g, 3.94 mmol) in MeOH (5 mL). The mixture was stirred for 30 min, then evaporated to give a yellow residue, which was subjected to flash silica gel column purification on a ISCO companion (ethyl acetate/hexane=0-40%) to give 1.09 g (100%) of the title compound S96 as colorless oil.

¹H NMR (500 MHz, CDCl₃): δ8.49 (1H, d, J 4.5 Hz), 7.64-7.58 (2H, m), 7.50 (1H, dd, J 7.0, 2.5 Hz), 7.11 (1H, m), 7.08-7.02 (2H, m), 3.91 (2H, t, J 7.0 Hz), 3.25 (2H, t, J 7.0 Hz), 2.38 (3H, s)

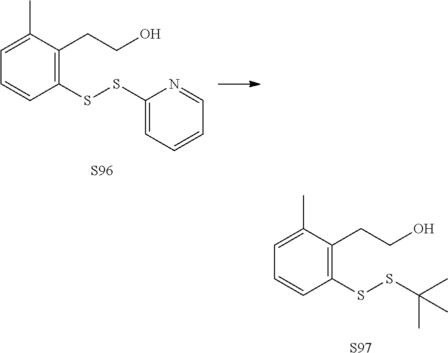 [SMILES: Cc1cccc(SSc2ccccn2)c1CCO ; "S96" ; Cc1cccc(SSC(C)(C)C)c1CCO ; "S97"]

To a solution of compound S96 (0.69 g, 2.5 mmol) in 10.0 mL of dichloromethane was added MeOTf (0.41 g, 2.5 mmol) at room temperature. The reaction mixture stirred for 10 minutes before tert-butylmercaptan (0.34 mL, 3.0 mmol) and diisopropylethylamine (0.5 mL) were added and stirring continued for another 30 min at room temperature before being condensed in vacuo. The crude mixture purified using flash silica gel column purification on ISCO companion (ethyl acetate/hexane solvent=0-40%) to give compound S97 as colorless oil (0.45 g, 70%).

¹H NMR (500 MHz): δ7.71 (1H, d, J 8.0 Hz), 7.12 (1H, t, J 8.0 Hz), 7.01 (1H, d, J 8.0 Hz), 3.86 (2H, t, J 7.0 Hz), 3.21 (2H, t, J 7.0 Hz), 2.37 (3H, s), 1.30 (s, 9H)

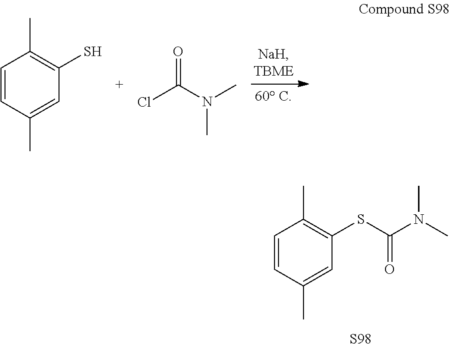

Sodium hydride (60% in oil) (1.80 g, 45.0 mmol) and t-butyl methyl ether (15 mL) was charged in to a round bottom flask under an argon atmosphere at 0° C., a solution of 2, 5-dimethylbenzenethiol (4.07 mL, 30.0 mmol) in t-butyl methyl ether (15 mL) was added drop wise followed by addition of a solution of dimethylcarbamoyl chloride (3.03 mL, 33.0 mmol) in t-butyl methyl ether (10 mL). The reaction mixture was heated to 60° C., stirred for 1.5 hours, and disappearance of the starting materials was confirmed. The mixture was cooled in an ice bath and neutralized with 1M hydrochloric acid (20 mL). The aqueous layer extracted with ether (2×30 mL), and the organic layers combined and washed with aqueous 1M sodium hydroxide, water and brine. After drying the organic layer over anhydrous sodium sulfate, the filtrate was evaporated to give a residue, which was subjected to flash silica gel column purification on a ISCO companion (ethyl acetate/hexane=5-50%) to give the title compound S98 as a colorless oil (5.15 g, 82%).

¹H NMR (500 MHz, CDCl₃): δ7.30 (1H, s), 7.18 (1H, d, J 8.0 Hz), 7.11 (1H, d, J 8.0 Hz), 3.15-3.00 (6H, br s), 2.36 (3H, s), 2.30 (3H, s)

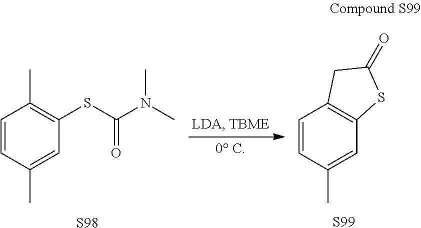

To a solution of LDA (12.5 mL, 2M in THF, 25 mmol) in t-butyl methyl ether (35 mL) was added a solution of a dimethyl-thiocarbamic acid S-(2,3-dimethylphenyl) ester (S98, 2.09 g, 10 mmol) in t-butyl methyl ether (8 mL) dropwise at 0° C. and the resulting mixture was stirred at 0° C. for 30 minutes. The reaction mixture quenched by addition of 6 mL of acetic acid followed by addition of 2 mL of 37% aqueous HCl solution, water, and the temperature was raised to near room temperature and the mixture separated. The aqueous layer extracted with ethyl acetate (2×50 mL), and the organic layers combined and washed with brine. After drying the organic layer over magnesium sulfate, the filtrate was concentrated under reduced pressure to give a residue, which was subjected to flash silica gel column purification on a ISCO companion (ethyl acetate/hexane=5-25%) to give the title compound S99 as a white solid (0.98 g, 60%).

¹H NMR (500 MHz, CDCl₃): δ7.16 (2H, s), 7.01 (1H, d, J 8.0 Hz), 3.92 (2H, s), 2.36 (3H, s)

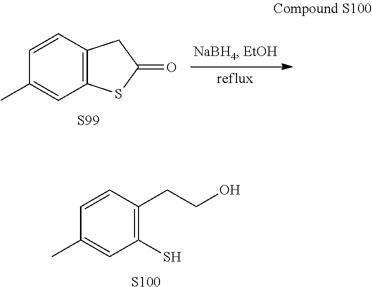

To a solution of S99 (0.98 g, 6.0 mmol) in EtOH (30 mL) was added NaBH₄ (1.13 g, 30 mmol) in one portion while refluxing. The mixture was refluxed for another 15 min then cooled to room temperature. The mixture was evaporated to give a slurry white solid, which was dissolved in water and acidified to pH 2 with 1M HCl. The mixture was extracted with dichloromethane (3×20 mL). The combined organic layers washed with brine and dried over anhydrous sodium sulfate and concentrated in vacuo to afford the crude title compound S100 as colorless oil.

¹H NMR (500 MHz, CDCl₃): δ7.14 (1H, s), 7.08 (1H, d, J 8.0 Hz), 6.94 (1H, d, J 8.0 Hz), 3.88 (2H, t, J 6.5 Hz), 3.36 (1H, s), 2.94 (2H, t, J 6.5 Hz), 2.28 (3H, s)

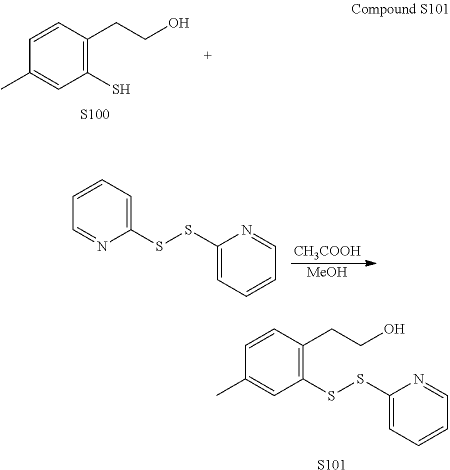

To a solution of dithiodipyridine (4.0 g, 18 mmol) and acetic acid (0.1 mL) in MeOH (70 mL) was added compound S100 in MeOH (10 mL). The reaction mixture was stirred for 30 min, evaporated to give a yellow residue, which was subjected to flash silica gel column purification on a ISCO companion (ethyl acetate/hexane=0-40%) to give 1.55 g (93% in two steps) of the title compound S101 as a colorless oil. ¹H NMR (500 MHz, CDCl₃): δ8.49 (1H, d, J 4.5 Hz), 7.65-7.61 (2H, m), 7.45 (1H, s), 7.13-7.11 (2H, m), 7.01 (1H, d, J 8.0 Hz), 3.92 (2H, t, J 6.5 Hz), 3.13 (2H, t, J 6.5 Hz), 2.25 (3H, s)

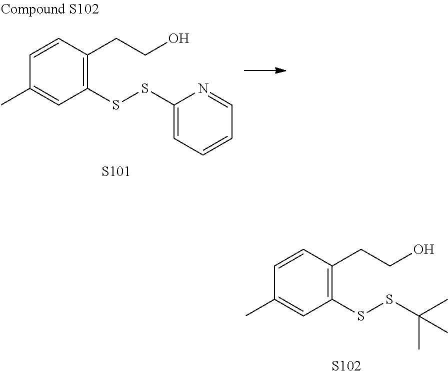 [SMILES: Cc1ccc(CCO)c(SSc2ccccn2)c1 ; "S101" ; Cc1ccc(CCO)c(SSC(C)(C)C)c1 ; "S102"]

To a solution of compound S101 (0.69 g, 2.5 mmol) in 10.0 mL of dichloromethane was added MeOTf (0.41 g, 2.5 mmol) at room temperature. The reaction mixture stirred for 10 minutes before tert-butylmercaptan (0.34 mL, 3.0 mmol) and diisopropylethylamine (0.5 mL) were added and stirring continued for another 30 min at room temperature before being condensed in vacuo. The crude mixture purified using flash silica gel column purification on ISCO companion (ethyl acetate/hexane solvent=0-40%) to give compound S102 as colorless oil (0.49 g, 77%).

¹H NMR (500 MHz): δ7.64 (1H, s), 7.06 (1H, d, J 8.0 Hz), 6.95 (1H, d, J 8.0 Hz), 3.89 (2H, t, J 7.0 Hz), 3.08 (2H, t, J 7.0 Hz), 2.36 (3H, s), 1.30 (s, 9H)

### Preparation of Benzimidazoles Linked to Disulfide Linkages

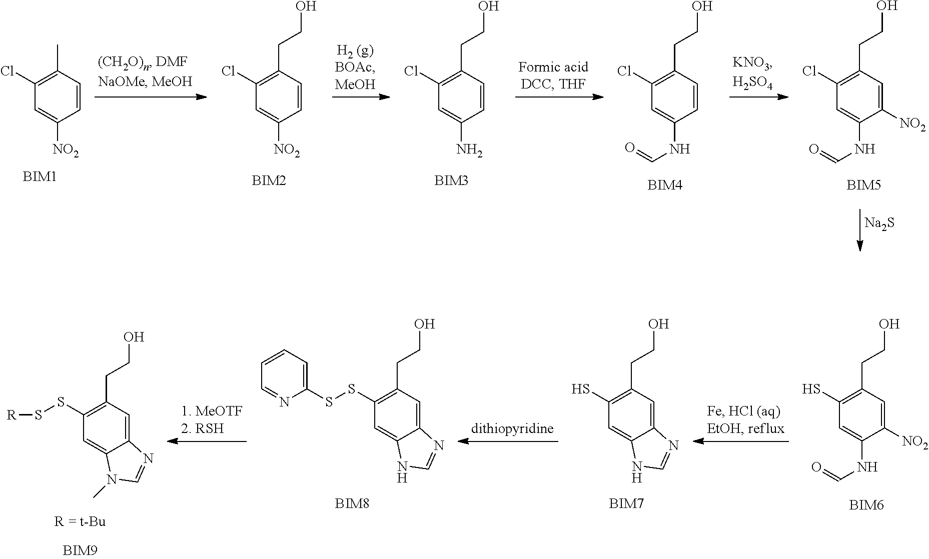

### Preparation of N-methyl 1-hydroxyethyl 2-mercapto 4, 5-benzimidazole linker (BIM9)

Commercially available 2-chloro 4-nitro-toluene (BIM1) can be homologated with paraformaldehyde under basic conditions to provide phenethylalcohol (BIM2). Other bases can include but are not-limited to NaOEt, KOtBu, DIEA, TEA, DBU, and inorganic bases. Hydrogenation of the 4-nitro group and formylation can afford BIM4. After nitration of BIM4 to BIM5, a thiol group can be introduced through treatment with Na₂S to give mercaptan (BIM6). Reduction of the 5-nitro through a reduced iron catalyst with heating can concomitantly afford 2-mercapto benzimidazole (BIM7). After conversion to the thiopyridine (BIM8), activation with MeOTf and treatment with t-butyl mercaptan (R=HS−tBu) can yield (BIM9).

### Preparation of PEG Chains Linked to Disulfide Linkages

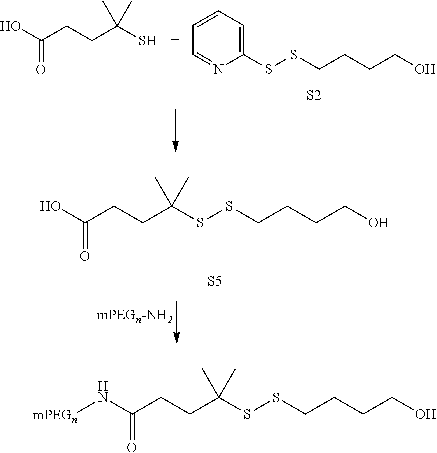

General Procedure for the Synthesis of Disulfide PEG Side Chains:

To a solution of carboxylic acid S5 (1.98 mmol) and mPEGₙ-NH₂ (1.98 mmol) in anhydrous dimethylformamide (5.0 mL) at room temp, HATU (2.97 mmol) and N, N-Diisopropylethylamine (2.97 mmol) was added in that order and stirred for 2 hours. TLC showed completion of reaction. Dimethylformamide was removed under vacuum and the residue was dissolved in CH₂Cl₂ (10.0 mL). The mixture was washed with brine (10 mL×2) and the organic layer dried over anhydrous Na₂SO₄, and evaporated to give crude compound. Silica gel column purification using ISCO companion (methanol/methylene chloride, 0-10%) gave the compound as thick syrup.

### Nucleosides

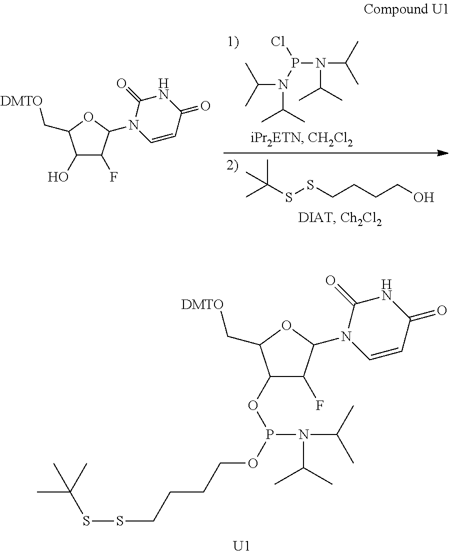

To a −78° C. cooled solution of 5′-O-(4,4′-dimethoxytrityl)-2′-F-uridine (3.9 g, 5.6 mmol) and N,N-diisopropylethylamine (1.1 mL, 6.16 mmol) in 25.0 mL of dry dichloromethane was added dropwise a solution of bis-(N,N-diisopropylamino)-chlorophosphine (1.64 g, 6.16 mmol) in 5.0 mL of dichloromethane under Argon atmosphere. The reaction mixture was allowed to warm to room temperature while stirring was maintained for 1 hour. A solution of S8 (1.0 g, 5.6 mmol) in 5.0 mL of dry dichloromethane was added dropwise and stirred for 10 minutes before a suspension of diisopropylammonium tetrazolide (1.0 g, 5.88 mmol) in 5.0 mL of dichloromethane was added portion wise. The reaction mixture was further stirred for 16 hours at room temperature. The crude mixture was diluted with 200 mL of dichloromethane and washed sequentially by saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 2.32 g (48%) of product U1 as white powder as diastereomeric mixture. ESI MS for C₄₄H₅₉FN₃O₈PS₂ Calculated 872.05. Observed 871.0 [M−H]⁺. ³¹P NMR (202 MHz, CDCl₃): δ150.7 (d, J 7.5 Hz), 150.0 (d, J 9.3 Hz).

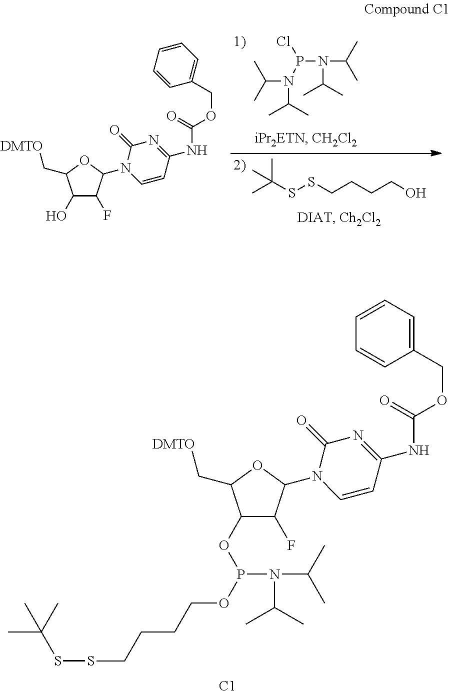

To a −78° C. cooled solution of 5′-O-(4,4′-Dimethoxytrityl)-2′-F-Cytidine (n-PAC) (3.8 g, 5.6 mmol) and N,N-diisopropylethylamine (1.1 mL, 6.16 mmol) in 25.0 mL of dry dichloromethane was added dropwise a solution of Bis-(N,N-diisopropylamino)-chlorophosphine (1.64 g, 6.16 mmol) in 5.0 mL of dichloromethane under Argon atmosphere. The reaction mixture was allowed to warm to room temperature while stirring was maintained (1 hour). A solution of S8 (1.0 g, 5.6 mmol) in 5.0 mL of dry dichloromethane was added dropwise and stirred for 10 minutes before a suspension of diisoproprylammonium tetrazolide (1.0 g, 5.88 mmol) in 5.0 mL of dichloromethane was added portion wise. The reaction mixture was further stirred for 16 hours at room temperature. The crude mixture was diluted with 200 mL of dichloromethane and washed sequentially by saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 1.43 g (26%) of product C1 as white powder as diastereomeric mixture. ESI MS for C₅₂H₆₆FN₄O₉PS₂ Calculated 1005.2. Observed 1004.0 [M−H]⁺. ³¹P NMR (202 MHz, CDCl₃): δ150.6 (d, J 6.5 Hz), 150.0 (d, J 5.5 Hz).

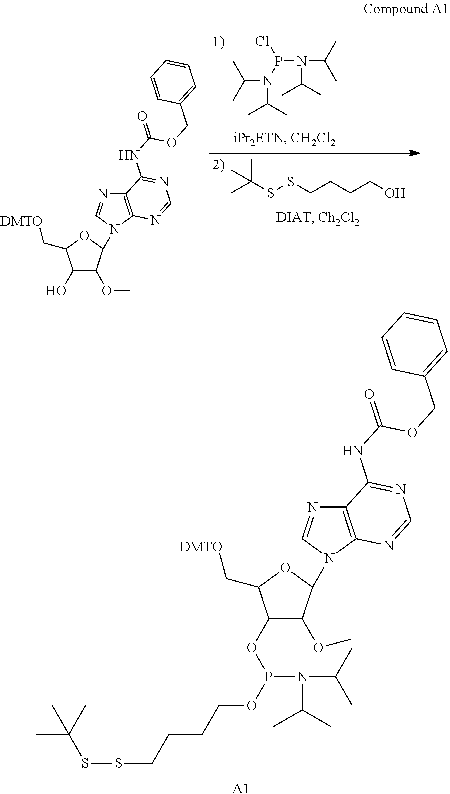

To a −78° C. cooled solution of 5′-O-(4,4′-Dimethoxytrityl)-2′-O-Methyl-Adenosine (n-PAC) (4.02 g, 5.6 mmol) and N,N-diisopropylethylamine (1.1 mL, 6.16 mmol) in 25.0 mL of dry dichloromethane was added dropwise a solution of Bis-(N,N-diisopropylamino) chlorophosphine (1.64 g, 6.16 mmol) in 5.0 mL of dichloromethane under Argon atmosphere. The reaction mixture was allowed to warm to room temperature while stirring was maintained (1 hour). A solution of S8 (1.0 g, 5.6 mmol) in 5.0 mL of dry dichloromethane was added drop wise and stirred for 10 minutes before a suspension of diisoproprylammonium tetrazolide (1.0 g, 5.88 mmol) in 5.0 mL of dichloromethane was added portion wise. The reaction mixture was further stirred for 16 hours at room temperature. The crude mixture was diluted with 200 mL of dichloromethane and washed sequentially by saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 1.99 g (35%) of product A1 as white powder as diastereomeric mixture. ESI MS for C₅₄H₆₉N₆O₉PS₂ Calculated 1041.26. Observed 1040.4 [M−H]⁺. ³¹P NMR (202 MHz, CDCl₃): δ150.4, 149.5.

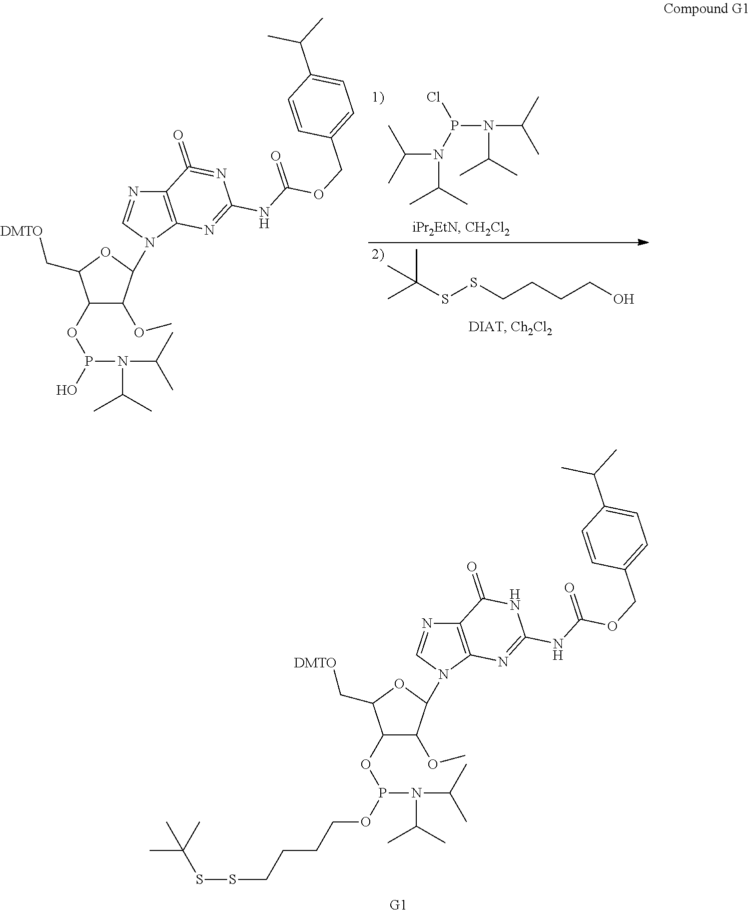

To a −78° C. cooled solution of 5′-O-(4,4′-Dimethoxytrityl)-2′-O-Methyl-Guanosine (n-isopropyl-PAC) (3.2 g, 4.1 mmol) and N,N-diisopropylethylamine (0.78 mL, 4.5 mmol) in 20.0 mL of dry dichloromethane was added dropwise a solution of bis-(N,N-diisopropylamino) chlorophosphine (1.2 g, 4.5 mmol) in 5.0 mL of dichloromethane under Argon atmosphere. The reaction mixture was allowed to warm to room temperature while stirring was maintained (1 hour). A solution of S8 (0.74 g, 4.1 mmol) in 5.0 mL of dry dichloromethane was added drop wise and stirred for 10 minutes before a suspension of diisoproprylammonium tetrazolide (0.74 g, 4.3 mmol) in 5.0 mL of dichloromethane was added portion wise. The reaction mixture was further stirred for 16 hours at room temperature. The crude mixture was diluted with 100 mL of dichloromethane and washed sequentially by saturated NaHCO₃ solution (25 mL) and brine (25 mL), then dried over anhydrous Na₂SO₄. The solvent was evaporated in vacuo and the crude mixture was purified by silica gel column chromatography using ethyl acetate/hexane solvent system (0-100% gradient on Combi Flash Rf Instrument) to give 0.60 g (13%) of product G1 as white powder as diastereomeric mixture. ESI MS for C₅₇H₇₅N₆O₁₀PS₂ Calculated 1099.34. Observed 1098.2[M]⁺. ³¹P NMR (202 MHz, CDCl₃): δ150.5, 149.9.

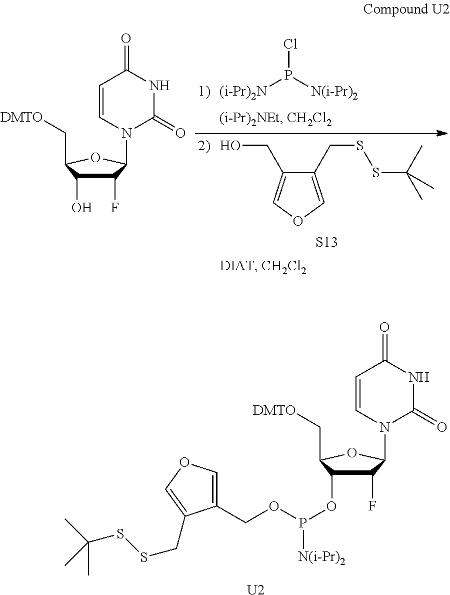

To a −78° C. cooled solution of 5′-O-(4,4′-dimethoxytrityl)-2′-F-uridine (0.36 g, 0.65 mmol) and N,N-diisopropylethylamine (0.13 mL, 0.72 mmol) in 10.0 mL of dry dichloromethane was added dropwise a solution of bis-(N,N-diisopropylamino)-chlorophosphine (0.19 g, 0.72 mmol) in 3.0 mL of dichloromethane under Argon atmosphere. The reaction mixture was allowed to warm to room temperature while stirring was maintained (1 hour). A solution of S13 (0.15 g, 0.65 mmol) in 3.0 mL of dry dichloromethane was added dropwise and stirred for 10 minutes before a suspension of diisoproprylammonium tetrazolide (0.11 g, 0.65 mmol) in 3.0 mL of dichloromethane was added portion wise. The reaction mixture was further stirred for 16 hours at room temperature. The crude mixture was diluted with 50 mL of dichloromethane and washed sequentially by saturated NaHCO₃ solution (20 mL) and brine (20 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.12 g (20%) of product U2 as white powder as diastereomeric mixture. ESI MS for C₄₆H₅₇FN₃O₉PS₂ Calculated 910.0. Observed 909 [M−H]⁺. ³¹P NMR (202 MHz, CDCl₃) δ151.3 (d, J 8.5 Hz), 151.2 (d, J 10.5 Hz).

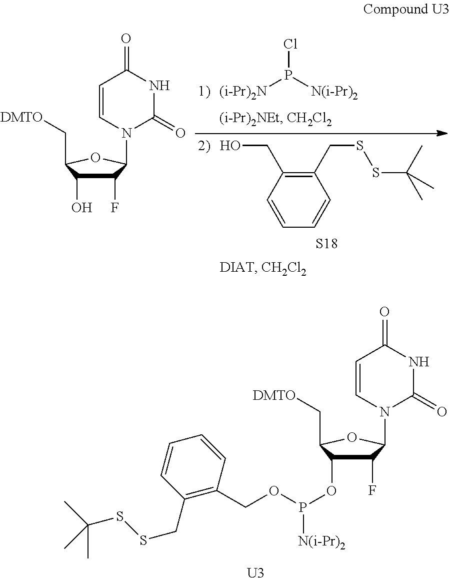

To a −78° C. cooled solution of 5′-O-(4,4′-dimethoxytrityl)-2′-F-uridine (0.73 g, 1.32 mmol) and N,N-diisopropylethylamine (0.25 mL, 1.45 mmol) in 15.0 mL of dry dichloromethane was added dropwise a solution of bis-(N,N-diisopropylamino) chlorophosphine (0.39 g, 1.45 mmol) in 5.0 mL of dichloromethane under Argon atmosphere. The reaction mixture was allowed to warm to room temperature while stirring was maintained (1 hour). A solution of S18 (0.32 g, 1.32 mmol) in 5.0 mL of dry dichloromethane was added dropwise and stirred for 10 minutes before a solution of ethylthiotetrazole in acetonitrile (0.25 M, 3.2 mL, 0.80 mmol) was added portion wise. The reaction mixture was further stirred for 3 hours at room temperature. The crude mixture was diluted with 100 mL of dichloromethane and washed sequentially by saturated NaHCO₃ solution (40 mL) and brine (40 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.17 g (20%) of product U3 as white powder as diastereomeric mixture. ESI MS for C₄₈H₅₉FN₃O₈PS₂ Calculated 920.0. Observed 943.0 [M+Na]⁺. ³¹P NMR (202 MHz, CDCl₃): δ156.3 (d, J 7.3 Hz), 155.6 (d, J 11.3 Hz).

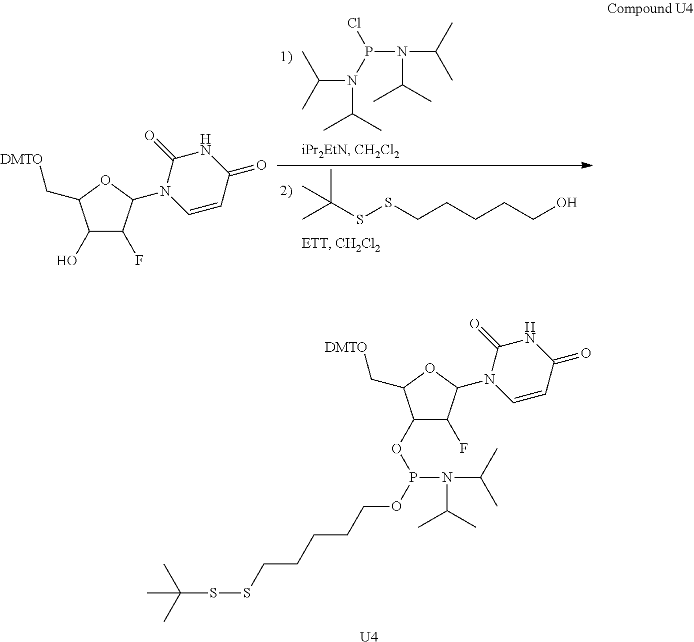

To a −78° C. cooled solution of 5′-O-(4,4′-dimethoxytrityl)-2′-F-uridine (1.77 g, 3.2 mmol) and N,N-diisopropylethylamine (0.62 mL, 3.54 mmol) in 20.0 mL of dry dichloromethane was added dropwise a solution of bis-(N,N-diisopropylamino)-chlorophosphine (0.94 g, 3.54 mmol) in 5.0 mL of dichloromethane under Argon atmosphere. The reaction mixture was allowed to warm to room temperature while stirring was maintained (1 hour). A solution of S20 (0.67 g, 3.22 mmol) in 5.0 mL of dry dichloromethane was added dropwise and stirred for 10 minutes before a solution of ethylthiotetrazole in acetonitrile (0.25M, 7.7 mL, 1.93 mmol) was added portion wise. The reaction mixture was further stirred for 3 hours at room temperature. The crude mixture was diluted with 100 mL of dichloromethane and washed sequentially by saturated NaHCO₃ solution (30 mL) and brine (30 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 1.48 g (52%) of product U4 as a white powder as diastereomeric mixture. ESI MS for C₄₅H₆₁FN₃O₈PS₂ Calculated 886.08. Observed 884.8 [M−H]⁺. ³¹P NMR (202 MHz, CDCl₃) δ150.6 (d, J 6.8 Hz), 149.9 (d, J 9.1 Hz).

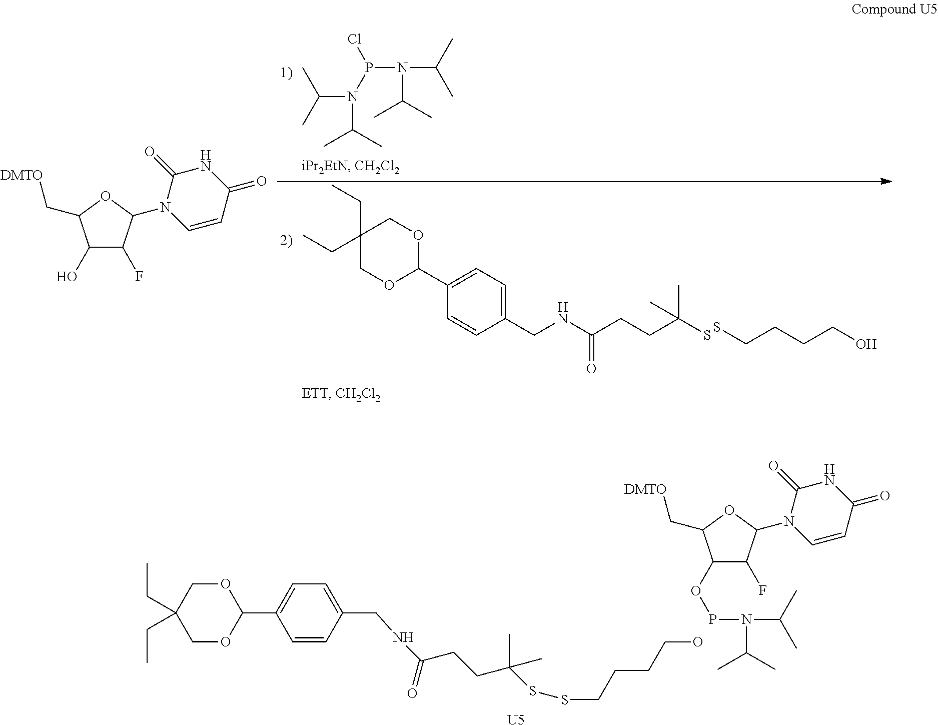

To a −78° C. cooled solution of 5′-O-(4,4′-dimethoxytrityl)-2′-F-uridine (0.66 g, 1.2 mmol) and N,N-diisopropylethylamine (0.23 mL, 1.32 mmol) in 10.0 mL of dry dichloromethane was added dropwise a solution of bis-(N,N-diisopropylamino)-chlorophosphine (0.35 g, 1.32 mmol) in 3.0 mL of dichloromethane under Argon atmosphere. The reaction mixture was allowed to warm to room temperature while stirring was maintained (1 hour). A solution of S23 (0.58 g, 1.2 mmol) in 3.0 mL of dry dichloromethane was added dropwise and stirred for 10 minutes before a solution of ethylthiotetrazole in acetonitrile (0.25M, 2.9 mL, 0.72 mmol) was added portion wise. The reaction mixture was further stirred for 3 hours at room temperature. The crude mixture was diluted with 50 mL of dichloromethane and washed sequentially by saturated NaHCO₃ solution (20 mL) and brine (20 mL), then dried over anhydrous Na₂SO₄. The solvent was evaporated in vacuo and the crude mixture was purified by silica gel column chromatography using ethyl acetate/hexane solvent system (0-40% gradient on Combi Flash Rf Instrument) to give 0.35 g (27%) of product U5 as white powder as diastereomeric mixture. ESI MS for C₆₁H₈₂FN₄O₁₁PS₂ Calculated 1161.42. Observed 1162 [M+H]⁺. ³¹P NMR (202 MHz, CDCl₃) δ154.87 (d, J 7.3 Hz), 154.53 (d, J 9.0 Hz).

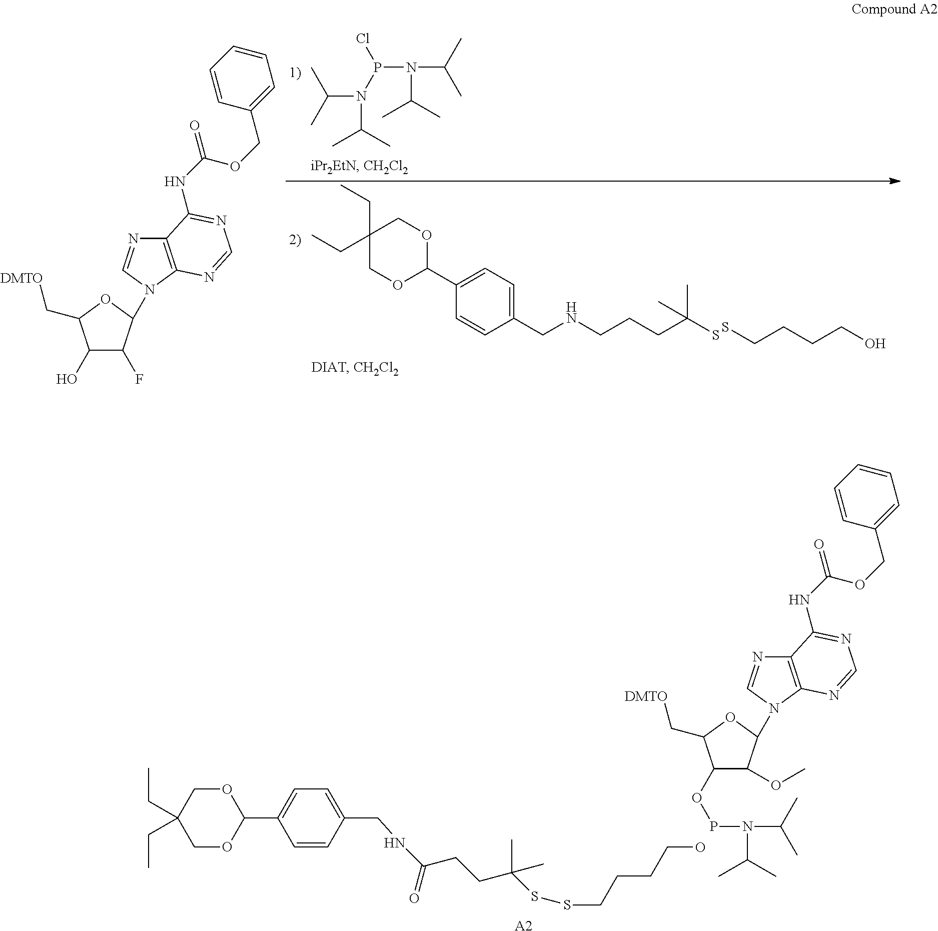

To a −78° C. cooled solution of 5′-O-(4,4′-Dimethoxytrityl)-2′-O-Methyl-Adenosine (n-PAC) (1.48 g, 2.1 mmol) and N—N-diisopropylethylamine (0.4 mL, 2.28 mmol) in 15.0 mL of dry dichloromethane was added dropwise a solution of Bis-(N,N-diisopropylamino) chlorophosphine (0.61 g, 2.28 mmol) in 5.0 mL of dichloromethane under Argon atmosphere. The reaction mixture was allowed to warm to room temperature while stirring was maintained (1 hour). A solution of S23 (1.0 g, 2.1 mmol) in 5.0 mL of dry dichloromethane was added drop wise and stirred for 10 minutes before a suspension of Diisoproprylammonium tetrazolide (0.35 g, 2.1 mmol) in 5.0 mL of dichloromethane was added portion wise. The reaction mixture was further stirred for 16 hours at room temperature. The crude mixture was diluted with 75.0 mL of dichloromethane and washed sequentially by saturated NaHCO₃ solution (25 mL) and brine (25 mL), then dried over anhydrous Na₂SO₄. The solvent was evaporated in vacuo and the crude mixture was purified by silica gel column chromatography using ethyl acetate/hexane solvent system (0-60% gradient on Combi Flash Rf Instrument) to give 1.01 g (37%) of product A2 as a white powder as diastereomeric mixture. ESI MS for O₇₁H₉₂N₇O₁₂PS₂ Calculated 1330.63. Observed 1331.3 [M+H]⁺. ³¹P NMR (202 MHz, CDCl₃) δ154.93 & 154.29.

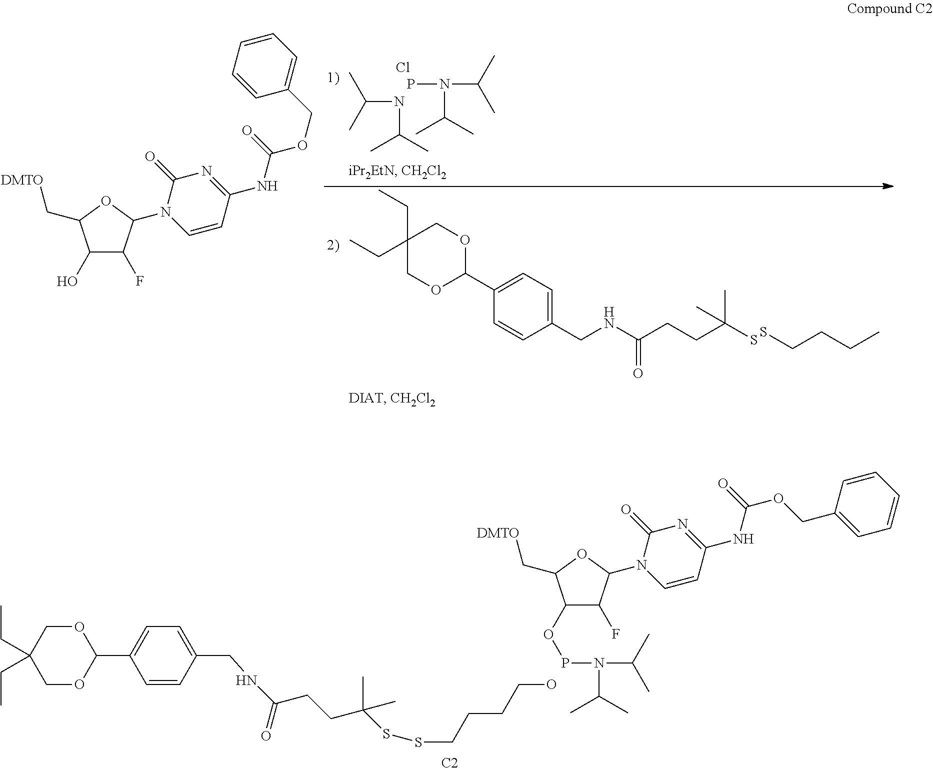

To a −78° C. cooled solution of 5′-O-(4,4′-Dimethoxytrityl)-2′-F-Cytidine (n-PAC) (1.4 g, 2.1 mmol) and N—N-diisopropylethylamine (0.4 mL, 2.28 mmol) in 15.0 mL of dry dichloromethane was added dropwise a solution of Bis-(N,N-diisopropylamino)-chlorophosphine (0.61 g, 2.28 mmol) in 5.0 mL of dichloromethane under Argon atmosphere. The reaction mixture was allowed to warm to room temperature while stirring was maintained (1 hour). A solution of S23 (1.0 g, 2.1 mmol) in 5.0 mL of dry dichloromethane was added drop wise and stirred for 10 minutes before a suspension of Diisoproprylammonium tetrazolide (0.35 g, 2.1 mmol) in 5.0 mL of dichloromethane was added portion wise. The reaction mixture was further stirred for 16 hours at room temperature. The crude mixture was diluted with 75 mL of dichloromethane and washed sequentially by saturated NaHCO₃ solution (25 mL) and brine (25 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.75 g (29%) of product C2 as a white powder as diastereomeric mixture. ESI MS for C₆₉H₈₉FN₅O₁₂PS₂ Calculated 1294.57. Observed 1295.2 [M+H]⁺. ³¹P NMR (202 MHz, CDCl₃) δ154.77 (d, J 5.6 Hz), 154.69 (d, J 7.7 Hz).

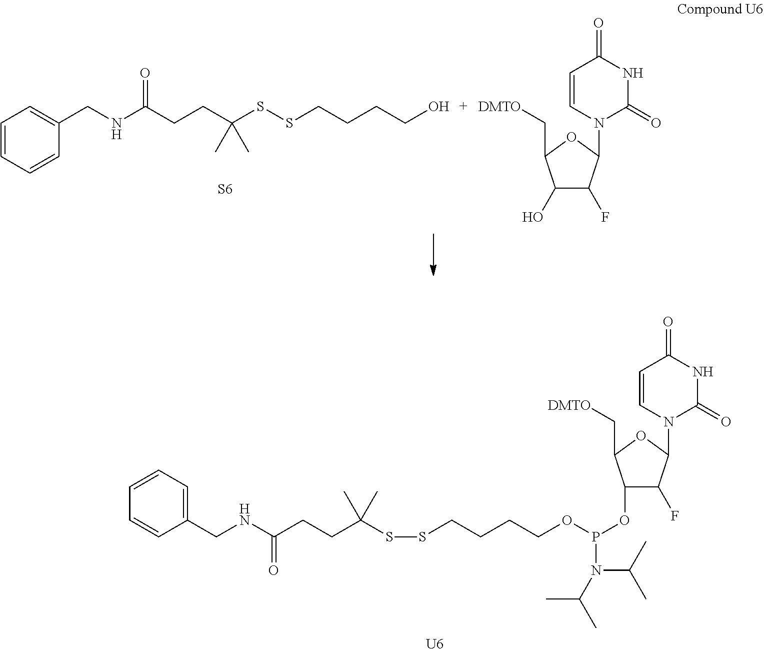

A solution of dis-(N,N-disiopropylamino) chlorophosphine (0.28 g, 1.05 mmol) in dry CH₂Cl₂ (1.0 mL) was added drop wise to a solution of 5′-O-(4,4′-dimethoxytrityl)-2′-F-Uridine (0.55 g, 1.0 mmol) and N,N-diisopropylethylamine (0.18 mL, 1.05 mmol) in dry CH₂Cl₂ (5 mL) at −78° C. The reaction mixture was warmed to room temperature and stirred for 1.5 hours. A solution of S6 (0.34 g, 1.0 mmol) in 1.0 mL of dry CH₂Cl₂ was added and stirred for 10 minutes. Then a solution of diisoproprylammonium tetrazolide (0.17 g, 1.0 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate. Volatiles evaporated in vacuo to afford a residue, which was subjected to flash silica gel column purification on ISCO companion (ethyl acetate with 5% methanol/hexane=20%-55%) to give 0.50 g (49%) of compound U6 as a colorless foam. ESI MS for C₅₃H₆₈FN₄O₉PS₂ Calculated 1018.4. Observed 1018.1 (M⁺). ³¹P NMR (202 MHz, CDCl₃): δ150.15 (d, J 6.9 Hz), 149.65 (d, J 8.7 Hz).

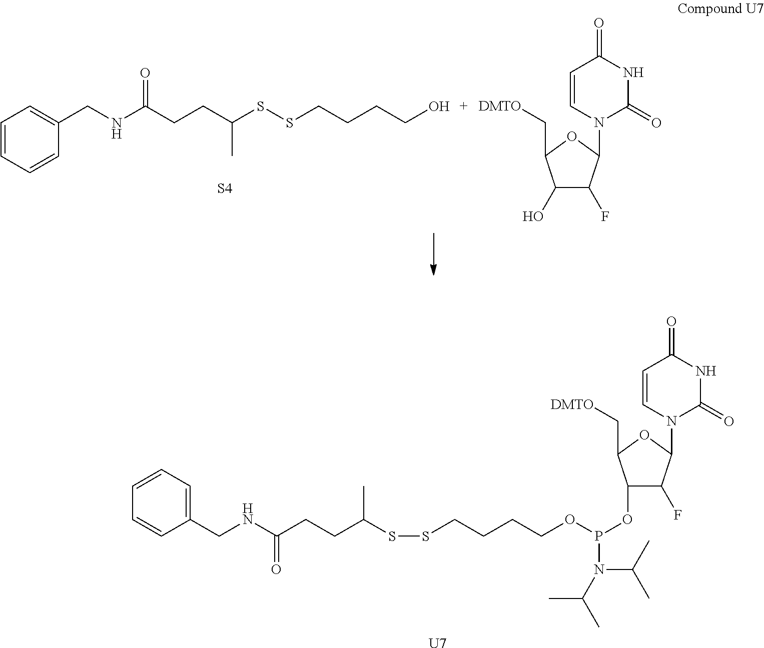

A solution of bis-(N, N-disiopropylamino)-chlorophosphine (0.28 g, 1.05 mmol) in dry CH₂Cl₂ (1.0 mL) was added drop wise to a solution of 5′-O-(4,4′-Dimethoxytrityl)-2′-F-Uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S4 (0.33 g, 1.0 mmol) in 1.0 ml of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate and the volatiles removed under vacuo to afford a residue, which was subjected to flash silica gel column purification on a ISCO companion (ethyl acetate with 5% methanol/hexane=20%-55%) to give 0.15 g (15% yield) of compound U7 as a colorless foam. ESI MS for C₅₂H₆₆FN₄O₉PS₂ Calculated 1004.4. Observed 1004.0 (M⁺). ³¹P NMR (202 MHz, CDCl3): δ 50.16 (d, J 7.9 Hz), 149.65 (d, J 10.7 Hz).

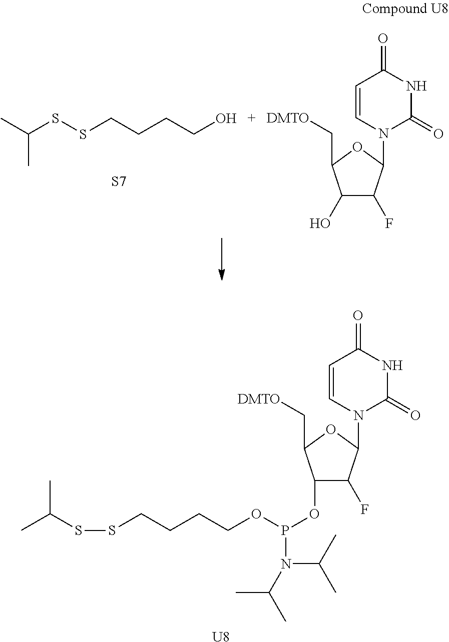

A solution of bis-(N, N-disiopropylamino)-chlorophosphine (0.28 g, 1.05 mmol) in dry CH₂Cl₂ (1.0 mL) was added dropwise to a solution of 5′-O-(4,4′-Dimethoxytrityl)-2′-F-Uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S7 (0.18 g, 1.0 mmol) in 1.0 mL of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate and evaporated in vacuo to afford a residue, which was subjected to flash silica gel column purification on a ISCO companion (ethyl acetate with 5% methanol/hexane=10%-55%) to give 0.30 g (35%) of the title compound U8 as a colorless foam. ESI MS for C₄₃H₅₇FN₃O₈PS₂ Calculated 857.3. Observed 856.9 (M⁺). ³¹P NMR (202 MHz, CDCl3): δ150.76 (d, J 7.7 Hz), 150.03 (d, J 9.3 Hz).

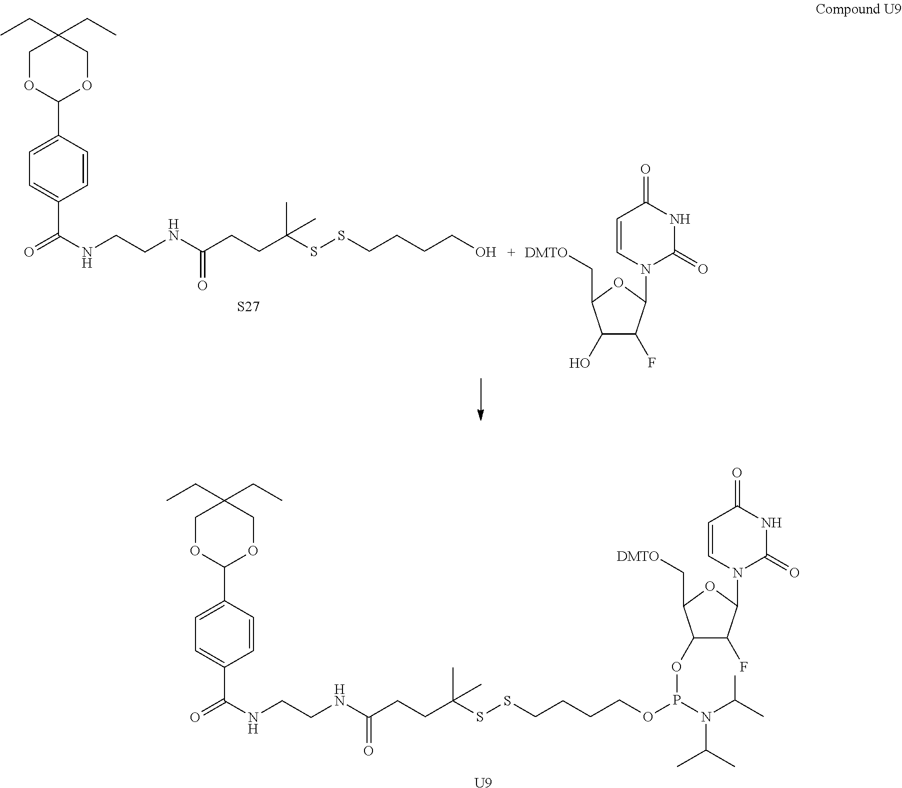

A solution of bis-(N, N-disiopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-Uridine (0.55 g, 1.0 mmol) and N,N-Diisopropylethylamine (0.18 mL, 1.05 mmol) in dry CH₂Cl₂ (5.0 mL) at −78° C. The reaction mixture warmed to room temperature and stirred for 1.5 hours. A solution of S27 (0.54 g, 1.0 mmol) in 20.0 ml of dry CH₂Cl₂ was added and stirred for 10 minutes. Then a solution of Diisopropylammonium tetrazolide (0.18 g, 1.05 mmol) in 8.0 mL of dry CH₂Cl₂ was added portion wise to the reaction mixture and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate and the filtrate was evaporated in vacuo to afford a residue, which was subjected to flash silica gel column purification on a ISCO companion instrument (acetonitrile/dichloromethane=30%-90%) to give 0.68 g (56%) of the title compound U9 as a colorless foam. ESI MS for C₆₃H₈₅FN₅O₁₂PS₂Calculated 1217.5. Observed 1217.2 (M⁺). ³¹P NMR (202 MHz, CDCl3): δ150.18 (d, J 5.7 Hz), 148.40 (d, J 11.1 Hz).

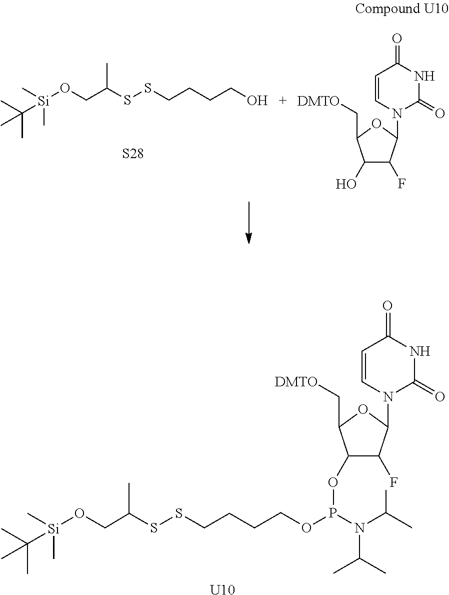

A solution of bis-(N,N-disiopropylamino)-chlorophosphine (0.16 g, 0.61 mmol) in dry CH₂Cl₂ (1.0 mL) was added dropwise to a solution of 5′-O-(4,4′-dimethoxytrityl)-2′-F-uridine (0.32 g, 0.58 mmol) and N,N-diisopropylethylamine (0.11 mL, 0.61 mmol) in dry CH₂Cl₂ (5 mL) at −78° C. The reaction mixture was warmed to room temperature and stirred for 1.5 hours. A solution of S28 (0.18 g, 0.58 mmol) in 1.0 mL of dry CH₂Cl₂ was added and stirred for 10 minutes. Then a solution of diisopropylammonium tetrazolide (0.10 g, 0.61 mmol) in 8.0 mL of dry CH₂Cl₂ was added portion wise to the reaction mixture and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate and the evaporated in vacuo to afford a residue, which was subjected to flash silica gel column purification on an ISCO companion instrument (ethyl acetate with 5% methanol/hexane=10%-55%) to give 0.15 g (26%) of the title compound U10 as a colorless foam. ESI MS for C₄₉H₇₁FN₃O₉PS₂Si Calculated 987.4. Observed 987.0 (M⁺). ³¹P NMR (202 MHz, CDCl3): δ150.88 (s), 150.08 (d, J 9.3 Hz).

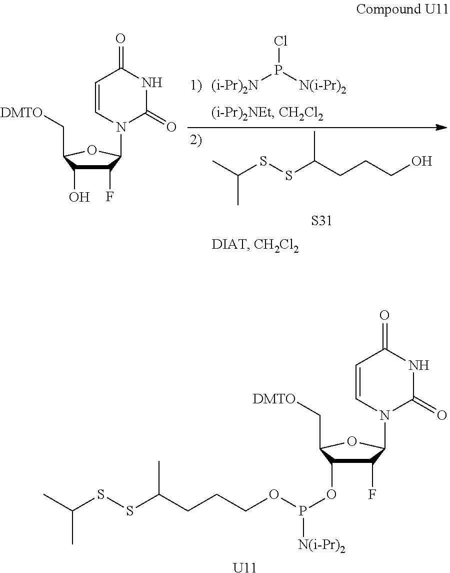

A solution of Bis-(N, N-diisopropylamino)-chlorophosphine (0.28 g, 1.05 mmol) in dry CH₂Cl₂ (1.0 mL) was added dropwise to a solution of 5′-O-(4,4′-Dimethoxytrityl)-2′-F-uridine (0.55 g, 1.0 mmol) and N,N-Diisopropylethylamine (0.18 ml, 1.05 mmol) in dry CH₂Cl₂ (5.0 mL) at −78° C. The reaction mixture was warmed to room temperature and stirred for 1.5 hours. A solution of S31 (0.18 g, 1.0 mmol) in 1.0 mL of dry CH2Cl2 was added and stirred for 10 minutes. Then a solution of diisopropylammonium tetrazolide (0.18 g, 1.05 mmol) in 8.0 mL of dry CH₂C₂ was added portion wise to the reaction mixture and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate and evaporated in vacuo to afford a residue, which was subjected to flash silica gel column purification on a ISCO companion (ethyl acetate with 5% methanol/hexane=10%-55%) to give 0.38 g (44%) the title compound U11 as a colorless foam. ESI MS for C₄₄H₅₉FN₃O₈PS₂ Calculated 871.3. Observed 870.8 (M⁺). ³¹P NMR (202 MHz, CDCl3): δ150.84 (d, J 7.6 Hz), 150.73 (d, J 7.6 Hz) 150.06 (d, J 9.1 Hz), 150.02 (d, J 9.1 Hz).

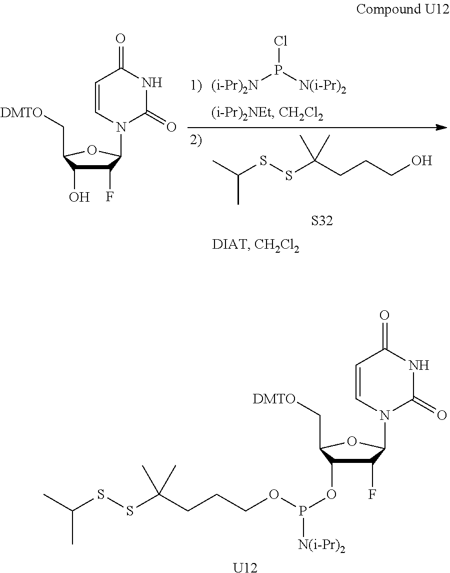

A solution of bis-(N, N-disiopropylamino)-chlorophosphine (0.28 g, 1.05 mmol) in dry CH₂Cl₂ (1.0 ml) was added dropwise to a solution of S32 (0.18 g, 1.0 mmol) and N,N-diisopropylethylamine (0.18 mL, 1.05 mmol) in dry CH₂Cl₂ (5.0 mL) at −78° C. The reaction mixture warmed to room temperature and stirred for 1.5 hours. A solution of 5′-O-(4,4′-Dimethoxytrityl)-2′-F-Uridine (0.55 g, 1.0 mmol) in 1.0 mL of dry CH₂Cl₂ was added and stirred for 10 minutes. Then a solution of 2-Ethylthiotetrazole (2.4 mL, 0.25M in acetonitrile, 0.6 mmol) was added portion wise to the reaction mixture and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate and the filtrate was evaporated in vacuo to afford a residue, which was subjected to flash silica gel column purification on a ISCO companion (ethyl acetate/hexane=10%-55%) to give 0.47 g (53%) of the title compound U12 as a colorless foam. ESI MS for C₄₅H₆₁FN₃O₈PS₂ Calculated 885.4. Observed 884.7 (M−1). ³¹P NMR (202 MHz, CDCl3): δ150.88 (d, J 7.7 Hz), 150.03 (d, J 9.5 Hz).

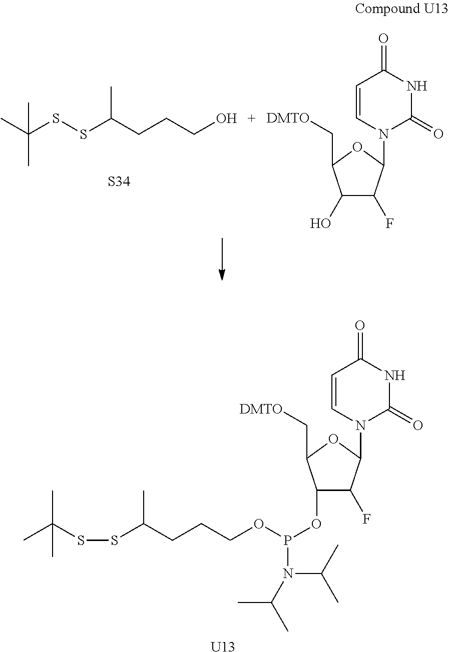

A solution of Bis-(N, N-diisopropylamino)-chlorophosphine (0.26 g, 0.97 mmol) in dry CH₂Cl₂ (1.0 ml) was added dropwise to a solution of S34 (0.19 g, 0.92 mmol) and N, N-Diisopropylethylamine (0.17 mL, 0.97 mmol) in dry CH₂Cl₂ (5.0 mL) at −78° C. The reaction mixture was warmed to room temperature and stirred for 1.5 hours. A solution of 5′-O-(4,4′-Dimethoxytrityl)-2′-F-Uridine (0.50 g, 0.92 mmol) in 1.0 mL of dry CH₂Cl₂ was added and stirred for 10 minutes. Then a solution of 2-Ethylthiotetrazole (2.6 mL, 0.25M in acetonitrile, 0.65 mmol) was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate and the filtrate was evaporated in vacuo to afford a residue, which was subjected to flash silica gel column purification on a ISCO companion (ethyl acetate/hexane=10%-55%) to give 0.29 g (36%) of the title compound U13 as a colorless foam. ESI MS for C₄₅H₆₁FN₃O₈PS₂ Calculated 885.4. Observed 885.2 (M⁺). ³¹P NMR (202 MHz, CDCl₃): δ150.91 (d, J 7.7 Hz), 150.76 (d, J 7.7 Hz), 150.07 (d, J 9.1 Hz), 150.02 (d, J 9.5 Hz).

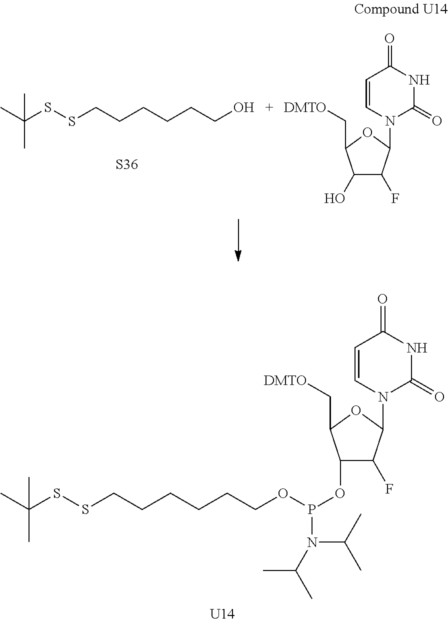

A solution of bis-(N,N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-Uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S36 (0.22 g, 1.0 mmol) in 1.0 mL of dry CH2Cl2 was added and stirred for 10 minutes. Then a solution of Diisopropylammonium tetrazolide (0.18 g, 1.05 mmol) in 8.0 mL of dry CH₂Cl₂ was added portion wise to the reaction mixture and the resulting mixture was stirred overnight. The reaction mixture was diluted with CH2Cl2 (20 mL) and washed with saturated aqueous sodium bicarbonate (20 mL) and brine (20 mL). The mixture was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.37 g (41%) of the title compound U14 as a colorless foam. ESI MS for C₄₆H₆₃FN₃O₈PS₂ Calculated 899.4. Observed 900.7 (M+1). ³¹P NMR (202 MHz, CDCl₃): δ155.32 (d, J 7.7 Hz), 154.72 (d, J 9.3 Hz).

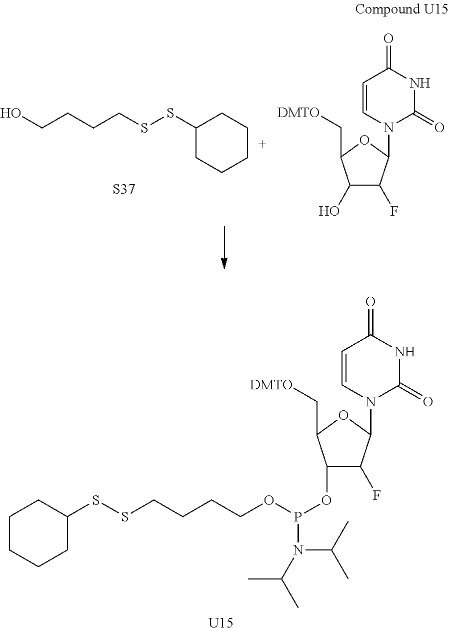

A solution of bis-(N, N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S37 (0.22 g, 1.0 mmol) in 1.0 mL of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The reaction mixture was diluted with CH₂Cl₂ (20 mL) and washed with saturated aqueous sodium hydrogen carbonate (20 mL) and brine (20 mL). The organic layer was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.34 g (38%) of the title compound U15 as a colorless foam. ESI MS for C₄₆H₆₁FN₃O₈PS₂Calculated 897.4. Observed 896.7 (M−1). ³¹P NMR (202 MHz, CDCl₃): δ150.73 (d, J 7.7 Hz), 150.01 (d, J 9.5 Hz).

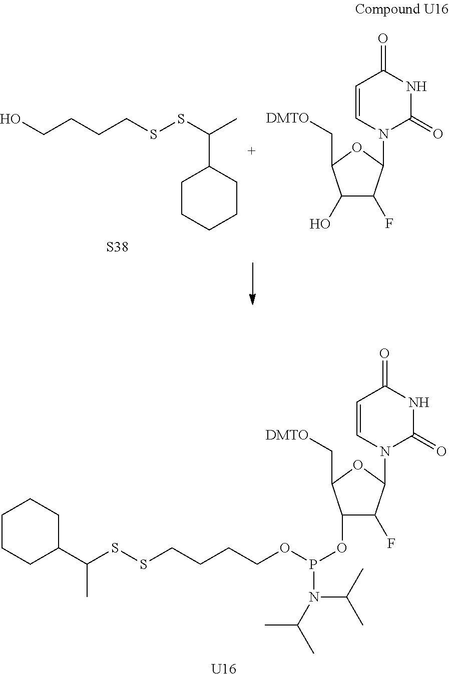

A solution of bis-(N, N-disiopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-Diisopropylethylamine (0.18 mL, 1.05 mmol) in dry CH₂Cl₂ (5.0 mL) at −78° C. The reaction mixture was warmed to room temperature and stirred for 1.5 hours. A solution of S38 (0.25 g, 1.0 mmol) in 1.0 ml of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium hydrogen carbonate (20 mL) and brine (20 mL). The organic layer was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.38 g (41%) of the title compound U16 as a colorless foam. ESI MS for C₄₈H₆₅FN₃O₃PS₂ Calculated 925.4. Observed 926.5 (M+1). ³¹P NMR (202 MHz, CDCl₃): δ150.78 (d, J 6.9 Hz), 150.02 (d, J 9.5 Hz).

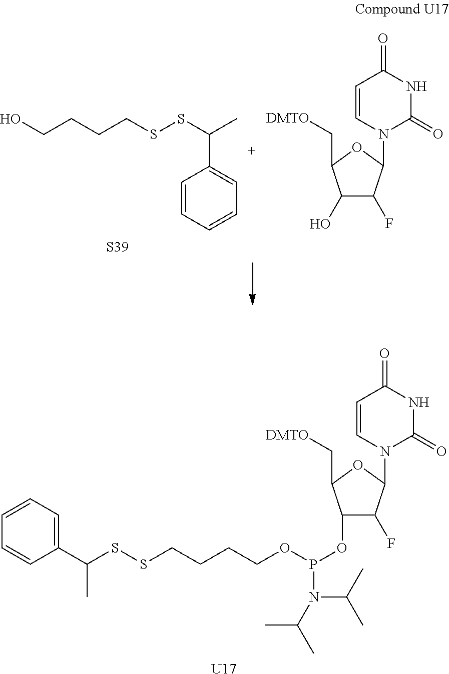

A solution of bis-(N, N-disiopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-Diisopropylethylamine (0.18 mL, 1.05 mmol) in dry CH₂Cl₂ (5.0 mL) at −78° C. The reaction mixture was warmed to room temperature and stirred for 1.5 hours. A solution of S39 (0.24 g, 1.0 mmol) in 1.0 mL of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.24 g (26%) of the title compound U17 as a colorless foam. ESI MS for C₄₈H₅₉FN₃O₈PS₂ Calculated 919.3. Observed 920.7 (M+1). ³¹P NMR (202 MHz, CDCl₃): δ155.41 (d, J 7.1 Hz), 154.73 (d, J 8.9 Hz).

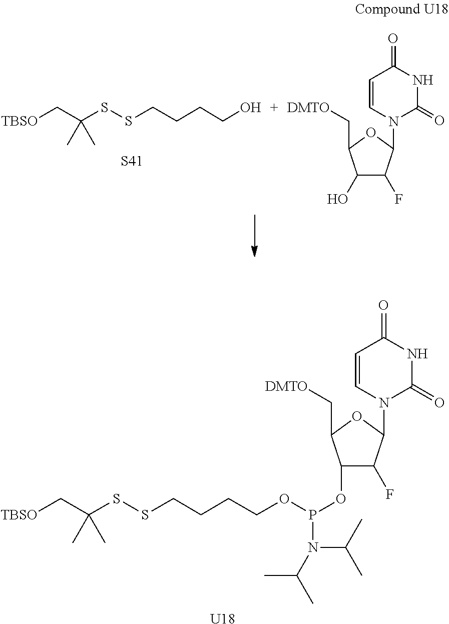

A solution of bis-(N, N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S41 (0.32 g, 1.0 mmol) in 1.0 mL of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The reaction mixture was diluted with CH₂Cl₂ (20 mL) and washed with saturated aqueous sodium bicarbonate (20 mL) and brine (20 mL). The organic layer was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.25 g (25%) of the title compound U18 as a colorless foam. ESI MS for C₅₀H₇₃FN₃O₉PS₂Si Calculated 1001.4. Observed 1003.1 (M+2). ³¹P NMR (202 MHz, CDCl₃): δ155.67 (d, J 7.7 Hz), 154.81 (d, J 9.7 Hz).

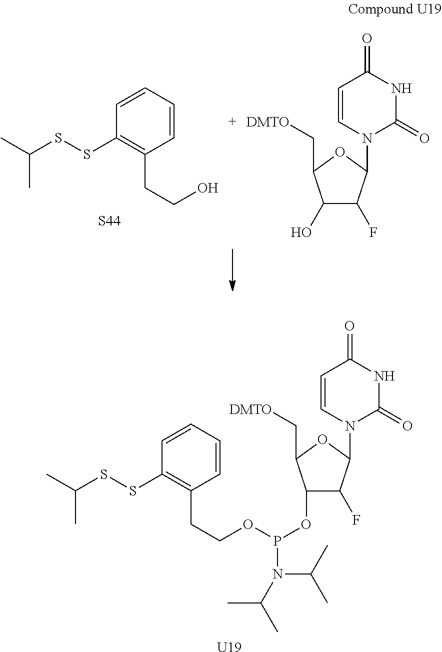

A solution of bis-(N, N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N, N-Diisopropylethylamine (0.18 mL, 1.05 mmol) in dry CH₂Cl₂ (5.0 mL) at −78° C. The reaction mixture was warmed to room temperature and stirred for 1.5 hours. A solution of S44 (0.23 g, 1.0 mmol) in 1.0 mL of dry CH₂Cl₂ was added and stirred for 10 minutes. Then a solution of Diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate and the filtrate was evaporated in vacuo to afford a residue, which was subjected to flash silica gel column purification on a ISCO companion (ethyl acetate with 5% methanol/hexane=10%-55%) to give 0.24 g (27%) of the title compound U19 as a colorless foam. ESI MS for C₄₇H₅₇FN₃O₈PS₂ Calculated 905.3. Observed 907.0 (M+2). ³¹P NMR (202 MHz, CDCl₃): δ154.74 (d, J 8.9 Hz), 154.53 (d, J 7.7 Hz).

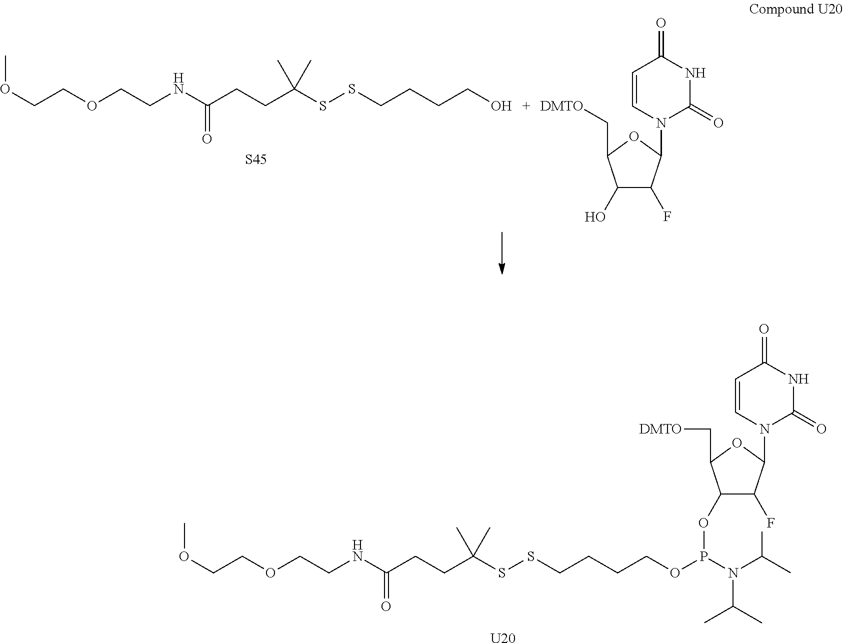

A solution of Bis-(N, N-diisopropylamino)-chlorophosphine (0.57 g, 2.14 mmol) in dry CH₂Cl₂ (2.0 mL) was added dropwise to a solution of 5′-O-(4,4′-dimethoxytrityl)-2′-F-uridine (1.11 g, 2.0 mmol) and N, N-diisopropylethylamine (0.37 mL, 2.14 mmol) in dry CH₂Cl₂ (10.0 mL) at −78° C. The reaction mixture was warmed to room temperature and stirred for 1.5 hours. A solution of S45 (0.72 g, 2.0 mmol) in 5.0 mL of dry CH₂Cl₂ was added and stirred for 10 minutes. Then a solution of diisopropylammonium tetrazolide (0.37 g, 2.14 mmol) in 8.0 mL of dry CH₂Cl₂ was added portion wise to the reaction mixture and the resulting mixture was stirred overnight. The mixture was diluted with CH2Cl2 (20 mL) and washed with saturated aqueous sodium bicarbonate (20 mL) and brine (20 mL). The organic layer was dried over anhydrous sodium sulfate and the filtrate was evaporated in vacuo to afford a residue, which was subjected to flash silica gel column purification on a ISCO companion (EtOAc/Hexane, containing 2.5% MeOH) to give 0.45 g (23%) of the title compound U20 as a colorless oil. ³¹P NMR (202 MHz, CDCl₃): δ150.13 (d, J 6.5 Hz), 149.13 (d, J 9.1 Hz)

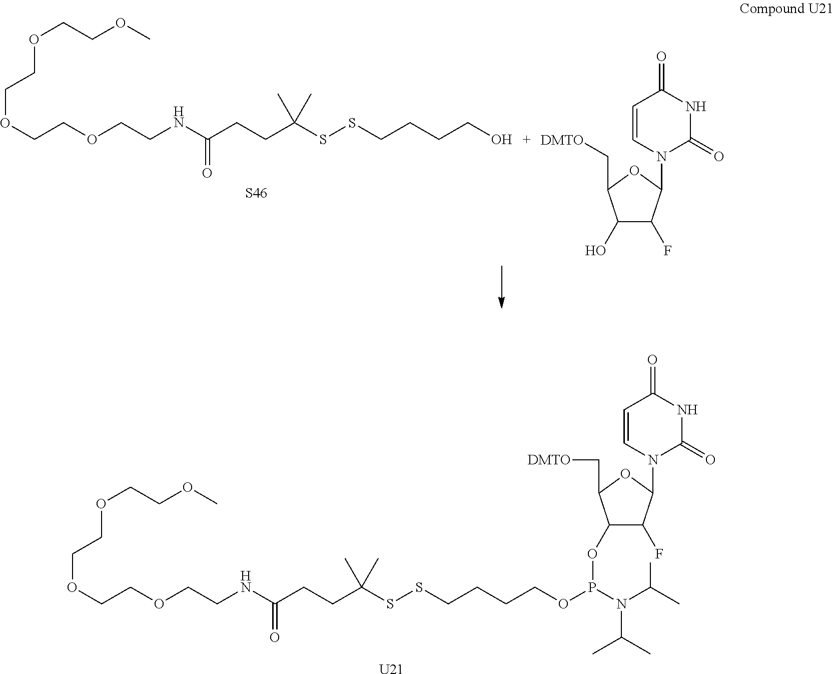

A solution of dis-(N,N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N, N-diisopropylethylamine (0.18 mL, 1.05 mmol) in dry CH₂Cl₂ (5.0 mL) at −78° C. The reaction mixture was warmed to room temperature and stirred for 1.5 hours. A solution of S46 (0.44 g, 1.0 mmol) in 1.0 ml of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate and the filtrate was evaporated in vacuo to afford a residue, which was subjected to flash silica gel column purification on a ISCO companion (methanol/dichloromethane=1%-8%) to give 0.30 g (27%) of the title compound U21 as a colorless oil. ESI MS for C₅₅H₈₀FN₄O₁₃PS₂ Calculated 1118.5. Observed 1118.3 (M⁺). ³¹P NMR (202 MHz, CDCl₃): δ150.15 (d, J 6.5 Hz), 149.23 (d, J 9.1 Hz).

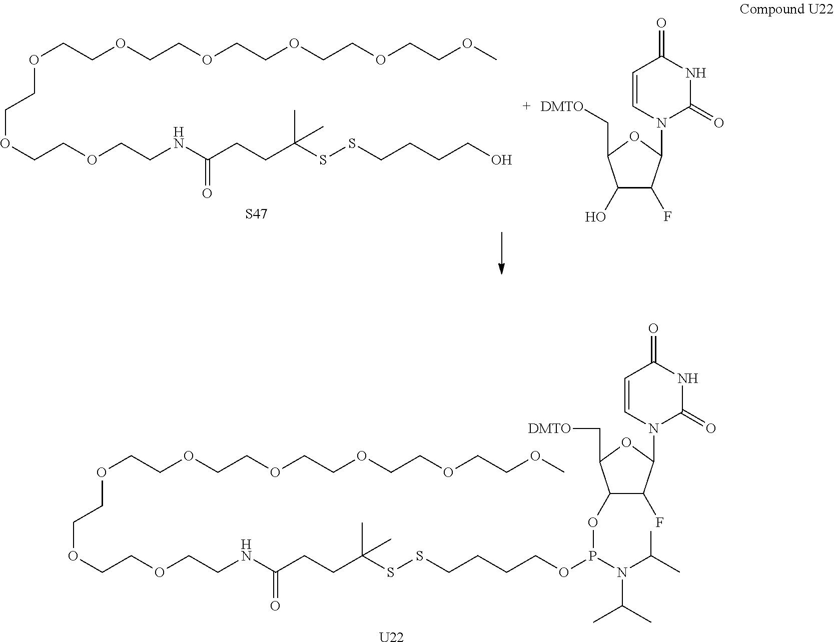

A solution of Bis-(N,N-diisopropylamino)-chlorophosphine (0.38 g, 1.41 mmol) in dry CH₂Cl₂ (1.0 ml) was added dropwise to a solution of 5′-O-(4,4′-dimethoxytrityl)-2′-F-uridine (0.74 g, 1.34 mmol) and N,N-diisopropylethylamine (0.25 mL, 1.41 mmol) in dry CH₂Cl₂ (5.0 mL) at −78° C. The reaction mixture was warmed to room temperature and stirred for 1.5 hours. A solution of S48 (0.75 g, 1.22 mmol) in 1.0 mL of dry CH₂CO₂ was added and stirred for 10 minutes. Then a solution of diisopropylammonium tetrazolide (0.24 g, 1.41 mmol) in 10 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate and the filtrate was evaporated in vacuo to afford a residue, which was subjected to flash silica gel column purification on a ISCO companion (methanol/dichloromethane=1%-8%) to give 0.56 g (32%) the title compound U22 as a colorless oil. ESI MS for C₆₃H₉₆FN₄O₁₇PS₂ Calculated 1294.6. Observed 1294.4 (M⁺). ³¹P NMR (202 MHz, CDCl₃): δ150.15 (d, J 7.1 Hz), 149.21 (d, J 9.5 Hz).

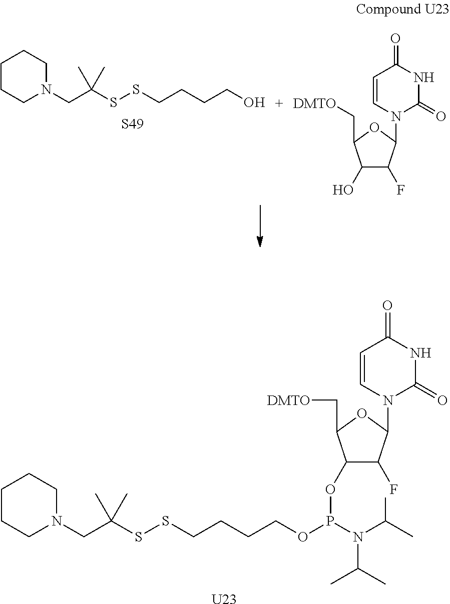

A solution of bis-(N, N-disiopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S49 (0.32 g, 1.0 mmol) in 1.0 ml of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate and the filtrate was evaporated in vacuum to afford a residue, which was subjected to flash silica gel column purification on a ISCO companion (ethyl acetate/hexane=5%-80%) to give 0.34 g (36%) of the title compound U23 as a colorless foam. ESI MS for C₄₉H₆₈FN₄O₈PS₂ Calculated 954.4. Observed 955.9 (M+1). ³¹P NMR (202 MHz, CDCl₃): δ155.54 (d, J 7.0 Hz), 154.80 (d, J 8.3 Hz).

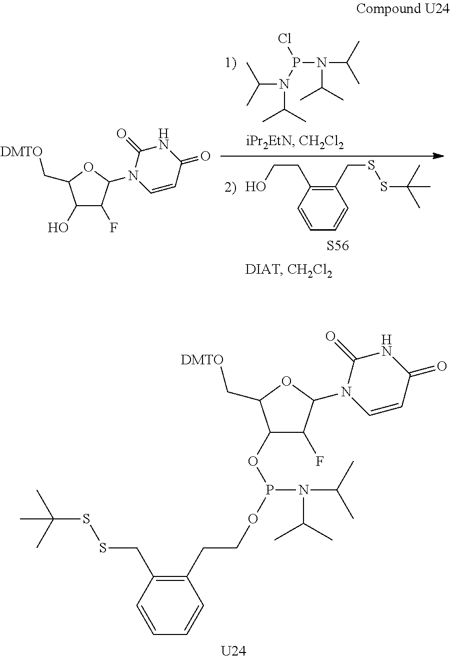

Procedure 1/Protocol 1:

To a cooled solution (−78° C.) of 5′-O-(4,4′-dimethoxytrityl)-2′-F-Uridine (1.93 g, 3.52 mmol) and N, N-diisopropylethylamine (680 μL, 3.87 mmol) in 20.0 mL of dry dichloromethane was added drop wise a solution of bis-(N,N-diisopropylamino)-chlorophosphine (1.03 g, 3.87 mmol) in 10.0 mL of dichloromethane under Argon atmosphere. The reaction mixture was allowed to warm to room temperature while stirring was maintained (1 hour). To this mixture, a solution of S56 (0.90 g, 3.52 mmol) in 5.0 mL of dry dichloromethane was added drop wise and stirred for 10 minutes before a suspension of diisoproprylammonium tetrazolide (0.66 g, 3.87 mmol) in 5.0 mL of dichloromethane was added portion wise. The reaction mixture was further stirred for 16 hours at room temperature. The reaction mixture was diluted with 200 mL of dichloromethane and washed sequentially by saturated NaHCO₃ solution (40.0 mL) and brine (40.0 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give product U24 as a white powder (1.1 g, 33% yield). ESI MS for C₄₉H₆₁FN₃O₈PS₂ calculated 934.1. observed 934.9 [M+H]⁺. ³¹P NMR (202 MHz, CDCl₃) δ155.3 (d, J 8.7 Hz), 154.7 (d, J 8.9 Hz)

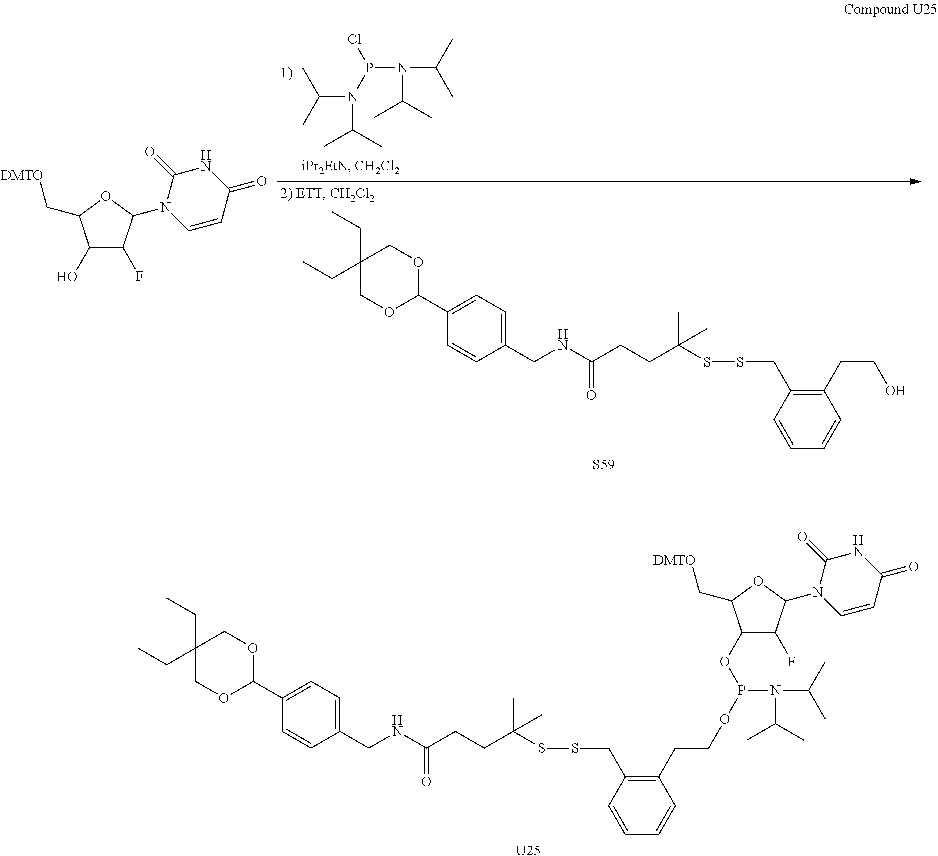

Procedure 2/Protocol 2:

To a cooled solution (−78° C.) of 5′-O-(4,4′-dimethoxytrityl)-2′-F-Uridine (0.60 g, 1.1 mmol) and N—N-diisopropylethylamine (211 μL, 1.21 mmol) in 10.0 mL of dry dichloromethane was added drop wise a solution of bis-(N,N-diisopropylamino)-chlorophosphine (0.32 g, 1.21 mmol) in 5.0 mL of dichloromethane under Argon atmosphere. The reaction mixture was allowed to warm to room temperature while stirring was maintained (1 hour). A solution of S59 (0.60 g, 1.1 mmol) in 5.0 mL of dry dichloromethane was added drop wise and stirred for 10 minutes before a solution of ethylthiotetrazole (ETT) in acetonitrile (0.25M, 2.6 mL, 0.66 mmol) was added portion wise. The reaction mixture was further stirred for 3 hours at room temperature. The crude mixture was diluted with 50.0 mL of dichloromethane and washed sequentially by saturated NaHCO₃ solution (25.0 mL) and brine (25.0 mL), then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give product U25 as white powder (0.77 g, 58% yield). ESI MS for C₆₆H₈₄FN₄O₁₁PS₂ calculated 1223.5. observed [M+H]⁺ 1224.2. ³¹P NMR (202 MHz, CDCl₃) δ154.8 (d, J 7.0 Hz), 154.6 (d, J 9.5 Hz)

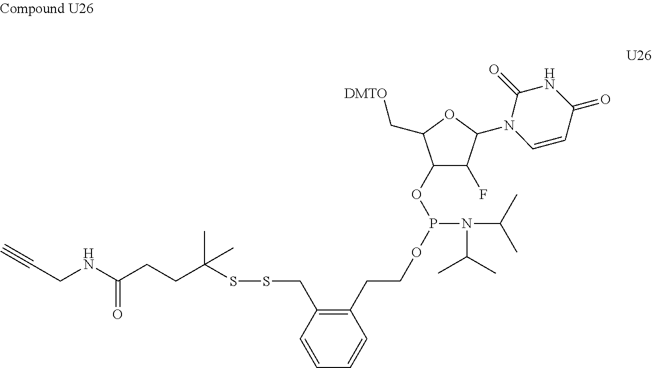

Compound U26 was prepared from alkyl disulfide (prepared from compounds S68 and S55 according to the procedure described for compound S59) and 5′-O-(4,4′-dimethoxytrityl)-2′-F-Uridine employing procedure 2.

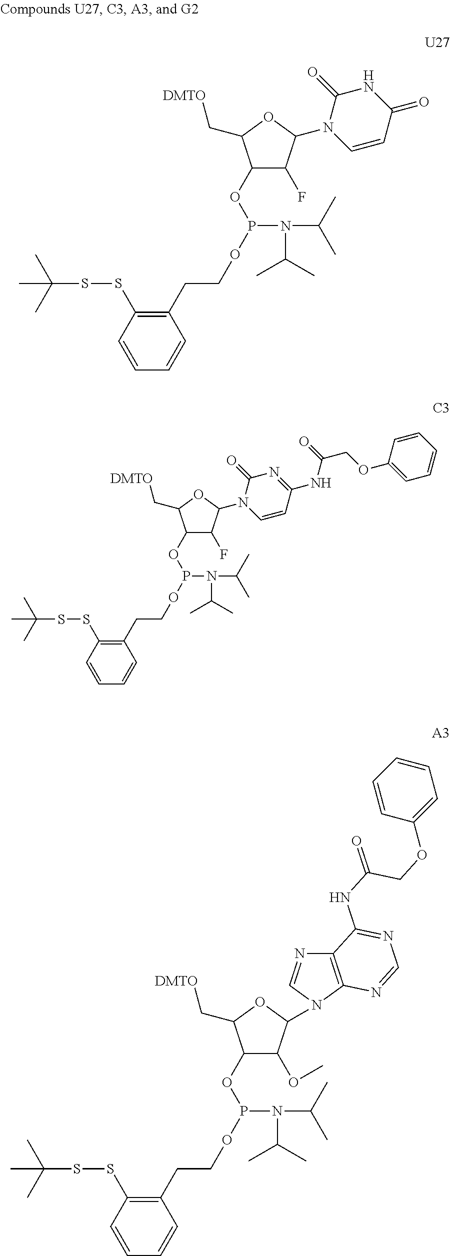

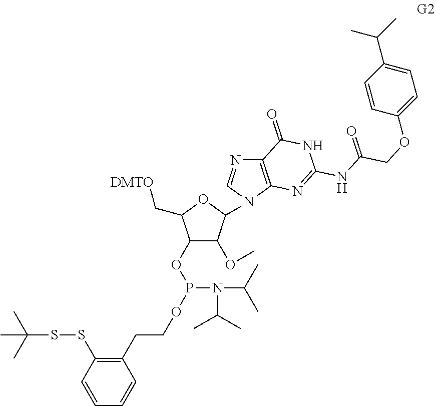

Compound U27 was prepared from compound S61 according to Protocol 1 (see compound U24) in 41% yield. ESI MS for C₄₈H₅₉FN₃O₈PS₂ calculated 920.1. observed 920.9 [M+H]⁺. ³¹P NMR (202 MHz, CDCl₃) δ154.7 (d, J 8.9 Hz), 154.5 (d, J 7.7 Hz)

Compound C3 was prepared according to Protocol 1 (see compound U24) in 59% yield. ESI MS for C₅₆H₆₆FN₄O₉PS₂ calculated 1053.2. observed 1051.5 [M−H]⁺. ³¹P NMR (202 MHz, CDCl₃) δ154.6 (d, J 5.45 Hz), 154.4 (d, J 8.3 Hz)

Compound A3 was prepared according to Protocol 1 (see compound U24) in 39% yield. ESI MS for C₅₈H₆₉FN₆O₉PS₂ calculated 1089.3. observed 1090.2[M+H]⁺. ³¹P NMR (202 MHz, CDCl₃) δ154.8 (s), 154.6 (s)

Compound G2 can be prepared from, e.g., compound S61, according to methods described herein.

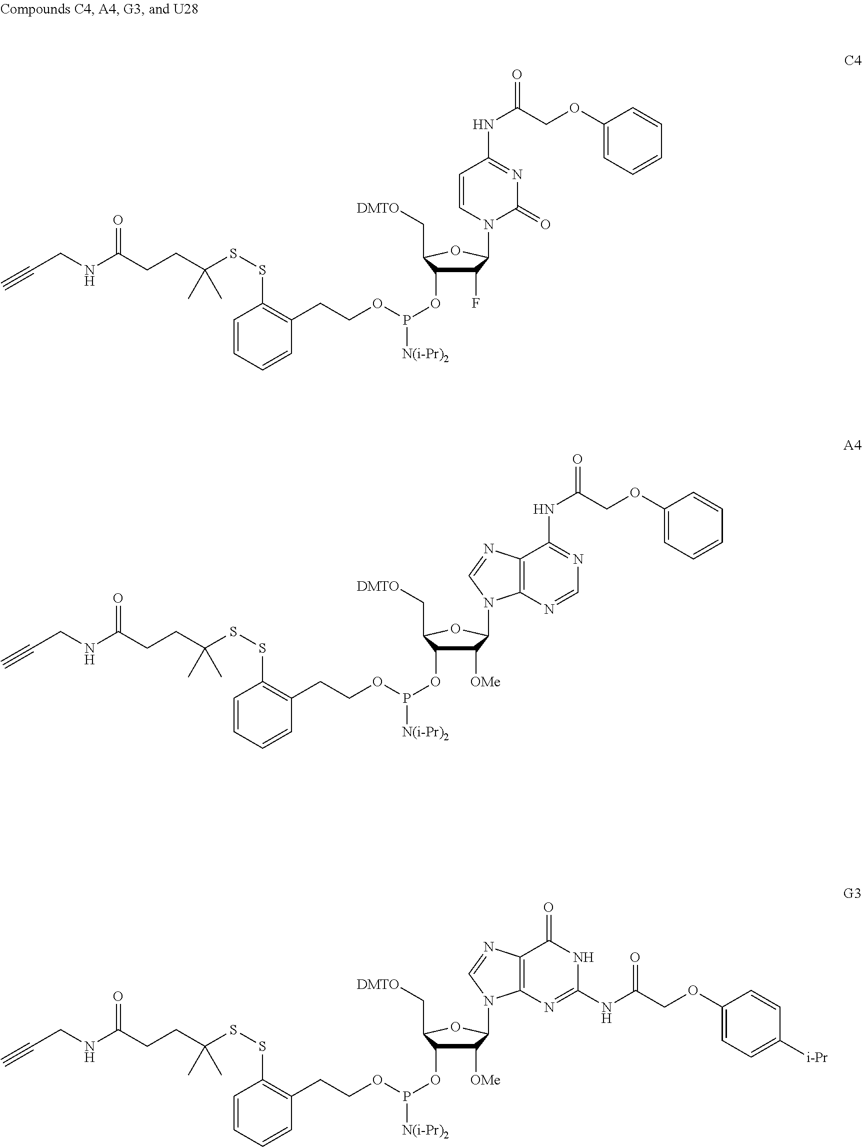

Compound C4 was prepared according to Procedure 2 (see compound U25) in 22% yield. ESI MS for C₆₁H₇₁FN₅O₁₀PS₂ calculated 1148.3. observed 1147.0 [M−H]⁺. ³¹P NMR (202 MHz, CDCl₃) δ 154.7 (d, J 5.05 Hz), 154.1 (d, J 10.7 Hz)

Compound A4 was prepared according to Procedure 2 (see compound U25) in 18% yield. ESI MS for C₆₃H₇₄N₇O₁₀O₁₀PS₂ calculated 1184.4. observed 1183.2 [M−H]⁺. ³¹P NMR (202 MHz, CDCl₃) δ154.7 (s), 154.1 (s)

Compound G3 was prepared according to Procedure 2 (see compound U25).

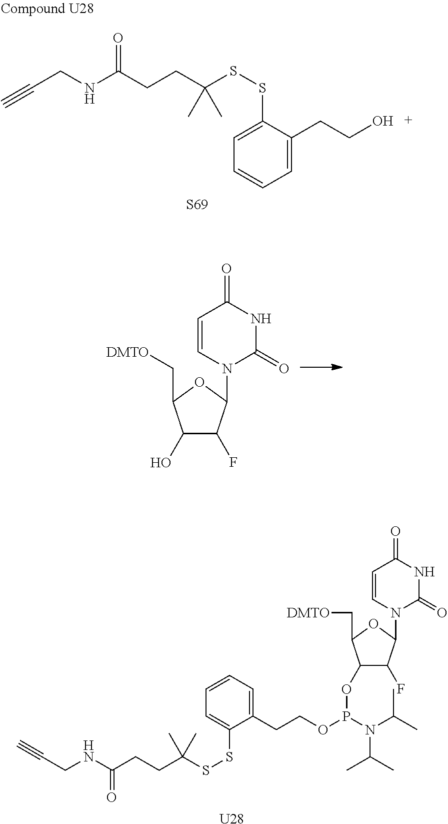

Compound U28 was prepared according to Procedure 1 (see compound U24). ESI MS for C₅₃H₆₄FN₄O₉PS₂ Calculated 1015.2. Observed 1016.2 (M+1). ³¹P NMR (202 MHz, CDCl₃): δ154.79 (d, J 7.5 Hz), 154.38 (d, J 10.5 Hz)

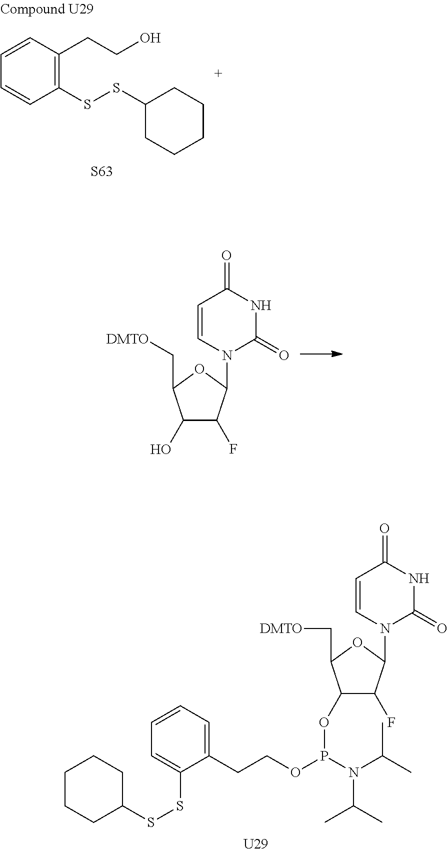

Compound U29 was prepared according to Procedure 1 (see compound U24). ESI MS for C₅₀H₆₁FN₃O₈PS₂ Calculated 946.1. Observed 947.6 (M+1). ³¹P NMR (202 MHz, CDCl₃): δ154.74 (d, J 7.7 Hz), 154.50 (d, J 7.7 Hz)

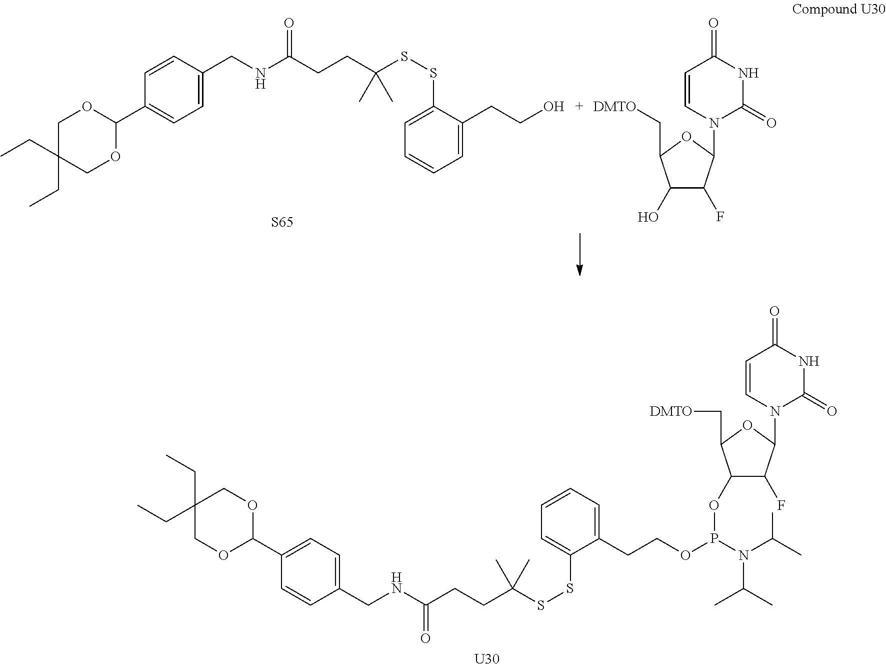

Compound U30 was prepared according to procedure 2 (see compound U25). ESI MS for C₆₅H₈₂FN₄O₁₁PS₂ Calculated 1209.5. Observed 1210.6 (M+1). ³P NMR (202 MHz, CDCl₃): δ154.74 (d, J 6.7 Hz), 154.34 (d, J 10.3 Hz)

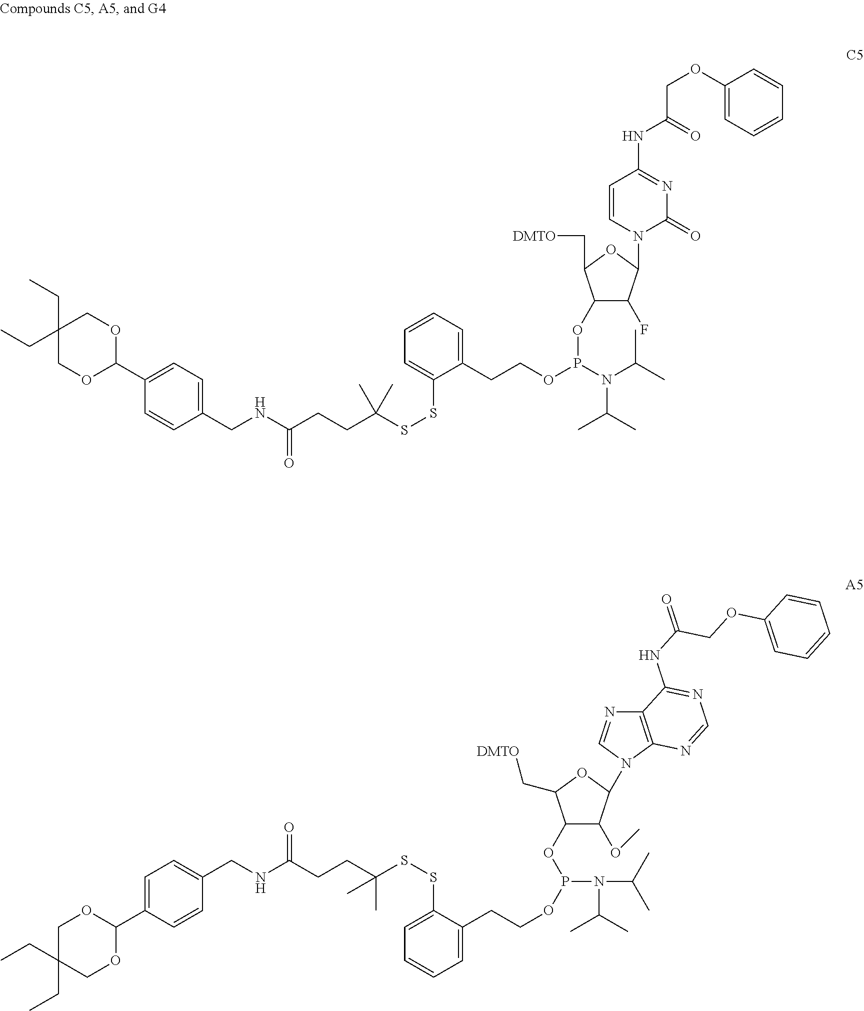

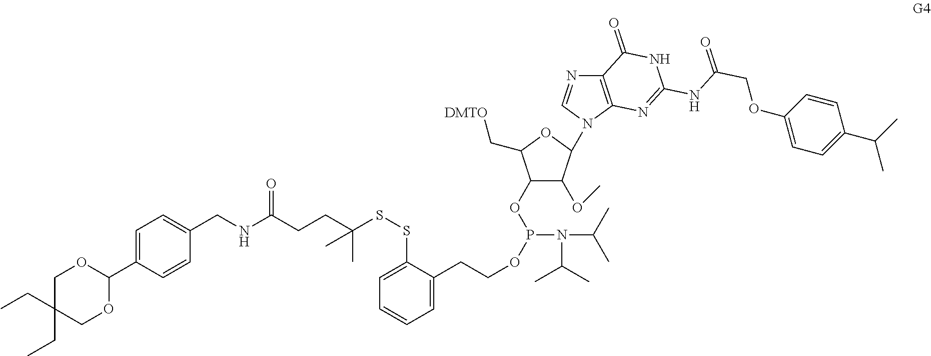

Compounds C5, A5, and G4 are prepared according to procedure 2 (see compound U25).

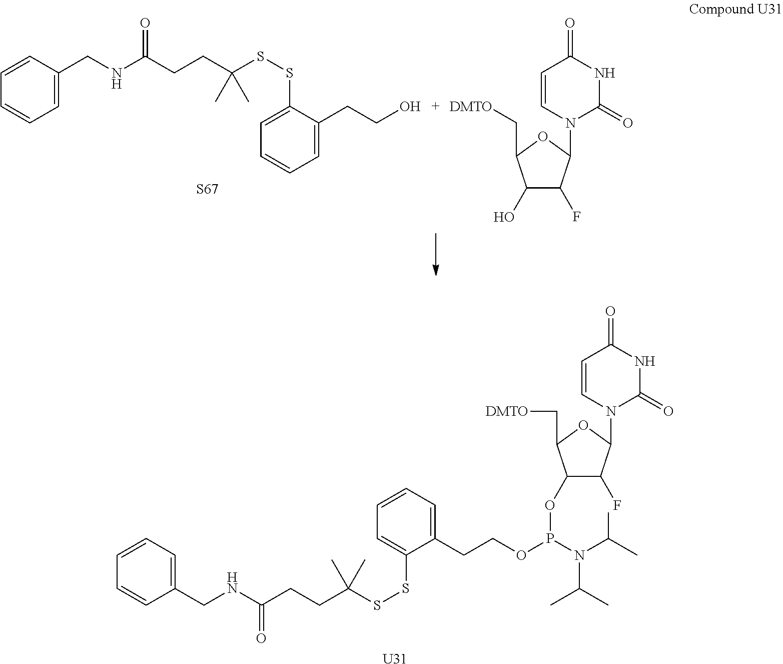

Compound U31 was prepared according to procedure 1 (see compound U24). ESI MS for C₅₇H₆₈FN₄O₉PS₂ Calculated 1067.3. Observed 1065.6 (M−1). ³¹P NMR (202 MHz, CDCl₃): δ154.76 (d, J 7.4 Hz), 154.49 (d, J 10.1 Hz)

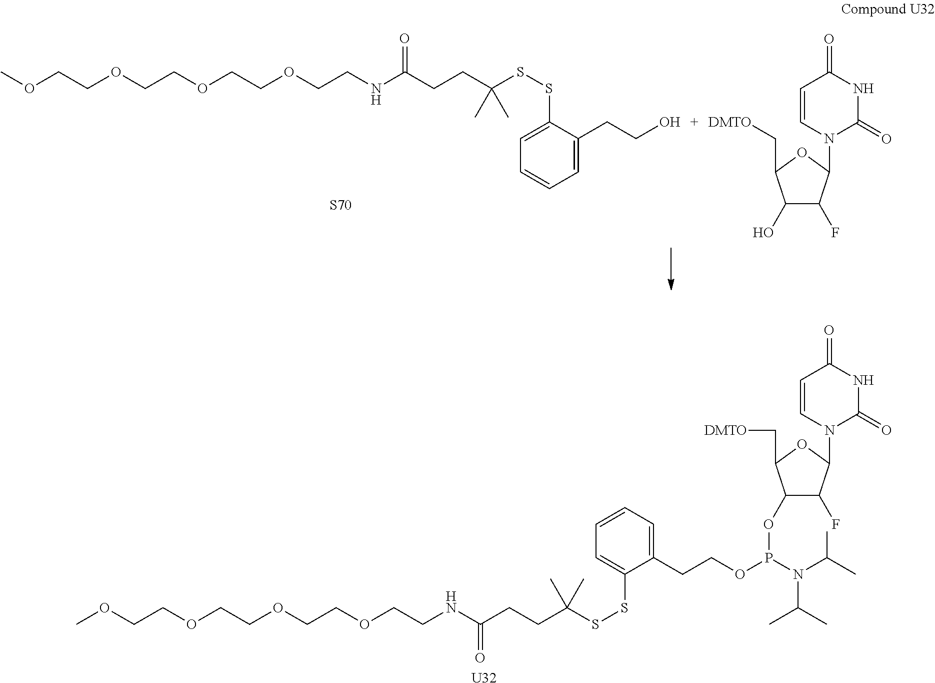

Compound U32 was prepared according to procedure 1 (see compound U24). ESI MS for C₅₉H₈₀FN₄O₁₃PS₂Calculated 1167.4. Observed 1166.5 (M−1). ³¹P NMR (202 MHz, CDCl₃): δ154.71 (d, J 7.3 Hz), 154.00 (d, J 10.9 Hz)

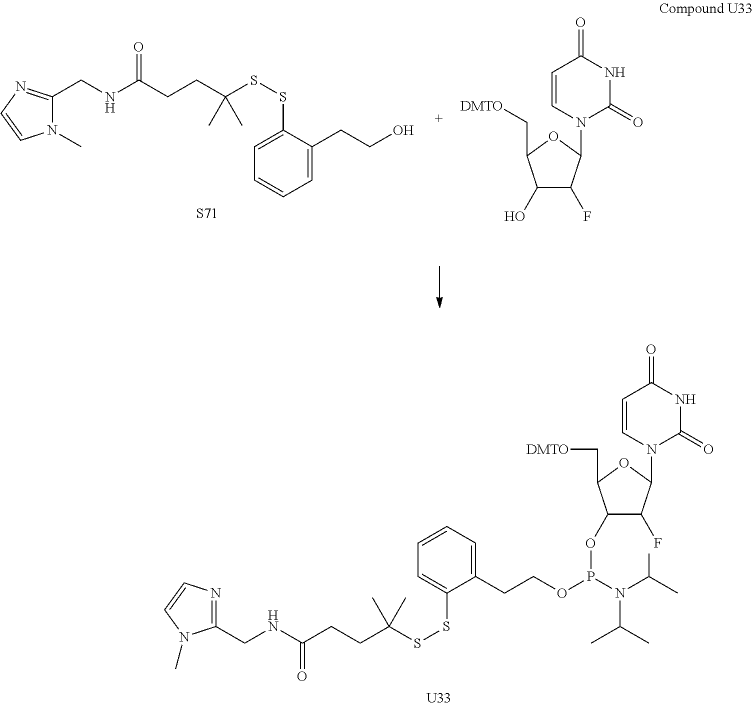

Compound U33 was prepared according to procedure 1 (see compound U24). ESI MS for C₅₅H₆₈FN₆O₉PS₂ Calculated 1071.3. Observed 1072.1 (M+1). ³¹P NMR (202 MHz, CDCl₃): δ 155.09 (s), 152.98 (d, J 14.9 Hz)

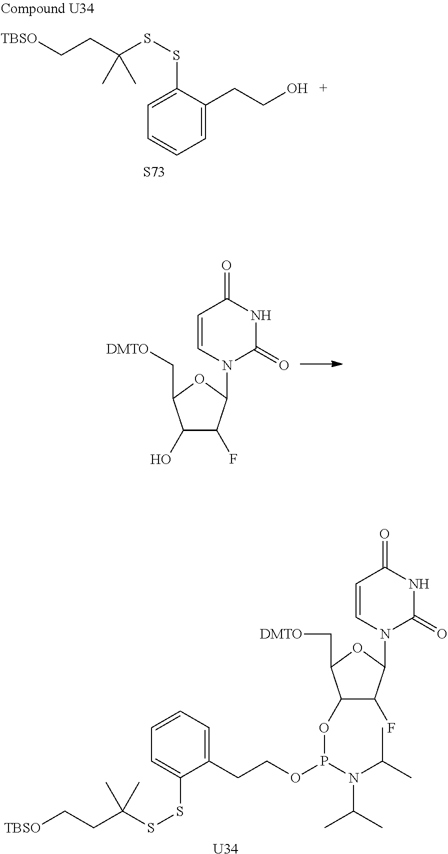

Compound U34 was prepared according to procedure 1 (see compound U24). ESI MS for C₅₅H₇₅FN₃O₉PS₂Si Calculated 1064.4. Observed 1065.1 (M+1). ³¹P NMR (202 MHz, CDCl₃): δ154.81 (d, J 8.9 Hz), 154.56 (d, J 7.9 Hz)

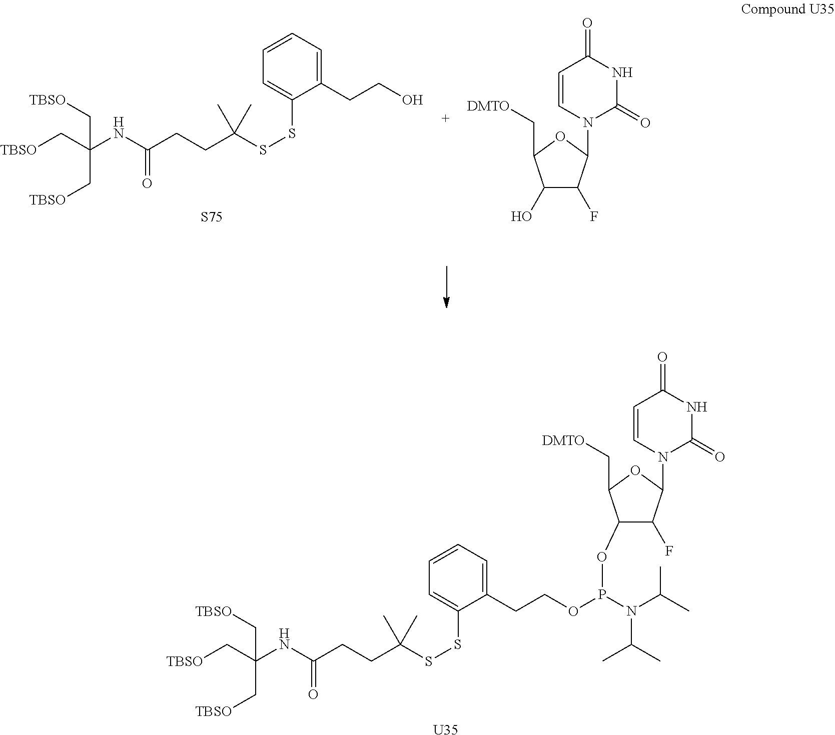

Compound U35 was prepared according to procedure 1 (see compound U24). ³¹P NMR (202 MHz, CDCl₃): δ154.62 (d, J 7.3 Hz), 154.50 (d, J 9.2 Hz)

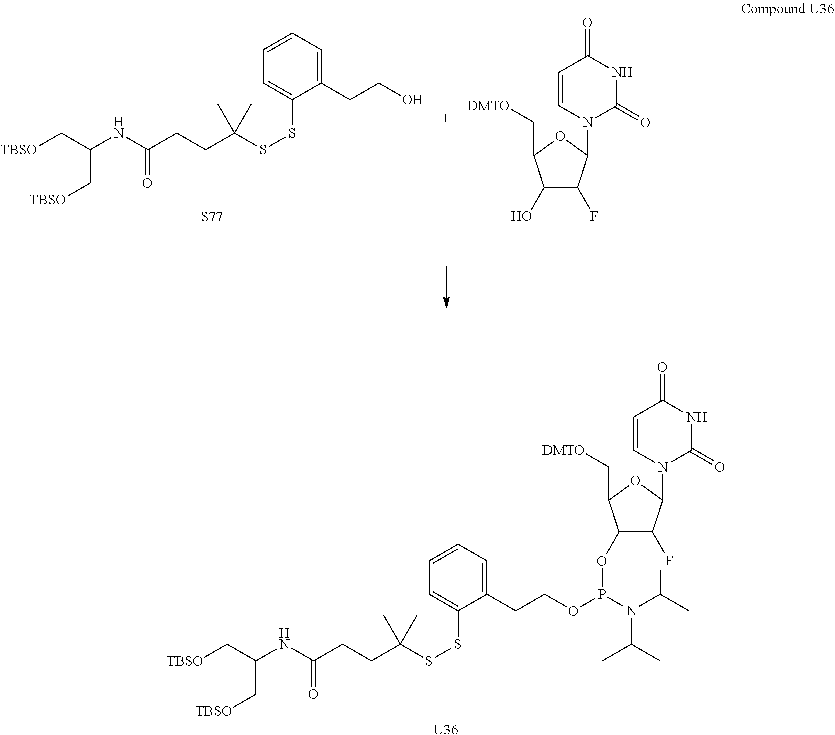

Compound U36 was prepared according to procedure 1 (see compound U24). ESI MS for C₆₅H₉₆FN₄O₁₁PS₂Si₂ Calculated 1279.8. Observed 1278.5 (M−1). ³¹P NMR (202 MHz, CDCl₃): δ154.72 (d, J 7.1 Hz), 154.60 (d, J 9.1 Hz)

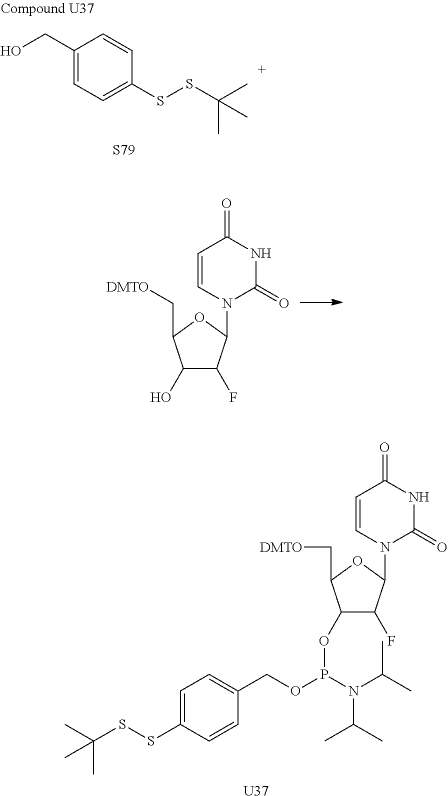

Compound U37 was prepared according to procedure 1 (see compound U24). ESI MS for C₄₇H₅₇FN₃O₈PS₂ Calculated 906.1. Observed 906.7 (M+1). ³¹P NMR (202 MHz, CDCl₃): δ156.35 (d, J 8.5 Hz), 155.98 (d, J 8.7 Hz)

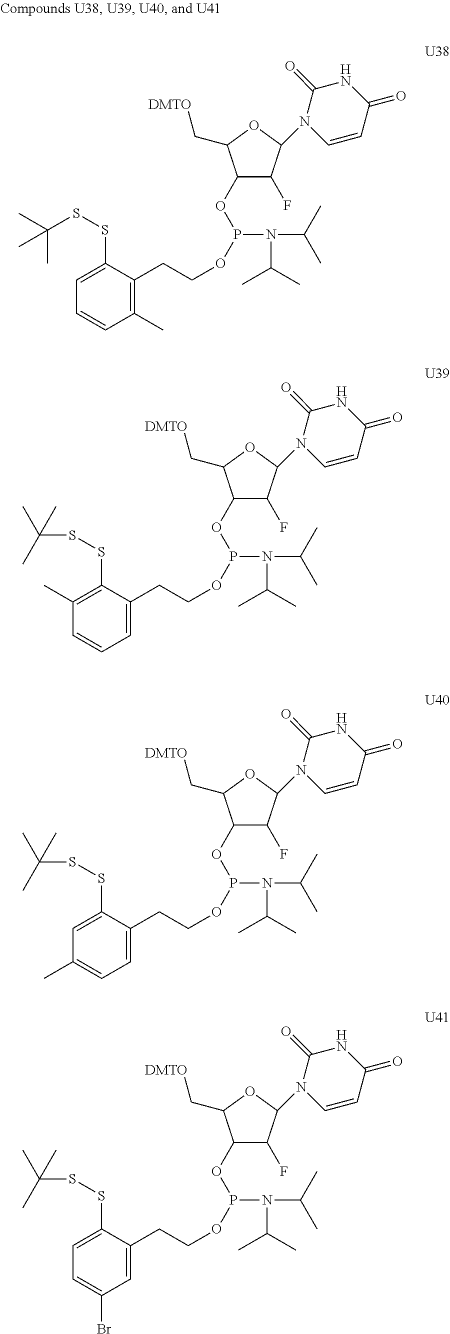

Compounds U38, U39, U40 and U41 were prepared according to procedure 1 (see compound U24).

U38: ESI MS for C₄₉H₆₁FN₃O₈PS₂ Calculated 934.1. Observed 933.1 (M−1). ³¹P NMR (202 MHz, CDCl₃): δ154.74 (d, J 7.7 Hz), 154.70 (d, J 7.9 Hz)

U39: ESI MS for C₄₉H₆₁FN₃O₈PS₂ Calculated 934.1. Observed 844.8 (M-t-BuS). ³¹P NMR (202 MHz, CDCl₃): δ154.81 (d, J 8.7 Hz), 154.58 (d, J 8.3 Hz)

U40: ESI MS for C₄₉H₆₁FN₃O₈PS₂ Calculated 934.1. Observed 933.5 (M−1)

³¹P NMR (202 MHz, CDCl₃): δ154.64 (d, J 8.3 Hz), 154.53 (d, J 7.9 Hz)

U41: ESI MS for C₄₈H₅₈BrFN₃O₈PS₂ Calculated 999.0. Observed 999.9 (M+1). ³¹P NMR (202 MHz, CDCl₃): δ155.47 (d, J 7.7 Hz), 154.74 (d, J 8.7 Hz)

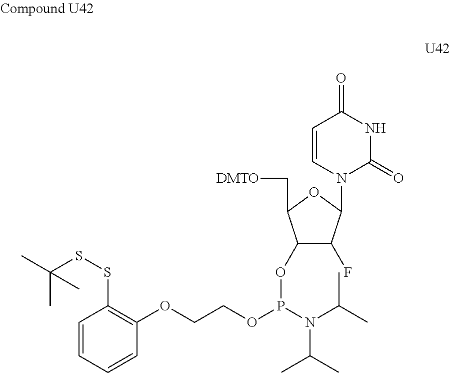

Compound U42 was prepared according to procedure 1 (see compound U24).

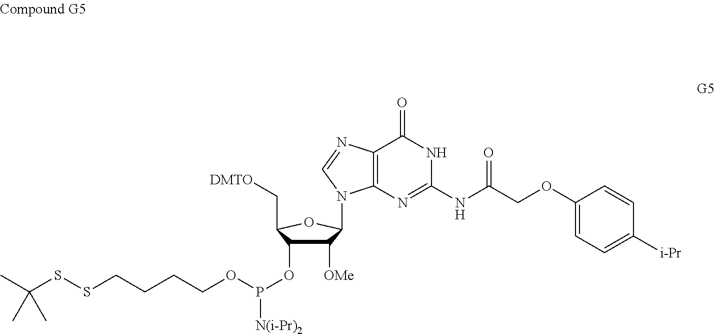

Compound G5 was prepared as described herein. ESI MS for C₅₇H₇₅N₆O₁₀PS₂ calculated 1099.34. observed [M−H]⁺1098.2. ³¹P NMR (202 MHz, CDCl₃) δ150.48 (s), 149.87 (s).

The synthetic routes described herein may be used to prepare other nucleotides of the invention, for example:

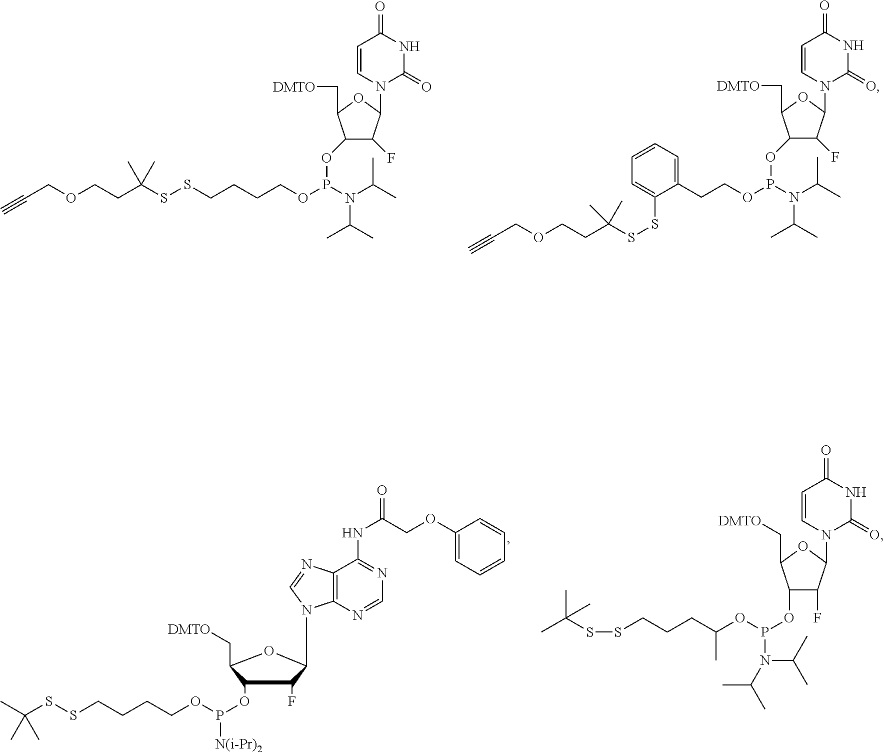

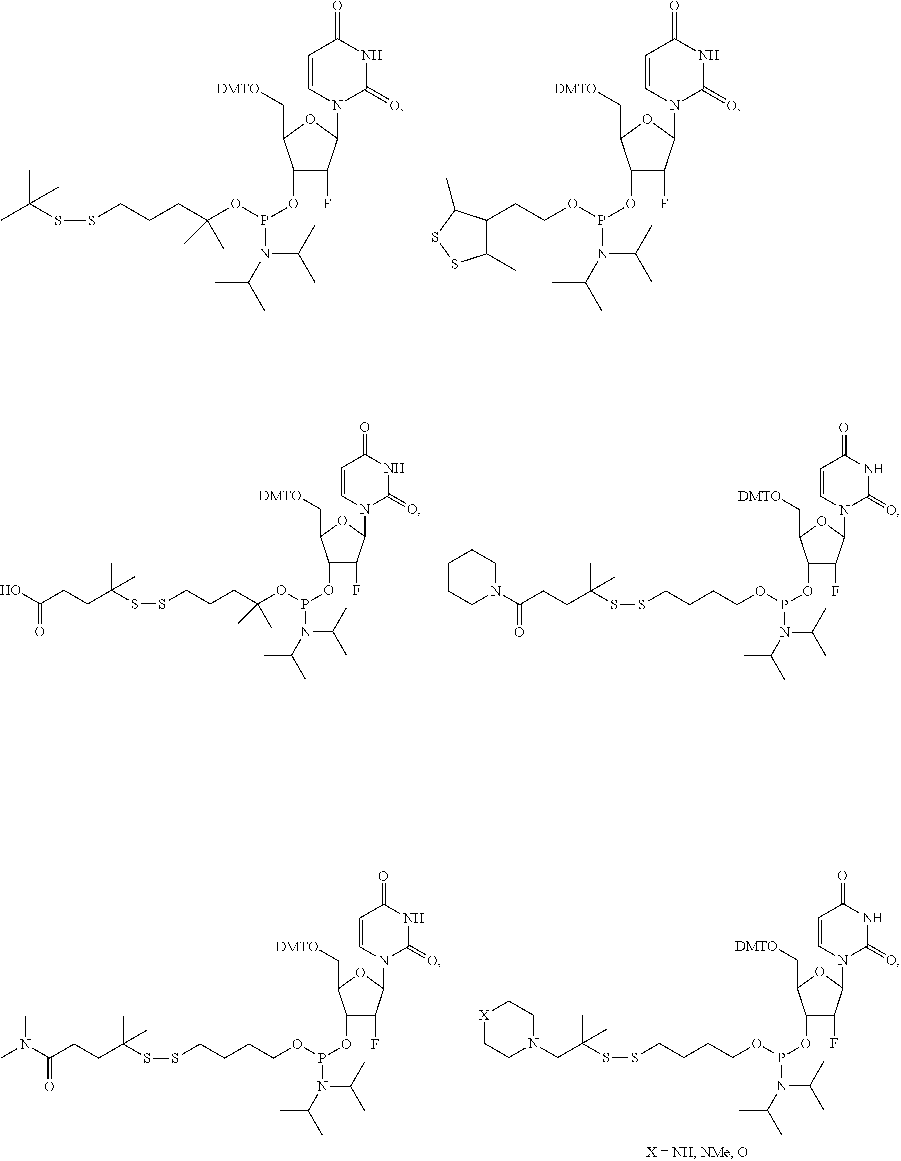

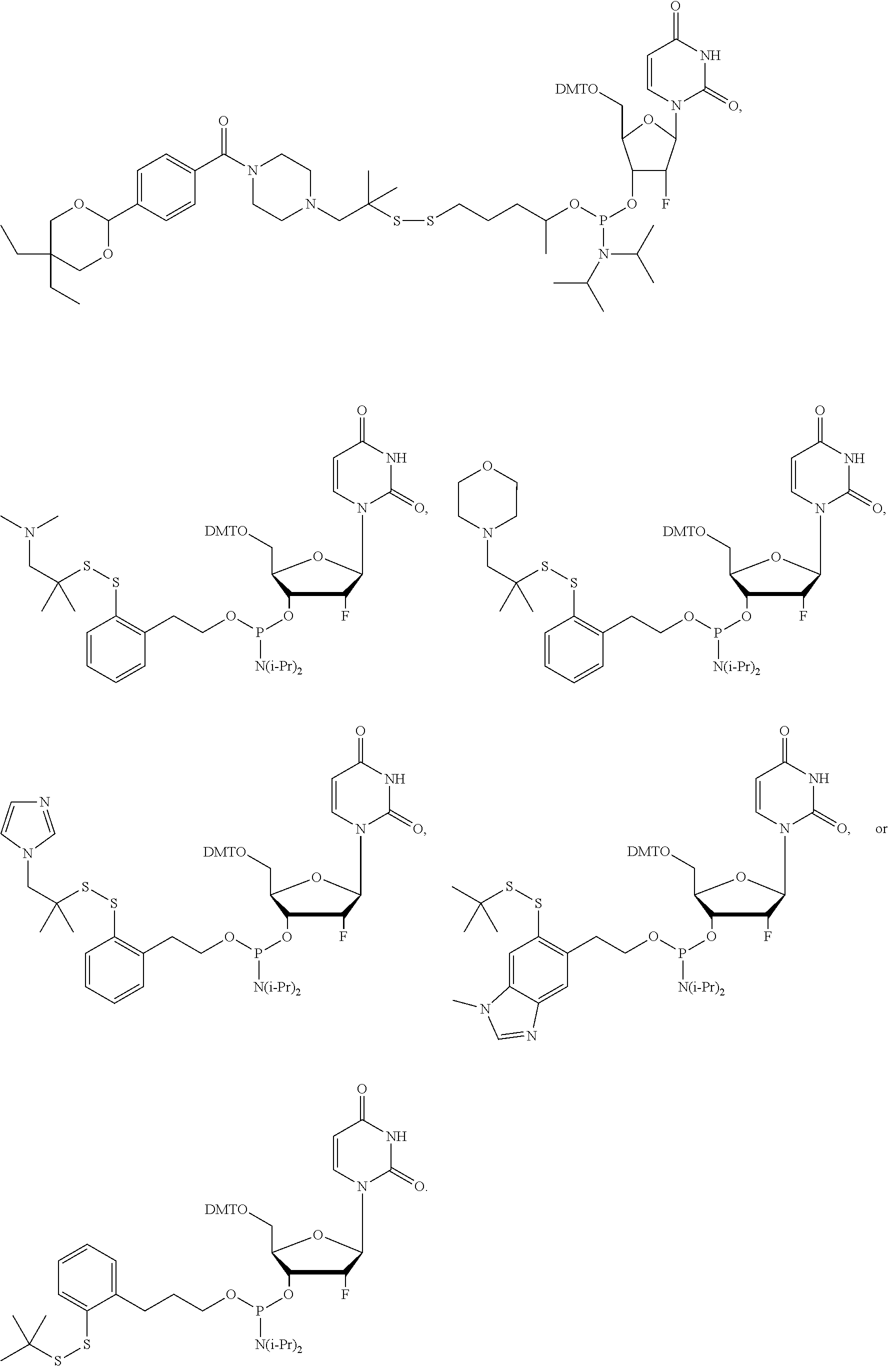

### Synthesis of Cell Penetrating Peptides (Protein Transduction Domains)

### Peptide Synthesis:

Synthesis:

Rink amide polystyrene resin (0.080 g, 0.61 mmol/g) was added to the reaction vessel, swelled three times in dimethylformamide (5 volumes) for 7 min. each time with nitrogen bubbling and then drained. The assembly of the peptide was carried out using the following cycles and employing standard Fmoc chemistry:
Fmoc deprotection with 20% piperidine in dimethylformamide (DMF) 3×4 min;
Resin washed with DMF, 6×1 min;
Couplings used 5 eq. protected amino acid, 15 eq. N-methylmorpholine (NMM), and 5 eq. HCTU. After adding the coupling solution, the reaction was allowed to proceed for 2×20 min;
On completion of coupling, the resin was washed with DMF for 6×1 min;
For the final assembly step, the N-terminus was capped by adding 5 eq. of Fmoc-6-Hydrazinoicotinic Acid; 5 eq. HATU and 15 eq. NMM in DMF and mixing until the reaction was complete (around 1 hr), as confirmed by the Kaiser (ninhydrin) test. The Fmoc removed by 20% piperidine in DMF 3×4 min; and
The completed resin-bound peptide was washed three times with DMF, three times with dichloromethane (DCM) and then dried under vacuum.

Cleavage:

The peptide was cleaved/deprotected from the resin using the following solution: trifluoroacetic acid/dithiothreitol/water/acetone/triisopropylsilane (10 ml, 90/3/2/3/2), with stirring for 2 hr. The resin was filtered through a medium frit, syringe filter and washed twice with neat trifluoroacetic acid (TFA). The filtrates were combined and the volume reduced to half by evaporation. The TFA solution was stirred and the crude peptide precipitated by the slow addition of 4 volumes of ice-cold ether. The precipitated crude peptide was collected by filtration.

Purification:

The crude material was analyzed by LC/MS using a 15-75% B (A=0.1% trifluoroacetic acid/water; B=0.1% trifluoroacetic acid/acetonitrile) over 20 min using a Phenomenex Luna C₁₈ (100×4.6 mm 5μ) column.

List of Cell Penetrating Peptides, Endosomolytic peptides, and certain targeting moieties synthesized is shown in Table 3.

TABLE 3

| Com- | SEQ | | | | |
|---|---|---|---|---|---|
| pound | ID | | C- | MW | MW |
| # | NO: | Structure | Terminus | Calcd | Observ |
| P01 | 26 | HyNic GGRK′RKRK′RK′RK′RK′RK′R | CONH₂ | 2412 | 2413 |
| P02 | 27 | HyNic GGRK′RKRK′RK′RK′RK′RK′RK′RK′RK′RK′R | CONH₂ | 3548 | 3547 |
| P03 | 28 | HyNic GGRK′RKRK′RK′RK′RK′RK′RK′RK′RK′RK′RK′RK′RK′RK′R | CONH₂ | 4665 | 4668 |
| P04 | 29 | HyNic GGESDSELEIKRYKNRVASRKSRAKFKQLLQHYREVAA | CONH₂ | 6557 | 6563 |
| | | AKSSENDRLRLLLKQSS | | | |
| P05 | 30 | HyNic GGSRRHHSRSKAKRSRHH | CONH₂ | 2312 | 2311 |
| P06 | 31 | HyNic GGAYDLRRRERQSRLRRRERQSR | CONH₂ | 3134 | 3132 |
| P07 | 32 | HyNic GGMAPQRDTVGGRTTPPSWGPAKAQLRNSCA | CONH₂ | 3344 | 3342 |
| P08 | 33 | HyNic GGMAPQRDTVGGRTTPPSWGPAKAQLRNSSA | CONH₂ | 3328 | 3327 |
| P09 | 34 | HyNic GGFCIGRL | CONH₂ | 997 | 997 |
| P10 | 35 | HyNic GGGVIGRL | CONH₂ | 994 | 993 |
| P11 | 36 | HyNic GGRAWMRWYSPTTRRYG | CONH₂ | 2277 | 2276 |
| P12 | 37 | HyNic GGPLILLRLLR | CONH₂ | 1396 | 1395 |
| P13 | 38 | HyNic GGMIIYRDLISH | CONH₂ | 1533 | 1532 |
| P14 | 39 | HyNic GGACTGSTQHQCG | CONH₂ | 1380 | 1378 |
| P15 | 40 | HyNic GGALFLGWLGAAGSTMGAPKSKRKV | CONH₂ | 2619 | 2618 |
| P16 | 41 | HyNic GGLIRLWSHLIHIWFQNRRLKWKKK | CONH₂ | 3214 | 3211 |
| P17 | 42 | HyNic GGIGAVLKVLTTGLPALISWIKRKRQQ | CONH₂ | 3081 | 3079 |
| P18 | 43 | HyNic GGLHKLLHHLLHHLHKLLHHLHHLLHKL | CONH₂ | 3559 | 3556 |
| P19 | 44 | HyNic GGRKKR | CONH₂ | 875 | 875 |
| P20 | 45 | HyNic GGRKKRRQRRR | CONH₂ | 1629 | 1627 |
| P21 | 46 | HyNic GGRKKRRQRRRGGRKKR | CONH₂ | 2311 | 2309 |
| P22 | 47 | N₃ GGRKKRRQRRR-Peg24-GGRKKRRQRRR-Peg24- | CONH₂ | 6459 | 6450 |
| | | GGRKKRRQRRR | | | |
| P23 | 48 | HyNic GGRKKRRQRRR-Peg24-GGRKKRRQRRR-Peg24- | CONH₂ | 6379 | 6385 |
| | | GGRKKRRQRRR | | | |
| P24 | 49 | HyNic GGRK′RKRK′RK′RK′RK′RK′RK′RK′RK′RK′RC(Peg24) | CONH₂ | 4928 | 4934 |
| P25 | 50 | HyNic GGRK′RKRK′RK′RK′RK′RK′RK′RK′RK′RK′RC(Peg48) | CONH₂ | 5980 | 5987 |
| P26 | 51 | HyNic GGRKKRRQRRR-Peg24-GGRKKRRQRRR-Peg24- | CONH₂ | 6754 | 6777 |
| | | GGRKKRRQRRRK(Hexanoic Acid) | | | |
| P27 | 52 | GGLHKLLHHLLHHLHKLLHHLHHLLHKL | CONH₂ | 3382 | 3380 |
| P28 | 53 | GGACTGSTQHQCG | CONH₂ | 1205 | 1203 |
| P29 | 54 | GGLIRLWSHLIHIWFQNRRLKWKKK | CONH₂ | 3214 | 3211 |
| P30 | 55 | GGALFLGWLGAAGSTMGAPKSKRKV | CONH₂ | 2444 | 2442 |
| P31 | 56 | GGIGAVLKVLTTGLPALISWIKRKRQQ | CONH₂ | 2904 | 2903 |
| P32 | 57 | HyNic GGLFGAIAGFIENGWEGMIDGWYG | CONH₂ | 2693 | 2695 |
| P33 | 58 | HyNic GGLFEAIEGFIENGWEGMIDGWYG | CONH₂ | 2821 | 2844 |
| P34 | 59 | HyNic GGLFEAIEGFIENGWEGMIDGWYGRKKRRQRRR | CONH₂ | 4144 | 4142 |
| P35 | 60 | HyNic GGLFEAIEGFIENGWEGLIEGWYG | CONH₂ | 2833 | 2856 |
| P36 | 61 | HyNic GGKWKLFKKIGAVLKVLTTGYGRKKRRQRRR | CONH₂ | 3862 | 3861 |
| P37 | 62 | AzidePEG4-ILSSLTVTQLLRRLHQWI | CONH₂ | 2449 | 2449 |
| P38 | 63 | AzidePEG4-MKWVTFISLLFLFFSSAYS | CONH2 | 2413 | 2411 |
| P39 | 64 | AzidePEG4-MIRTLLLSTLVAGALS | CONH2 | 1932 | 1931 |
| P40 | 65 | AzidePEG4-RLIEDICLPRWGCLWEDD | CONH2 | 2503 | 2502 |
| P41 | | Azide-C18 | - | 267 | 267 |
| P42 | 66 | AzidePEG4-KDEL | CONH₂ | 777 | 776 |
| P43 | 67 | AzidePEG4-LFEAIEGFIENGWEGMIDGWYGKDEL | CONH₂ | 3291 | |
| P44 | 68 | AzidePEG4-LFEAIEGFIENGWEGMIDGWYGRKKRRQRRRKDEL | CONH₂ | 4614 | |
| P45 | 69 | Azide-PEG4 RLIEDICLPRWGCLWEDD (Albumin binding) | CONH₂ | 2503 | 2502 |
| P46 | 70 | Azide-PEG4 MKLSLVAAMLLLLSAARA (ER targeting) | CONH₂ | 2145 | 2144 |
| P47 | 71 | Azide-PEG4 MKLAVTLTLVTLALSSSSASA (ER targeting) | CONH₂ | 2332 | 2348 |
| P48 | 72 | Azide-PEG4 FFKKLAHALHLLALLALHLAHALKKA (Endosomolytic) | CONH₂ | 3161 | 3161 |
| P49 | 73 | Azide-PEG4 PSQPTYPGDDAPVRDLIRFYRDLRRYLNVVTRHRY | CONH₂ | 4578 | 4579 |
| P50 | 74 | Azide-PEG4 RLIEDICLPRWGCLWEDDKDEL (ER targeting) | CONH₂ | 2988 | 2987 |
| P51 | 75 | Azide-PEG4 LFEAIEGFIENGWGMIDGWYG (Endosomolytic) | CONH₂ | 2804 | 2802 |
| P52 | 76 | Azide-PEG4 LFEAIEGFIENGWEGMIDGWYGRKKRRQRRR | CONH₂ | 4127 | 4127 |
| | | (Endosomolytic) | | | |
| P53 | 77 | Azide-PEG4 MIRTLLLSTLVAGALSKDEL (ER targeting) | CONH₂ | 2417 | 2416 |
| P54 | 78 | Ac YEQDPWGVKWWYK(Peg4-N3) | CONH₂ | 2100 | 2099 |
| P55 | 79 | NH2 MIRTLLLSTLVAGALSK(Peg4-N3) (ER targeting) | CONH₂ | 2057 | 2059 |
| P56 | 80 | NH2 YEQDPWGVKWWYK(Peg4-N3) | CONH₂ | 2058 | 2057 |
| P57 | 81 | Azide-PEG4 R-Bip-R-Bip-R (Albumin binding) | CONH₂ | 1205 | 1205 |
| P58 | 82 | Azide-PEG4 R-Bip-R (Albumin binding) | CONH₂ | 826 | 827 |
| P59 | 83 | NH2 ILSSLTVTQLLRRLHQWIK(Peg4-N3) (ER targeting) | CONH₂ | 2577 | 2579 |
| P60 | 84 | NH2 MIRTLLLSTLVAGALSKDEL(Peg4-N3) (ER targeting) | CONH₂ | 2544 | 2544 |
| P61 | 85 | Azide-PEG4 LFEAIEGFIENGWEGMIDGWYGRKKRRQRRRKDEL | CONH₂ | 4610 | 4609 |
| P62 | 86 | Azide-PEG4 IGAVLKVLTTGLPALISWIKRKRQQ (Endosomolytic) | CONH₂ | 3062 | 3061 |
| P63 | 87 | Azide-PEG4 IGAVLKVLTTGLPALISWIKRKRQQKDEL | CONH₂ | 3550 | 3548 |
| P01 | 26 | HyNic GGRK′RK′RK′RK′RK′RK′RK′R | CONH₂ | 2412 | 2413 |
| P02 | 27 | HyNic GGRK′RK′RK′RK′RK′RK′RK′RK′RK′RK′RK′R | CONH₂ | 3548 | 3547 |
| P03 | 28 | HyNic GGRK′RK′RK′RK′RK′RK′RK′RK′RK′RK′RK′RK′RK′RK′R | CONH₂ | 4665 | 4668 |
| | | K′R | | | |
| P04 | 29 | HyNic GGESDSELEIKRYKNRVASRKSRAKFKQLLQHYREVAA | CONH₂ | 6557 | 6563 |
| | | AKSSENDRLRLLLKQSS | | | |
| P05 | 30 | HyNic GGSRRHHSRSKAKRSRHH | CONH₂ | 2312 | 2311 |
| P06 | 31 | HyNic GGAYDLRRRERQSRLRRRERQSR | CONH₂ | 3134 | 3132 |
| P07 | 32 | HyNic GGMAPQRDTVGGRTTPPSWGPAKAQLRNSCA | CONH₂ | 3344 | 3342 |
| P08 | 33 | HyNic GGMAPQRDTVGGRTTPPSWGPAKAQLRNSSA | CONH₂ | 3328 | 3327 |
| P09 | 34 | HyNic GGFCIGRL | CONH₂ | 997 | 997 |
| P10 | 35 | HyNic GGGVIGRL | CONH₂ | 994 | 993 |
| P11 | 36 | HyNic GGRAWMRWYSPTTRRYG | CONH₂ | 2277 | 2276 |
| P12 | 37 | HyNic GGPLILLRLLR | CONH₂ | 1396 | 1395 |
| P13 | 38 | HyNic GGMIIYRDLISH | CONH₂ | 1533 | 1532 |
| P14 | 39 | HyNic GGACTGSTQHQCG | CONH₂ | 1380 | 1378 |
| P15 | 40 | HyNic GGALFLGWLGAAGSTMGAPKSKRKV | CONH₂ | 2619 | 2618 |
| P16 | 41 | HyNic GGLIRLWSHLIHIWFQNRRLKWKKK | CONH₂ | 3214 | 3211 |
| P17 | 42 | HyNic GGIGAVLKVLTTGLPALISWIKRKRQQ | CONH₂ | 3081 | 3079 |
| P18 | 43 | HyNic GGLHKLLHHLLHHLHKLLHHLHHLLHKL | CONH₂ | 3559 | 3556 |
| P19 | 44 | HyNic GGRKKR | CONH₂ | 875 | 875 |
| P20 | 45 | HyNic GGRKKRRQRRR | CONH₂ | 1629 | 1627 |
| P21 | 46 | HyNic GGRKKRRQRRRGGRKKR | CONH₂ | 2311 | 2309 |
| P22 | 47 | N₃ GGRKKRRQRRR-Peg24-GGRKKRRQRRR-Peg24- | CONH₂ | 6459 | 6450 |
| | | GGRKKRRQRRR | | | |
| P23 | 48 | HyNic GGRKKRRQRRR-Peg24-GGRKKRRQRRR-Peg24- | CONH₂ | 6379 | 6385 |
| | | GGRKKRRQRRR | | | |
| P24 | 49 | HyNic GGRK′RK′RK′RK′RK′RK′RK′RK′RK′RK′RK′RC(Peg24) | CONH₂ | 4928 | 4934 |
| P25 | 50 | HyNic GGRK′RK′RK′RK′RK′RK′RK′RK′RK′RK′RK′RC(Peg48) | CONH₂ | 5980 | 5987 |
| P26 | 51 | HyNic GGRKKRRQRRR-Peg24-GGRKKRRQRRR-Peg24- | CONH₂ | 6754 | 6777 |
| | | GGRKKRRQRRRK(Hexanoic Acid) | | | |
| P27 | 52 | GGLHKLLHHLLHHLHKLLHHLHHLLHKL | CONH₂ | 3382 | 3380 |
| P28 | 53 | GGACTGSTQHQCG | CONH₂ | 1205 | 1203 |
| P29 | 54 | GGLIRLWSHLIHIWFQNRRLKWKKK | CONH₂ | 3214 | 3211 |
| P30 | 55 | GGALFLGWLGAAGSTMGAPKSKRKV | CONH₂ | 2444 | 2442 |
| P31 | 56 | GGIGAVLKVLTTGLPALISWIKRKRQQ | CONH₂ | 2904 | 2903 |
| P32 | 57 | HyNic GGLFGAIAGFIENGWEGMIDGWYG | CONH₂ | 2693 | 2695 |
| P33 | 58 | HyNic GGLFEAIEGFIENGWEGMIDGWYG | CONH₂ | 2821 | 2844 |
| P34 | 59 | HyNic GGLFEAIEGFIENGWEGMIDGWYGRKKRRQRRR | CONH₂ | 4144 | 4142 |
| P35 | 60 | HyNic GGLFEAIEGFIENGWEGLIEGWYG | CONH₂ | 2833 | 2856 |
| P36 | 61 | HyNic GGKWKLFKKIGAVLKVLTTGYGRKKRRQRRR | CONH₂ | 3862 | 3861 |
| P37 | 62 | AzidePEG4-ILSSLTVTQLLRRLHQWI | CONH₂ | 2449 | 2449 |
| P38 | 63 | AzidePEG4-MKWVTFISLLFLFFSSAYS | CONH2 | 2413 | 2411 |
| P39 | 64 | AzidePEG4-MIRTLLLSTLVAGALS | CONH2 | 1932 | 1931 |
| P40 | 65 | AzidePEG4-RLIEDICLPRWGCLWEDD | CONH2 | 2503 | 2502 |
| P41 | | Azide-C18 | - | 267 | 267 |
| P42 | 66 | AzidePEG4-KDEL | CONH₂ | 777 | 776 |
| P43 | 67 | AzidePEG4-LFEAIEGFIENGWEGMIDGWYGKDEL | CONH₂ | 3291 | |
| P44 | 68 | AzidePEG4-LFEAIEGFIENGWEGMIDGWYGRKKRRQRRRKDEL | CONH₂ | 4614 | |

In Table 3:
(1) HyNic = hydrazine-nicotinamide, K′ = Boc-Lys(Fmoc)-OH; Bip: Bis-phenylalanine;
(2) compounds P01, P02, P03, P04, P05, P06, P07, P08, P09, P10, P11, P12, P13, P14, P15, P16, P19, P20, P21, P22, P23, P24, P25, and P26 include cell-penetrating peptides; compounds P16, P17, P18, P27, P28, P29, P31, P32, P33, P34, P35, and P36 include endosomolytic peptides; compounds P37, P38, and P39 include peptides targeting the endoplasmic reticulum; compounds P40 and P41 are albumin-binding moieties, and compound P 42 includes a KDEL receptor targeting moiety.

### Synthesis of Targeting Ligands

### GalNAc (NAG) Ligand Synthesis:

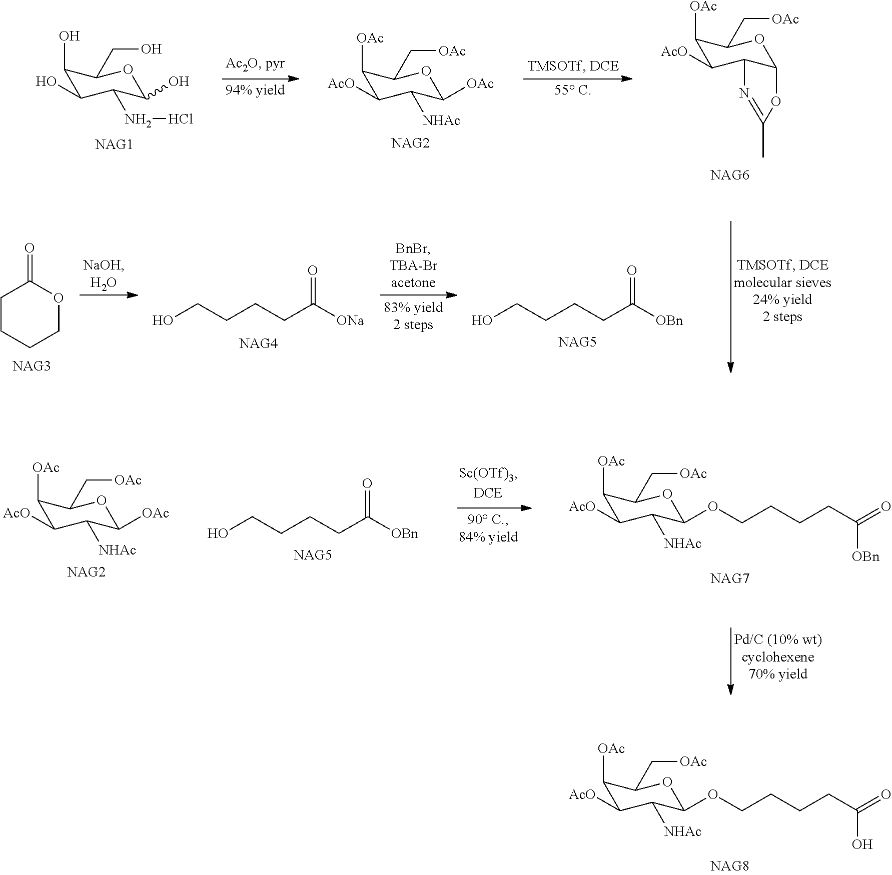

Preparation of D-galactosamine pentaacetate (NAG2). D-Galactosamine (25.0 g, 116 mmol) was suspended in anhydrous pyridine (250 mL) and cooled to 0° C. under an inert atmosphere. Acetic anhydride (120 mL, 1160 mmol) was added over the course of 2 h. After stirring overnight, the reaction mixture was concentrated in vacuo. Upon addition of methanol, a white solid precipitated and was collected via filtration to provide the desired product (42.1 g, 93% yield). ¹H NMR (CDCl₃, 500 MHz): δ 5.69 (d, 1H, J 9.0 Hz), 5.40 (m, 1H), 5.37 (d, 1H, J 3.0 Hz), 5.08 (dd, 1H, J 3.0 Hz, 11 Hz), 4.44 (dt, 1H, J 9.5 Hz, 11 Hz), 4.17 (dd, 1H, J 7.0 Hz, 11.5 Hz), 4.11 (dd, 1H, J 7.0 Hz, 11.5 Hz), 4.01 (t, 1H, J 7.0 Hz), 2.17 (s, 3H), 2.13 (s, 3H), 2.05 (s, 3H), 2.02 (s, 3H), 1.94 (s, 3H), 1.57 (s, 3H).

Preparation of benzyl 5-hydroxy pentanoate (NAG5). A solution of delta-valerolactone (10.0 g, 100 mmol) and NaOH (4.00 g, 100 mmol) in water (100 mL) was stirred overnight at 70° C. The reaction mixture was cooled to rt and concentrated in vacuo to give white solid NAG4. This solid was suspended in acetone (100 mL) and refluxed overnight with benzyl bromide (20.5 g, 120 mmol) and tetrabutylammonium bromide (1.61 g, 0.50 mmol). Acetone was removed in vacuo to afford an oily residue, which was dissolved in EtOAc and washed with sat NaHCO₃ (aq.) and brine. The organic layer was dried over Na₂SO₄ and concentrated in vacuo give the oily product NAG5 (17.1 g, 82% yield). ¹H NMR (CDCl₃, 500 MHz): δ 7.35 (m, 5H), 3.64 (q, 2H, J 6 Hz, 11.5 Hz), 2.41 (t, 2H, J 7.5 Hz), 1.75 (m, 2H), 1.60 (m, 2H), 1.44 (t, 1H, J 6 Hz).

Preparation of benzyloxycarbonylbutyl 2-deoxy 2-N-acetyl-3,4,6-tri-O-acetyl-3-D-galactopyranoside (NAG7)—Method A. Under an inert atmosphere, TMSOTf (8.56 g, 38.4 mmol) was added to a solution of NAG2 (10.0 g, 25.6 mmol) in DCE (100 mL) at ambient temperature. The mixture was stirred at 55° C. for 2 h, removed from heat, and stirred overnight. The reaction mixture was poured onto ice cold sat NaHCO₃ (aq.) and extracted with CH₂Cl₂. The organic layer was dried over Na₂SO₄ and concentrated in vacuo to give syrup NAG6. A solution NAG6 in DCE (60 mL) was charged with alcohol NAG5 (8.00 g, 38.4 mmol) and molecular sieves. The mixture was placed under an inert atmosphere, treated with TMSOTf (2.85 g, 12.8 mmol), and stirred overnight at rt. The mixture was poured over ice cold sat NaHCO₃ (aq.) and extracted with CH₂Cl₂. The organic layer was dried over Na₂SO₄ and concentrated in vacuo to give syrup. This crude material was purified via SiO₂ gel chromatography to afford glycoside NAG7 (3.3 g, 24% yield). ¹H NMR (CDCl₃, 500 MHz): δ 7.35 (m, 5H), 5.98 (d, 1H, J 7.0 Hz), 5.57 (m, 1H), 5.34 (d, 1H, J 3.0 Hz), 5.25 (dd, 1H, J 3.0 Hz, 11 Hz), 5.10 (s, 2H), 4.63 (d, 1H, J 8.5 Hz), 4.11 (m, 2H), 3.95 (m, 1H), 3.88 (m, 2H), 3.49 (m, 1H), 2.37 (m, 2H), 2.13 (s, 3H), 2.03 (s, 3H), 1.99 (s, 3H), 1.90 (s, 3H), 1.70 (m, 2H), 1.61 (m, 2H).

Preparation of benzyloxycarbonylbutyl 2-deoxy 2-N-acetyl-3,4,6-tri-O-acetyl-3-D-galactopyranoside (NAG7)—Method B. To a solution of NAG2 (5.00 g, 12.8 mmol) and alcohol NAG5 (5.33 g, 25.6 mmol) in DCE (50 mL) was added Sc(OTf)₃ (0.44 g, 0.90 mmol) in one portion. The mixture was placed under an inert atmosphere and refluxed for 3 h. Upon cooling the mixture was diluted with CH₂Cl₂, washed with sat NaHCO3 (aq.), dried over MgSO₄, and concentrated in vacuo. Purification via SiO₂ gel chromatography afforded glycoside NAG7 (5.53 g, 80% yield).

Preparation of carboxybutyl 2-deoxy 2-N-acetyl-3,4,6-tri-O-acetyl-β-D-galactopyranoside (NAG8). A solution of glycoside NAG7 (1.50 g, 2.41 mmol) in EtOH (25 mL) was degassed under vacuum and purged with Argon. The Palladium catalyst (10% wt. on activated carbon, 0.50 g) was added in one portion and the mixture was degassed under vacuum purged with argon. The heterogeneous mixture was charged with cyclohexene (25 mL) and refluxed for 6 h. Upon cooling the catalyst was removed by filtration and the mother liquor concentrated in vacuo. The crude was purified via SiO₂ gel chromatography to afford a white foam NAG8 (0.76 g, 70% yield). 1H NMR (CDCl₃, 500 MHz): δ 5.72 (d, 1H, J 8.5 Hz), 5.35 (d, 1H, J 3.5 Hz), 5.26 (dd, 1H, J 3.5 Hz, 11.5 Hz), 4.67 (d, 1H, J 8.5 Hz), 4.17 (dd, 1H, J 6.5 Hz, 11.5 Hz), 4.12 (dd, 1H, 6.5 Hz, 11.5 Hz), 4.00 (dt, 1H, J 8.5 Hz, 11.5 Hz), 3.92 (m, 2H), 3.53 (m, 1H), 2.39 (m, 2H), 2.15 (s, 3H), 2.05 (s, 3H), 2.01 (s, 3H), 1.97 (s, 3H), 1.71 (m, 2H), 1.65 (m, 2H).

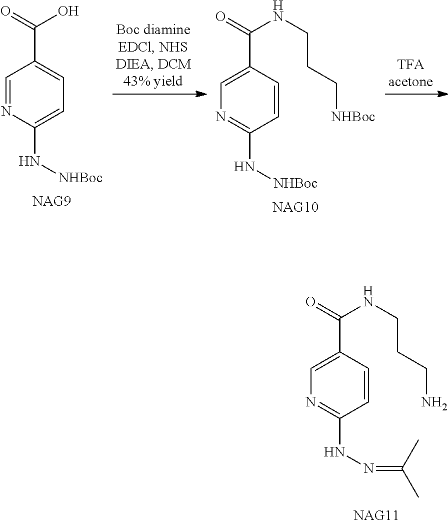 [SMILES: CC(C)(C)OC(=O)NNc1ccc(C(=O)O)cn1 ; CC(C)(C)OC(=O)NCCCNC(=O)c1ccc(NNC(=O)OC(C)(C)C)nc1 ; CC(C)=NNc1ccc(C(=O)NCCCN)cn1]

Preparation of aminopropyl 6-hydrazinonicotamide acetone hydrazone (NAG11). Boc 6-hydrazinonicotinic acid (520 mg, 2.1 mmol) in DCM (20 mL) was treated to EDCl (440 mg, 2.3 mmol), N-hydroxysuccinimide (NHS; 260 mg, 2.3 mmol), Boc-diamine (650 mg, 2.6 mmol), and DIEA (1.1 mL, 6.2 mmol) for 3 h. The reaction was concentrated in vacuo and purified by silica gel chromatography to afford NAG10 (364 mg, 43% yield). ¹H NMR (CDCl₃, 500 MHz): δ 8.55 (br, 1H), 7.93 (d, 2H, J 7.5 Hz), 7.45 (br, 1H), 7.12 (br, 1H), 6.62 (d, 1H, J 8.5 Hz), 5.17 (br, 1H), 3.42 (m, 2H), 3.13 (m, 2H), 1.65 (m, 2H), 1.41 (s, 18H). The HyNic acetone hydrazone was formed through treatment of NAG10 (160 mg, 0.4 mmol) with TFA (9 m L) and acetone (1 mL) for 1 h. The reaction mixture was concentrated in vacuo and placed on the high vacuum to afford NAG11.

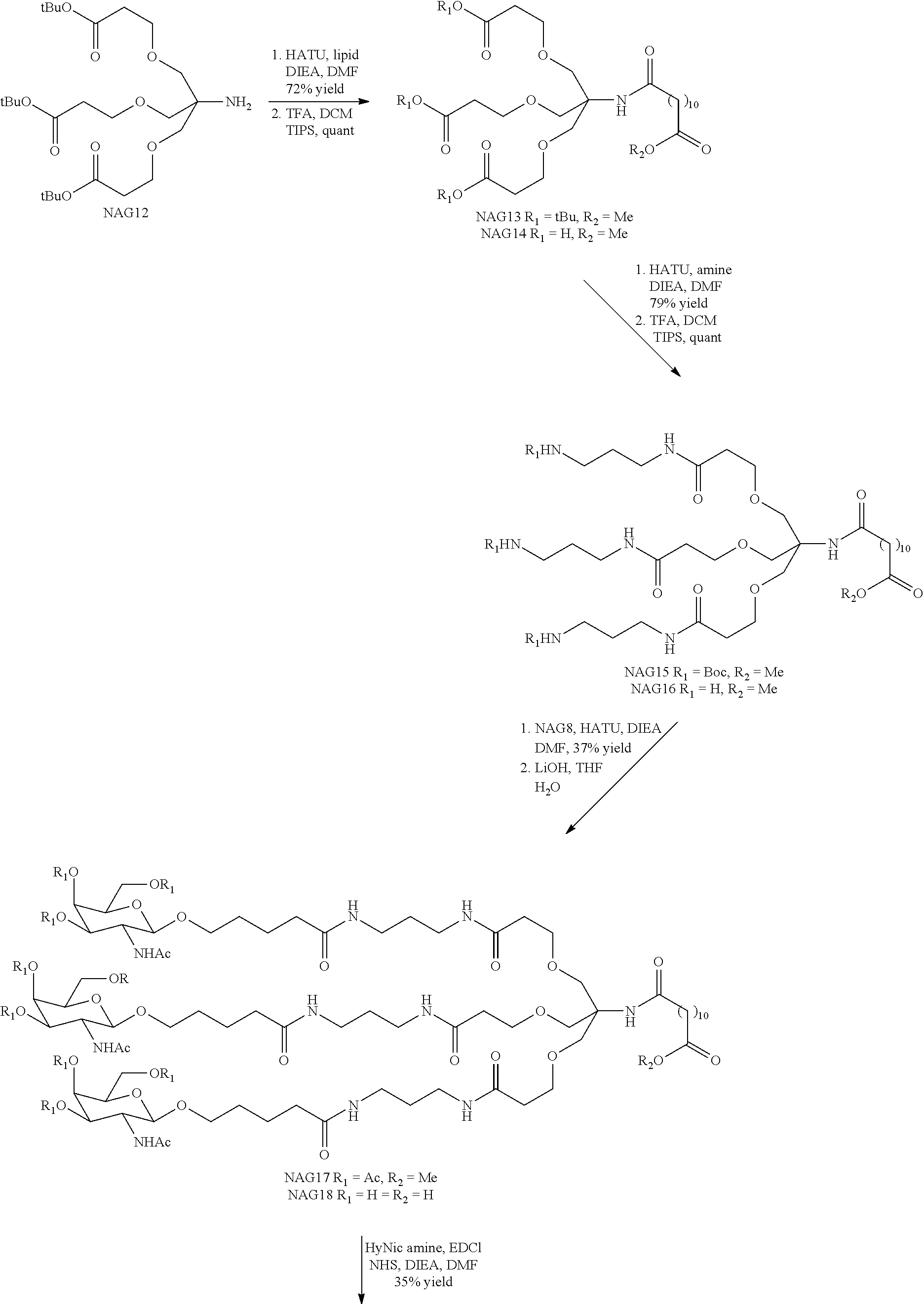

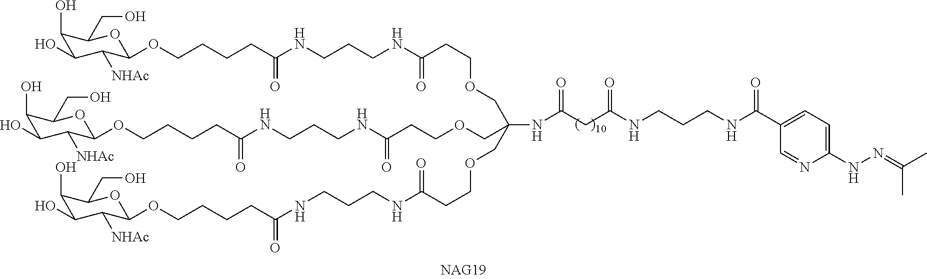

Preparation of tris-(carboxyethoxymethyl)-methylamido-dodecanedioate methyl ester (NAG14). To a solution of dodecanedioic acid methyl ester (211 mg, 0.42 mmol) activated with HATU (122 mg, 0.50 mmol) and DIEA (218 uL, 1.25 mmol) in DMF (2 mL) was added tris linker NAG12. After 1 h, the reaction mixture was concentrated in vacuo and purified by SiO₂ gel chromatography to afford NAG13 (214 mg, 70% yield). MALDI-TOF mass calcd C₃₈H₆₉NO₁₂: 731.48. Found: 755.10 [M+Na]. Tris t-butyl ester NAG13 was hydrolyzed with a TFA:TIPS:DCM (9:0.25:1) cocktail (10.25 mL) for 4 h and concentrated in vacuo to give tris acid NAG14. MALDI-TOF mass calcd C₂₆H₄₅NO₁₂: 563.29. Found: 565.33 [M+H].

Preparation of tris-(aminopropamido-ethoxymethyl)-methylamido-dodecanedioate methyl ester (NAG16). To a solution of tris acid NAG14 (230 mg, 0.41 mmol) activated with HATU (557 mg, 1.35 mmol) and DIEA (470 uL, 2.70 mmol) in DMF (4 mL) was added monoBoc 1,3-diaminopropane (250 mg, 1.44 mmol). After 1 h, the reaction was concentrated in vacuo and purified by SiO₂ gel chromatography to afford NAG15 (335 mg, 79% yield). MALDI-TOF mass calcd C₅₀H₉₃N₇O₁₅: 1031.67. Found: 1056.40 [M+Na]. Tris Boc linker NAG15 was treated with a TFA:TIPS:DCM (9:0.25:1) cocktail (10.25 mL) for 1 h and concentrated in vacuo to give tris amine NAG16. MALDI-TOF mass calcd C₃₅H₆₉N₇O₉: 731.51. Found: 733.18 [M+H].

Preparation of tris-GalNAc (NAG18): Monosaccharide NAG8 (192 mg, 0.43 mmol) was treated with HATU (163 mg, 0.43 mmol) and DIEA (150 uL, 0.86 mmol) in DMF (2 mL). After 30 min, a solution of NAG16 (80 mg, 0.11 mmol) in DMF (1 mL) was added and the mixture stirred for 1 h. The crude mixture was purified by SiO₂ gel chromatography to afford NAG17 (82 mg, 37% yield). Mass calcd C₉₂H₁₅₀N₁₀O₃₉: 2019.00. Found: 2041.85 [M+Na]. The peracetylated trimer GalNAc (82 mg, 0.04 mmol) was hydrolyzed upon treatment with LiOH.H₂O (34 mg, 0.81 mmol) in a THF:H₂O (3:1) solution (8 mL) to afford NAG18. MALDI-TOF mass calcd C₇₃H₁₃₀N₁₀O₃₀: 1626.89. Found: 1634.52 [M+Li]. Preparation of HyNic trimer GalNAc (NAG19). A solution GalNAc trimer NAG18 (32 mg, 0.02 mmol) and HyNic amine NAG11 (20.0 mg, 0.08 mmol) in DMF (1 mL) was treated with EDCl (16.2 mg, 0.08 mmol), NHS (2.5 mg, 0.02 mmol), and DIEA (28 uL, 0.16 mmol) and stirred for 4 h. Upon concentration in vacuo, the crude was dissolved in DMSO and purified by RP-HPLC to afford NAG19 (12.6 mg, 35% yield). MALDI-TOF mass calcd C₈₅H₁₄₇N₁₅O₃₀: 1858.04. Found: 1859.83 [M+H].

### Synthesis of Trivalent GalNAc Azide

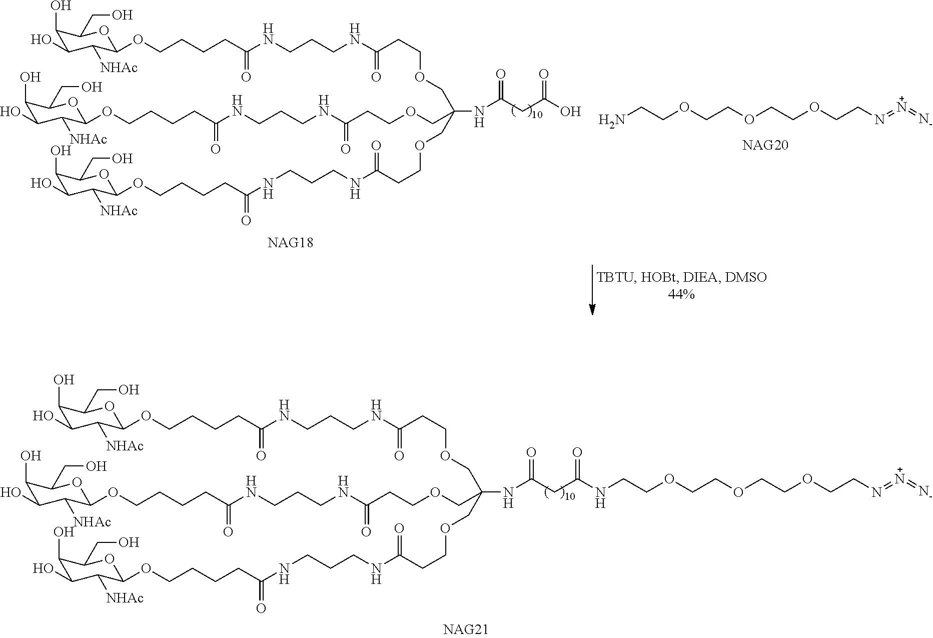

Preparation of azido-Peg3-trimer GalNAc (NAG21). GalNAc trimer carboxylic acid NAG18 (60 mg, 0.03 mmol), azido-Peg3-amine NAG20 (45.6 mg, 0.21 mmol), TBTU (23.8 mg, 0.07 mmol), HOBt (11.5 mg, 0.03 mmol), and DIEA (34 uL) were dissolved in DMSO (0.5 mL) and stirred 2 h. The base was removed in vacuo and the crude purified by RP-HPLC to afford NAG21 (24 mg, 44%).

AP-ESI+ Mass calcd C₈₁H₁₄₆N₁₄O₃₂: 1827.02. Found: 914.8 [M+2H]²⁺.

### Synthesis of Folate Ligand:

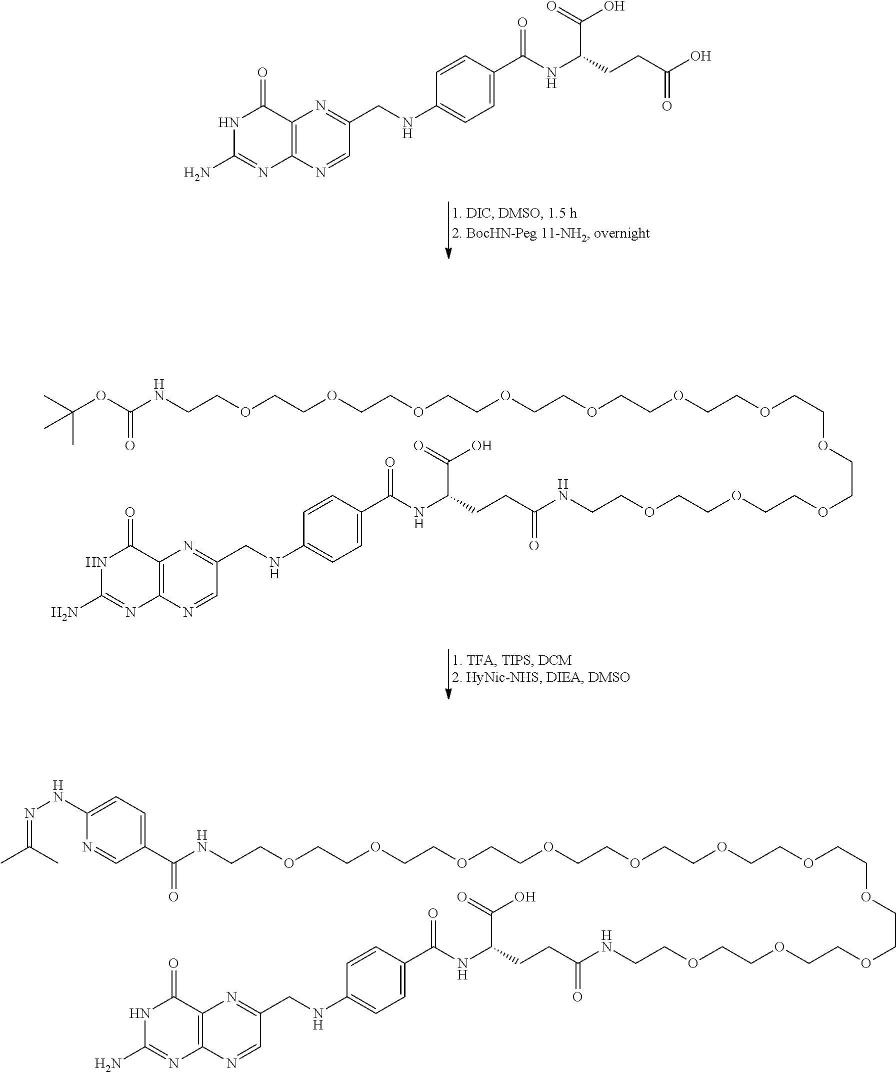 [SMILES: Nc1nc2ncc(CNc3ccc(C(=O)N[C@@H](CCC(=O)O)C(=O)O)cc3)nc2c(=O)[nH]1 ; CC(C)(C)OC(=O)NCCOCCOCCOCCOCCOCCOCCOCCOCCOCCOCCOCCNC(=O)CC[C@H](NC(=O)c1ccc(NCc2cnc3nc(N)[nH]c(=O)c3n2)cc1)C(=O)O ; CC(C)=NNc1ccc(C(=O)NCCOCCOCCOCCOCCOCCOCCOCCOCCOCCOCCOCCNC(=O)CC[C@H](NC(=O)c2ccc(NCc3cnc4nc(N)[nH]c(=O)c4n3)cc2)C(=O)O)cn1]

Preparation of N-Boc-Peg11 folate (F2). To a solution of folic acid (225 mg, 0.51 mmol) in DMSO (4 mL) was added diisopropylcarbodiimide (80 uL, 0.51 mmol). After stirring for 1.5 h, a solution of Boc-Peg11-diamine (220 mg, 0.34 mmol) in DMSO (1 mL) was added and the reaction stirred overnight. Addition of water (35 mL), precipitated a solid which collected via filtration and purified by RP-HPLC to afford F2 (364 mg, 67% yield). MALDI-TOF mass calcd C₄₈H₇₇N₉O₁₈: 1067.54. Found: 1069.89 [M+H]. Preparation of folate-peg11-HyNic acetone hydrazone (F3). MonoBoc F2 (210 mg, 0.2 mmol) was treated with TFA (9 mL) and acetone (1 mL) for 1.5 h, concentrated in vacuo, and dried under a high vacuum. MALDI-TOF mass calcd C₄₃H₆₉N₉O₁₆: 967.48. Found: 969.86 [M+H]. The crude yellowish solid was dissolved in DMSO (200 uL) and treated with a solution of HyNic-NHS ester (10.0 mg, 0.03 mmol) and DIEA (40 uL, 0.23 mmol) for 1.5 h. The crude was purified by RP-HPLC to afford F3 (1.2 mg, 3.5% yield). MALDI-TOF mass calcd C₅₂H₇₈N₁₂O₁₇: 1142.56. Found: 1144.03 [M+H].

### Synthesis of Monovalent Folate Azide

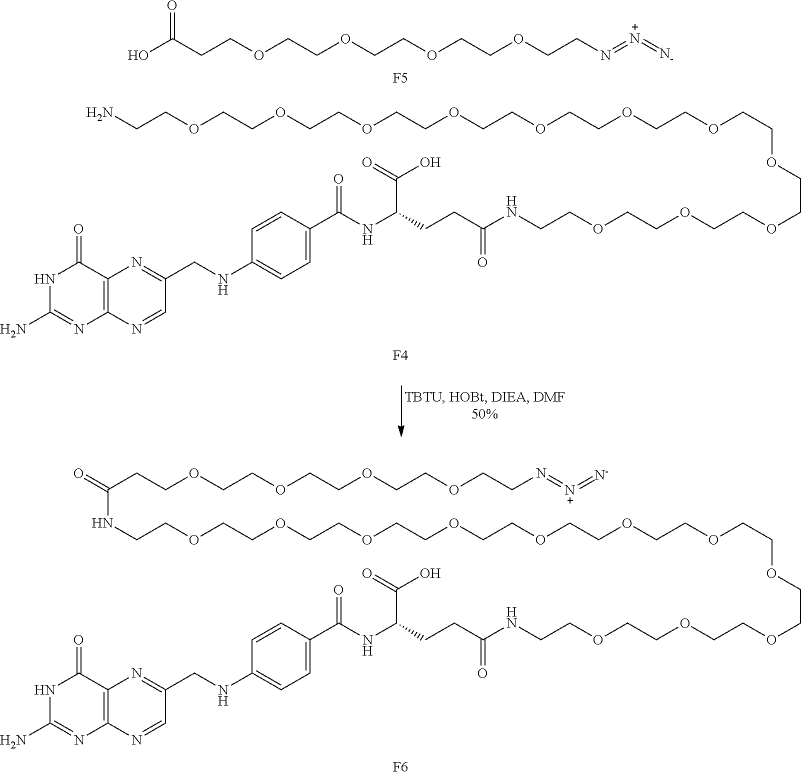

Preparation of azido-Peg4-amido-Peg11 folate (F6). Amino-Peg11 folate F4 (115 mg, 0.12 mmol) in DMSO (1.0 mL) was added to a solution of azido-Peg4 acid (38 mg, 0.13 mmol) activated with TBTU (42 mg, 0.13 mmol), HOBt (20 mg, 0.13 mmol), and DIEA (63 uL, 0.36 mmol) in DMSO (1.0 mL). After 2 h, base was removed in vacuo and the crude purified by RP-HPLC to afford F6 (75 mg, 50%). AP-ESI+ Mass calcd C₅₄H₈₈N₁₂O₂₁: 1240.61. Found: 1241.7 [M+H]⁺, 621.5 [M+2H]²⁺.

### Synthesis of PSMA Ligands

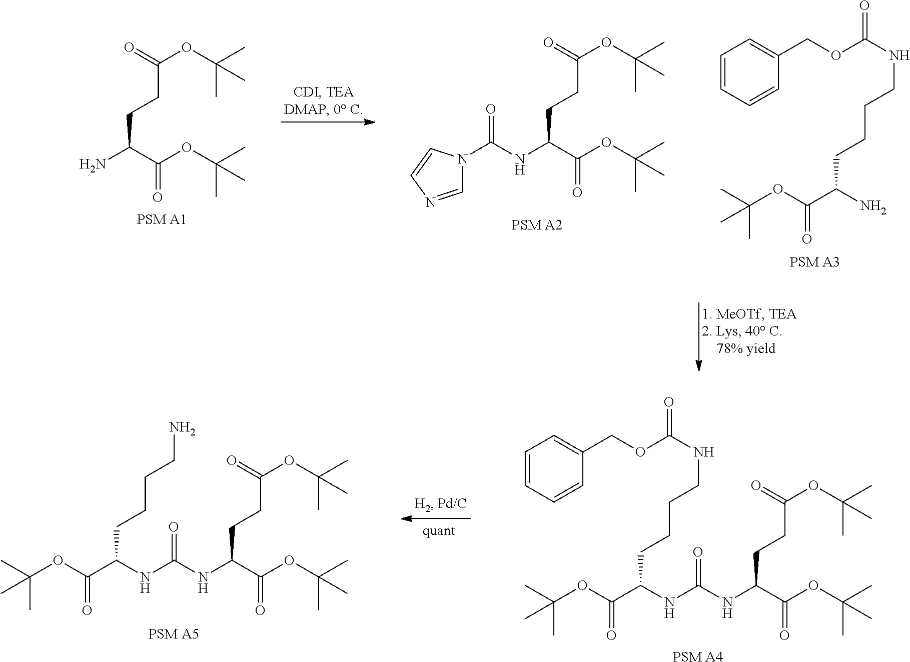

Preparation of Cbz-Lys ureido Glu tris-t-butyl ester (PSMA4). To an ice cold solution of glutamic di-tert-butyl ester (1.06 g, 3.58 mmol), DMAP (27 mg), and TEA (1.25 mL, 8.95 mmol) in CH₂Cl₂ (10.0 mL) was added CDI (638 mg, 3.94 mmol) in one portion. After 30 min, the reaction was removed from the ice bath and stirred overnight. The reaction was diluted with CH₂Cl₂ and washed with sat. NaHCO₃ (aq.), water, and brine. After drying over Na₂SO₄, the organic layer was concentrated in vacuo and dried under high vacuum to give PSMA2. A solution of PSMA2 in DCE (10 mL) was cooled to 0° C. and treated sequentially with MeOTf (0.59 g, 3.58 mmol) and TEA (1.00 mL, 7.16 mmol). After 45 min, Cbz-Lys t-butyl ester PSMA3 (1.34 g, 3.58 mmol) in DCE (2 mL) was added and the mixture was heated at 40° C. After 2 h, the reaction was diluted with CH₂Cl₂ and washed with sat. NaHCO₃ (aq.), water, and brine. The organic layer was dried over Na₂SO₄ and concentrated in vacuo to thick syrup. The crude material was purified through SiO₂ gel chromatography to afford PSMA4 (1.73 g, 78%) as a white foam.

AP-ESI+ Mass calcd C₃₂H₅₁N₃O₉: 621.36. Found: 622.4 [M+H]⁺, 644.4 [M+Na]⁺.

Preparation of Lys ureido Glu tris-t-butyl ester (PSMA5). A solution of PSMA4 (1.73 g, 2.79 mmol) in EtOAc (100 mL) was degassed under vacuum and purged with Argon. Palladium (10% wt on activated carbon, 0.15 g) was added in one portion and the mixture was degassed under vacuum purged with H₂ (g) and stirred for 6 h. The catalyst was removed by filtration and the mother liquor concentrated in vacuo to give PSMA5 quantitatively. AP-ESI+ Mass calcd C₂₄H₄₅N₃O₇: 487.32. Found: 488.4 [M+H]⁺.

### Synthesis of Monovalent PSMA Azide (PSMA7)

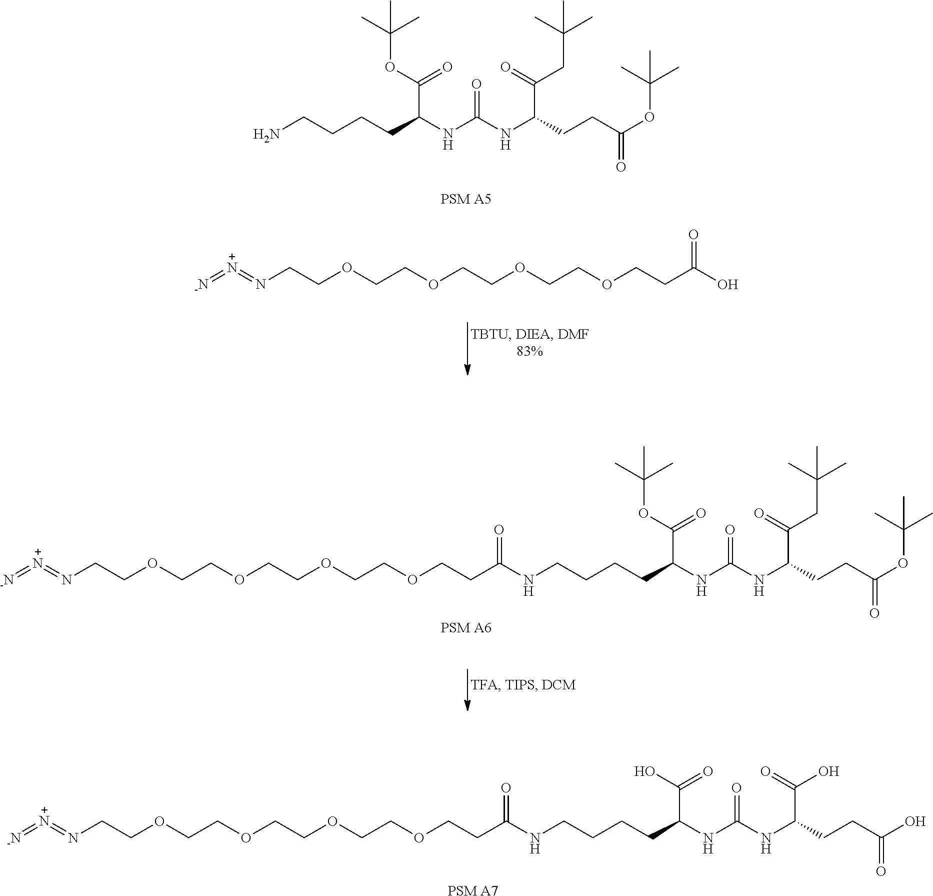

Preparation of azido Peg4 Lys ureido Glu tris-t-butyl ester (PSMA6). Azido Peg4 acid (133 mg, 0.45 mmol) was activated with TBTU (146 mg, 0.45 mmol), HOBt (69 mg, 0.45 mmol), and DIEA (216 uL, 1.24 mmol) in DMF (3.0 mL). After 15 min, a solution of PSMA5 (202 mg, 0.41 mmol) was delivered and the reaction stirred at RT for 1.5 h. RP-HPLCMS showed formation of desired product. The reaction was concentrated in vacuo and purified through SiO₂ gel chromatography to afford PSMA6 (257 mg, 83%). AP-ESI+ Mass calcd C₃₅H₆₄N₆O₁₂: 760.46. Found: 761.5 [M+H]⁺, 783.5 [M+Na]⁺.

Preparation of azido Peg4 Lys ureido Glu (PSMA7). Tris-tert-butyl ester PSMA6 (257 mg, 0.34 mmol) was treated with a solution of TFA:TIPS (10 mL, 97.5:2.5, v/v) for 30 min. RP-HPLCMS showed complete conversion to the desired product. The reaction was concentrated in vacuo and purified by RP-HPLC to afford PSMA7 (112 mg, 56%). AP-ESI+ Mass calcd C₂₃H₄₀N₆O₁₂: 592.27. Found: 593.3 [M+H]⁺.

### Synthesis of Monovalent PSMA HyNic (PSMA10)

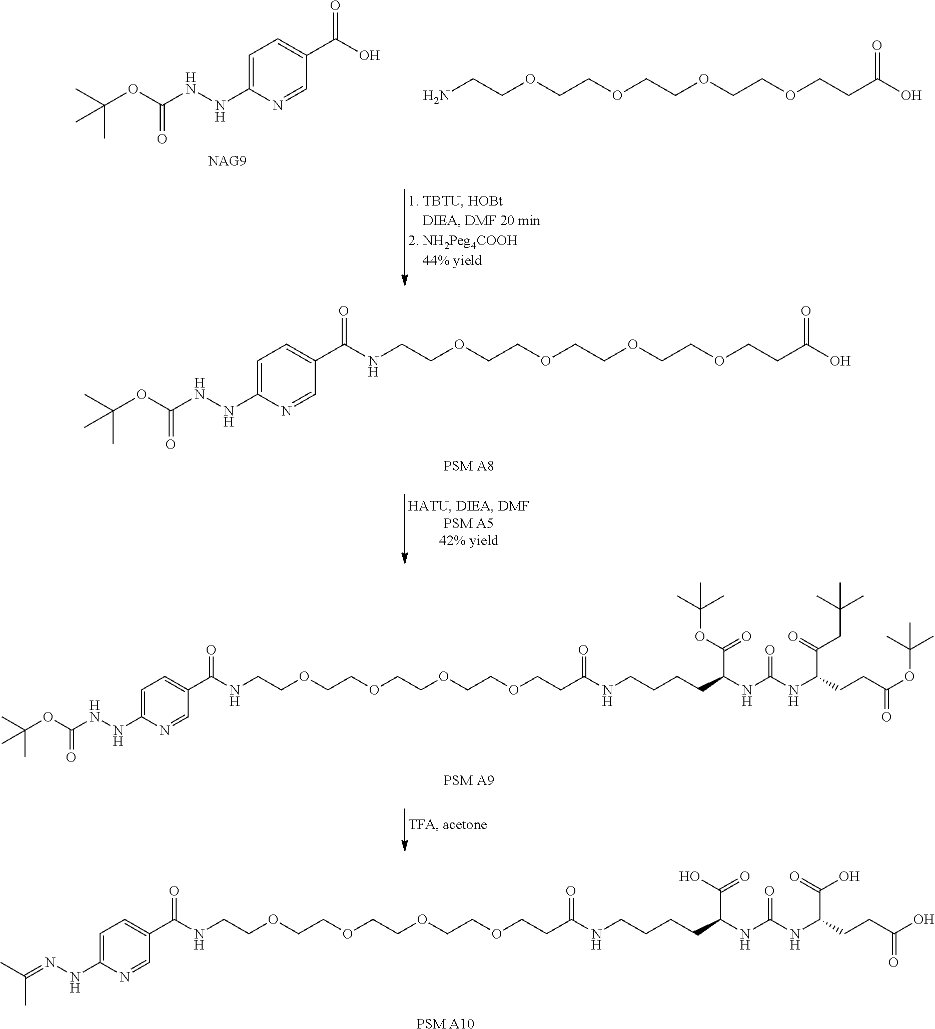

Preparation of N-Boc 4-hydrazino-nicotinamido Peg4 acid (PSMA8). N-Boc 4-hydrazino nicotinic acid NAG9 (137 mg, 0.54 mmol) was treated with TBTU (124 mg, 0.49 mmol), HOBt (83 mg, 0.54 mol), and DIEA (128 uL, 0.74 mmol) in DMF for 20 min. To the activated ester, was added a solution of amino-Peg4-acid (130 mg, 0.49 mmol) and the mixture was stirred for 2 h. The reaction was concentrated in vacuo and purified through SiO₂ gel chromatography to afford PSMA8 (107 mg, 44%). AP-ESI+ Mass calcd C₂₂H₃₆N₄O₉: 500.25. Found: 501.3 [M+H]⁺.

Preparation of N-Boc 4-hydrazino-nicotinamido Peg4-epsilon-amido lys-alpha-ureido-glu tri-t-butyl ester (PSMA9). PSMA8 (107 mg, 0.21 mmol) was treated with HATU (81 mg, 0.21 mmol) and DIEA (93 uL, 0.53 mmol) in the presence of amine PSMA5 (104 mg, 0.21 mmol) in DMF for 1 h. After which, the reaction was concentrated in vacuo and purified through SiO₂ gel chromatography to afford PSMA9 (85 mg, 42%). AP-ESI+ Mass calcd C₄₆H₇₉N₇O₁₅: 969.46. Found: 760.6 [M+H]⁺.

Preparation of dimethyl 4-hydrazono nicotinamido Peg4-epsilon-amido lys-alpha-ureido-glu (PSMA10). Tris-t-butyl ester PSMA9 (85 mg, 0.09 mmol) was treated with a solution of TFA:acetone (10 mL, 97.5:2.5, v/v) for 30 min. RP-HPLCMS showed complete conversion to the desired product. The reaction was concentrated in vacuo and purified by RP-HPLC to afford PSMA10 (55 mg, 84%). AP-ESI+Mass calcd C₃₂H₅₁N₇O₁₃: 741.35. Found: 742.4 [M+H]⁺.

### Synthesis of Bivalent PSMA Azide (PSMA18)

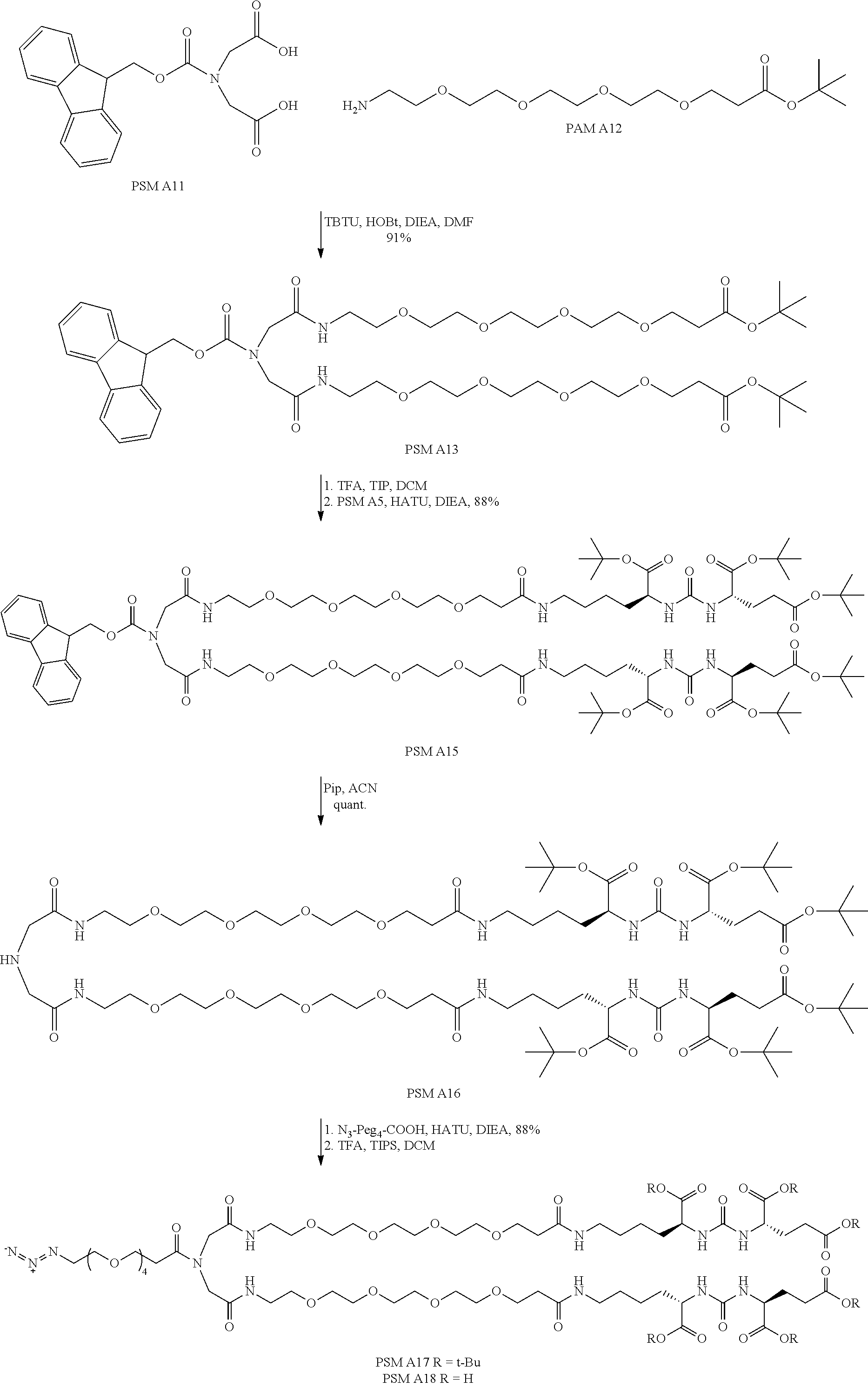

Preparation of N-Fmoc bis-imino-(acetamido-Peg4 t-butyl ester) (PSMA13). N-Fmoc imino diacetic acid, PSMA11, (107 mg, 0.30 mmol) was treated with PSMA12 (212 mg, 0.66 mmol), TBTU (193 mg, 0.60 mmol), HOBt (92 mg, 0.60 mmol), and DIEA (209 uL, 1.20 mmol) in DMF for 2 h. The reaction was concentrated in vacuo and purified through SiO₂ gel chromatography to afford PSMA13 (250 mg, 91%). AP-ESI+ Mass calcd C₄₉H₇₅N₃O₁₆: 961.51. Found: 962.6 [M+H]⁺, 984.6 [M+Na]⁺.

Preparation of N-Fmoc bis-imino-(acetamido-Peg4-epsilon-amido lys-alpha-ureido-glu tri-t-butyl ester) (PSMA15). Di-t-butyl ester PMSA13 (250 mg, 0.26 mmol) in DCM (1 mL) was treated with TFA (10 mL) and TIPS (111 uL, 0.54 mmol). After 30 min, the reaction was concentrated in vacuo to afford a syrup, which was washed with hexanes to afford di-acid PSMA14 as a thick syrup. PSMA14 was treated with HATU (198 mg, 0.54 mmol), PSMA5 (292 mg, 0.57 mmol), and DIEA (362 uL, 2.08 mmol) in DMF for 1 h. The reaction was concentrated in vacuo and purified through SiO₂ gel chromatography to afford PSMA15 (408 mg, 88%). PSMA14: AP-ESI+ Mass calcd C₄₁H₅₉N₃O₁₆: 849.39. Found: 850.5 [M+H]⁺, 872.5 [M+Na]⁺. PSMA15: AP-ESI+ Mass calcd C₈₉H₁₄₅N₉O₂₈: 1788.02. Found: 895.3 [M+2H]²+, 917.2 [M+2Na]²⁺.

Preparation of bis-imino-(acetamido-Peg4-epsilon-amido lys-alpha-ureido-glu tri-t-butyl ester) (PSMA16). N-Fmoc PMSA15 (408 mg, 0.22 mmol) in ACN (10 mL) was treated with piperidine for 30 min. The reaction was concentrated in vacuo, azeotroped with PhMe (3×10 mL), washed with hexanes (3×20 mL), and dried under high vacuum to afford PSMA16. AP-ESI+ Mass calcd C₇₄H₁₃₅N₉O₂₆: 1565.95. Found: 895.3 [M+2H]²+, 917.2 [M+2Na]²⁺.

Preparation of azido-Peg4-imido-bis-(acetamido-Peg4-epsilon-amido lys-alpha-ureido-glu tri-t-butyl ester) (PSMA17). Amine PMSA16 (172 mg, 0.11 mmol) was added to N₃-Peg4-COOH (40 mg, 0.14 mmol) activated with HATU (52 mg, 0.14 mmol) and DIEA (116 uL, 0.66 mmol) in DMF (2 mL). After 1 h, the reaction was concentrated in vacuo and purified by SiO₂ gel chromatography to afford PSMA17 (194 mg, 91%). AP-ESI+ Mass calcd C₈₅H₁₅₄N₁₂O₃₁: 1839.08. Found: 895.3 [M+2H]²+, 917.2 [M+2Na]²⁺.

Preparation of azido-Peg4-imido-bis-(acetamido-Peg4-epsilon-amido lys-alpha-ureido-glu) (PSMA18). Hexa-t-butyl ester PSMA17 (194 mg, 0.10 mmol) was treated with a solution of TFA:acetone (10 mL, 97.5:2.5, v/v) for 30 min. RP-HPLCMS showed complete conversion to the desired product. The reaction was concentrated in vacuo and purified by RP-HPLC to afford PSMA18 (69.4 mg, 44%). AP-ESI+Mass calcd C₆₁H₁₀₆N₁₂O₃₁: 1502.70. Found: 752.5 [M+2H]²⁺.

### Synthesis of Bivalent PSMA HyNic (PSMA20)

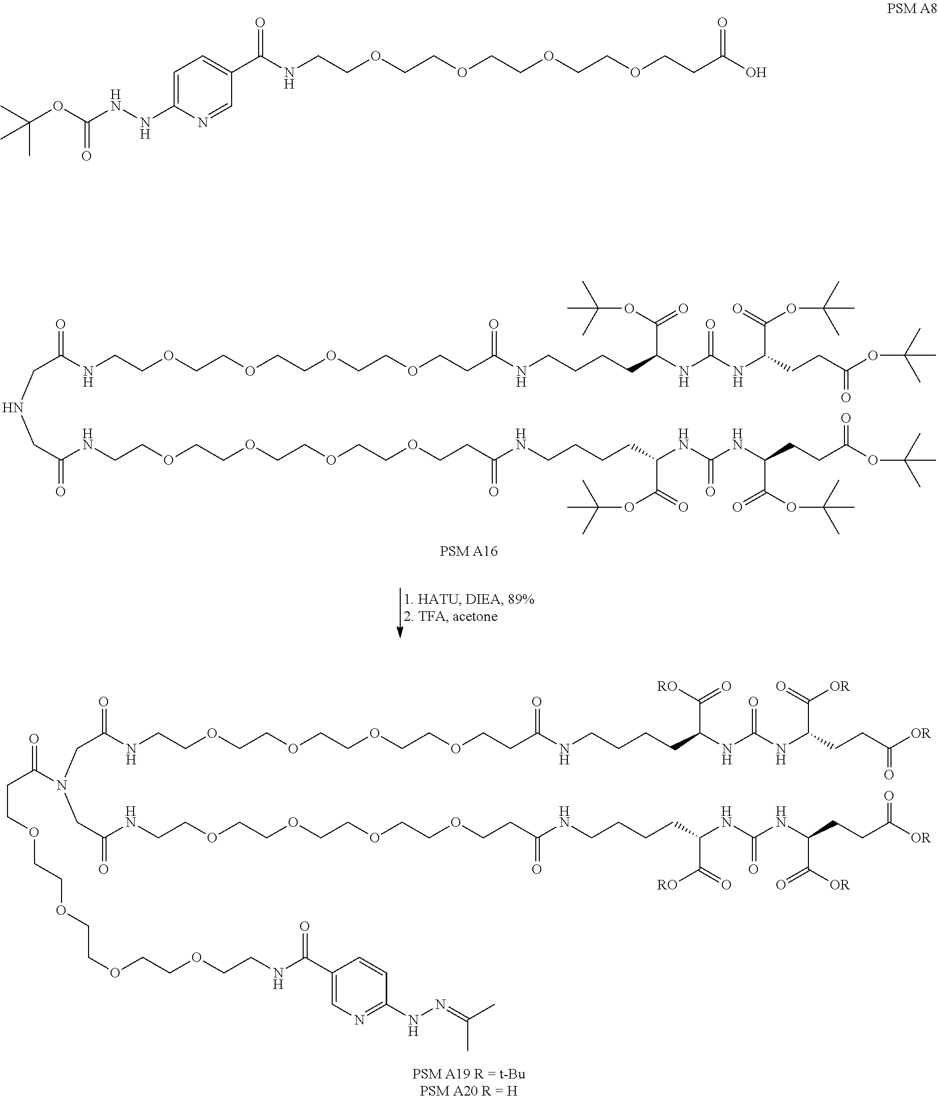

Preparation of N-Boc 4-hydrazino-nicotinamido Peg4-imido-bis-(acetamido-Peg4-epsilon-amido lys-alpha-ureido-glu tri-t-butyl ester) (PSMA19). Amine PMSA16 (172 mg, 0.11 mmol) was added to PSMA8 (61 mg, 0.12 mmol) activated with HATU (46 mg, 0.12 mmol) and DIEA (116 uL, 0.66 mmol) in DMF (2 mL). After 1 h, the reaction was concentrated in vacuo and purified by SiO₂ gel chromatography to afford PSMA19 (201 mg, 89%). AP-ESI+ Mass calcd C₉₆H₁₆₉N₁₃O₃₄: 2048.19. Found: 1025.3 [M+2H]²⁺, 684.0 [M+3H]³⁺.

Preparation of dimethyl 4-hydrazono-nicotinamido-Peg4-imido-bis-(acetamido-Peg4-epsilon-amido lys-alpha-ureido-glu) (PSMA20). Hexa-t-butyl ester PSMA19 (201 mg, 0.10 mmol) was treated with a solution of TFA:acetone (10 mL, 9:1, v/v) for 60 min. RP-HPLCMS showed complete conversion to the desired product. The reaction was concentrated in vacuo and purified by RP-HPLC to afford PSMA20 (69.4 mg, 44%). AP-ESI+ Mass calcd C₇₀H₁₁₇N₁₃O₃₂: 1651.79. Found: 827.1 [M+2H]²⁺.

### Synthesis of Mannose Ligand:

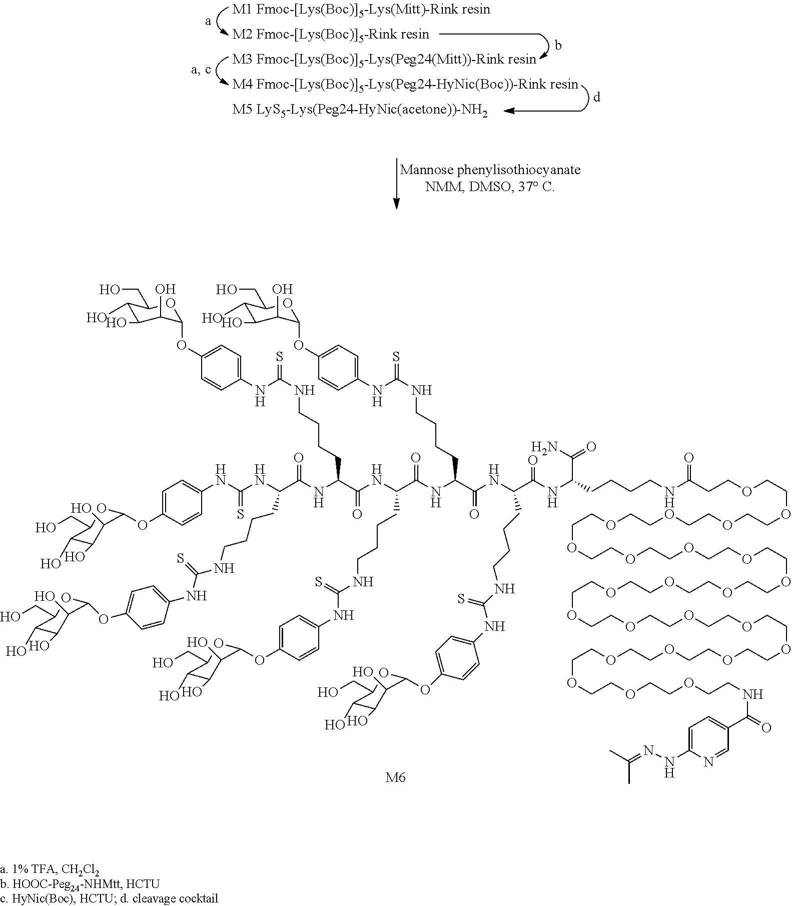 [SMILES: CC(C)=NNc1ccc(C(=O)NCCOCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@H](CCCCNC(=S)Nc2ccc(OC3OC(CO)C(O)C(O)C3O)cc2)NC(=O)[C@H](CCCCNC(=S)Nc2ccc(OC3OC(CO)C(O)C(O)C3O)cc2)NC(=O)[C@H](CCCCNC(=S)Nc2ccc(OC3OC(CO)C(O)C(O)C3O)cc2)NC(=O)[C@H](CCCCNC(=S)Nc2ccc(OC3OC(CO)C(O)C(O)C3O)cc2)NC(=O)[C@H](CCCCNC(=S)Nc2ccc(OC3OC(CO)C(O)C(O)C3O)cc2)NC(=S)Nc2ccc(OC3OC(CO)C(O)C(O)C3O)cc2)C(N)=O)cn1 ; "M6"]

Preparation of Lys6-Peg24-HyNic (M5). Peptide scaffold was synthesized using standard Fmoc chemistry on a Rink amide resin (0.61 mmol/g) with HCTU coupling and 20% piperidine deprotection. In short, peptide M1 was prepared on an automated synthesizer on a 25 umol scale. After deprotection of Lys(Mtt), Peg24 amino(Mtt) acid was coupled to provide M3. Removal of the Mtt group and subsequent coupling of BocHyNic provided M4. Release of the peptide from the resin using trifluoroacetic acid:triisopropylsilane:water:acetone:dithithreitol (90:2:2:3:3) and purification by RP-HPLC afforded M5 (7.0 mg). AP-ESI+ Mass calcd C₉₆H₁₈₅N₁₇O₃₂: 2088.33. Found: 1046 m/2z, 698 m/3z, 524 m/4z.

Preparation of Man6-Lys6-Peg24-HyNic (M6). Peptide scaffold M5 (7.0 mg) in DMSO (1 mL) was treated with mannose isothiocyanate (8.0 mg) and N-methylmorpholine (NMM; 200 μL). The reaction was stirred for 4 h at 37° C. and purified by RP-HPLC to afford M6 (1.2 mg). MALDI-TOF mass calcd C₁₇₄H₂₇₅N₂₃O₆₈S₆: 3966.70. Found: 3987.39 [M+Na].

### Synthesis of Hexavalent Mannose Azide (M9)

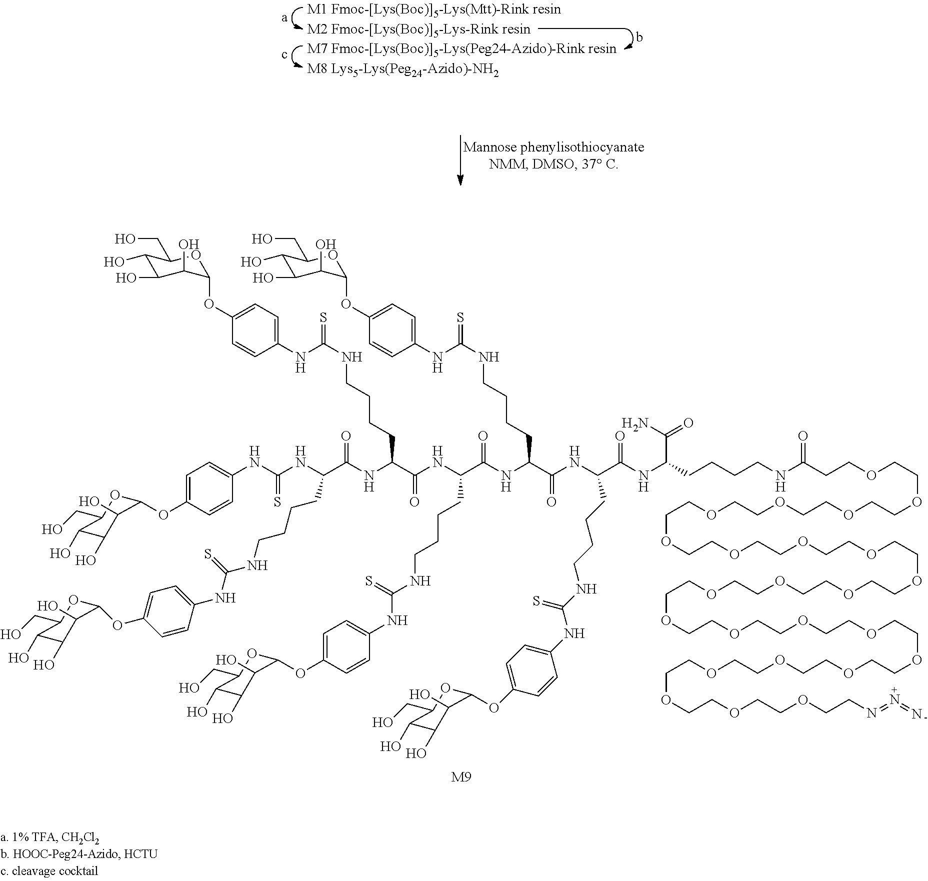

Preparation of Lys6-Peg24-Azide (M8). Peptide scaffold was synthesized using standard Fmoc chemistry on a Rink amide resin (0.61 mmol/g) with HCTU coupling and 20% piperidine deprotection. In short, peptide M1 was prepared on an automated synthesizer on a 100 umol scale. After deprotection of Lys(Mtt), Azido Peg24 acid was coupled to provide M7. Release of the peptide from the resin using the cocktail TFA:TIPS:H₂O (92.5:2.5:5) afforded M8 (167.0 mg). MALDI TOF Mass calcd C₈₇H₁₇₄N₁₆O₃₁: 1940.4. Found: 1941.1.

Preparation of Man6-Lys6-Peg24-Azide (M9). Peptide scaffold M4 (167.0 mg) in DMSO (2 mL) was treated with mannose isothiocyanate and NMM (500 uL). The reaction was stirred at 37° C. and monitored by MALDI TOF until full conversion to the desired product was achieved (a total of 58 mgs of mannose isothyocyanate was added). The final product was purified by RP-HPLC to afford M9 (22 mg). MALDI-TOF mass calcd C₁₆₅H₂₆₄N₂₂O₆₇S₆: 3820.37. Found: 3843.79 [M+Na].

### Synthesis of Trivalent Mannose Azide (M15)

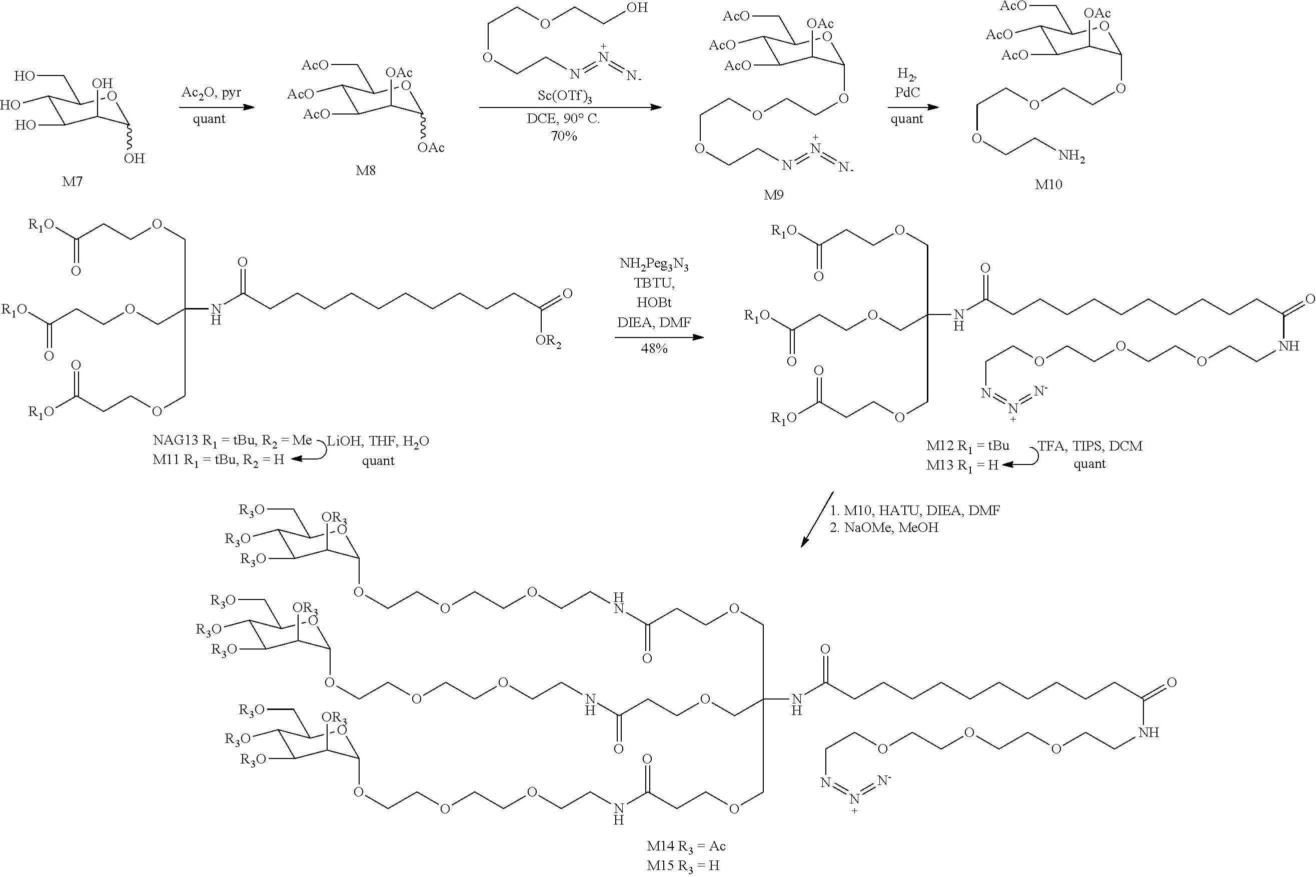

Preparation of azido tri-mannose (M15):D-Mannose was peracetylated by Ac₂O in pyridine overnight. Concentration by rotary evaporation followed by azeotroping with PhMe provided the pentaacetate (M8) in quantitative yield. Activation of M8 with Sc(OTf)₃ in the presence of commercially available azido Peg2 alcohol afforded azido-Peg2 mannoside (M9), which was hydrogenated quantitatively to amine (M10). In the meanwhile, the methyl ester of tris linker (NAG13) was hydrolyzed to selectively to acid (M11). Coupling of commercially available azido Peg3 amine to M11 by TBTU activation provided azido tris linker (M12). Treatment of tri t-butyl ester M12 with TFA gave tri-acid M13. Coupling of M10 to M13 was mediated by HATU and the crude mixture was globally de-acetylated to afford azido tri-mannose (M15).

### Synthesis of Monovalent Mannose Phosphoramidite (M21)

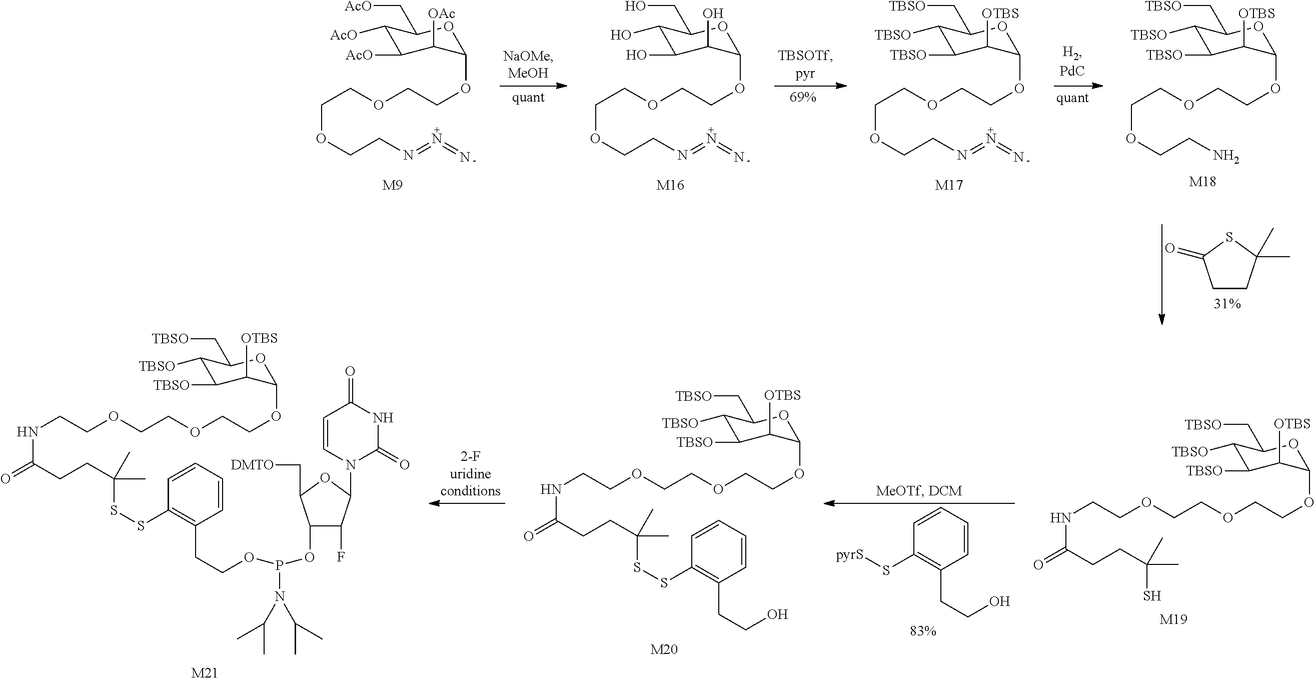

Preparation of mannose disulfide 2-fluoro uridine phosphoramidite (M21): Through standard protection/deprotection chemistry, the acetates of M9 were converted to t-butyl silyl (TBS) M17 through deacetylated intermediate M16. Reduction of azide M17 to amine M18 by hydrogenation facilitated N-acylation with the hindered thiolactone to afford thiol M19. Disulfide M20 was cleanly formed through addition of aryl mercapto-thiopyridine, pre-activated with MeOTf. Phosphoramidite M21 was to be formed in a standard 2-step one-pot manner by treatment of 2-fluoro uridine with bis(diisopropylamino) chlorophosphine followed by addition of sugar disulfide M20.

### Synthesis of Hexavalent Mannose Azide (M30)

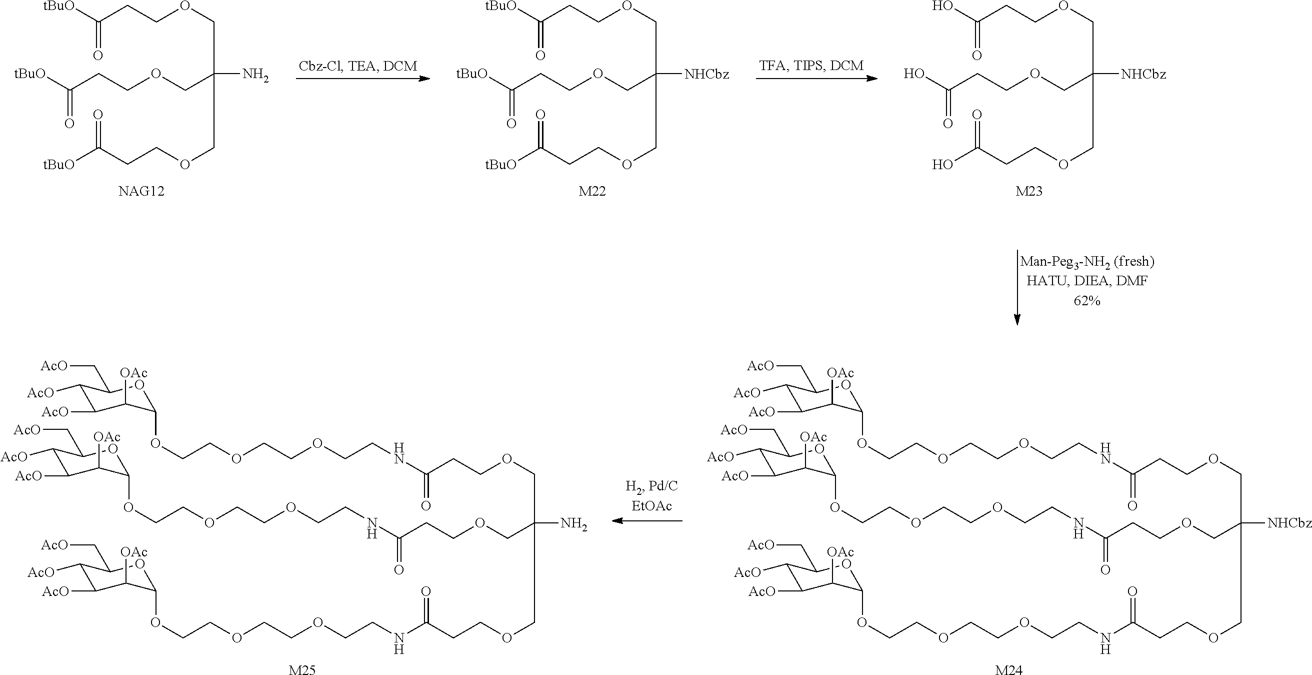

Preparation of N-carbobenzyloxy tris-(t-butoxycarboethoxymethyl)-methylamide (M22): To a solution of NAG12 (3.55 g, 7.02 mmol) in CH₂Cl₂ (12 mL) cooled in an ice bath was added Cbz-CL (35% in PhMe, 7.3 mL) and TEA (3.9 mL). The reaction was warmed to rt and stirred overnight. The mixture was diluted with CH₂Cl₂ and washed with saturated NaHCO₃ (aq), dried over Na₂SO₄, concentrated in vacuo. The crude oil purified by SiO₂ chromatography to afford M22 (0.98 g, 22% yield).

AP-ESI+ Mass calcd C₃₃H₅₃NO₁₁: 639.4. Found: 662.4 [M+Na]⁺.

Preparation of N-carbobenzyloxy tris-((2,3,4,6-tetra-O-acetyl-1-O-α-D-mannopyranosyl)-Peg3-amidoethoxymethyl)-methylamide (M24): Tris-t-butyl ester M22 (0.97 g, 1.51 mmol) and TIPS (0.93 mL, 4.55 mmol) in CH₂Cl₂ (5 mL) was treated with TFA (20 mL) for 5 h. The mixture was concentrated in vacuo, the oily residue was washed with hexanes and dried under high vacuum to provide M23.

AP-ESI+ Mass calcd O₂₁H₂₉NO₁₁: 471.2. Found: 493.9 [M+Na]⁺.

Crude M23 in DMF (5 mL) was cooled on an ice bath and treated with HATU (0.62 g, 1.63) and DIEA (0.65 mL, 3.71 mmol). After stirring for 20 min, a solution of M10 (0.89 g, 1.86 mmol) in DMF (5 mL) was added and the mixture was warmed to rt and stirred for 3 h. The solvent was removed in vacuo and the crude was dissolved in EtOAc and washed with saturated NaHCO₃ (aq), dried over Na₂SO₄, concentrated in vacuo. Purification by SiO₂ chromatography afforded M24 (0.49 g, 62% yield). MALDI-TOF Mass calcd O₈₁H₁₂₂N₄O₄₄: 1854.74. Found: 1850.14.

Preparation of tris-((2,3,4,6-tetra-O-acetyl-1-O-α-D-mannopyranosyl)-Peg3-amidoethoxymethyl)-methylamine (M25): A solution of M24 (0.49 g, 0.26 mmol) was dissolved in EtOAc (50 mL) with HOAc (0.2 mL) was degassed under vacuum and purged with Ar (g). Pd on activated carbon (0.16 g) was added and the mixture was evacuated and then purged with H₂ (g) thrice. Reaction was stirred for 2 days, catalyst removed by filtration, and mother liquor concentrated in vacuo to afford M25.

AP-ESI+ Mass calcd C₇₃H₁₁₆N₄O₄₂: 1720.7. Found: 1723.42.

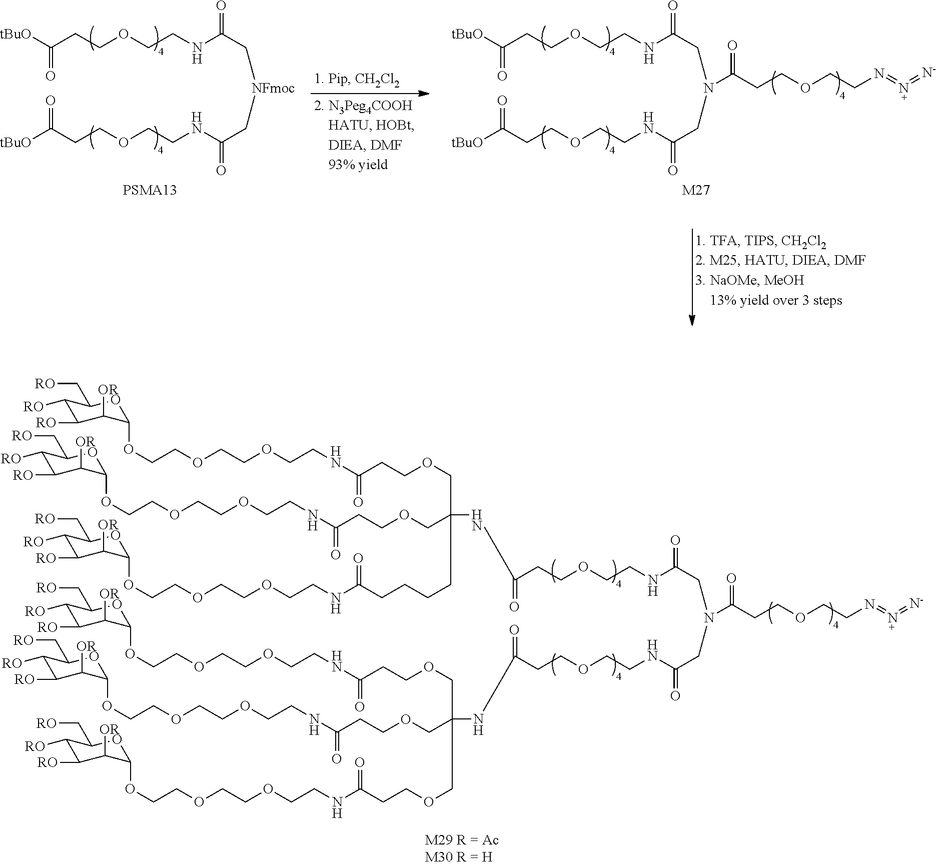

Preparation of azido-Peg4-imido-bis-(acetamido-Peg4-t-butyl ester) (M27): N-Fmoc PSMA13 (0.72 g, 0.75 mmol) in CH₂Cl₂ was treated with piperidine (0.75 mL) for 1 h. HPLCMS showed complete conversion to M26, AP-ESI+ Mass calcd C₃₄H₆₅N₃O₁₄: 739.4. Found: 740.5 [M+H]⁺.

The mixture was concentrated in vacuo and azeotroped with PhMe. Crude M26 was reacted with solution of azido Peg4 acid (0.44 g, 1.51 mmol), HATU (0.57 g, 1.51 mmol), and DIEA (0.52 mL) in DMF (5 mL) for 1 h. After solvent removal in vacuo, the crude was dissolved in EtOAc, washed with sat NaHCO₃ (aq.), dried over Na₂SO₄, and concentrated in vacuo. Purification by SiO2 chromatography afforded M27 (0.71 g, 93% yield, 2 steps).

AP-ESI+ Mass calcd C₄₅H₈₄N₆O₁₉: 1012.6. Found: 1013.6 [M+H]⁺.

Preparation of azido-Peg4-imido-bis-(trimer mannose) (M30): Imido linker M27 (0.69 g, 0.68 mmol) was treated with TIPS (0.28 mL, 1.36 mmol) and TFA (10 mL) to afford tri acid M28; AP-ESI+Mass calcd C₃₇H₆₈N₆O₁₉: 900.5. Found: 900.9 [M+H]⁺, 922.9 [M+Na]⁺. Volatiles were removed in vacuo and M28 dried under high vacuum. Di-acid M28 (82.0 mg, 0.09 mmol) was activated with HATU (75 mg, 0.2 mmol) and DIEA (0.28 mL) in DMF (2 mL) at 0° C. After 30 min, a solution of M25 (0.26 mmol) in DMF (2 mL) was added and the mixture was warmed to rt and stirred for 2 h. RP-HPLCMS showed complete conversion to M29; Mass calcd C₁₈₃H₂₉₆N₁₄O₁₀₁: 4305.84. MALDI-TOF Found: 4303.36 AP-ESI+ Found: 1436.1 [M+3H]³⁺, 1077.3 [M+4H]⁴⁺. The reaction was diluted with CH₂Cl₂ washed with sat NaHCO₃ (aq.), dried over Na₂SO₄, and concentrated in vacuo. The crude M29 oil (538 mg) dissolved in MeOH (20 mL) was treated with NaOMe (25 wt % in MeOH, 0.5 mL) for 1 h. RP-HPLCMS showed complete conversion to M30. The reaction was quenched by addition of Dowex H+ resin to neutralize. The crude material was purified by HPLC to afford M30 (38.1 mg, 13% yield over 3 steps). Mass calcd C₁₃₅H₂₄₈N₁₄O₇₇: 3297.59, MALDI-TOF Found: 3318.61 [M+Na]⁺ AP-ESI+ Found: 1100.0 [M+3H]³⁺, 825.3 [M+4H]⁴⁺.

### Synthesis of ABL Ligands

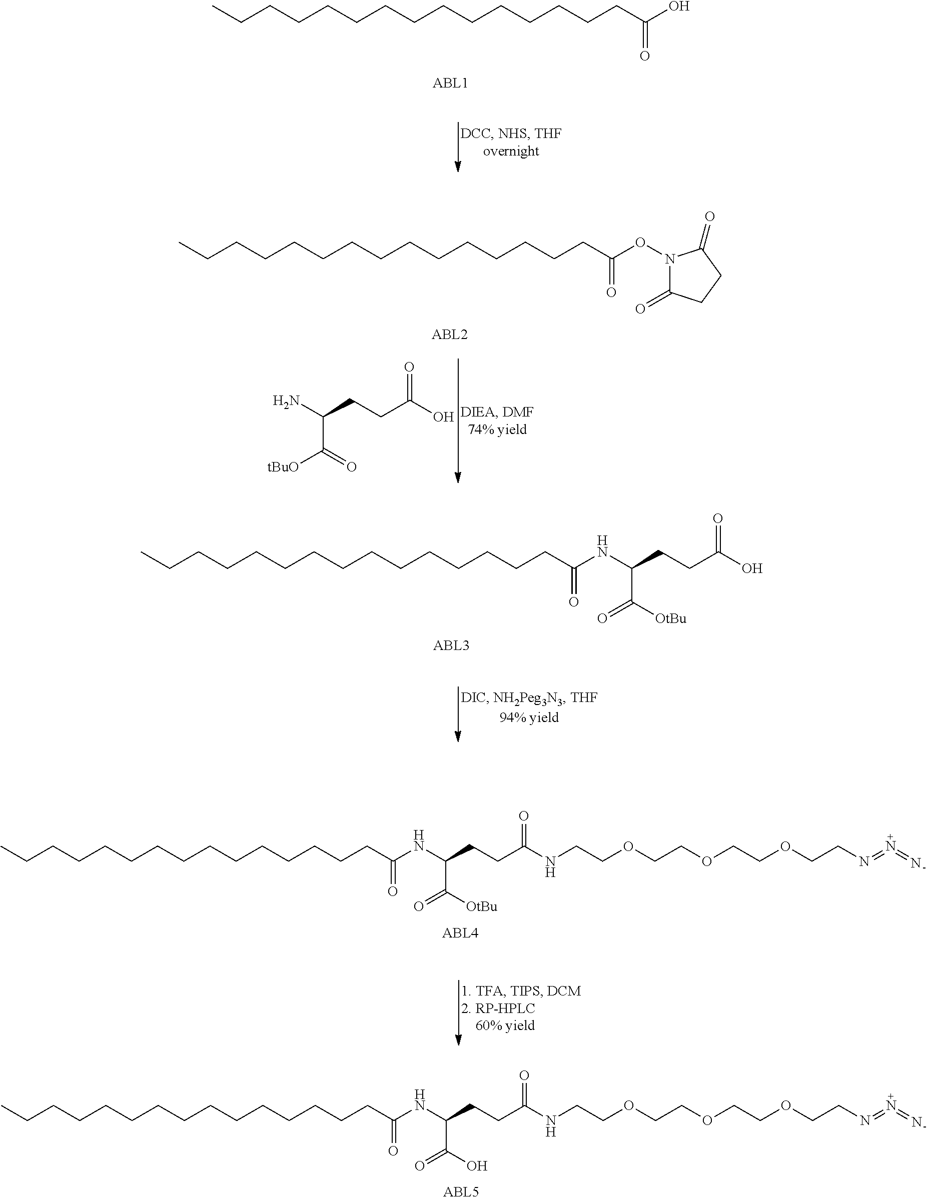

Preparation of N-palmitoyl L-glutamic acid α-t-butoxy ester (ABL3): Palmitic acid ABL1 (1.0 g, 3.8 mmol) in THF (10 mL) was treated with N-hydroxy succinimide (0.9 g, 7.6 mmol) and diisopropylcarbodiimide (1.2 mL, 7.6 mmol) overnight to afford ester (ABL2). The ppt was removed by filtration and THF evaporated in vacuo. The resulting residue was dissolved in DMF (6 mL) and treated with glutamic acid α-t-butyl ester (0.7 g, 3.4 mmol) and DIEA (1.8 mL, 10 mmol). After 2 h, the reaction was diluted with water and the desired product was extracted with Et₂O. The ether layer was dried over Na₂SO₄, concentrated in vacuo, and the crude mass purified through SiO₂ chromatography to afford an off-white solid ABL3 (1.2 g, 74% yield).

AP-ESI+ Mass calcd C₂₅H₄₇NO₅: 441.3. Found: 464.0 [M+Na]⁺.

Preparation of N-palmitoyl 6-(amido Peg3 azide) L-glutamic acid α-t-butoxy ester (ABL4): To a solution of ABL3 (1.24 g, 2.8 mmol) in THF (10 mL) was added 11-azido-Peg3 amine (0.92 g, 4.2 mmol) and diisopropylcarbodiimide (0.87 mL, 5.6 mmol). After stirring overnight, the ppt was removed by filtration, mother liquor concentrated in vacuo, and the crude mass purified through SiO₂ chromatography to afford an off-white solid ABL4 (1.7 g, 94% yield).

AP-ESI+ Mass calcd C₃₃H₆₃N₅O₇: 641.5. Found: 642.4 [M+H]⁺.

Preparation of N-palmitoyl 6-(amido Peg3 azide) L-glutamic acid (ABL5): A solution of t-butyl ester ABL4 (1.71 g, 2.66 mmol) and TIPS (0.54 mL, 2.66 mmol) in DCM (2 mL) was treated with TFA (10 mL). After 1.5 h, the mixture was concentrated in vacuo. The oily crude was washed with hexanes, dried in vacuo, and purified by RP-HPLC to afford ABL5 (930 mg, 60% yield).

AP-ESI+ Mass calcd C₂₉H₅₅N₅O₇: 585.4. Found: 586.0 [M+H]⁺.

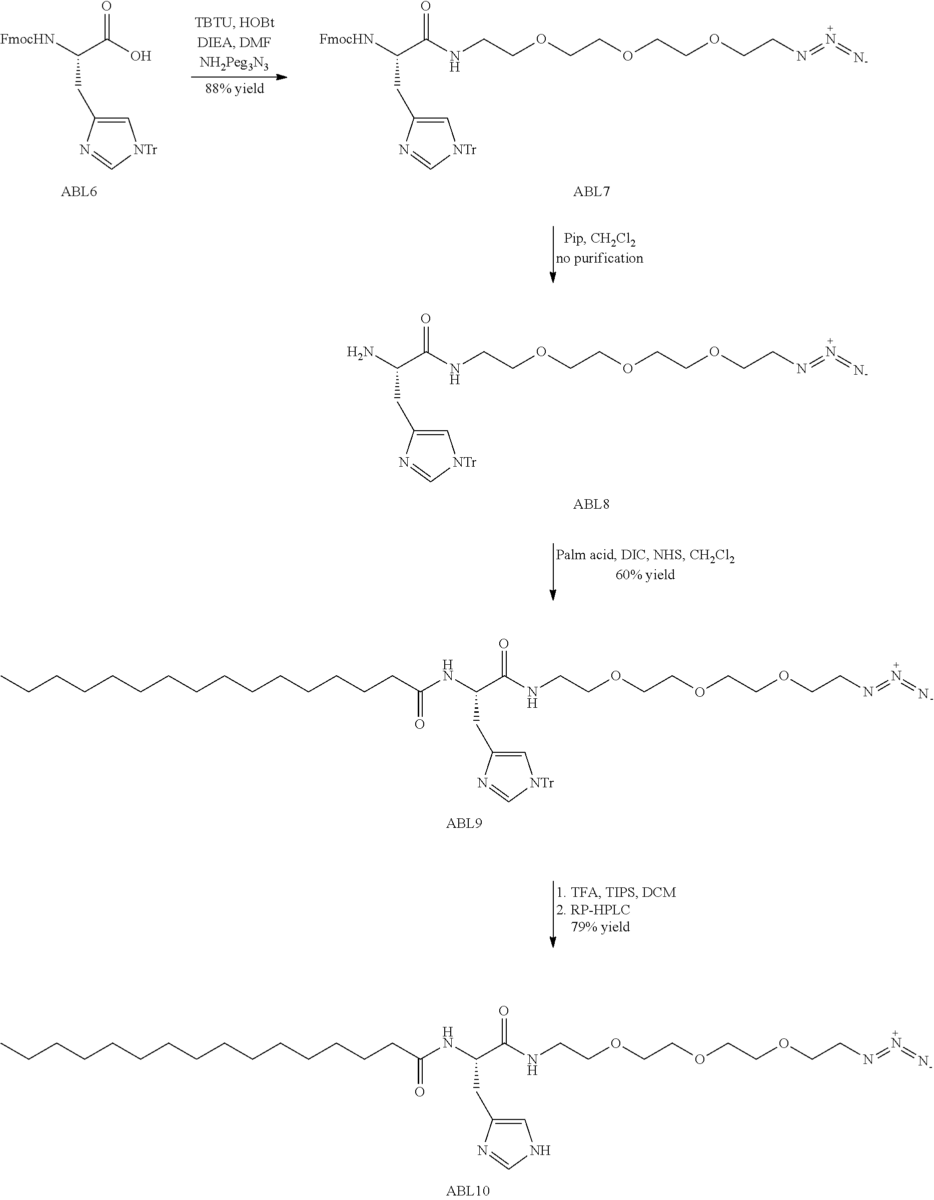

Preparation of N-α-Fmoc N-imidazyl-trityl α-(amido Peg3 azide) L-histidine (ABL7): N-α-Fmoc N-imidazyl-trityl L-histidine (1.00 g, 1.61 mmol) in DMF (5 mL) was activated with TBTU (0.57 g, 1.77 mmol), HOBt (0.27 g, 1.77 mmol), and DIEA (0.84 mL, 4.84 mmol) for 20 min. A solution of 11-azido-Peg3 amine (0.35 g, 1.61 mmol) in DMF (1.0 mL) was added, and the mixture stirred for 3 h. The reaction was diluted with H₂O and extracted into Et₂O. The ether layer was dried over Na₂SO₄, concentrated in vacuo, and the crude mass purified through SiO₂ chromatography to afford a pale yellow solid ABL7 (1.17 g, 88% yield).

AP-ESI+ Mass calcd C₄₈H₄₉N₇O₆: 819.4. Found: 819.8 [M+H]⁺.

Preparation of N-α-palmitoyl N-imidazyl-trityl α-(amido Peg3 azide) L-histidine (ABL9): N-Fmoc ABL7 (1.17 g, 1.42 mmol) in CH₂Cl₂ (5 mL) was treated with piperidine (0.56 mL) and stirred for 1 h to cleanly provide ABL8; AP-ESI+ Mass calcd C₃₃H₃₉N₇O₄: 597.3. Found: 597.9 [M+H]⁺. The mixture was concentrated in vacuo and the residue was washed with hexanes. Crude ABL8 was dissolved in CH₂Cl₂ (5 mL) and treated with palmitic acid (0.73 g, 2.84 mmol), diisopropylcarbodiimide (0.36 g, 2.84 mmol), and NHS (0.43 g, 2.84 mmol). The precipitate was removed by filtration and the crude purified through SiO₂ chromatography to afford an off-white solid ABL9 (0.71 g, 60% yield).

AP-ESI+ Mass calcd C₄₉H₆₉N₇O₅: 835.5. Found: 835.9 [M+H]⁺.

Preparation of N-α-palmitoyl α-(amido Peg3 azide) L-histidine (ABL10): A solution of N-imidazyl-trityl ABL9 (0.71 g, 0.85 mmol) and TIPS (0.17 mL, 0.85 mmol) in DCM (2 mL) was treated with TFA (10 mL). After 1.5 h, the mixture was concentrated in vacuo. The oily crude was washed with hexanes, dried in vacuo, and purified by RP-HPLC to afford ABL10 (394 mg, 79% yield).

AP-ESI+ Mass calcd C₃₀H₅₅N₇O₅: 593.4. Found: 594.3 [M+H]⁺.

### Disulfide Phosphotriester Oligonucleotide Synthesis:

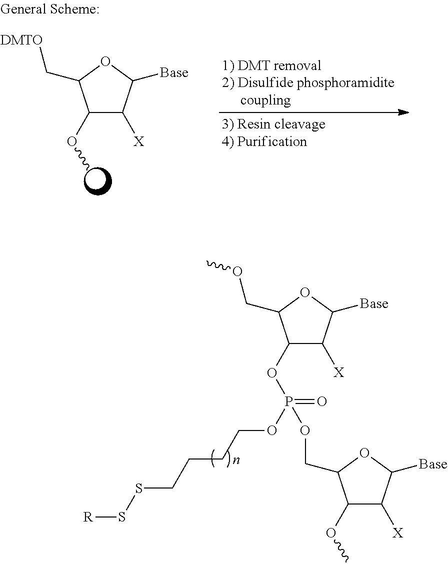

### Experimental Details:

All the oligonucleotide sequences synthesized were modified at 2′-ribose sugar position with 2′-F and 2′-OMe modifications to improve serum stability and to minimize off-target effects. Automated oligonucleotide synthesis (1 μmol scale) was carried out with the following reagents/solvents:

Oxidizer—0.02 M l₂ in THF/Pyridine/H₂O (60 s oxidation per cycle)

Deblock—3% Trichloroacetic Acid (2×40 s deblocks per cycle)

Cap Mix A—THF/Pyridine/Pac₂O (60 s capping per cycle)

Cap Mix B—16% Methyl imidazole in THF (60 s capping per cycle)

Exceptions to standard oligonucleotide synthesis conditions were as follows:
CPG supports with Q-linkers (hydroquinone-O,O′-diacetic acid linker arm) for milder deprotection were used
All disulfide phosphoramidites were resuspended to 100 mM in 100% anhydrous acetonitrile prior to synthesis
Phosphoramidite activation was performed with 2.5-fold molar excess of 5-Benzylthio-1-H-tetrazole (BTT). Activated phosphoramidites were coupled for 2×3 minute coupling steps per insertion.

### Disulfide Phosphotriester Oligonucleotide Deprotection & Purification Protocol:

Following automated oligonucleotide synthesis, disulfide phosphotriester oligonucleotides were cleaved and deprotected in 1 ml of 10% diisopropylamine in methanol (10% DIA/MeOH) for 4 h at room temperature. Following the 4 h deprotection, oligo samples were dried by centrifugal evaporation.
Crude oligo pellets were resuspended in 100 μl of 50% acetonitrile, briefly heated to 65° C. and vortexed thoroughly. Total 100 μl crude oligo samples were injected onto RP-HPLC with the following buffers/gradient:
   Buffer A=50 mM TEAA in Water
   Buffer B=90% Acetontrile
   Flow Rate=1 ml/min
      Gradient:
         0-2 min (100% Buffer A/0% Buffer B)
         2-42 min (0% to 60% Buffer B)
         42-55 min (60% to 100% Buffer B)
Across the dominant RP-HPLC peaks, 0.5 ml fractions were collected and analyzed by MALDI-TOF mass spectrometry to confirm presence of desired mass. Purified fractions containing correct mass were frozen and lyophilized. Once dry, fractions were resuspended, combined with corresponding fractions, frozen and lyophilized for final product.

Disulfide insertions requiring additional deprotection were initially isolated as described above followed by the necessary secondary deprotection steps (see below):

### Aldehyde-Disulfide Phosphotriester Secondary Deprotection:

RP-HPLC purified oligo products were resuspended in 100 μl of 80% formic acid. Reaction was allowed to proceed at room temperature for ˜1 h per aldehyde modification. Reaction was monitored by MALDI-TOF mass spectrometry to confirm complete deprotection. Once deprotection was complete, samples were frozen and lyophilized until dry. Lyophilized samples were then resuspended in 1 ml of 20% acetonitrile and gel filtered for isolation of final oligo product.

### Hydroxyl-Disulfide Phosphotriester Secondary Deprotection:

RP-HPLC purified oligo products were resuspended in 219 μl of anhydrous DMSO, heated briefly to 65° C. and vortexed thoroughly. To the DMSO solutions, 31 μl of 6.1 M triethylamine trihydrofluoride (TEA.3HF) was added for final concentration of 0.75 M. Reaction was allowed to proceed at room temperature for ˜1 h per TBDMS-protected hydroxyl modification. Reaction was monitored by MALDI-TOF mass spectrometry to confirm complete deprotection. Once deprotection was complete, 35 μl of 3 M sodium acetate followed by 1 ml of butanol was added. Samples were vortexed thoroughly then placed at −80° C. for 2 h. After 2 h, samples were centrifuged to pellet oligonucleotides. Butanol layer was removed and the oligo pellet was resuspended in 1 ml of 20% acetonitrile. Samples were gel filtered for isolation of final oligo product.

### Disulfide Phosphotriester Oligonucleotide Conjugation Through Condensation Reaction—General Protocol (See Conjugation General Schemes 1-3):

Disulfide phosphotriester duplexes were generated by equimolar mixing of desired passenger and guide strand oligos. Following the addition of sodium chloride to a final concentration of 50 mM, samples were heated to 65° C. for 5 minutes and allowed to cool to room temperature to complete annealing.
For aldehyde-modified disulfide phosphotriester oligos, siRNA duplexes were diluted into 1× conjugation buffer prior to the addition of the desired HyNic conjugation moiety.

### Conjugation Buffer: 10 mM HEPES (pH 5.5), 20 mM Aniline, 50 mM NaCl, 50% Acetonitrile

Once the above reaction was mixed, a two-fold molar excess of HyNic conjugation component was added to the mixture. Reaction was allowed to proceed at room temperature for 1 h.
After 1 h, conjugated siRNA oligonucleotides were isolated by either gel filtration, HPLC purification or centrifugal spin filtration for final products prior to cellular treatment.

### Disulfide Phosphotriester Oligonucleotide Conjugation Through Click Reaction—General Protocol (See Conjugation General Schemes 4 and 5):

### Copper-THPTA Complex Preparation:

A 5 mM aqueous solution of copper sulfate pentahydrate (CuSO₄-5H₂O) and a 10 mM aqueous solution of Tris(3-hydroxypropyltriazolylmethyl)amine (THPTA) were mixed 1:1 (v/v) (1:2 molar ratio) and allowed to stand at room temperature for 1 hour.

### Click Reaction (100 nM Scale)

To a solution of 710 uL of water and 100 uL tert-butanol (10% of final volume) in a 1.7 mL eppendorf tube was added 60 uL of the copper-THPTA complex followed by 50 uL of a 2 mM solution of the oligo, 60 ul of a 20 mM aqueous sodium ascorbate solution and 20 uL of a 10 mM solution of GalNAc-azide. After thorough mixing the solution was allowed to stand at room temperature for 1 hour. Completion of the reaction was confirmed by gel analysis.

The reaction mixture is added to a screw cap vial containing 5-10 fold molar excess of SiliaMetS®TAAcONa (resin bound EDTA sodium salt). The mixture is stirred for 1 hour. This mixture is then eluted through an Illustra™ Nap™-10 column Sephadex™. The solution is then frozen and lyophilized overnight.

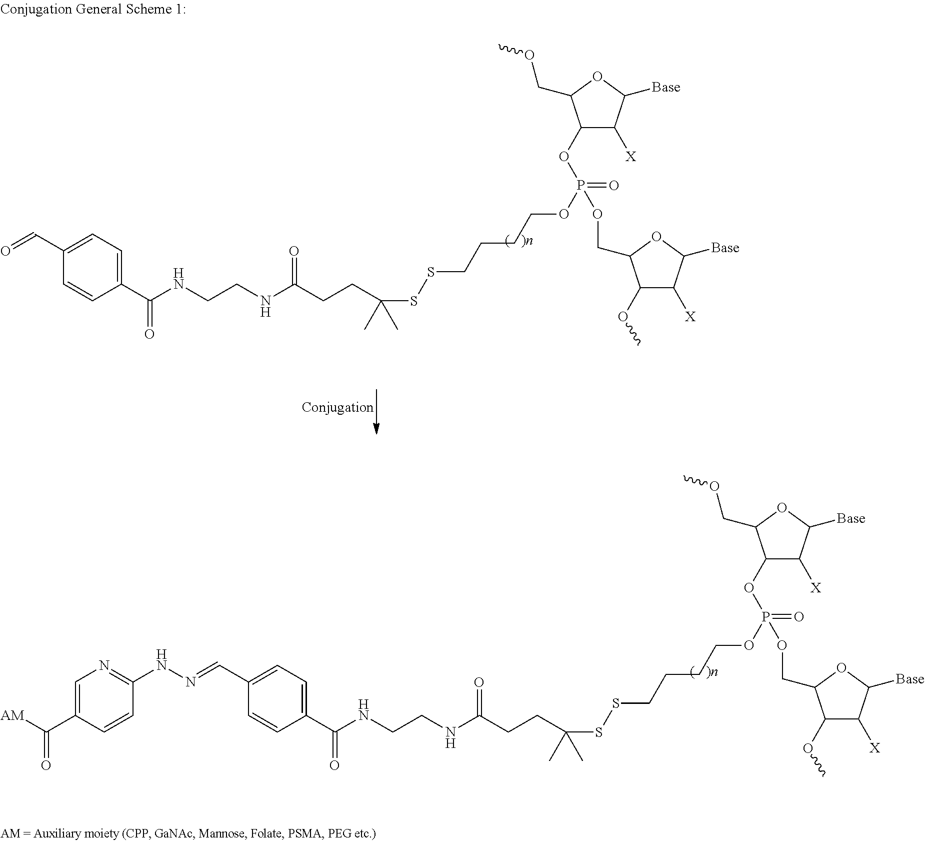

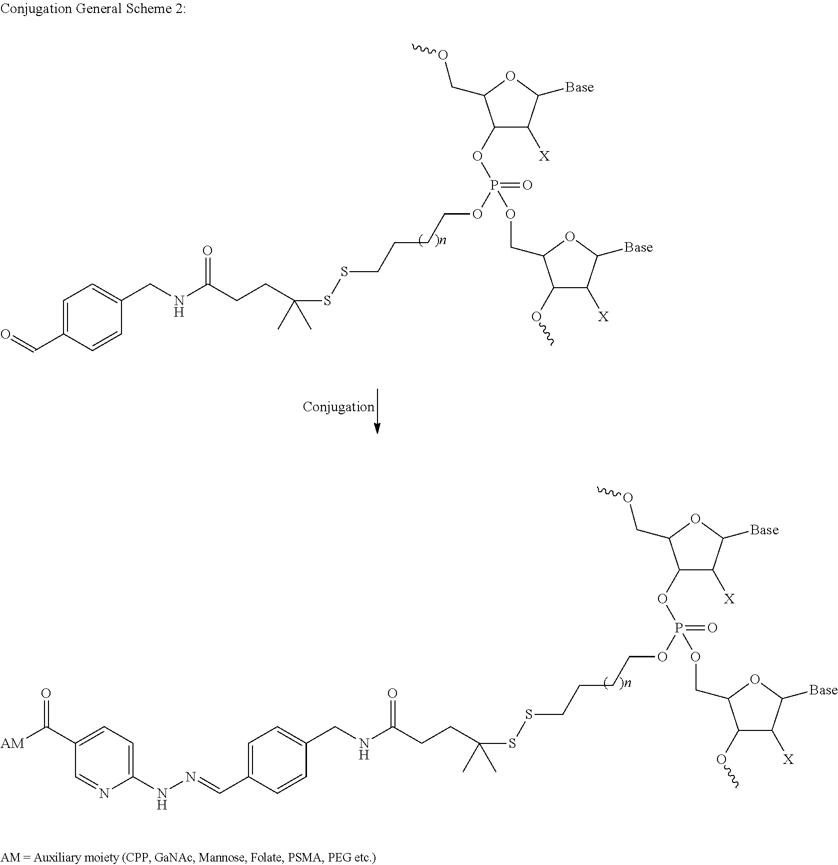

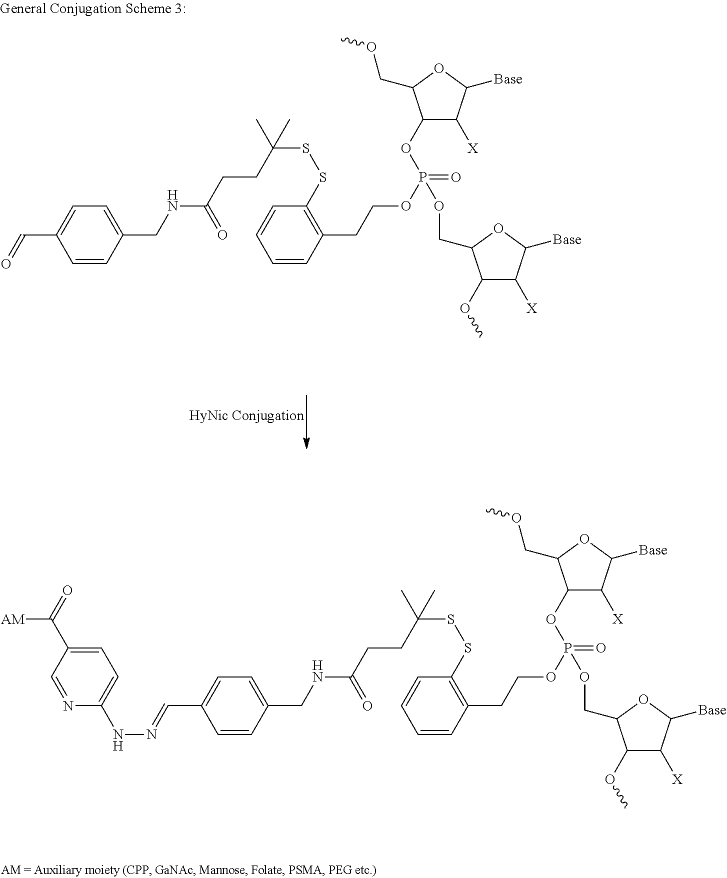

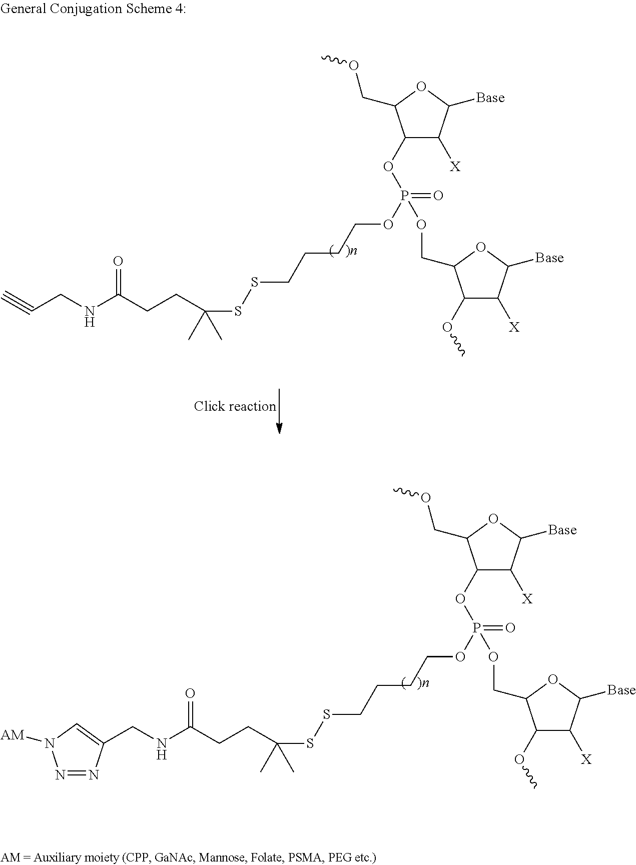

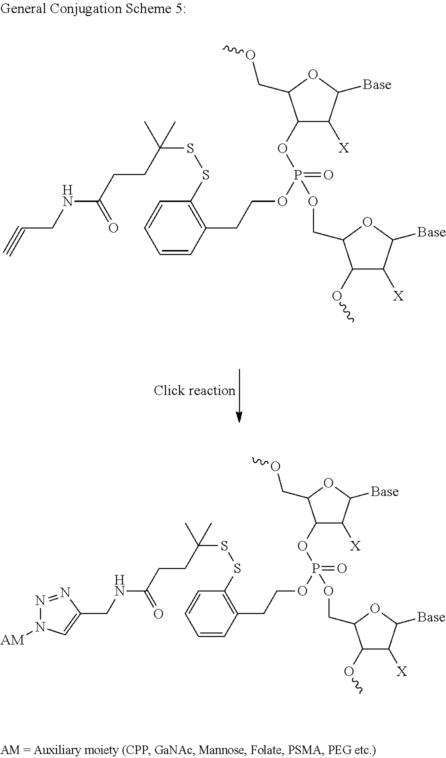

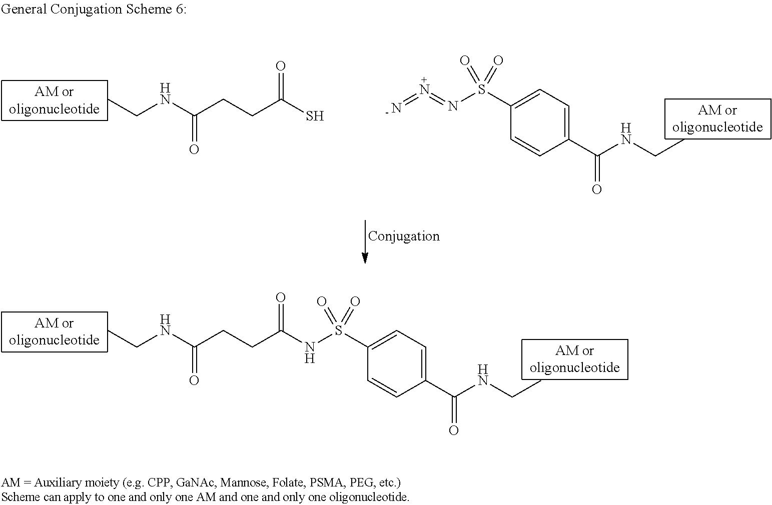

### Specific Syntheses of the Polynucleotides of the Invention

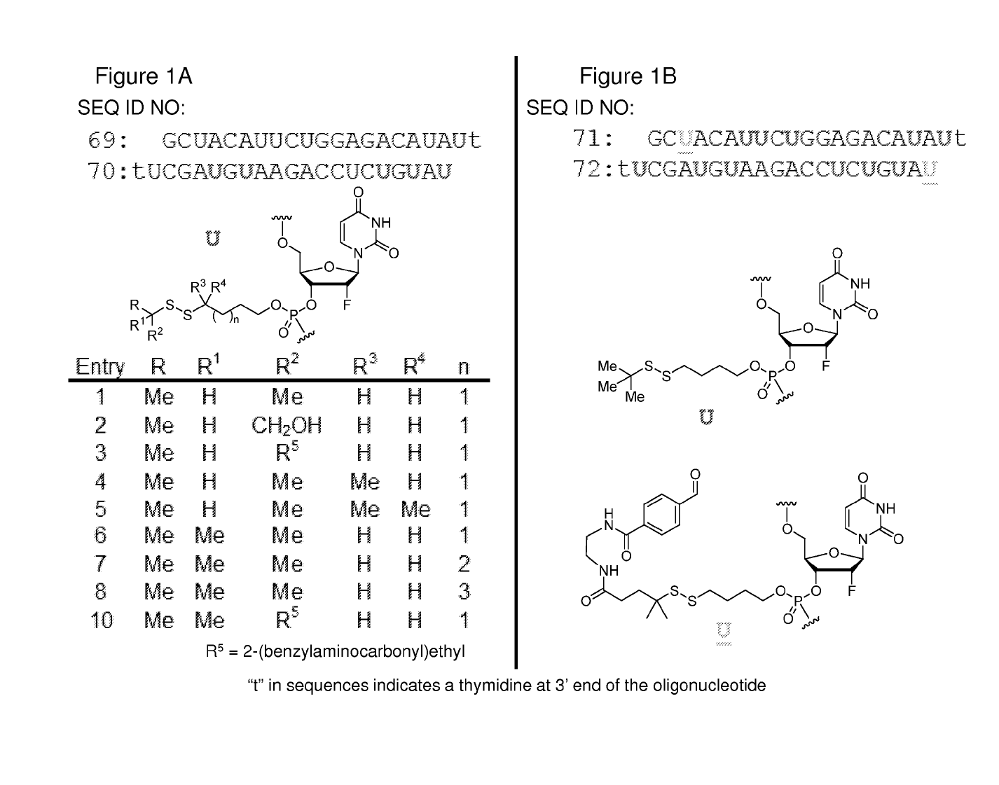

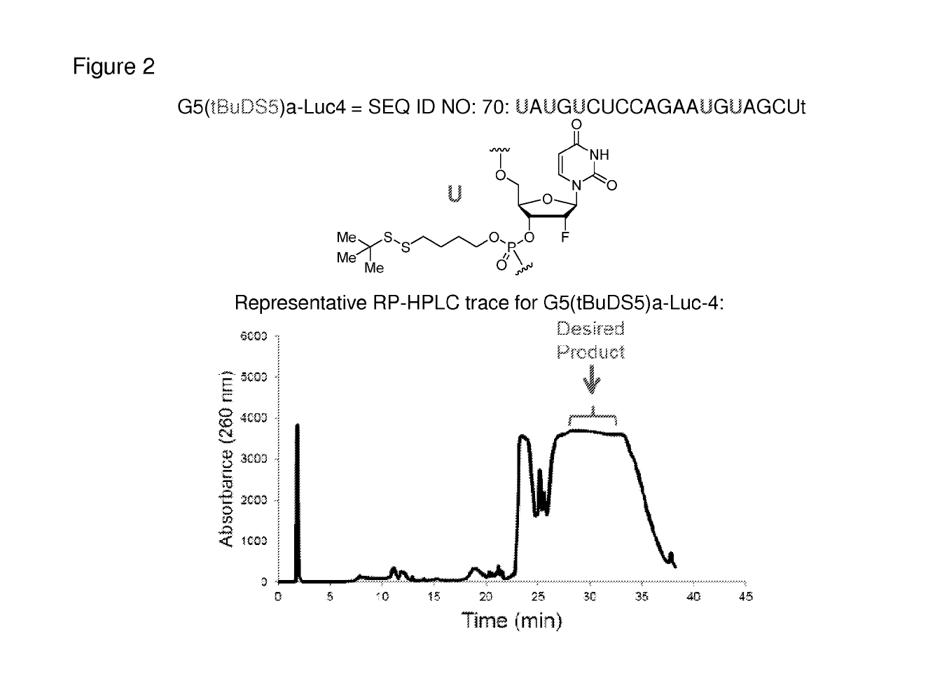

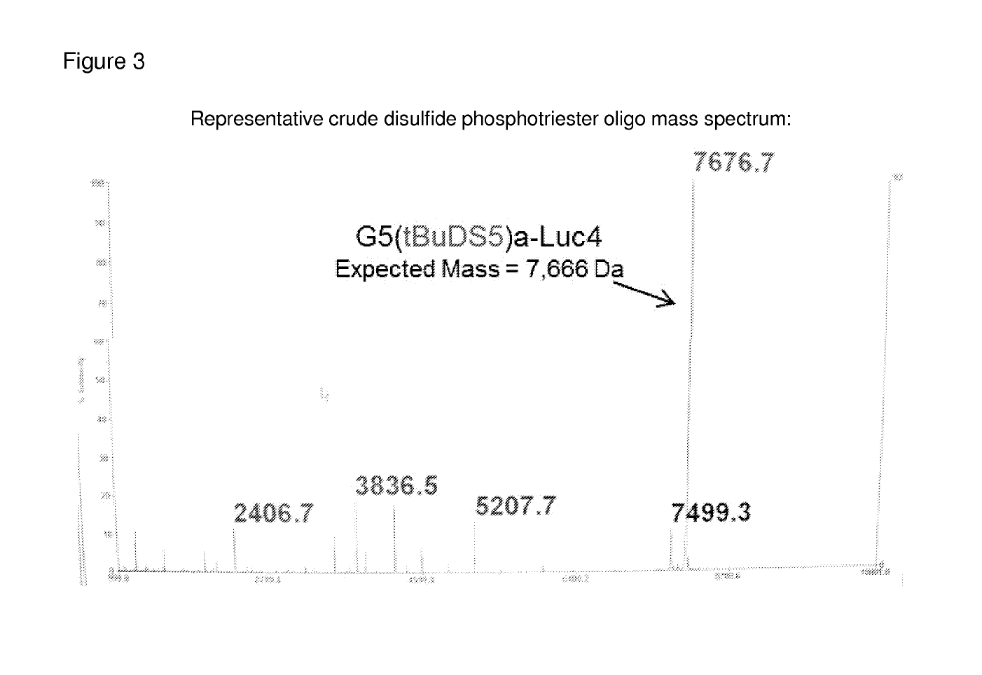

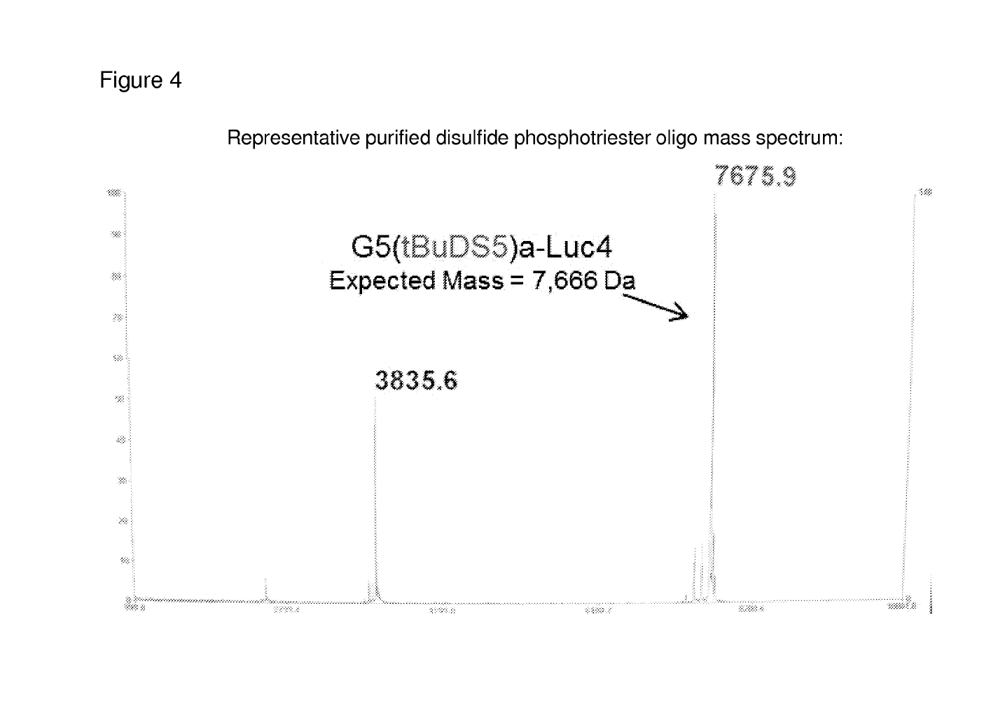

Polynucleotides of the invention have been prepared according to methods described herein. The exemplary polynucleotides are siRNA constructs having the sequences of SEQ ID NO: 69 and 70 (see FIG. 1A) or the sequences of SEQ ID NO: 71 and 72 (see FIG. 1B). Exemplary RP-HPLC trace of SEQ ID NO: 70 is shown in FIG. 2. The mass spectrum of the crude reaction mixture containing the oligonucleotide having the sequence of SEQ ID NO: 70 is shown in FIG. 3. The mass spectrum of the purified containing the oligonucleotide having the sequence of SEQ ID NO: 70 is shown in FIG. 4.

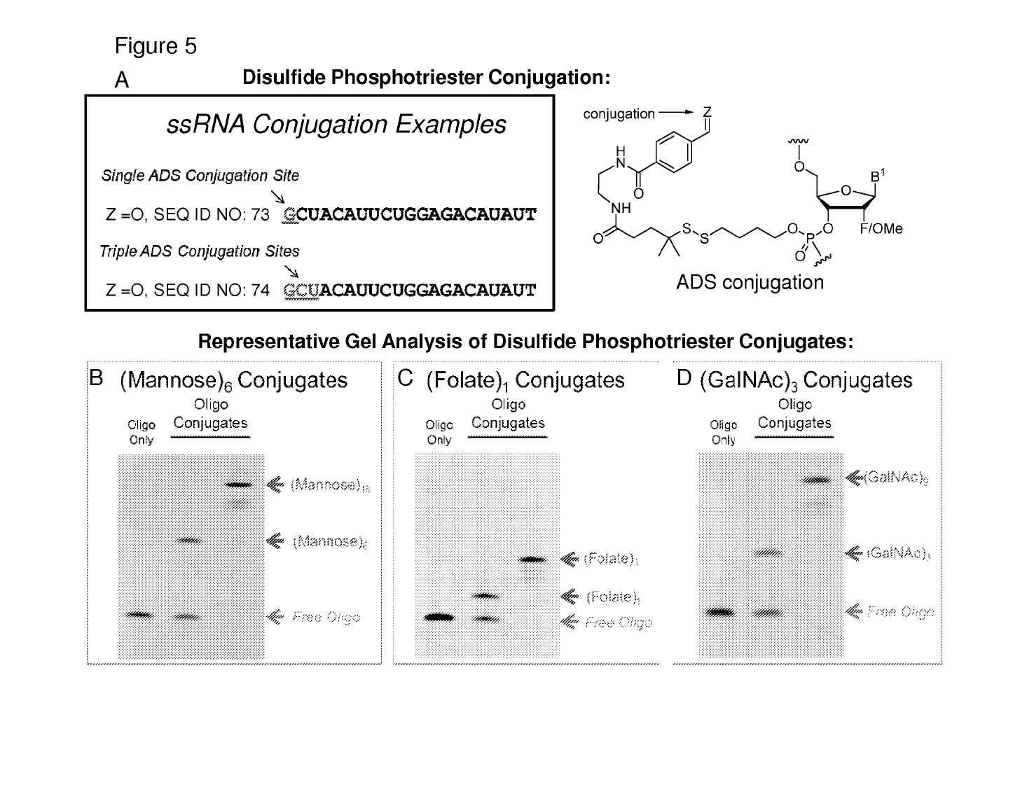

Other polynucleotides of the invention have been prepared according to the methods described herein. For example, FIG. 5A shows the sequences SEQ ID NOs: 73 and 74 (when Z is oxygen), the former containing one 5′-terminal ADS conjugation site having the structure of “ADS conjugation,” and the latter containing three ADS conjugation sites, each having the structure of “ADS conjugation.” FIGS. 5B-5D show gel analyses of some of the polynucleotides of the invention having one or three nucleotides with conjugated targeting moieties contained in Z of the ADS conjugation structure.

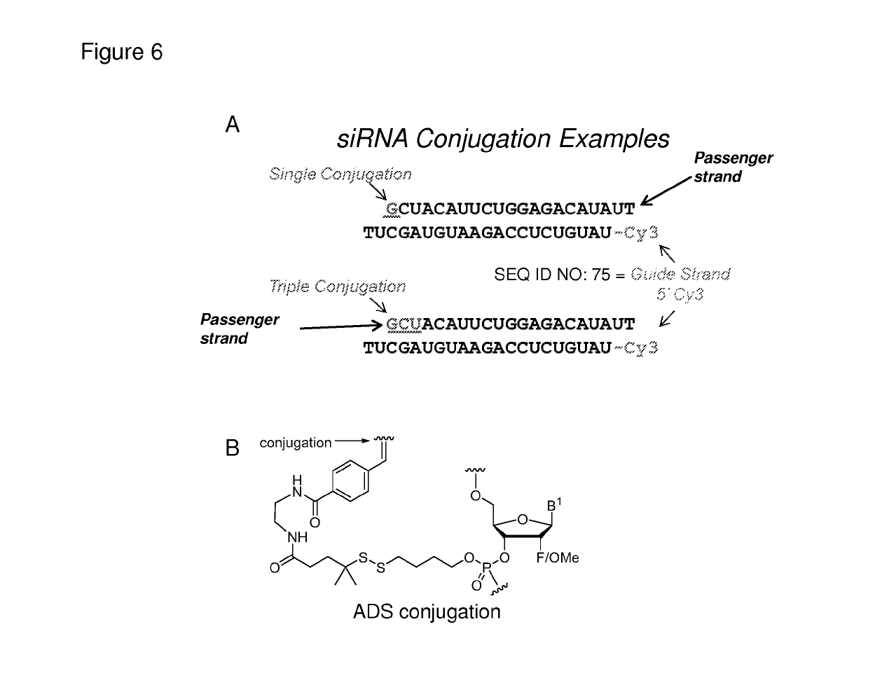

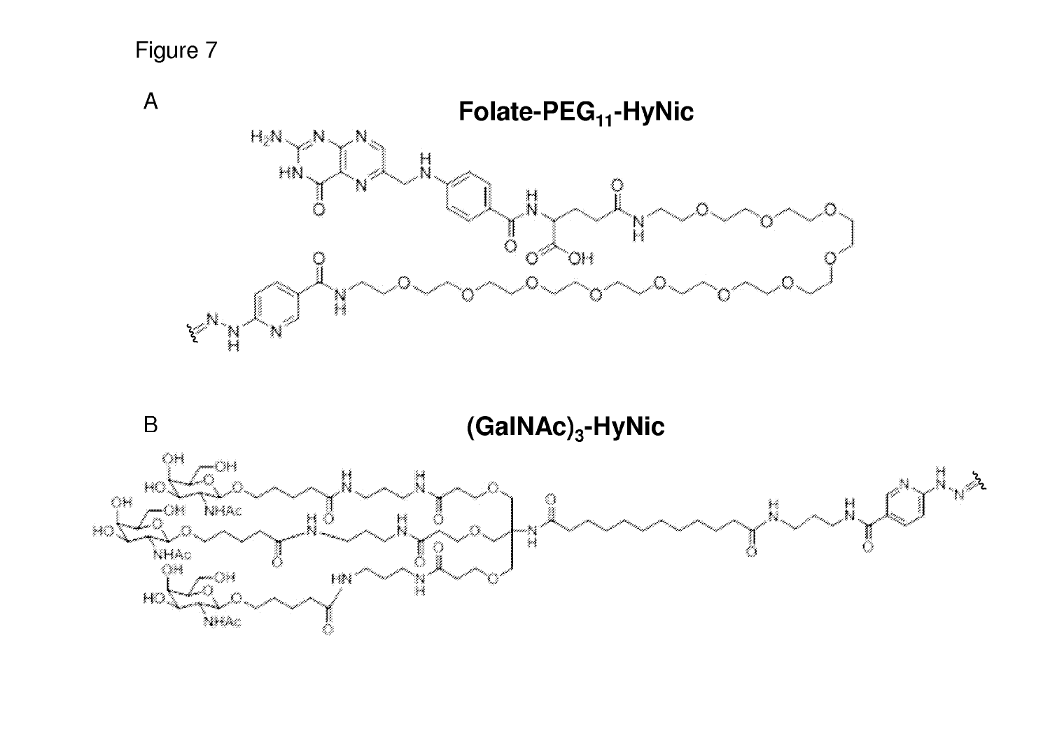

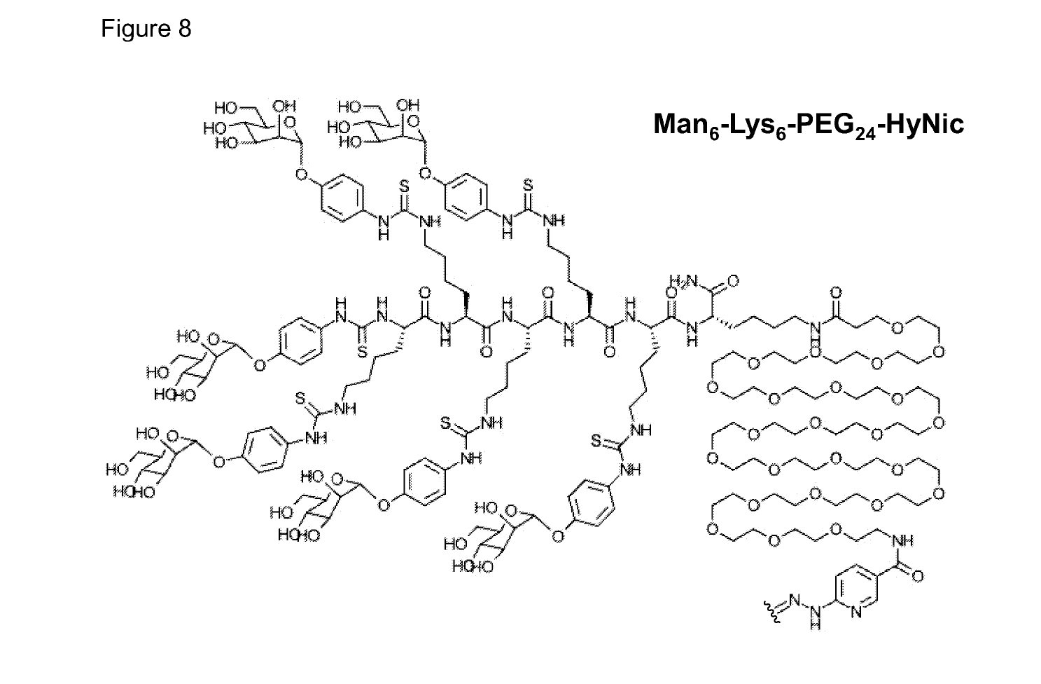

The general structures of the prepared siRNA molecules containing a passenger strand having one or three groups containing targeting moieties are shown in FIGS. 6A and 6B. The guiding strand in FIG. 6A has a 5′-terminal Cy3 moiety (SEQ ID NO: 75). Two exemplary polynucleotides of the invention contain one or three Folate-PEG₁₁-HyNic groups shown in FIG. 7A. (Folate)ₜ-siRNN-Cy3 (SEQ ID NO: 67) is a polynucleotide construct having a sequence 5′-GCUACAUUCUGGAGACAUAUt (lower-case t is thymidine) containing one Folate-PEG₁₁-HyNic group conjugated to the internucleotide bridging group of 5′-terminal G. (Folate)₃-siRNN-Cy3 is a polynucleotide construct having a sequence 5′-GCUACAUUCUGGAGACAUAUt containing three Folate-PEG₁₁-HyNic groups conjugated to the three internucleotide bridging groups of 5′-GCU. Two exemplary polynucleotides of the invention contain one or three (GalNAc)₃-HyNic groups shown in FIG. 7B. (GalNAc)₃-siRNN-Cy3 is a polynucleotide construct having a sequence 5′-GCUACAUUCUGGAGACAUAUt containing one (GalNAc)₃-HyNic group conjugated to the internucleotide bridging group of 5′-terminal G. (GalNAc)₉-siRNN-Cy3 is a polynucleotide construct having a sequence 5′-GCUACAUUCUGGAGACAUAUT containing three (GalNAc)₃-HyNic groups conjugated to the three internucleotide bridging groups of 5′-GCU. Two exemplary polynucleotides of the invention contain one or three Man₆-Lys₆-PEG₂₄-HyNic groups shown in FIG. 8. (Mannose)₆-siRNN-Cy3 is a polynucleotide construct having a sequence 5′-GCUACAUUCUGGAGACAUAUT containing one Man₆-Lys₆-PEG₂₄-HyNic group conjugated to the internucleotide bridging group of 5′-terminal G. (Mannose)₁₈-siRNN-Cy3 is a polynucleotide construct having a sequence 5′-GCUACAUUCUGGAGACAUAUT containing three Man₆-Lys₆-PEG₂₄-HyNic groups conjugated to the three internucleotide bridging groups of 5′-GCU.

Other prepared polynucleotides of the invention contain one to three GalNAc monomers (see below) conjugated to one to ten (e.g., one to four) internucleotide bridging groups.

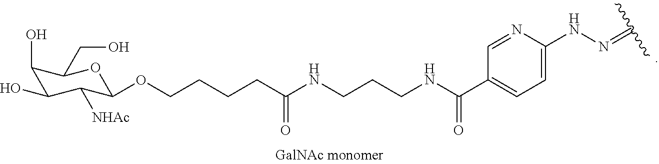 [SMILES: *=NNc1ccc(C(=O)NCCCNC(=O)CCCCOC2OC(CO)C(O)C(O)C2NC(C)=O)cn1]

The list of exemplary siRNA triesters and conjugates is provided in Table 4:

TABLE 4

| Compound | | | | Strand | | |
|---|---|---|---|---|---|---|
| # | Ligand | Target | Strand | # | Sequences (5′-3′) | Conjugation-Prodrug Linker |
| SB-0068 | P20 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0069 | P21 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0070 | P36 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0071 | M6 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0072 | M6 | GAPDH | P | P3271 | UCUACAUGUUCCAGUAUGAUt | Aldehyde-Disulfide (4-carbon) |
| | | | G | G3102 | UCAUACUGGAACAUGUAGAUt | |
| SB-0073 | NAG19 | ApoB | P | P3276 | Ald-UCAUCACACUGAAUACC | 5′ Hydrazone |
| | | | | | AAUt | |
| | | | G | G3258 | UUGGUAUUCAGUGUGAUGAUt | |
| SB-0074 | NAG19 | ApoB | P | P3277 | UCAUCACACUGAAUACCAAUt | Aldehyde-Disulfide (ortho) |
| | | | G | G3258 | UUGGUAUUCAGUGUGAUGAUt | |
| SB-0075 | NAG19 | ApoB | P | P3279 | UCAUCACACUGAAUACCAAUt | Aldehyde-Disulfide (ortho) |
| | | | G | G3282 | UUGGUAUUCAGUGUGAUGAUt | |
| SB-0076 | F3 | GAPDH | P | P3270 | UCUACAUGUUCCAGUAUGAUt | Aldehyde-Disulfide (4-carbon) |
| | P17 | | G | G3272 | UCAUACUGGAACAUGUAGAUt | Aldehyde-Disulfide (4-carbon) |
| SB-0077 | F3 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0078 | | GAPDH | P | P3101 | UCUACAUGUUCCAGUAUGAUt | |
| | P17 | | G | G3272 | UCAUACUGGAACAUGUAGAUt | Aldehyde-Disulfide (4-carbon) |
| SB-0080 | F3 | GAPDH | P | P3270 | UCUACAUGUUCCAGUAUGAUt | Aldehyde-Disulfide (4-carbon) |
| | P33 | | G | G3272 | UCAUACUGGAACAUGUAGAUt | Aldehyde-Disulfide (4-carbon) |
| SB-0081 | NAG21 | ApoB | P | P3287 | Hex-UCAUCACACUGAAUACC | 5′ Click |
| | | | | | AAUt | |
| | | | G | G3282 | UUGGUAUUCAGUGUGAUGAUt | |
| SB-0082 | F3 | GAPDH | P | P3270 | UCUACAUGUUCCAGUAUGAUt | Aldehyde-Disulfide (4-carbon) |
| | P35 | | G | G3272 | UCAUACUGGAACAUGUAGAUt | Aldehyde-Disulfide (4-carbon) |
| SB-0083 | F3 | GAPDH | P | P3270 | UCUACAUGUUCCAGUAUGAUt | Aldehyde-Disulfide (4-carbon) |
| | P32 | | G | G3272 | UCAUACUGGAACAUGUAGAUt | Aldehyde-Disulfide (4-carbon) |
| SB-0085 | NAG21 | ApoB | P | P3297 | Hex-UCAUCACACUGAAUACC | 5′ Click |
| | | | | | AAUt | |
| | | | G | G3258 | UUGGUAUUCAGUGUGAUGAUt | |
| SB-0088 | P34 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0089 | P34 | GAPDH | P | P3270 | UCUACAUGUUCCAGUAUGAUt | Aldehyde-Disulfide (4-carbon) |
| | P34 | | G | G3272 | UCAUACUGGAACAUGUAGAUt | Aldehyde-Disulfide (4-carbon) |
| SB-0090 | F3 | GAPDH | P | P3270 | UCUACAUGUUCCAGUAUGAUt | Aldehyde-Disulfide (4-carbon) |
| | P34 | | G | G3272 | UCAUACUGGAACAUGUAGAUt | Aldehyde-Disulfide (4-carbon) |
| SB-0094 | NAG21 | ApoB | P | P3290 | Hex-uCAUCACACUGAAUACC | 5′ Click |
| | | | | | AAut | |
| | | | G | G3292 | uUGGUAUUCAGUGUGAUGAut | |
| SB-0095 | NAG21 | NTC | P | P3291 | Hex-AGUACUGCUUACGAUAC | 5′ Click |
| | | | | | GGut | |
| | | | G | G3293 | CCGUAUCGUAAGCAGUACUut | |
| SB-0096 | NAG19 | ApoB | P | P3294 | UCAUCACACUGAAUACCAAut | Aldehyde-Disulfide (ortho) |
| | | | G | G3292 | uUGGUAUUCAGUGUGAUGAut | |
| SB-0097 | NAG21 | ApoB | P | P3295 | UCAUCACACUGAAUACCAAut | Alkyne-Disulfide (ortho) |
| | | | G | G3292 | uUGGUAUUCAGUGUGAUGAut | |
| SB-W98 | NAG19 | ApoB | P | P3296 | Ald-uCAUCACACUGAAUACC | 5′ Hydrazone |
| | | | | | AAut | |
| | | | G | G3292 | uUGGUAUUCAGUGUGAUGAut | |
| SB-0099 | PSMA10 | Luc | P | P3182 | GCUACAUUCUGGAGACAUAUt | Aldehyde-Disulfide (4-carbon) |
| | | | G | G3247 | Cy3-UAUGUCUCCAGAAUGUA | |
| | | | | | GCUt | |
| SB-0100 | PSMA20 | Luc | P | P3182 | GCUACAUUCUGGAGACAUAUt | Aldehyde-Disulfide (4-carbon) |
| | | | G | G3247 | Cy3-UAUGUCUCCAGAAUGUA | |
| | | | | | GCUt | |
| SB-0101 | NAG21 | ApoB | P | P3298 | Hex-UCAUCACACUGAAUACC | 5′ Click |
| | | | | | AAUt | |
| | | | G | G3299 | UUGGUAUUCAGUGUGAUGAUt | |
| SB-0102 | NAG21 | ApoB | P | P3307 | uCAUCACACUGAAUACCAAut | Alkyne-Disulfide (ortho) |
| | | | G | G3292 | uUGGUAUUCAGUGUGAUGAut | |
| SB-0103 | NAG21 | ApoB | P | P3308 | uCAUCACACUGAAUACCAAUt | Alkyne-Disulfide (ortho) |
| | | | G | G3292 | uUGGUAUUCAGUGUGAUGAut | |
| SB-0104 | NAG21 | ApoB | P | P3309 | UCAUCACACUGAAUACCAAUt | Alkyne-Disulfide (ortho) |
| | | | G | G3292 | uUGGUAUUCAGUGUGAUGAut | |
| SB-0105 | F6 | ApoB | P | P3290 | Hex-uCAUCACACUGAAUACC | 5′ Click |
| | | | | | AAut | |
| | | | G | G3310 | IR700-uUGGUAUUCAGUGUG | |
| | | | | | AUGAut | |
| SB-0106 | NAG21 | ApoB | P | P3312 | UCAUcACACUGAAUAcCAAut | Alkyne-Disulfide (ortho) |
| | | | G | G3314 | uUGGcAUUCAGUGUGaUGAut | |
| SB-0107 | NAG21 | ApoB | P | P3313 | UCAUcACAcUGaAUAcCAAut | Alkyne-Disulfide (ortho) |
| | | | G | G3292 | uUGGCAUUCAGUGUGAUGAut | |
| SB-0108 | NAG21 | ApoB | P | P3318 | Hex-UCAUcACACUGAAUAcC | 5′ Click |
| | | | | | AAUt | |
| | | | G | G3319 | UUGGuAUUCAGUGUGaUGAUt | |
| SB-0109 | NAG21 | ApoB | P | P3320 | Hex-UsCAUCACACUGAAUAC | 5′ Click |
| | | | | | CAAUst | |
| | | | G | G3306 | UsUCGUAUUCACUCUGAUCAU | |
| | | | | | st | |
| SB-0110 | NAG21 | ApoB | P | P3295 | UCAUCACACUGAAUACCAAut | Alkyne-Disulfide (ortho) |
| | | | G | G3315 | uUGGuAUUcAGuGUGaUGAut | |
| SB-0111 | NAG21 | ApoB | P | P3295 | UCAUCACACUGAAUACCAAut | Alkyne-Disulfide (ortho) |
| | P34 | | G | G3303 | UUGGUAUUCAGUGUGAUGAut | Aldehyde-Disulfide (ortho) |
| SB-0112 | NAG21 | ApoB | P | P3295 | UCAUCACACUGAAUACCAAut | Alkyne-Disulfide (ortho) |
| | P32 | | G | G3303 | UUGGUAUUCAGUGUGAUGAut | Aldehyde-Disulfide (ortho) |
| SB 0113 | NAG21 | ApoB | P | P3295 | UCAUCACACUGAAUACCAAut | Alkyne-Disulfide (ortho) |
| | P17 | | G | G3303 | UUGGUAUUCAGUGUGAUGAut | Aldehyde-Disulfide (ortho) |
| SB-0114 | NAG21 | ApoB | P | P3295 | UCAUCACACUGAAUACCAAut | Alkyne-Disulfide (ortho) |
| | P18 | | G | G3303 | UUGGUAUUCAGUGUGAUGAut | Aldehyde-Disulfide (ortho) |
| SB-0115 | NAG21 | ApoB | P | P3295 | UCAUCACACUGAAUACCAAut | Alkyne-Disulfide (ortho) |
| | P33 | | G | G3303 | UUGGUAUUCAGUGUGAUGAut | Aldehyde-Disulfide (ortho) |
| SB-0116 | NAG21 | ApoB | P | P3295 | UCAUCACACUGAAUACCAAut | Alkyne-Disulfide (ortho) |
| | P35 | | G | G3303 | UUGGUAUUCAGUGUGAUGAut | Aldehyde-Disulfide (ortho) |
| SB-0117 | NAG21 | ApoB | P | P3295 | UCAUCACACUGAAUACCAAut | Alkyne-Disulfide (ortho) |
| | P05 | | G | G3303 | UUGGUAUUCAGUGUGAUGAut | Aldehyde-Disulfide (ortho) |
| SB-0118 | NAG21 | ApoB | P | P3295 | UCAUCACACUGAAUACCAAut | Alkyne-Disulfide (ortho) |
| | P04 | | G | G3303 | UUGGUAUUCAGUGUGAUGAut | Aldehyde-Disulfide (ortho) |
| SB-0119 | NAG21 | ApoB | P | P3295 | UCAUCACACUGAAUACCAAut | Alkyne-Disulfide (ortho) |
| | P20 | | G | G3303 | UUGGUAUUCAGUGUGAUGAut | Aldehyde-Disulfide (ortho) |
| SB-0120 | NAG21 | Factor | P | P3316 | AGGAUCAUCUCAAGUCUUAut | Alkyne-Disulfide (ortho) |
| | | VII | G | G3317 | uAAGACUUGAGAUGAUCCUut | |
| SB-0121 | NAG21 | ApoB | P | P3363 | Hex-UCAUCACACUGAAUACC | 5′ Click |
| | | | | | AAUt | |
| | | | G | G3366 | UUGGUAUUCAGUGUGAUGAUt | |
| SB-0122 | NAG21 | ApoB | P | P3359 | Hex-uCAUCACACUGAAUACC | 5′ Click |
| | | | | | AAut | |
| | | | G | G3360 | uUGGUAUUCAGUGUGAUGAut | |
| SB-0123 | NAG21 | ApoB | P | P3361 | [symbol] CAUCACACUGAAUACCAA [symbol] t | Alkyne-Disulfide (ortho) |
| | | | G | G3362 | [symbol] UCCUAUUCACUCUCAUCA [symbol] t | |
| SB-0124 | M9 | GAPDH | P | | UCUACAUGUUCCAGUAUGAut | Alkyne-Disulfide (ortho) |
| | | | G | | uCAUACUGGAACAUGUAGAut | |
| SB-0130 | NAG21 | ApoB | P | P3373 | uCAUCACACUGAAUACCAAut | Alkyne-Disulfide (ortho) |
| | | | G | G3372 | UUGGUAUUCAGUGUGAUGAut | |
| SB-0132 | M9 | ApoB | P | | UCAUCACACUGAAUACCAAut | Alkyne-Disulfide (ortho) |
| | | | G | | uUGGUAUUCAGUGUGAUGAut | |
| SB-0133 | NAG21 | ApoB | P | P3295 | UCAUCACACUGAAUACCAAut | Alkyne-Disulfide (ortho) |
| | | | G | G3378 | uUGGUAUUCAGUGUGAUGAUs | |
| | | | | | t | |
| SB-0134 | NAG21 | ApoB | P | P3376 | UCAUCACACUGAAUACCAAUs | Alkyne-Disulfide (ortho) |
| | | | | | t | |
| | | | G | G3378 | uUGGUAUUCAGUGUGAUGAUs | |
| | | | | | t | |
| SB-0135 | NAG21 | ApoB | P | P3376 | UCAUCACACUGAAUACCAAUs | Alkyne-Disulfide (ortho) |
| | | | | | t | |
| | | | G | G3379 | UsUGGUAUUCAGUGUGAUGAu | |
| | | | | | t | |
| SB-0136 | NAG21 | ApoB | P | P3376 | UCAUCACACUGAAUACCAAUs | Alkyne-Disulfide (ortho) |
| | | | | | t | |
| | | | G | G3306 | UsUGGUAUUCAGUGUGAUGAU | |
| | | | | | st | |
| SB-0137 | NAG21 | ApoB | P | P3377 | UCAUCACACUGAAUACCAAut | Alkyne-Disulfide (ortho) |
| | | | G | G3380 | uUGGUAUUCAGUGUGAUGAut | |
| SB-0138 | M9 | GAPDH | P | P3382 | Hex-UsCUACAUGUUCCAGUA | 5′ Click |
| | | | | | UGAUst | |
| | | | G | G3383 | UsCAUACUGGAACAUGUAGAU | |
| | | | | | st | |
| SB-0139 | M9 | GAPDH | P | P3384 | Hex-uCUACAUGUUCCAGUAU | 5′ Click |
| | | | | | GAut | |
| | | | G | G3365 | uCAUACUGGAACAUGUAGAut | |
| SB-0140 | NAG21 | ApoB | P | P3454 | Hex-UsCAUCACACUGAAUAC | 5′ Click |
| | | | | | CAAUst | |
| | | | G | G3457 | UsUGGUAUUCAGUGUGAUGAU | |
| | | | | | st | |
| SB-0141 | P45 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0142 | NAG21 | ApoB | P | P3376 | UCAUCACACUGAAUACCAAUs | Alkyne-Disulfide (ortho) |
| | | | | | t | |
| | P45 | | G | G3381 | UUGGUAUUCAGUGUGAUGAUs | Alkyne-Disulfide (ortho) |
| | | | | | t | |
| SB-0146 | NAG21 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0154 | NAG21 | ApoB | P | P3458 | Hex-UiCAUCACACUGAAUAC | 5′ Click |
| | | | | | CAAUit | |
| | | | G | G3459 | ULUGGUAUUCAGUGUGAUGUi | |
| | | | | | t | |
| SB-0155 | NAG21 | ApoB | P | P3460 | Hex-UpCAUCACACUGAAUAC | 5′ Click |
| | | | | | CAAUpt | |
| | | | G | G3461 | UpUGGUAUUCAGUGUGAUGUp | |
| | | | | | t | |
| SB-0156 | NAG21 | ApoB | P | P3452 | Hex-UmCAUCACACUGAAUAC | 5′ Click |
| | | | | | CAAUmt | |
| | | | G | G3453 | UmUGGUAUUCAGUGUGAUGUm | |
| | | | | | t | |
| SB-0157 | NAG21 | ApoB | P | P3462 | Hex-UbCAUCACACUGAAUAC | 5′ Click |
| | | | | | CAAUbt | |
| | | | G | G3463 | UbUGGUAUUCAGUGUGAUGUb | |
| | | | | | t | |
| SB-0162 | | ApoB | P | P3307 | uCAUCACACUGAAUACCAAut | |
| | NAG21 | | G | G3372 | UUGGUAUUCAGUGUGAUGAut | Alkyne-Disulfide (ortho) |
| SB-0163 | P42 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB 0164 | P50 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0222 | NAG21 | ApoB | P | P3522 | UCAUCACACUGAAUACCAAut | Alkyne-Disulfide (ortho) |
| | | | G | G3292 | uUGGUAUUCAGUGUGAUGAut | |
| SB-0223 | NAG21 | ApoB | P | P3523 | UCAUCACACUGAAUACCAAut | Alkyne-Disulfide (ortho) |
| | | | G | G3292 | uUGGUAUUCAGUGUGAUGAut | |
| SB-0224 | NAG21 | ApoB | P | P3524 | UCAUCACACUGAAUACCAAut | Alkyne-Disulfide (ortho) |
| | | | G | G3292 | uUGGUAUUCAGUGUCAUGAut | |
| SB-0225 | NAG21 | ApoB | P | P3295 | UCAUCACACUGAAUACCAAut | Alkyne-Disulfide (ortho) |
| | NAG21 | | G | G3525 | uUGGUAUUCAGUGUGAUGAut | Alkyne-Disulfide (ortho) |
| SB-0226 | NAG21 | ApoB | P | P3523 | UCAUCACACUGAAUACCAAut | Alkyne-Disulfide (ortho) |
| | NAG21 | | G | G3525 | uUGGUAUUCAGUGUGAUGAut | Alkyne-Disulfide (ortho) |
| SB-0227 | M30 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0228 | M30 | GAPDH | P | P3527 | UCUACAUCUUCCACUAUGAut | Alkyne-Disulfide (ortho) |
| | | | G | G3365 | uCAUACUGGAACAUGUAGAut | |
| SB-0229 | M30 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0230 | M30 | GAPDH | P | P3529 | UCUACAUGUUCCAGUAUGAUt | Alkyne-Disulfide (ortho) |
| | | | G | G3365 | uCAUACUGGAACAUGUAGAut | |
| SB-0231 | M30 | GAPDH | P | P3530 | UCUACAUGUUCCAGUAUGAut | Alkyne-Disulfide (ortho) |
| | | | G | G3365 | uCAUACUGGAACAUGUAGAut | |
| SB-0232 | M30 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0233 | PSMA10 | GAPDH | P | P3526 | UCUACAUGUUCCAGUAUGAut | Aldehyde-Disulfide (ortho) |
| | | | G | G3365 | uCAUACUGGAACAUGUAGAut | |
| SB-0234 | P51 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0235 | P52 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0236 | P49 | ApoB | P | P3307 | uCAUCACACUGAAUACCAAut | Alkyne Disulfide (ortho) |
| | NAG21 | | G | G3372 | UUGGUAUUCAGUGUGAUGAut | Alkyne-Disulfide (ortho) |
| SB-0237 | P37 | ApoB | P | P3307 | uCAUCACACUGAAUACCAAut | Alkyne-Disulfide (ortho) |
| SB-0238 | P38 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0239 | P39 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0240 | P47 | ApoB | P | P3307 | uCAUCACACUGAAUACCAAut | Alkyne-Disulfide (ortho) |
| | NAG21 | | G | G3372 | UUCCUAUUCACUCUGAUGAut | Alkyne-Disulfide (ortho) |
| SB-0241 | P46 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0242 | M15 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0248 | M15 | GAPDH | P | P3527 | UCUACAUGUUCCAGUAUGAut | Alkyne-Disulfide (ortho) |
| | | | G | G3365 | uCAUACUGGAACAUGUAGAut | |
| SB-0249 | M15 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0250 | M15 | GAPDH | P | P3529 | UCUACAUGUUCCAGUAUGAUt | Alkyne-Disulfide (ortho) |
| | | | G | G3365 | uCAUACUGGAACAUGUAGAut | |
| SB-0251 | M15 | GAPDH | P | P3530 | UCUACAUGUUCCAGUAUGAut | Alkyne-Disulfide (ortho) |
| | | | G | G3365 | uCAUACUGGAACAUGUAGAut | |
| SB-0252 | M15 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0253 | PSMA20 | GAPDH | P | P3526 | UCUACAUGUUCCAGUAUGAut | Aldehyde-Disulfide (ortho) |
| | | | G | G3365 | uCAUACUCCAACAUGUAGAut | |
| SB-0259 | P45 | ApoB | P | P3551 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0260 | P45 | ApoB | P | P3551 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0285 | NAG21 | ApoB | P | P3553 | Hex-U(m1)CAUCACACUGAA | 5′ Click |
| | | | | | UACCAAU(m1)t | |
| | | | G | G3554 | U(m1)UGGUAUUCAGUGUGAU | |
| | | | | | GU(m1)t | |
| SB-0286 | NAG21 | ApoB | P | P3555 | Hex-U(m2)CAUCACACUGAA | 5′ Click |
| | | | | | UACCAAU(m2)t | |
| | | | G | G3556 | U(m2)UGGUAUUCAGUGUGAU | |
| | | | | | GU(m2)t | |

The mixed siRNA conjugates of the invention are provided in Table 5:

TABLE 5

| Compound | | | | Strand | Sequences | Conjugation-Prodrug |
|---|---|---|---|---|---|---|
| # | Ligand | Target | Strand | # | (5′-3′) | Linker |
| SB-0097 | NAG21 | ApoB | P | P3295 | UCAUCACACUGAAUACCAAut | Alkyne-Disulfide (ortho) |
| | | | G | G3292 | uUGGUAUUCAGUGUGAUGAut | N/A |
| SB-0111 | NAG21 | ApoB | P | P3295 | UCAUCACACUGAAUACCAAut | Alkyne-Disulfide (ortho) |
| | P34 | | G | G3303 | UUGGUAUUCAGUGUGAUGAut | Aldehyde-Disulfide (ortho) |
| SB-0112 | NAG21 | ApoB | P | P3295 | UCAUCACACUGAAUACCAAut | Alkyne-Disulfide (ortho) |
| | P32 | | G | G3303 | UUGGUAUUCAGUGUGAUGAut | Aldehyde-Disulfide (ortho) |
| SB-0113 | NAG21 | ApoB | P | P3295 | UCAUCACACUGAAUACCAAut | Alkyne-Disulfide (ortho) |
| | P17 | | G | G3303 | UUGGUAUUCAGUGUGAUGAut | Aldehyde-Disulfide (ortho) |
| SB-0114 | NAG21 | ApoB | P | P3295 | UCAUCACACUGAAUACCAAut | Alkyne-Disulfide (ortho) |
| | P18 | | G | G3303 | UUGGUAUUCAGUGUGAUGAut | Aldehyde-Disulfide (ortho) |
| SB-0115 | NAG21 | ApoB | P | P3295 | UCAUCACACUGAAUACCAAut | Alkyne-Disulfide (ortho) |
| | P33 | | G | G3303 | UUGGUAUUCAGUGUGAUGAut | Aldehyde-Disulfide (ortho) |
| SB-0116 | NAG21 | ApoB | P | P3295 | UCAUCACACUGAAUACCAAut | Alkyne-Disulfide (ortho) |
| | P35 | | G | G3303 | UUGGUAUUCAGUGUGAUGAut | Aldehyde-Disulfide (ortho) |
| SB-0117 | NAG21 | ApoB | P | P3295 | UCAUCACACUGAAUACCAAut | Alkyne-Disulfide (ortho) |
| | P05 | | G | G3303 | UUGGUAUUCAGUGUGAUGAut | Aldehyde-Disulfide (ortho) |
| SB-0118 | NAG21 | ApoB | P | P3295 | UCAUCACACUGAAUACCAAut | Alkyne-Disulfide (ortho) |
| | P04 | | G | G3303 | UUGGUAUUCAGUGUGAUGAut | Aldehyde-Disulfide (ortho) |
| SB-0119 | NAG21 | ApoB | P | P3295 | UCAUCACACUGAAUACCAAut | Alkyne-Disulfide (ortho) |
| | P20 | | G | G3303 | UUGGUAUUCAGUGUGAUGAut | Aldehyde-Disulfide (ortho) |

For Tables 4 and 5: UPPER CASE=2′OMe Purines, 2′F Pyrimidines; lower case=deoxy; lower case bold=DMB; BOLD=iPrDS (ortho) (2′OMe Purines, 2′F Pyrimidines); ITALICS=tBuDS (2′OMe Purines, 2′F Pyrimidines); lower case italics=tBuDS-Ph (ortho) (2′OMe Purines, 2′F Pyrimidines); BOLD ITALICS=tBuDS-Ph (ortho)-Phosphorothioate (2′OMe Purines, 2′F Pyrimidines); UNDERLINE=Conjugated Prodrug Location; s=Phosphorothioate; i=NMI-DS-Ph; p=PEG4-DS-Ph; m=tBuDS-Ph(Me); b=tBuDS-Ph(Br); m1=tBuDS-(m1)Me-Ph; m2=tBuDS-(m2)Me-Ph; Ald=5′ Benzaldehyde; Hex=5′ Hexynyl; DS means disulfide, Ph means phenethyl.

Any of the groups disclosed herein may be linked to an internucleotide bridging phosphate or a terminal phosphate through one of the following non-limiting exemplary groups:

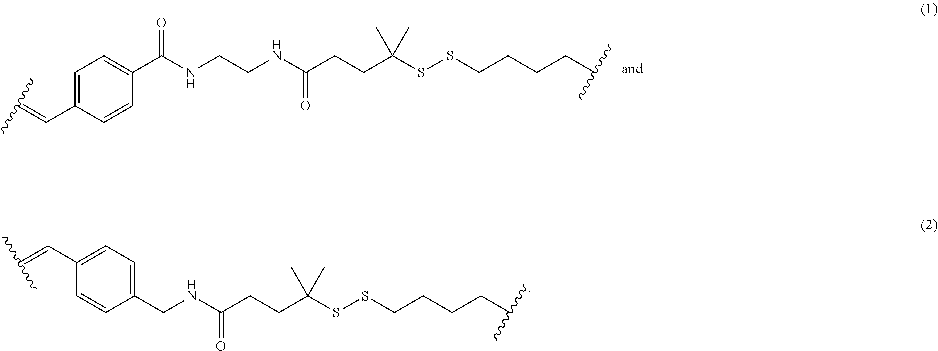 [SMILES: *CCCCSSC(C)(C)CCC(=O)NCCNC(=O)c1ccc(C=*)cc1 ; *CCCCSSC(C)(C)CCC(=O)NCc1ccc(C=*)cc1]

Other polynucleotides of the invention may be prepared according to the methods described herein. Such polynucleotides may be as follows:

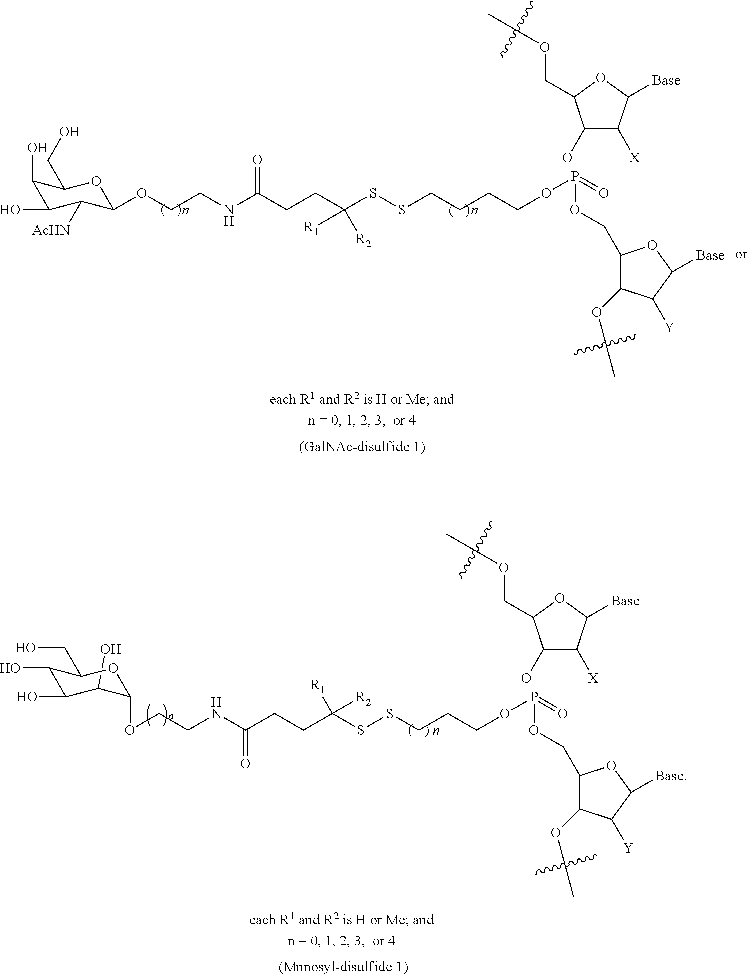

Polynucleotides containing auxiliary moieties directly bound to the disulfide linkage may also be prepared; exemplary polynucleotides are shown below:

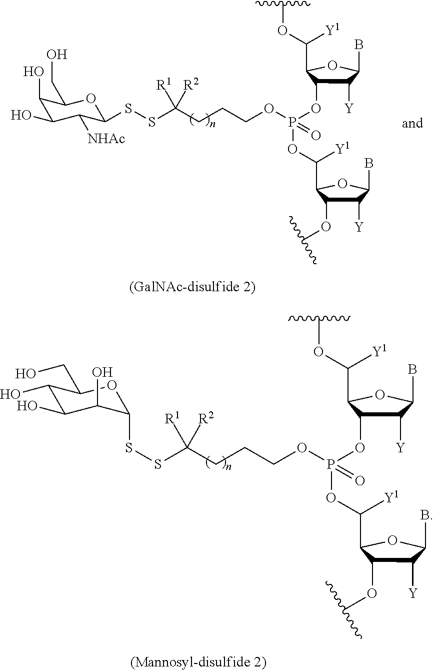

### Example 2

### In Vitro Activity Assays

Polynucleotides targeting the luciferase gene (GL3) were synthesized and were used to generate the polynucleotide constructs having one or more disulfide linkages attached to internucleotide bridging groups (phosphotriesters) and/or terminal groups (phosphodiester or phosphotriester).

To assess the in vitro activity of these disulfide phosphotriesters, human ovarian SKOV-3 cells, stably expressing luciferase (GL3) were utilized. Cells were grown in McCoy's 5A culture medium (Life Technologies) supplemented with 10% fetal bovine serum (FBS), 100 μg/ml of streptomycin, and 100 U/ml of penicillin. Cells (1×10⁴/well) were plated in 96-well microtiter plates and incubated overnight at 37° C. under 5% CO₂.

Control:

The control siRNAs targeting the luciferase gene or a non-targeting control gene were transfected into cells at the indicated concentrations (typically 0.01-30 nM) using lipofectamine RNAiMax (Life Technologies) according to the manufacturer's recommendations.

Polynucleotide Constructs of the Invention:

The polynucleotide constructs were added to cells and incubated for two hours, after which an equal volume of OptiMEM (life Technologies) containing 4% FBS was added and the cells were incubated for 24-48 hours. The cells were then lysed and the intracellular luciferase activity was measured after the addition of luciferin (Britelite, Perkin Elmer) and the luminescence signal was captured using Victor2 luminometer (Perkin Elmer). Cellular toxicity was assessed using the Cell Titer Fluor assay kit (Promega) and the knockdown of the luciferase gene was corrected for cellular toxicity and was expressed as percent of vehicle control treated wells. Luciferase knock-down EC₅₀ values were generated using GraphPad Prism Software.

Results of this assay for the hybridized polynucleotide of the invention (SEQ ID NOs: 69 and 70) are shown in Table 6 (for the structures see FIG. 1A). In Table 6, R⁴ is 2-(benzylaminocarbonyl)ethyl.

TABLE 6

| | | | | | | | EC50^{(a)} | | | |
|---|---|---|---|---|---|---|---|---|---|---|
| | | | | | | | (nM) | | EC50^{(b)} (nM) | |
| Entry | R | R¹ | R² | R³ | R⁴ | n | 24 h | 48 h | 24 h | 48 h |
| 1 | Me | H | Me | H | H | 1 | 0.76 | 0.33 | 0.42 | 0.18 |
| 2 | Me | H | CH₂OH | H | H | 1 | ND | ND | 0.13 | 0.07 |
| 3 | Me | H | R⁴ | H | H | 1 | ND | ND | 0.68 | 0.34 |
| 4 | Me | H | Me | Me | H | 1 | 1.6 | 0.58 | ND | ND |
| 5 | Me | H | Me | Me | Me | 1 | 1.0 | 0.40 | ND | ND |
| 6 | Me | Me | Me | H | H | 1 | 1.0 | 0.34 | 0.38 | 0.15 |
| 7^{(c)} | Me | Me | Me | H | H | 2 | NA | ND | ND | ND |
| 8^{(c)} | Me | Me | Me | H | H | 3 | NA | ND | ND | ND |
| 9 | Me | Me | R⁴ | H | H | 1 | ND | ND | 0.49 | 0.20 |
| DMB^{(d)} | — | — | — | — | — | — | ND | ND | — | — |

^{(a)}Annealing to form siRNA duplexes was carried out at room temperature.
^{(b)}Annealing to form siRNA duplexes was carried out at 65° C.
^{(c)}Annealing to form siRNA duplexes was carried out at room temperature, followed by overnight freezing.
^{(d)}Negative control: the siRNA containing the same sequences, with the exception that the groups containing disulfides are replaced with 3,3-dimethylbutyl (DMB); DMB is linked irreversibly (under physiological conditions) to phosphate.
ND = not determined.
NA = not active.

EC₅₀ (at 48 h) of the hybridized polynucleotide of the invention (SEQ ID NOs: 71 and 72; for the structures see FIG. 1B) was measured to be 1.1 nM.

Table 7 shows she data for other hybridized polynucleotides of the invention having the polynucleotide sequences of SEQ ID NOs: 69 and 70, in which certain uridines (labeled as bold U) have an internucleotide 3′-phosphotriester having the structure shown in Table 7.

### Transfection Data in SKOV-3-Luc Cells:

TABLE 7
SEQ ID NO: 69: GCUACAUUCUGGAGACAUAUt
SEQ ID NO: 70: tUCGAUGUAAGACCUCUGUAU

| | EC50 (nM) | |
|---|---|---|
| Compound | 24h | 48h |
| Control siRNA | 0.01 | 0.01 |
| 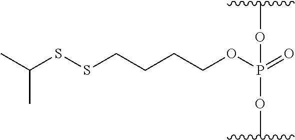 [SMILES: *OP(=O)(O*)OCCCCSSC(C)C] | 0.43 | 0.11 |
| 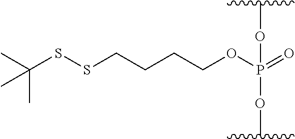 [SMILES: *OP(=O)(O*)OCCCCSSC(C)(C)C] | 0.66 | 0.13 |
| 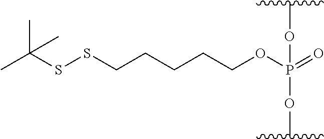 [SMILES: *OP(=O)(O*)OCCCCCSSC(C)(C)C] | >10 | >10 |
| 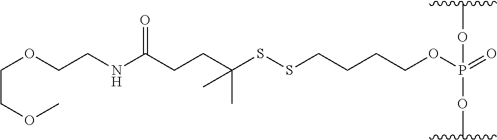 [SMILES: *OP(=O)(O*)OCCCCSSC(C)(C)CCC(=O)NCCOCCOC] | 0.43 | — |
| 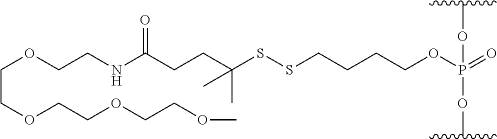 [SMILES: *OP(=O)(O*)OCCCCSSC(C)(C)CCC(=O)NCCOCCOCCOCCOC] | 0.78 | 0.19 |
| 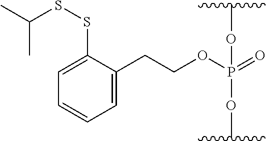 [SMILES: *OP(=O)(O*)OCCc1ccccc1SSC(C)C] | 0.15 | 0.06 |
| 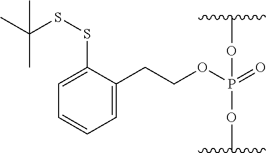 [SMILES: *OP(=O)(O*)OCCc1ccccc1SSC(C)(C)C] | 0.73 | 0.13 |
| 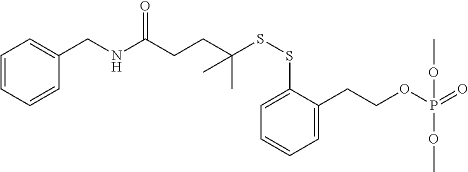 [SMILES: COP(=O)(OC)OCCc1ccccc1SSC(C)(C)CCC(=O)NCc1ccccc1] | 0.16 | 0.06 |
| 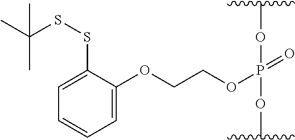 [SMILES: *OP(=O)(O*)OCCOc1ccccc1SSC(C)(C)C] | 1.8 | 0.70 |
| 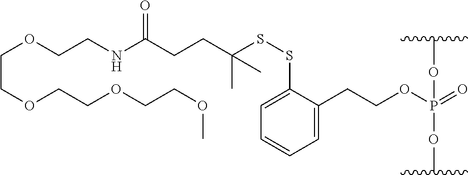 [SMILES: *OP(=O)(O*)OCCc1ccccc1SSC(C)(C)CCC(=O)NCCOCCOCCOCCOC] | 1.01 | 0.42 |
| 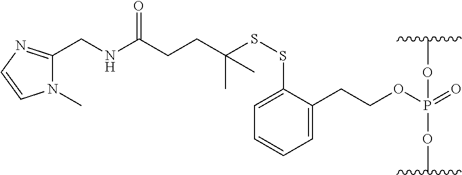 [SMILES: *OP(=O)(O*)OCCc1ccccc1SSC(C)(C)CCC(=O)NCc1nccn1C] | 0.82 | 0.22 |

### Mouse Primary Hepatocyte Isolation and In Vitro Experiments:

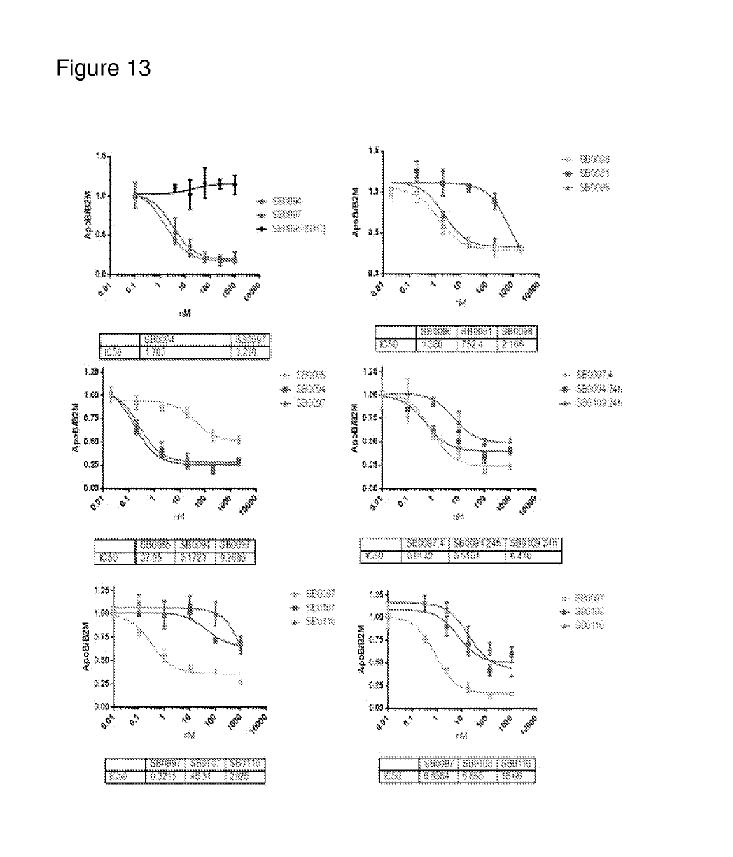

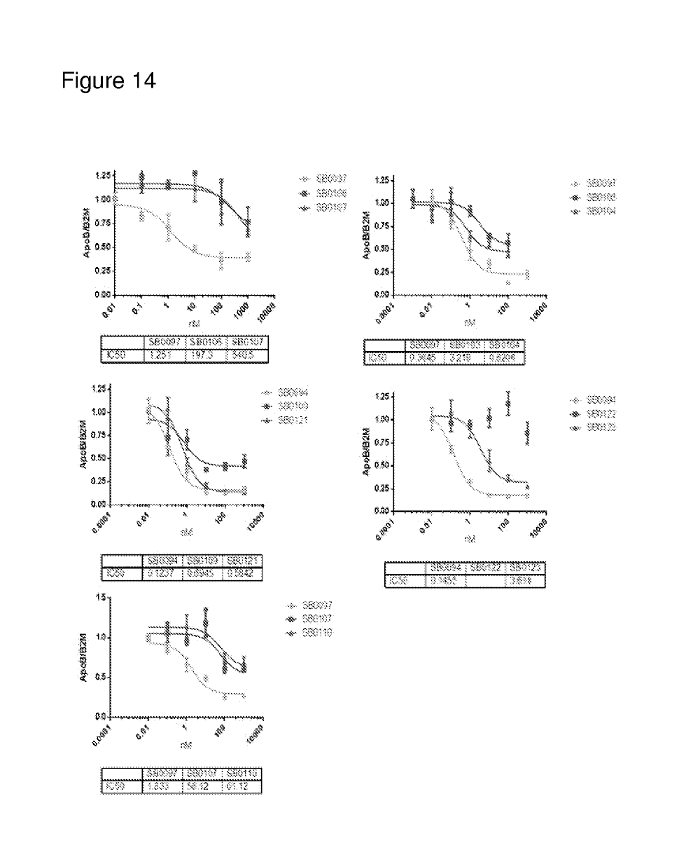

Primary mouse hepatocytes were isolated using the standard two-step collagenase perfusion technique (Li et. al.). Briefly, a 6-10 week old female C57/Bl6 mouse was anesthetized by intraperitoneal injection of a mixture of Ketamine (80-100 mg/kg)/Xylazine (5-10 mg/kg). The abdominal cavity was then exposed and the visceral vena cava was cannulated using a 22 G needle. The hepatic vein was severed and the liver was immediately perfused for 5-10 min using a solution of phosphate-buffered saline (PBS) containing 0.5 mM ETDA. This solution was immediately switched to a solution of collagenase (100 IU/ml) in Dulbecco's Modified Eagle's Medium (DMEM, Gibco) for another 5-10 min. At the end of perfusion, the liver was removed and the hepatocytes were collected in DMEM containing 10% fetal bovine serum at 4 C.°. The cells were then filtered through a 70 μm sterile filter, washed three times in the same solution, and cell viability was assessed using Trypan Blue staining. Cells were then seeded in 96-well plates coated with 0.1% rat tail collagen or 2% matrigel and incubated for 3-4 hours at 37 C.° in a 5% CO₂ incubator. Test compounds were then added to cells and incubated at 37 C.° in a 5% CO₂ incubator. At the end of the incubation period, the cells were lysed, the mRNA was isolated and the expression of the target gene was measured by qPCR and normalized to a house-keeping gene using standard protocols. The results are graphed in FIGS. 13 and 14. EC₅₀ values are provided in Table 8.

TABLE 8

| | ApoB | GAPDH |
|---|---|---|
| Compound # | EC₅₀ (nM) | EC₅₀ (nM) |
| SB00130 | 2 | |
| SB00141 | 6 | |
| SB0134 | 1 | |
| SB0142 | 2.5 | |
| SB0146 | | 0.2 |
| SB0147 | | 1 |
| SB0148 | | 0.05 |
| SB00150 | 172 | |
| (unconjugated 97) | | |
| SB0154 | 0.1 | |
| SB0155 | 0.2 | |
| SB0156 | 0.5 | |
| SB0157 | 0.2 | |
| SB0162 | 1 | |
| SB0163 | 2.5 | |
| SB0164 | 1.5 | |
| SB0234 | 0.5 | |
| SB0235 | 0.6 | |
| SB0236 | 3.2 | |

### Example 3

### Cell Binding Experiments

Disulfide Phosphotriester Oligonucleotide-Cy3 Cell Binding General Protocol:

polynucleotide constructs of the invention containing disulfide groups linked to one or more internucleotide bridging groups and/or terminal groups were annealed to G^{2′ Mod}-Cy3 (guide strand) at a final concentration of 10 mM.

Cell Treatment Setup:

40,000 cells were plated per well in a 48 well plate; cells were allowed to adhere overnight. Then, cells were washed once with 500 μl of PBS then 150 ul treatments were added (Note: for free folic acid samples, cells were treated with media containing 2.3 mM folic acid for 1 h prior to treatment). Cells were treated for 4 h; after 4 h, cells were washed once with PBS, trypsinized, and analyzed by flow cytometry for siRNA-Cy3 cell association.

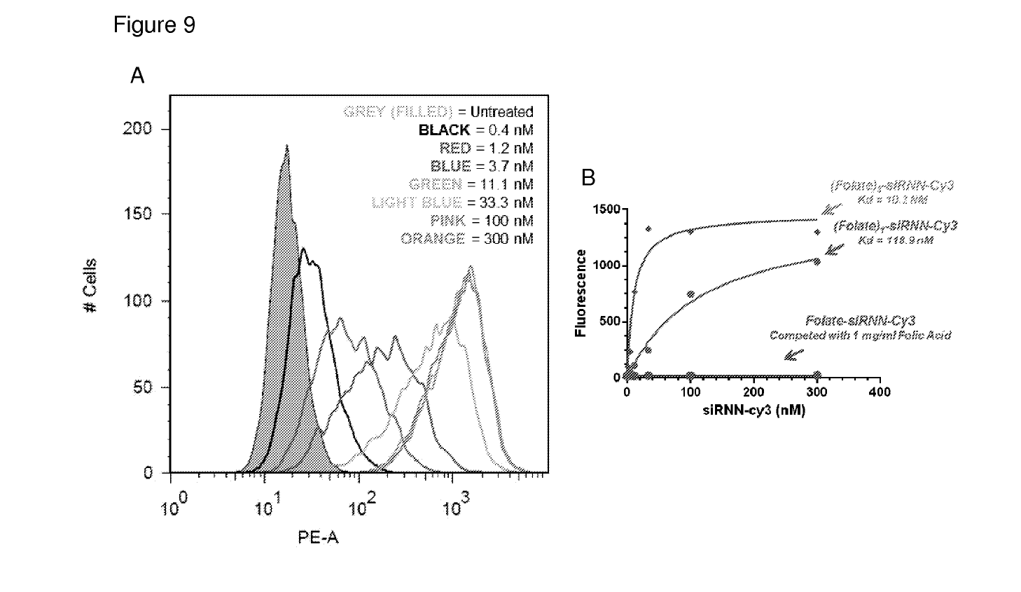

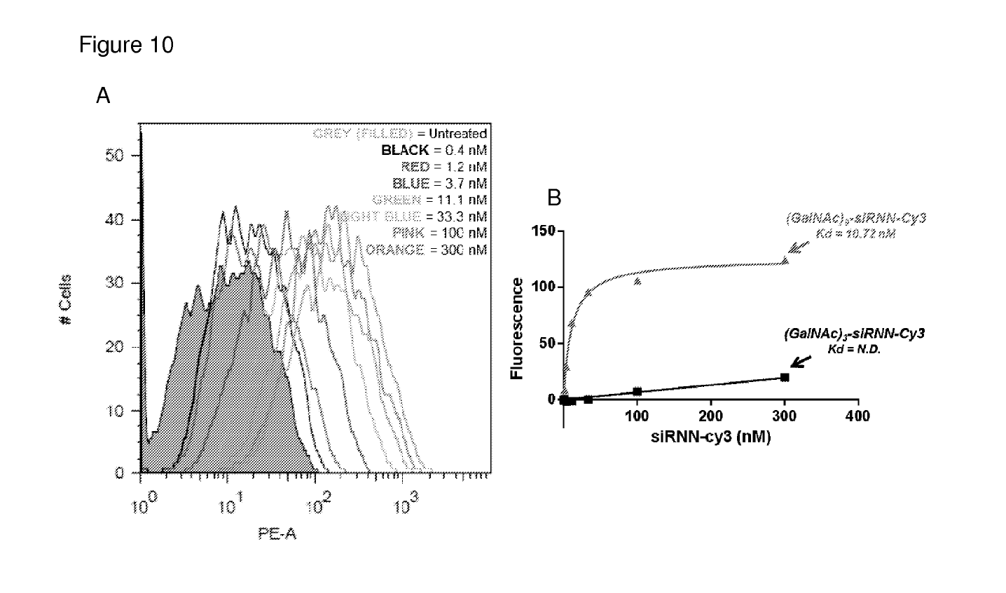

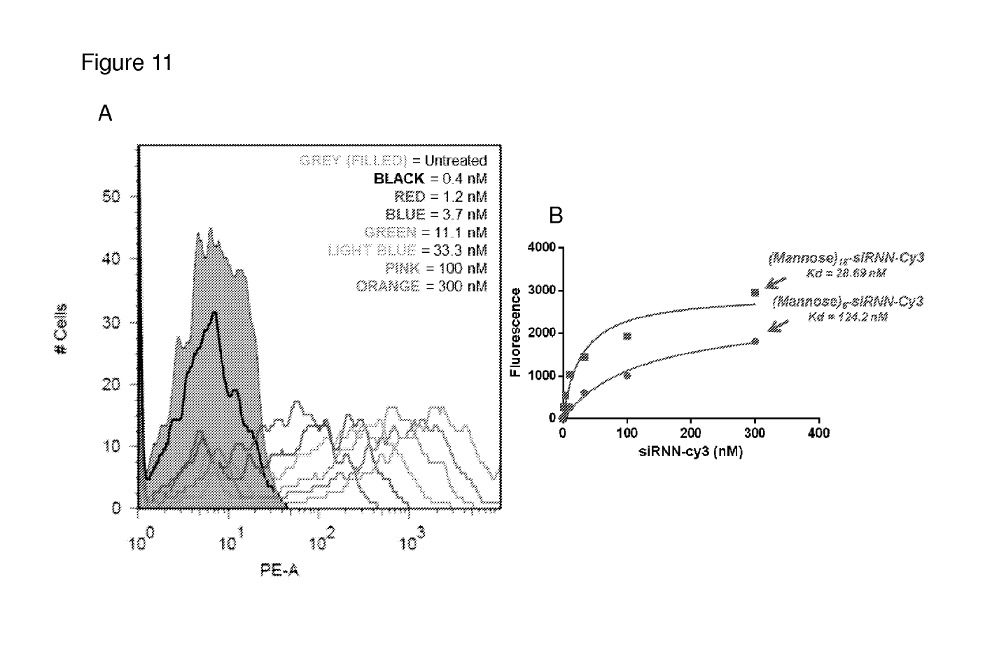

Results of these experiments are shown in FIGS. 9A, 9B, 10A, 10B, 11A, and 11B. FIG. 9A shows dose curves for (Folate)₃-siRNN-Cy3 conjugate binding to KB cell. FIG. 9B shows a graph determining dissociation constants (K_{d}) for (Folate)₃-siRNN-Cy3 and (Folate)₁-siRNN-Cy3 conjugates. FIG. 10A shows dose curves for (GalNAc)₉-siRNN-Cy3 conjugate binding to HepG2 cells. FIG. 10B shows a graph determining dissociation constants (K_{d}) for (GalNAc)₉-siRNN-Cy3 and (GalNAc)₃-siRNN-Cy3 conjugates. FIG. 11A shows dose curves for (Mannose)₁₈-siRNN-Cy3 conjugate binding to primary peritoneal macrophages. FIG. 11B shows a graph determining dissociation constants (K_{d}) for (Mannose)₁₈-siRNN-Cy3 and (Mannose)₆-siRNN-Cy3 conjugates.

### Example 4

### In Vivo Activity Assays

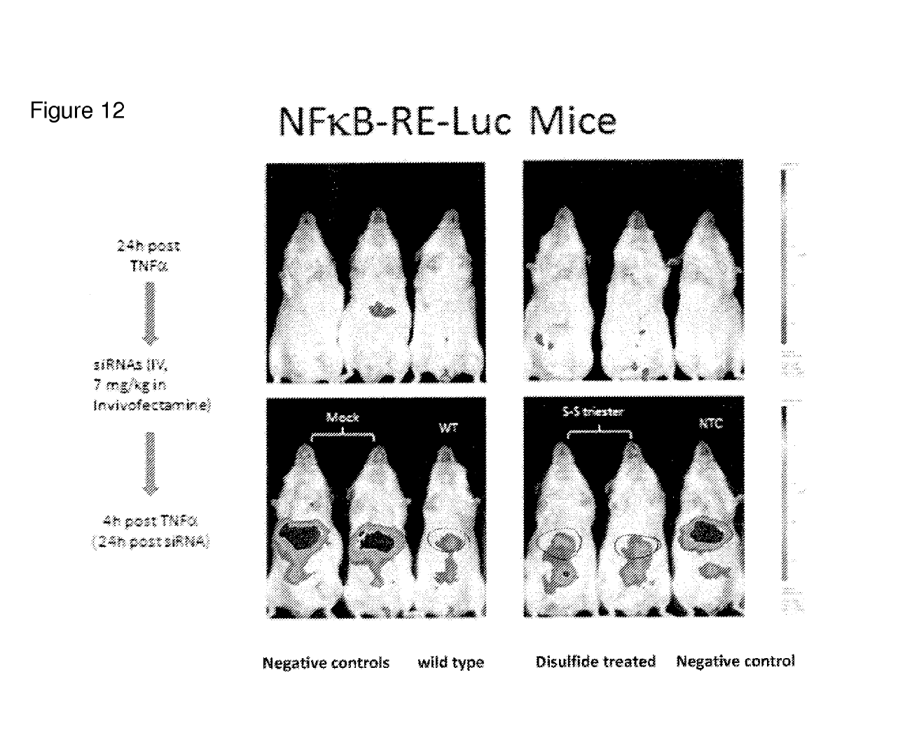

The in vivo activity of a luciferase disulfide phosphotriester molecule was tested using male NFκB-RE-Luc mice (Taconic). These mice express the luciferase gene (GL3) throughout the body, including the liver, and the luciferase activity is inducible by NFκB activators such as TNFα. Test agents (luciferase disulfide phosphotriester, wild-type luciferase siRNA sequence, and a non-targeting control siRNA sequence) were complexed with Invivofectamine 2.0 Reagent (Life Technologies) according to the manufacturer's recommendations and injected (˜200 μl, 7 mg/kg body weight) into the tail vein using sterile insulin syringes (n=1-2 mice/treatment). Two additional mice were injected with the same volume of vehicle and served as a mock treatment control. Twenty-four hours post injection, mice were subjected to intraperitoneal injection of murine TNFα (0.03 μg/g) to induce liver luciferase activity. Four hours after TNFα injection, mice were injected D-luciferin (150 mg/kg) intraperitoneally, and liver luciferase activity was measured using the IVIS Lumina whole body imager (Perkin Elmer) approximately 10 minutes after D-luciferin injection. Mice were imaged again 3, 6, and 8 days after siRNA administration to assess liver luciferase activity as described above. Results of this assay are shown in FIG. 12.

### In Vivo Experiments:

Test compounds were administered to female C57Bl6 mice via either subcutaneous or intravenous (lateral tail vein) injection (200 ul; 3 mice/treatment). At the appropriate time point post injection, mice were sacrificed and blood samples were collected by cardiac puncture. Approximately 50-100 mg piece of liver sample was collected and was immediately frozen in liquid nitrogen. Total mRNA was isolated from liver homogenates using standard protocols and the expression of target gene was quantitated by qPCR and normalized to a house-keeping gene using standard protocols.

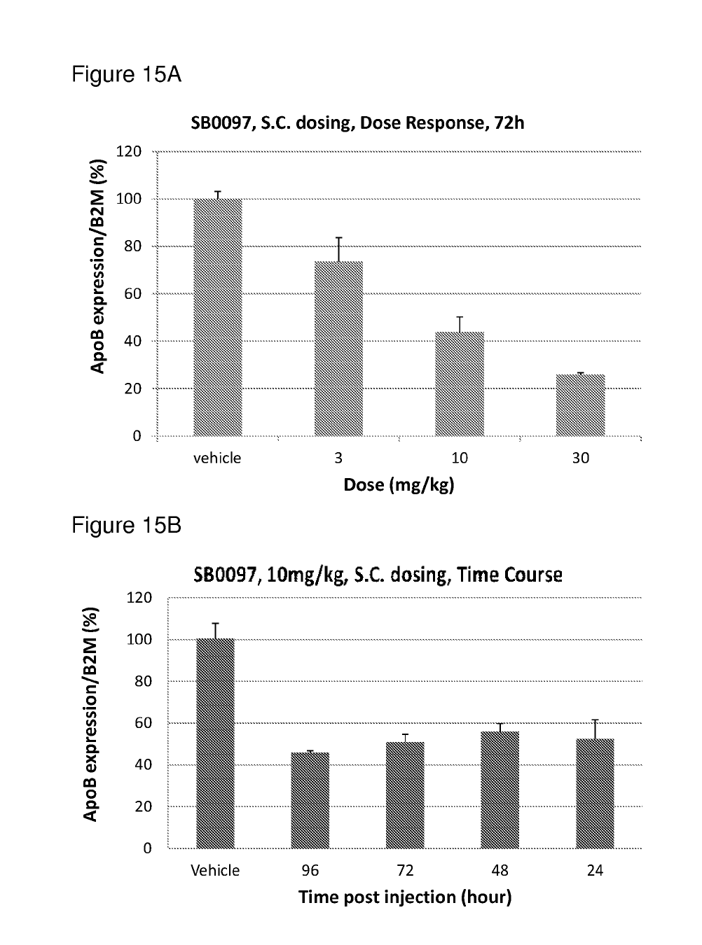

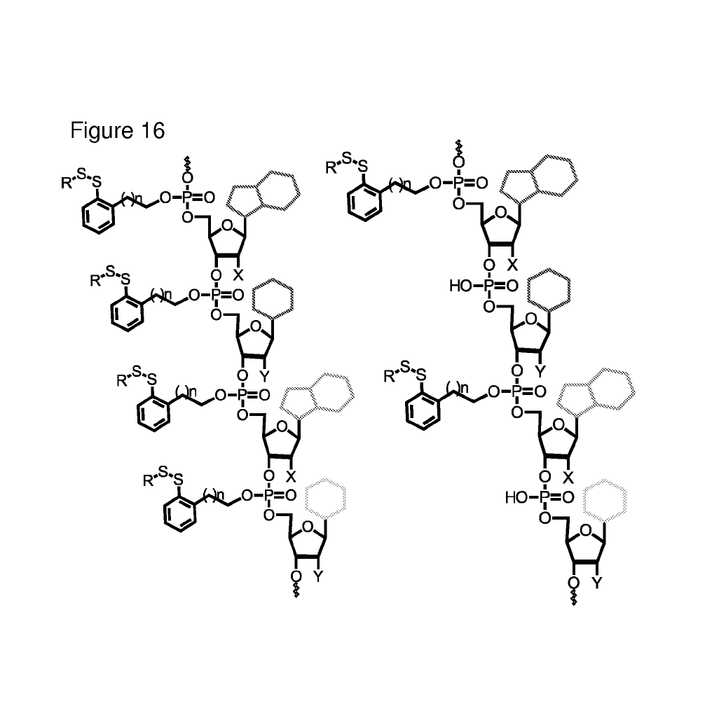

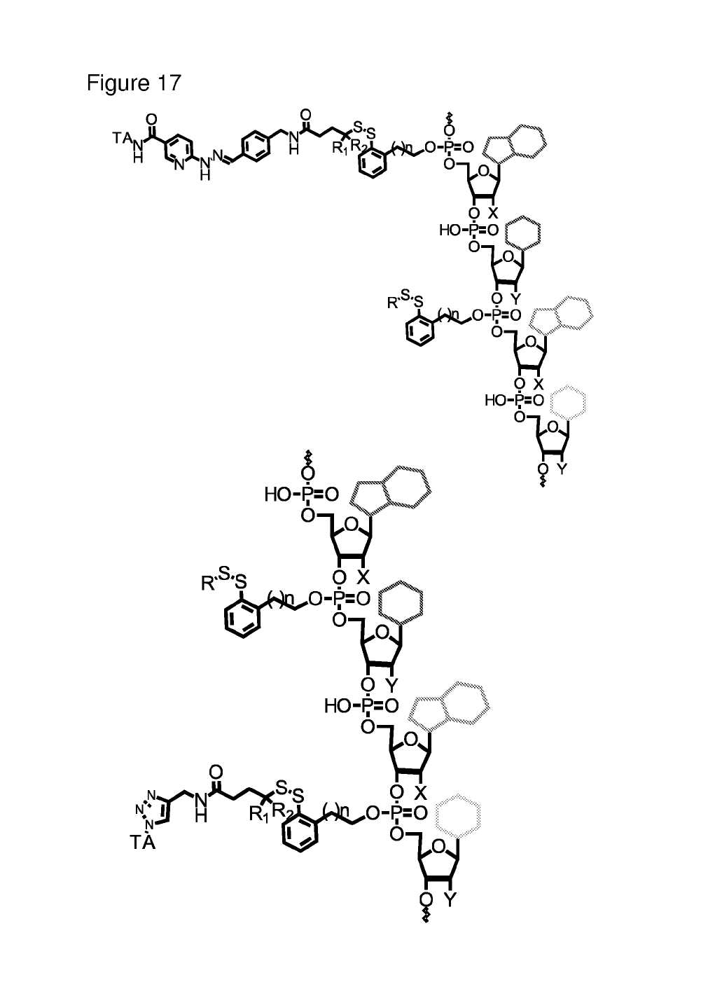

The results are shown in FIGS. 15A and 15B, For an exemplary procedure for isolation and culture of mouse hepatocytes, see: Li et al., Methods Mol. Biol., 633:185-196; 2010; the disclosure of which is incorporated herein by reference in its entirety.

### Pharmacology:

TABLE 9

| | % ApoB mRNA Remaining | | | |
|---|---|---|---|---|
| | S.C. dosing | | I.V. dosing | |
| Compound # | (mg/kg) | % SEM | (mg/kg) | % SEM |
| SB-0081 | 48.7 (30) | 3.8 | | |
| SB-0085 | 114 (30) | 8.6 | | |
| SB-0094 | 27.7 (30) | 0.8 | 32.5 (20) | 4.6 |
| SB-0094 | 63.2 (10) | 2.2 | | |
| SB-0095 | 91.8 (30) | 7 | | |
| SB-0096 | 25.0 (30) | 4.1 | | |
| SB-0097 | 22.3 (30) | 4.3 | 44.8 (20) | 2.8 |
| SB-0097 | 49.5 (10) | 1.3 | | |
| SB-0098 | 17.3 (30) | 2.6 | | |
| SB-0102 | 73.5 (15) | 3.9 | | |
| SB-0106 | 84.1 (10) | 6 | | |
| SB-0107 | 68.2 (10) | 3.8 | 53.3 (30) | 3.2 |
| SB-0108 | 78.5 (10) | 9.5 | | |
| SB-0109 | 97.8 (12.5) | 12.5 | 91.2 (20) | 11.9 |
| SB-0121 | 75.5 (10) | 1.5 | | |
| SB-0122 | 92.3 (20) | 6.5 | | |
| SB-0123 | 58.6 (20) | 8.2 | | |
| SB-0130 | | | 56.5 (4.3) | 6 |
| SB-0141 | | | 42.8 (4.3) | 7.8 |
| SB-0097 | 60.1 (10) | 7.9 | | |
| SB-0222 | 36.5 (10) | 9.3 | | |
| SB-0223 | 38 (10) | 1.6 | | |
| SB-0224 | 36.4 (10) | 5.2 | | |
| SB-0225 | 39.9 (10) | 1 | | |
| SB-0226 | 34.6 (10) | 2.2 | | |
| SB-0162 | | | 40.3 (10) | 1.5 |
| SB-0234 | | | 40.9 (10) | 3 |
| SB-0235 | | | 45.4 (10) | 3.3 |
| SB-0236 | | | 45.0 (10) | 5.5 |

### Example 5

### Mouse Primary Bone Marrow Progenitor Cells Isolation and In Vitro Experiments Using Macrophages

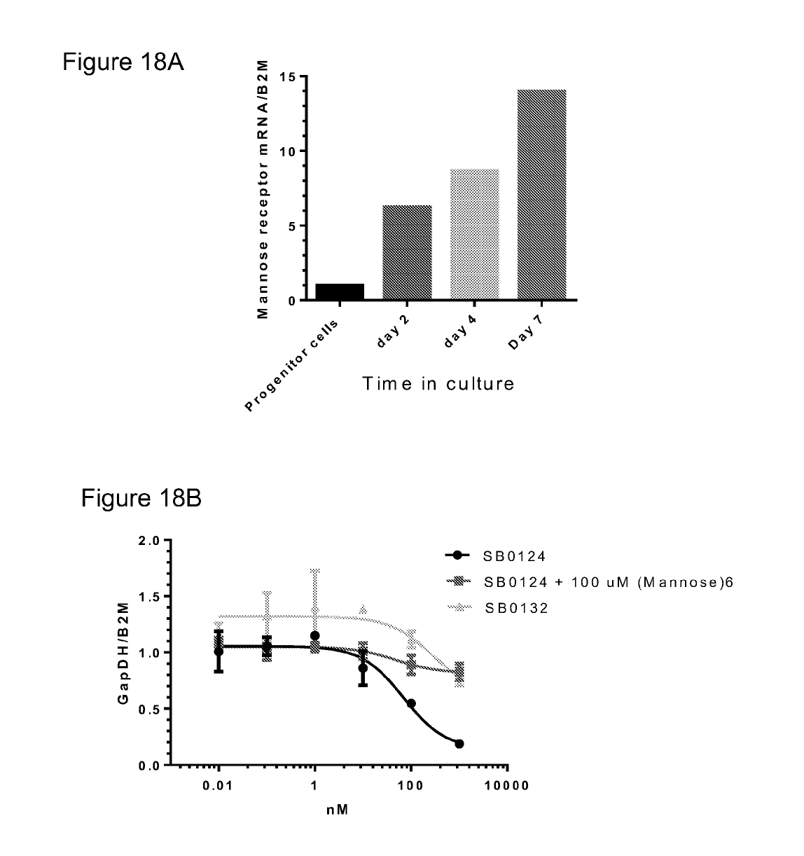

Mouse primary bone marrow progenitor cells were isolated from the femurs and tibias of C57Bl6 mice according to published protocols. Cells were immediately washed with PBS at 4° C. and suspended at 2×10⁶ cells/ml in RPMI containing 10% fetal calf serum and 20 ng/ml recombinant mouse M-CSF. Cells were seeded in 96-well plates and incubated for 7 days at 37° C., 5% CO₂ to allow differentiation to macrophages. Cells were washed every 24 hrs to remove potential non-macrophage cells contamination. Cells were used on day 7 based on mannose receptor expression. Mannose receptor expression over time is graphed in FIG. 18A. Test compounds from Table 4 were diluted in serum-free optiMEM and incubated with cells for 48 hrs. Cells were then lysed, total mRNA extracted and the expression of GAPDH gene was quantitated RTqPCR and normalized to a house-keeping gene. Results are shown in FIG. 18B.

### OTHER EMBODIMENTS

All publications, patents, and patent applications mentioned in the above specification are hereby incorporated by reference. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A polynucleotide construct comprising one or more components (i) comprising a disulfide linkage, wherein each of said one or more components is attached to an internucleotide bridging group or a terminal group of said polynucleotide construct, and each of said one or more components (i) comprises one or more bulky groups proximal to said disulfide group;
wherein when said one or more components (i) comprises an alkylene group linking said disulfide linkage to said terminal group, the number of atoms between said terminal group and said disulfide group is 2, 3, 4, or 5; and
wherein said one or more components (i) does not comprise an alkenylene within a group linking said disulfide linkage to said internucleotide bridging group.

2. A polynucleotide construct comprising one or more components (i) comprising a disulfide linkage, wherein each of said one or more components (i) is attached to an internucleotide bridging group or a terminal group of said polynucleotide construct, and each of said one or more components (i) comprises at least 4 atoms in a chain between said disulfide linkage and the phosphorus atom of said internucleotide bridging group or said terminal group; and
wherein said chain does not comprise a phosphate, an amide, an ester, or an alkenylene; and
wherein when said chain comprises an alkylene group, the number of atoms between said terminal group and said disulfide group is 4 or 5.

3. The polynucleotide of any one of claims 1 to 2, wherein at least one of said one or more components (i) comprises the following structure: 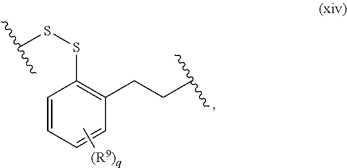
wherein
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, wherein q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and
each q is independently 0, 1, 2, 3, or 4.

4. The polynucleotide construct of claim 1 or 2, wherein each said one or more components (i), independently, comprises a group having the structure of (R⁴)ᵣ-L-A¹-S—S-A²-A³-A⁴-,
wherein A¹ a bond or a linker comprising one or more optionally substituted N, O, —S, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A comprises one or more of optionally substituted N, O, and S, none of said optionally substituted N, O, and S is directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;
A³ is selected from the group consisting of a bond, optionally substituted C₁₋₆ alkylene; optionally substituted C₃₋₈ cycloalkylene; optionally substituted C₃₋₈ cycloalkenylene; optionally substituted C₁₋₉ heterocyclylene having 1 to 4 heteroatoms selected from N, O, and S; optionally substituted C₆₋₁₄ arylene, optionally substituted C₁₋₉ heteroarylene having 1 to 4 heteroatoms selected from N, O, and S; O; optionally substituted N; and S;
A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;
L is absent or a conjugating group comprising one or more conjugating moieties; and
R⁴ is hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and a combination thereof;
wherein A⁴ is proximal to said internucleotide bridging group or said terminal group; and
wherein A¹ or A² comprises one or more bulky groups proximal to —S—S—.

5. The polynucleotide construct of claim 4, wherein said one or more components (i) consists of a group having the structure of (R⁴)ᵣ-L-A¹-S—S-A²-A³-A⁴-.

6. The polynucleotide of any one of claims 1 to 5, wherein at least one of said one or more components (i) further comprises one or more of a polypeptide, a carbohydrate, a neutral organic polymer, a positively charged polymer, a therapeutic agent, a targeting moiety, or an endosomal escape moiety;

7. The polynucleotide construct of any one of claims 1 to 6, wherein at least one of said one or more components (i) comprises a carbohydrate.

8. The polynucleotide construct of claim 7, wherein said carbohydrate comprises N-acetyl galactosamine or mannose.

9. The polynucleotide construct of any one of claims 1 to 8, wherein at least one of said one or more components (i) comprises a neutral organic polymer or a positively charged polymer.

10. The polynucleotide construct of claim 9, wherein said neutral organic polymer comprises 1 to 200 alkylene oxide units.

11. The polynucleotide construct of claim 10, wherein said alkylene oxide is ethylene glycol.

12. The polynucleotide construct of any one of claims 1 to 11, wherein at least one of said one or more components (i) comprises a targeting moiety.

13. The polynucleotide construct of claim 12, wherein said targeting moiety is a folate ligand.

14. The polynucleotide construct of any one of claims 1 to 13, wherein at least one of said one or more components (i) comprises a polypeptide.

15. The polynucleotide construct of claim 14, wherein said polypeptide comprises a protein transduction domain.

16. The polynucleotide construct of any one of claims 1 to 15, wherein at least one of said one or more components (i) comprises an endosomal escape moiety.

17. The polynucleotide construct of any one of claims 1 to 16, wherein said polynucleotide construct comprises 2 to 150 nucleotides.

18. The polynucleotide construct of claim 17, wherein said polynucleotide construct comprises 5 to 50 nucleotides.

19. The polynucleotide construct of claim 18, wherein said polynucleotide construct comprises 8 to 40 nucleotides.

20. The polynucleotide construct of claim 19, wherein said polynucleotide construct comprises 10 to 32 nucleotides.

21. The polynucleotide construct of any one of claims 1 to 20, wherein said disulfide linkage is not bonded to pyridyl.

22. A polynucleotide construct having the structure of Formula I: 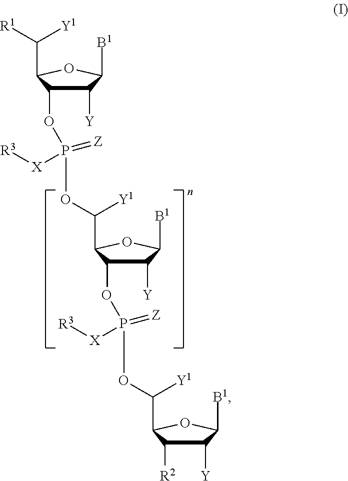
or a salt thereof,
wherein n is a number from 0 to 150;
each B¹ is independently a nucleobase;
each X is independently selected from the group consisting of O, S, and optionally substituted N;
each Y is independently selected from the group consisting of hydrogen, hydroxyl, halo, optionally substituted C₁₋₆ alkoxy, and a protected hydroxyl group;
each Y¹ is independently H or optionally substituted C₁₋₆ alkyl;
each Z is independently O or S;
R¹ is selected from the group consisting of H, hydroxyl, optionally substituted C₁₋₆ alkoxy, a protected hydroxyl group, a monophosphate, a diphosphate, a triphosphate, a tetraphosphate, a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and any combination thereof, or R¹ is 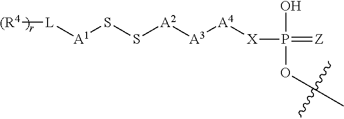
or a salt thereof;
R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an optionally substituted C₁₋₆ alkyl, an amino containing group, a biotin containing group, a digoxigenin containing group, a cholesterol containing group, a quencher containing group, a phosphothiol, a polypeptide, a carbohydrate, a neutral organic polymer, a positively charged polymer, a therapeutic agent, a targeting moiety, an endosomal escape moiety, and any combination thereof, or R² is 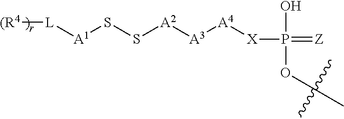
or a salt thereof; and
each R³ is independently absent, a hydrogen, optionally substituted C₁₋₆ alkyl, or a group having the structure of Formula II: 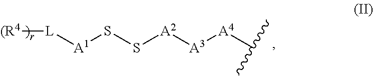
wherein each A¹ is independently a bond or a linker comprising one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ comprises one or more of optionally substituted N, O, and S, said optionally substituted N, O, or S is not directly bonded to the disulfide; and each A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;
each A³ is independently selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;
each A⁴ is independently selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;
each L is independently absent or a conjugating group comprising one or more conjugating moieties;
each R⁴ is independently hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and any combination thereof;
each r is independently an integer from 1 to 10;
wherein, in at least one of R¹, R², and R³, A², A³, and A⁴ combine to form a group having at least three atoms in the shortest chain connecting —S—S— and X;
wherein at least one R³ has the structure of formula (II) or at least one of R¹ and R² is 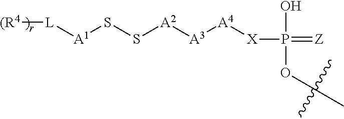
or a salt thereof; and
wherein,
when R¹ or R² is 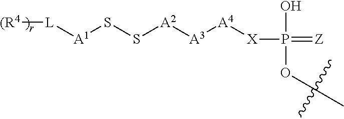
or a salt thereof in which A², A³, and A⁴ combine to form an alkylene group, said alkylene group is C₄₋₅ alkylene; or
when R¹ or R² is 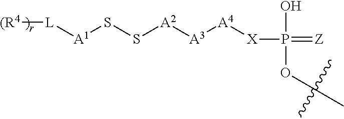
or a salt thereof, the group -A²-A³-A⁴-X— does not comprise an amide, an ester, or an alkenylene.

23. The polynucleotide construct of claim 22, wherein —S—S-A²-A³-A⁴- group has the following structure: 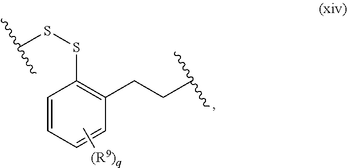
wherein
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, wherein q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and
each q is independently 0, 1, 2, 3, or 4.

24. The polynucleotide construct of claim 22 or 23, wherein each X is O, and/or each Z is O.

25. The polynucleotide construct of any one of claims 22 to 24, wherein, when a nucleoside is linked via its 3′-O—P—X— chain to R³ having the structure of formula (II), Y of said nucleoside is halo, optionally substituted C₁₋₆ alkoxy, or hydroxyl.

26. The polynucleotide construct of claim 25, wherein at least one Y is F.

27. The polynucleotide construct of claim 25, wherein at least one Y is OMe.

28. The polynucleotide construct of any one of claims 22 to 27, wherein R⁴ is bound to L, to A¹, or to disulfide via a bond formed by a reaction selected from the group consisting of a pericyclic reaction; an alkylation or arylation of a hydroxyl, thiol, or amino moiety; and a reaction of a hydroxyl, thiol, or amino nucleophile with an electrophile.

29. The polynucleotide construct of claim 28, wherein said pericyclic reaction is a cycloaddition.

30. The polynucleotide construct of claim 29, wherein said cycloaddition is Hüisgen cycloaddition.

31. The polynucleotide construct of any one of claims 22 to 27, wherein R⁴ is bound to L, to A¹, or to the disulfide via an amide bond, a sulfonamide bond, a carboxylic ester, a thioester, optionally substituted C₆₋₁₄ aryl, optionally substituted C₁₋₉ heterocyclyl having 1 to 4 heteroatoms selected from N, O, and S; optionally substituted C₁₋₉ heteroaryl having 1 to 4 heteroatoms selected from N, O, and S; an imine; a hydrazone; an oxime; or a succinimide.

32. The polynucleotide construct of any one of claims 22 to 31, wherein one or more of the hydrophilic functional groups and conjugating moieties are protected with protecting groups.

33. The polynucleotide construct of any one of claims 22 to 32, wherein L is formed through a condensation reaction with an aldehyde conjugating moiety to form an imine, enamine, oxime, or hydrazone bond.

34. The polynucleotide construct of any one of claims 22 to 33, wherein at most 90% of the disulfides are linked to one or more auxiliary moieties.

35. The polynucleotide construct of claim 34, wherein at most 75% of the disulfides are linked to one or more auxiliary moieties.

36. The polynucleotide construct of claim 35, wherein at most 50% of the disulfides are linked to one or more auxiliary moieties.

37. The polynucleotide construct of claim 36, wherein at most 25% of the disulfides are linked to one or more auxiliary moieties.

38. The polynucleotide construct of any one of claims 22 to 37, wherein at most 75% of the nucleotides in said polynucleotide construct are linked to the disulfide.

39. The polynucleotide construct of claim 38, wherein at most 65% of the nucleotides in said polynucleotide construct are linked to the disulfide.

40. The polynucleotide construct of claim 39, wherein at most 45% of the nucleotides in said polynucleotide construct are linked to the disulfide.

41. The polynucleotide construct of claim 40, wherein at most 25% of the nucleotides in said polynucleotide construct are linked to the disulfide.

42. The polynucleotide construct of any one of claims 22 to 41, wherein said polynucleotide construct comprises 1 to 100 groups formula (II).

43. The polynucleotide construct of claim 42, wherein said polynucleotide construct comprises 2 to 50 groups of formula (II).

44. The polynucleotide construct of claim 43, wherein said polynucleotide construct comprises 2 to 30 groups of formula (II).

45. The polynucleotide construct of claim 44, wherein said polynucleotide construct comprises 2 to 10 groups of formula (II).

46. The polynucleotide construct of any one of claims 22 to 45, wherein said polynucleotide construct comprises 5 to 50 nucleotides.

47. The polynucleotide construct of claim 46, wherein said polynucleotide construct comprises 8 to 40 nucleotides.

48. The polynucleotide construct of claim 47, wherein said polynucleotide construct comprises 10 to 32 nucleotides.

49. The polynucleotide construct of any one of claims 22 to 48, wherein at least one R⁴ comprises a targeting moiety.

50. The polynucleotide construct of any one of claims 22 to 49, wherein at least one R⁴ comprises a carbohydrate.

51. The polynucleotide construct of any one of claims 22 to 50, wherein at least one R⁴ comprises mannose.

52. The polynucleotide construct of any one of claims 22 to 51, wherein at least one R⁴ comprises N-acetyl galactosamine.

53. The polynucleotide construct of any one of claims 22 to 52, wherein at least one R⁴ comprises a group comprising a folate ligand.

54. The polynucleotide construct of any one of claims 22 to 53, wherein at least one R⁴ comprises a protein transduction domain.

55. The polynucleotide construct of any one of claims 22 to 54, wherein at least one R⁴ comprises an endosomal escape moiety.

56. The polynucleotide construct of any one of claims 22 to 55, wherein at least one R⁴ comprises a prostate specific membrane antigen (PSMA).

57. The polynucleotide construct of any one of claims 22 to 56, wherein the ratio of R³ groups that are absent or H to R³ groups that have the structure of formula (II) is from 1:10 to 10:1.

58. The polynucleotide construct of claim 57, wherein the ratio of R³ groups that are absent or H, to R³ groups that have the structure of formula (II) is from 1:5 to 5:1.

59. The polynucleotide construct of claim 58, wherein the ratio of R³ groups that are absent or H, to R³ groups that have the structure of formula (II) is from 1:3 to 3:1.

60. The polynucleotide construct of claim 59, wherein the ratio of R³ groups that are absent or H, to R³ groups that have the structure of formula (II) is from 1:2 to 2:1.

61. The polynucleotide construct of claim 60, wherein the ratio of R³ groups that are absent or H, to R³ groups that have the structure of formula (II) is about 1:1.

62. The polynucleotide construct of any one of claims 22 to 61, wherein L comprises 1 to 500 monomers, each of said monomers is, independently, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; carbonyl; thiocarbonyl; imino; optionally substituted N; O; or S(O)ₘ, wherein m is 0, 1, or 2.

63. The polynucleotide construct of any one of claims 22 to 62, wherein L comprises one or more C₁₋₆ alkyleneoxy groups.

64. The polynucleotide construct of claim 63, wherein L comprises fewer than 100 C₁₋₆ alkyleneoxy groups.

65. The polynucleotide construct of any one of claims 22 to 64, wherein L comprises one or more ethyleneoxy groups.

66. The polynucleotide construct of claim 65, wherein L comprises fewer than 100 ethyleneoxy groups.

67. The polynucleotide construct of any one of claims 22 to 64, wherein L comprises one or more poly(alkylene oxide).

68. The polynucleotide construct of claim 67, wherein said poly(alkylene oxide) is selected from polyethylene oxide, polypropylene oxide, poly(trimethylene oxide), polybutylene oxide, poly(tetramethylene oxide), and diblock or triblock co-polymers thereof.

69. The polynucleotide construct of claim 67 or 68, wherein said poly(alkylene oxide) is polyethylene oxide.

70. The polynucleotide construct of any one of claims 22 to 69, wherein L comprises one or more amino acid residues.

71. The polynucleotide construct of claim 70, wherein at least one of said amino acid residues is selected from the group consisting of Arg, Asn, Asp, Cys, Glu, Gin, His, Lys, Ser, Thr, Trp, and Tyr.

72. The polynucleotide construct of any one of claims 22 to 71, wherein L comprises a group having the structure of formula (III): 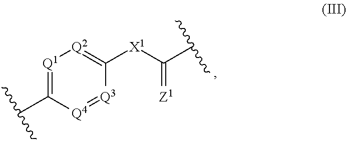
wherein each Q¹, Q², Q³, and Q⁴ is independently N or CR⁷;
X¹ is O or NR⁶;
Z¹ is O or S;
each R⁷ is independently selected from the group consisting of H; optionally substituted C₁₋₆ alkyl; optionally substituted C₂₋₆ alkenyl; optionally substituted C₂₋₆ alkynyl; halo; hydroxyl; —CHO; optionally substituted C₁₋₆ alkanoyl; carboxyl; cyano; nitro; amino; thiol; optionally substituted C₁₋₉ heterocyclyl having 1 to 4 heteroatoms selected from N, O, and S; optionally substituted C₁₋₉ heteroaryl having 1 to 4 heteroatoms selected from N, O, and S; optionally substituted C₆₋₁₄ aryl; optionally substituted C₃₋₈ cycloalkyl; and optionally substituted C₃₋₈ cycloalkenyl.

73. The polynucleotide construct of claim 72, wherein Q¹ is CR⁷.

74. The polynucleotide construct of claim 72 or 73, wherein Q² is CR⁷.

75. The polynucleotide construct of any one of claims 72 to 74, wherein Q³ is CR⁷.

76. The polynucleotide construct of any one of claims 72 to 75, wherein Q⁴ is CR⁷.

77. The polynucleotide construct of any one of claims 72 to 76, wherein each R⁷ is independently H, optionally substituted C₁₋₆ alkyl, or halo.

78. The polynucleotide construct of claim 77, wherein R⁷ is H.

79. The polynucleotide construct of any one of claims 72 to 78, wherein X¹ is NR⁶.

80. The polynucleotide construct of any one of claims 72 to 79, wherein Z¹ is S.

81. The polynucleotide construct of any one of claims 22 to 80, wherein L comprises one or more groups having the structure of formula (IV): 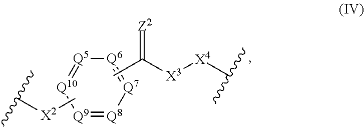
wherein each Q⁵, Q⁶, Q⁷, Q⁸, Q⁹, and Q¹⁰ is, independently, N, CR⁷, or C bonded to —X² or —C(Z²)X³X⁴, wherein one and only one of Q, Q⁶, Q⁷, Q⁸, Q⁹, and Q¹⁰ is C bonded to —X², and one and only one of Q⁵, Q⁶, Q⁷, Q⁸, Q⁹, and Q¹⁰ is C bonded to —C(Z²)X³X⁴;
X² is optionally substituted C₁₋₆ alkylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted diazaalkenylene; optionally substituted saturated diaza; unsaturated diaza; optionally substituted azacarbonyl; or oxacarbonyl;
X³ is a bond, O, NR⁷, or S;
X⁴ is absent, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O; optionally substituted C₁₋₉ heterocyclylene having 1 to 4 heteroatoms selected from N, O, and S; or optionally substituted (C₁₋₉ heterocyclyl)-C₁₋₄-alkylene having 1 to 4 heteroatoms selected from N, O, and S; and
Z² is O, S, or NR⁷; and
each R⁷ is independently selected from the group consisting of H, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; and optionally substituted C₁₋₆ alkoxy; and
wherein the two of Q⁵, Q⁶, Q⁷, Q⁸, Q⁹, and Q¹⁰ linked to X² and —C(Z¹)X³X⁴ are not N.

82. The polynucleotide construct of claim 81, wherein Q⁵ is N.

83. The polynucleotide construct of claim 81 or 82, wherein Q⁶ is CR⁷.

84. The polynucleotide construct of any one of claims 81 to 83, wherein Q⁷ is C bonded to —C(Z²)X³X⁴.

85. The polynucleotide construct of any one of claims 81 to 84, wherein Q⁸ is CR⁷.

86. The polynucleotide construct of any one of claims 81 to 85, wherein Q⁹ is CR⁷.

87. The polynucleotide construct of any one of claims 81 to 86, wherein Q¹⁰ is C bonded to X².

88. The polynucleotide construct of any one of claims 81 to 87, wherein each R⁷ is independently selected from the group consisting of H, halo, and optionally substituted C₁₋₆ alkyl.

89. The polynucleotide construct of claim 88, wherein R⁷ is H.

90. The polynucleotide construct of any one of claims 81 to 89, wherein X² is optionally substituted diazaalkenylene or optionally substituted saturated diaza.

91. The polynucleotide construct of any one of claims 81 to 90, wherein X³ is NR⁷.

92. The polynucleotide construct of any one of claims 81 to 91, wherein X⁴ is absent.

93. The polynucleotide construct of any one of claims 81 to 92, wherein Z² is O.

94. The polynucleotide construct of any one of claims 22 to 93 wherein L comprises one or more groups having the formulae (IVa) or (IVb): 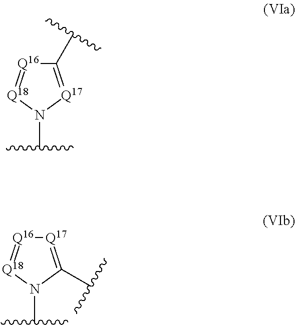
wherein each of Q¹⁶, Q¹⁷, and Q¹⁸ is independently N or CR⁷;
each of R⁷ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁₋₆ alkyl, C₆₋₁₀ aryl, and (C₆₋₁₀ aryl)-C₁₋₄-alkyl.

95. The polynucleotide construct of claim 94, wherein Q¹⁶ is N.

96. The polynucleotide construct of any one of claim 94 or 95, wherein Q¹⁸ is N.

97. The polynucleotide construct of any one of claims 94 to 96, wherein Q¹⁷ is CR⁷.

98. The polynucleotide construct of claim 97, wherein R⁷ is H, halo, or C₁₋₆ alkyl.

99. The polynucleotide construct of claim 98, wherein R⁷ is H.

100. The polynucleotide construct of any one of claims 22 to 93, wherein L comprises one or more groups having the structure:

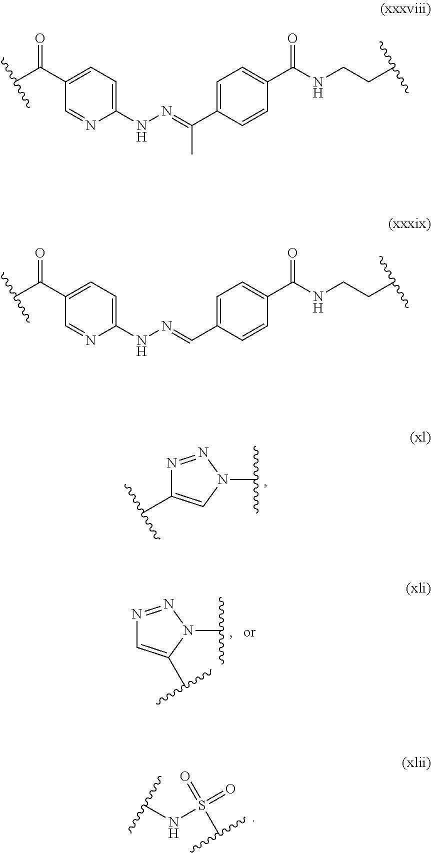 [SMILES: *CCNC(=O)c1ccc(/C(C)=N/Nc2ccc(C(*)=O)cn2)cc1 ; *CCNC(=O)c1ccc(/C=N/Nc2ccc(C(*)=O)cn2)cc1 ; *c1cn(*)nn1 ; *c1cnnn1* ; *NS(*)(=O)=O]

101. The polynucleotide construct of any one of claims 22 to 61, wherein L is a bond.

102. The polynucleotide construct of any one of claims 22 to 101, wherein A³ is selected from the group consisting of a bond, optionally substituted C₁₋₆ alkylene; optionally substituted C₆₋₁₄ arylene; O; optionally substituted N; and S.

103. The polynucleotide construct of any one of claims 22 to 102, wherein A³ is selected from the group consisting of a bond, optionally substituted C₁₋₆ alkylene; optionally substituted C₆₋₁₄ arylene; and O.

104. The polynucleotide construct of claim 103, wherein A³ has a structure of formula (VI): 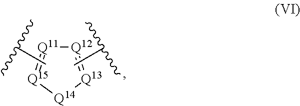
wherein
Q¹¹ is N, or C bonded to R¹⁰ or to A²;
Q¹² is N, or C bonded to R¹¹ or to A⁴;
Q¹³ is N or C bonded to R¹² or to A⁴;
Q¹⁴ is O, S, N bonded to R¹³ or to A⁴, or —C(R¹⁴ or bond to A⁴)=C(R¹⁵ or bond to A⁴)-;
Q¹⁵ is N, or C bonded to R¹⁶ or to A²;
each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁₋₆ alkyl, C₆₋₁₀ aryl, and (C₆₋₁₀ aryl)-C₁₋₄-alkyl; and
wherein
one and only one of Q¹¹ and Q¹⁵ is bonded to A², and
one and only one of Q¹², Q¹³, and Q¹⁴ is bonded to A⁴.

105. The polynucleotide construct of claim 104, wherein Q¹¹ is C bonded to A².

106. The polynucleotide construct of claim 104 or 105, wherein Q¹² is C bonded to A⁴.

107. The polynucleotide construct of any one of claims 104 to 106, wherein Q¹³ is C bonded to R¹².

108. The polynucleotide construct of claim 107, wherein R¹² is H, halo, or C₁₋₆ alkyl.

109. The polynucleotide construct of any one of claims 104 to 108, wherein Q¹⁴ is O.

110. The polynucleotide construct of any one of claims 104 to 109, wherein Q¹⁴ is —C(R¹⁴)═C(R¹⁵)—.

111. The polynucleotide construct of claim 110, wherein R¹⁴ is H, halo, or C₁₋₆ alkyl.

112. The polynucleotide construct of claim 110 or 111, wherein R¹⁵ is H, halo, or C₁₋₆ alkyl.

113. The polynucleotide construct of any one of claims 104 to 112, wherein Q¹⁵ is C bonded to R¹⁶.

114. The polynucleotide construct of claim 113, wherein R¹⁶ is H, halo, or C₁₋₆ alkyl.

115. The polynucleotide construct of any one of claims 22 to 114, wherein A⁴ is optionally substituted C₁₋₆ alkylene.

116. The polynucleotide construct of any one of claims 22 to 115, wherein A¹ comprises a group having the structure:

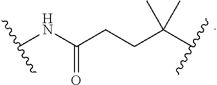 [SMILES: *NC(=O)CCC(*)(C)C]

117. The polynucleotide construct of any one of claims 22 to 115, wherein
A¹ is a bond or comprises one or more groups selected independently from the group consisting of optionally substituted C₁₋₆ alkylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; optionally substituted (C₁₋₉ heterocyclyl)-C₁₋₄-alkylene having 1 to 4 heteroatoms selected from N, O, and S; optionally substituted N; and O.

118. The polynucleotide construct of claim 117, wherein A¹ is a bond or comprises one or more groups selected independently from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

119. The polynucleotide construct of claim 118, wherein A¹ is a bond or comprises one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

120. The polynucleotide construct of claim 119, wherein A¹ is a bond or comprises one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

121. The polynucleotide construct of any one of claims 22 to 115, wherein A¹ is a bond.

122. The polynucleotide construct of any one of claims 22 to 121, wherein A² is optionally substituted C₁₋₆ alkylene, optionally substituted C₃₋₈ cycloalkylene; optionally substituted C₃₋₈ cycloalkenylene; optionally substituted C₆₋₁₄ arylene; or optionally substituted C₁₋₉ heteroarylene having 1 to 4 heteroatoms selected from N, O, and S.

123. The polynucleotide construct of claim 122, wherein A² is optionally substituted C₁₋₆ alkylene, optionally substituted C₃₋₈ cycloalkylene; optionally substituted C₆₋₁₄ arylene; or optionally substituted C₁₋₉ heteroarylene having 1 to 4 heteroatoms selected from N, O, and S.

124. The polynucleotide construct of claim 123, wherein A² is optionally substituted optionally substituted C₆₋₁₄ arylene or optionally substituted C₁₋₉ heteroarylene having 1 to 4 heteroatoms selected from N, O, and S.

125. The polynucleotide construct of claim 124, wherein A² has a structure of formula (VI): 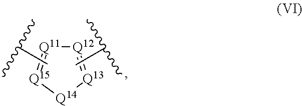
wherein
Q¹¹ is N, or C bonded to R¹⁰ or to the disulfide linkage;
Q¹² is N, or C bonded to R¹¹ or to A³;
Q¹³ is N or C bonded to R¹² or to A³;
Q¹⁴ is O, S, N bonded to R¹³ or to A³, or —C(R¹⁴ or bond to A³)=C(R¹⁵ or bond to A³)-;
Q¹⁵ is N, or C bonded to R¹⁶ or to the disulfide linkage;
each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and
wherein
one and only one of Q¹¹ and Q¹⁵ is bonded to the disulfide linkage, and
one and only one of Q¹², Q¹³, and Q¹⁴ is bonded to A³.

126. The polynucleotide construct of claim 125, wherein Q¹¹ is C bonded to the disulfide linkage.

127. The polynucleotide construct of claim 125 or 126, wherein Q¹² is C bonded to A³.

128. The polynucleotide construct of any one of claims 125 to 127, wherein Q¹³ is C bonded to R¹².

129. The polynucleotide construct of claim 128, wherein R¹² is H, halo, or C₁₋₆ alkyl.

130. The polynucleotide construct of any one of claims 125 to 129, wherein Q¹⁴ is O.

131. The polynucleotide construct of any one of claims 125 to 130, wherein Q¹⁴ is —C(R¹⁴)═C(R¹⁵)—.

132. The polynucleotide construct of claim 131, wherein R¹⁴ is H, halo, or C₁₋₆ alkyl.

133. The polynucleotide construct of claim 131 or 132, wherein R¹⁵ is H, halo, or C₁₋₆ alkyl.

134. The polynucleotide construct of any one of claims 125 to 133, wherein Q¹⁵ is C bonded to R¹⁶.

135. The polynucleotide construct of claim 134, wherein R¹⁶ is H, halo, or C₁₋₆ alkyl.

136. The polynucleotide construct of any one of claims 22 to 135, wherein, when the carbon atom attached to the sulfur atom of —S—S-A²-A³-A⁴- is an alkylene carbon atom, said alkylene carbon atom is connected to at most one hydrogen atom.

137. The polynucleotide construct of any one of claims 22 to 136, wherein, when the carbon atom attached to the sulfur atom of —S—S-A²-A³-A⁴- is an alkylene carbon atom, said alkylene carbon atom is not connected to a hydrogen atom.

138. The polynucleotide construct of any one of claims 22 to 135, wherein, when the carbon atom attached to the sulfur atom of —S—S-A²-A³-A⁴- is an alkenylene carbon atom, said alkenylene carbon atom is not connected to a hydrogen atom.

139. The polynucleotide construct of any one of claims 22 to 135, wherein the carbon atom attached to the sulfur atom of —S—S-A²-A³-A⁴- is not an alkynylene carbon atom.

140. The polynucleotide construct of any one of claims 22 to 139, wherein, when the carbon atom attached to the sulfur atom of (R⁴)ᵣ-L-A¹-S—S— is an alkylene carbon atom, said carbon atom is connected to at most one hydrogen atom.

141. The polynucleotide construct of any one of claims 22 to 139, wherein, when the carbon atom attached to the sulfur atom of (R⁴)ᵣ-L-A¹-S—S— group is an alkylene carbon atom, said carbon atom is not connected to a hydrogen atom.

142. The polynucleotide construct of any one of claims 22 to 115, wherein A¹ and A² together with —S—S— to which they are attached, join to form an optionally substituted 5 to 16 membered ring.

143. The polynucleotide construct of claim 142, wherein A¹ and A², together with —S—S— to which they are attached, join to form an optionally substituted 5 to 7 membered ring.

144. The polynucleotide construct of any one of claims 22 to 141, wherein A¹, A², A³, and A⁴ or A², A³, and A⁴ and the disulfide linkage combine to form a group having the structure of any one of: 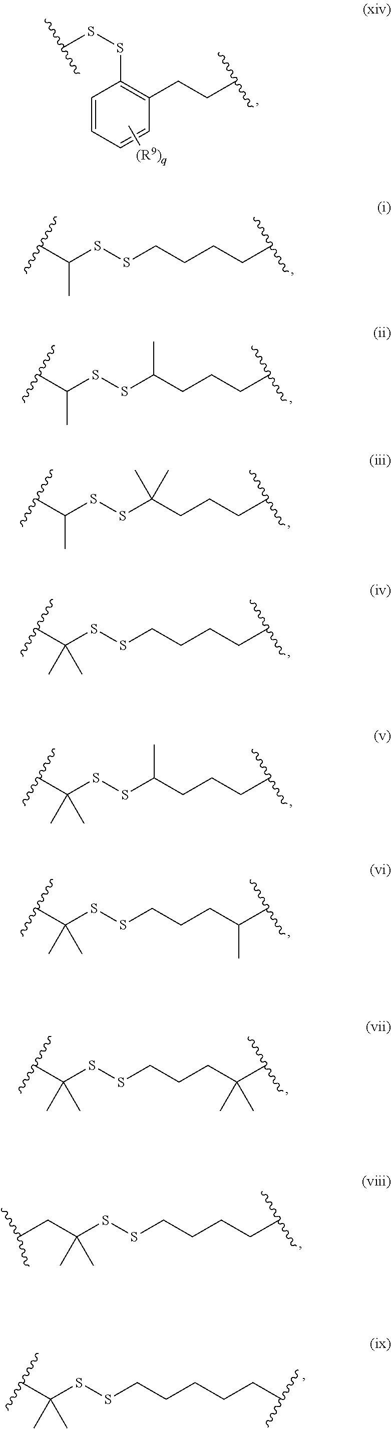 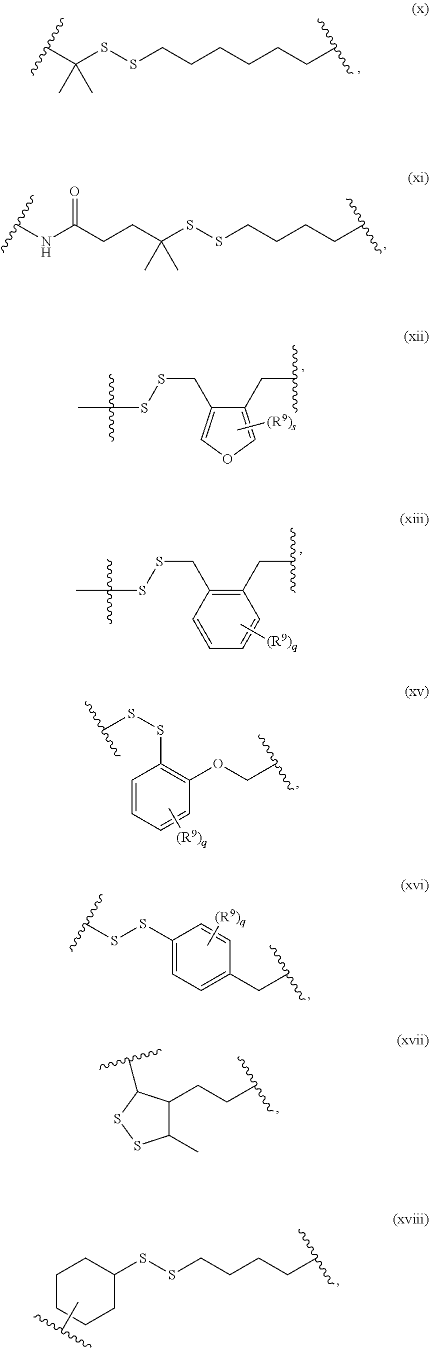 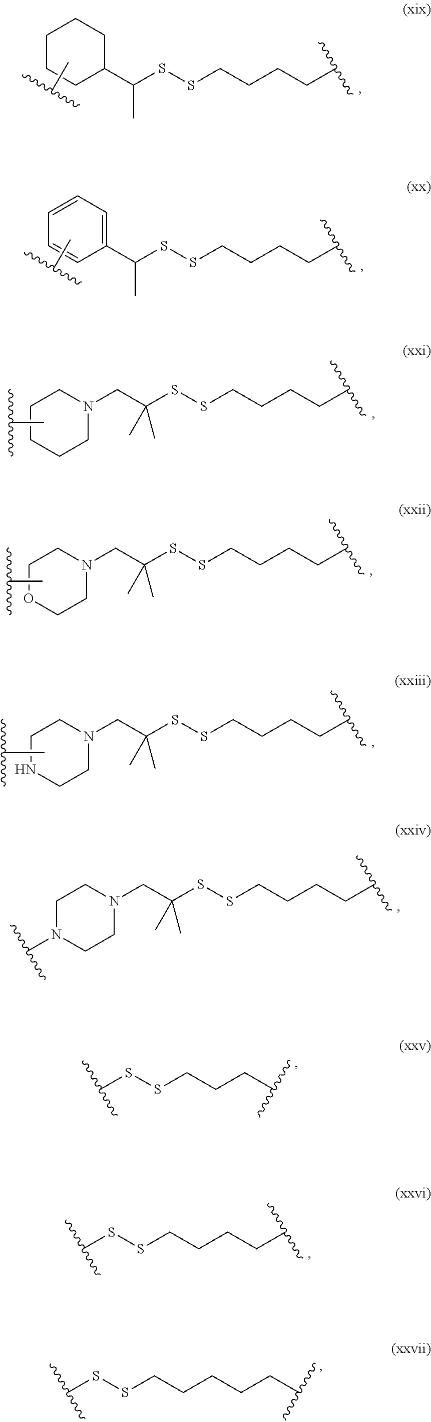 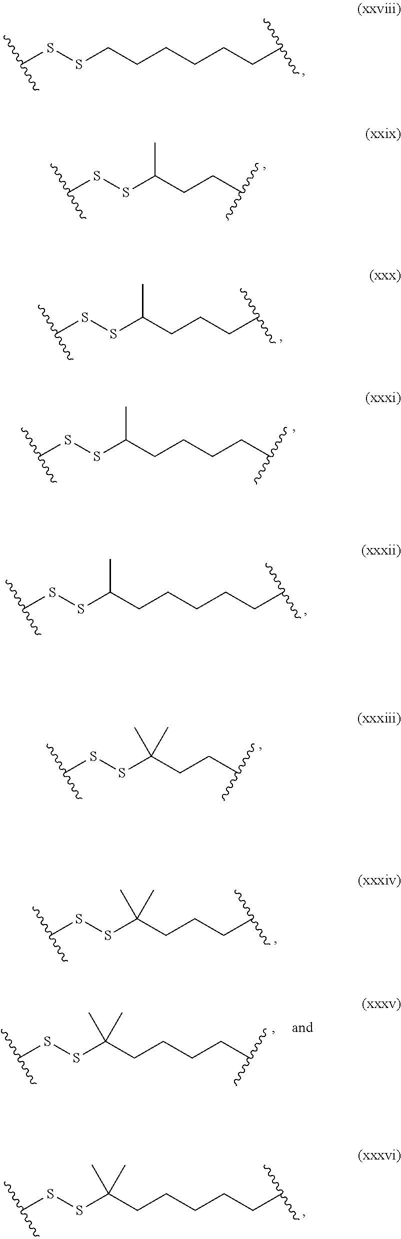
wherein,
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;
q is 0, 1, 2, 3, or 4; and
s is 0, 1, or 2.

145. The polynucleotide construct of claim 144, wherein R⁹ is halo or optionally substituted C₁₋₆ alkyl.

146. The polynucleotide construct of claim 144, wherein two adjacent R⁹ groups, together with the atoms to which each said R⁹ is attached, combine to form C₂₋₅ heteroaryl optionally substituted with 1, 2, or 3 C₁₋₆ alkyl groups.

147. The polynucleotide construct of claim 144, wherein A², A³, A⁴ and —S—S— combine to form a structure: 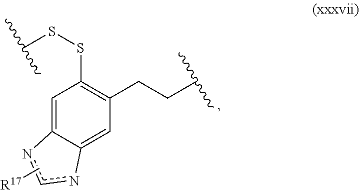
wherein the dotted lines represent one and only one double bond, and
R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

148. The hybridized polynucleotide construct of claim 147, wherein R¹⁷ is H or C₁₋₆ alkyl.

149. The polynucleotide construct of any one of claims 144 to 148, wherein s is 0 or 1.

150. The polynucleotide construct of any one of claims 144 to 147, wherein q is 0, 1, or 2.

151. The polynucleotide construct of claim 150, wherein q is 0 or 1.

152. The polynucleotide construct of claim 20, wherein A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A¹ and A², together with —S—S—, join to form an optionally substituted 5 to 16 membered ring.

153. The polynucleotide of claim 22, wherein A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A¹ and A², together with —S—S—, join to form an optionally substituted 5 to 16 membered ring.

154. The polynucleotide construct of any one of claims 22 to 153, wherein R¹ is selected from the group consisting of H, hydroxyl, optionally substituted C₁₋₆ alkoxy, a protected hydroxyl group, a monophosphate, a diphosphate, a triphosphate, an optionally substituted C₁₋₆ alkyl, an amino containing group, a biotin containing group, a polypeptide, a carbohydrate, a neutral organic polymer, a positively charged polymer, a therapeutic agent, a targeting moiety, an endosomal escape moiety, and any combination thereof.

155. The polynucleotide construct of any one of claims 22 to 154, wherein R² is selected from the group consisting of H, hydroxyl, optionally substituted C₁₋₆ alkoxy, a protected hydroxyl group, a monophosphate, a diphosphate, a triphosphate, an optionally substituted C₁₋₆ alkyl, an amino containing group, a biotin containing group, a polypeptide, a carbohydrate, a neutral organic polymer, a positively charged polymer, a therapeutic agent, a targeting moiety, an endosomal escape moiety, and any combination thereof.

156. The polynucleotide construct of any one of claims 22 to 155, wherein Y¹ is H.

157. A polynucleotide construct comprising one or more groups of Formula (V) attached to one or more internucleotide bridging groups or terminal nucleotide groups of said polynucleotide: 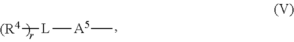
or a salt thereof,
wherein
each L is independently a bond or a conjugating group comprising one or more conjugating moieties;
each R⁴ is independently hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;
each r is independently an integer from 1 to 10; and
each A⁵ is independently selected from the group consisting of: 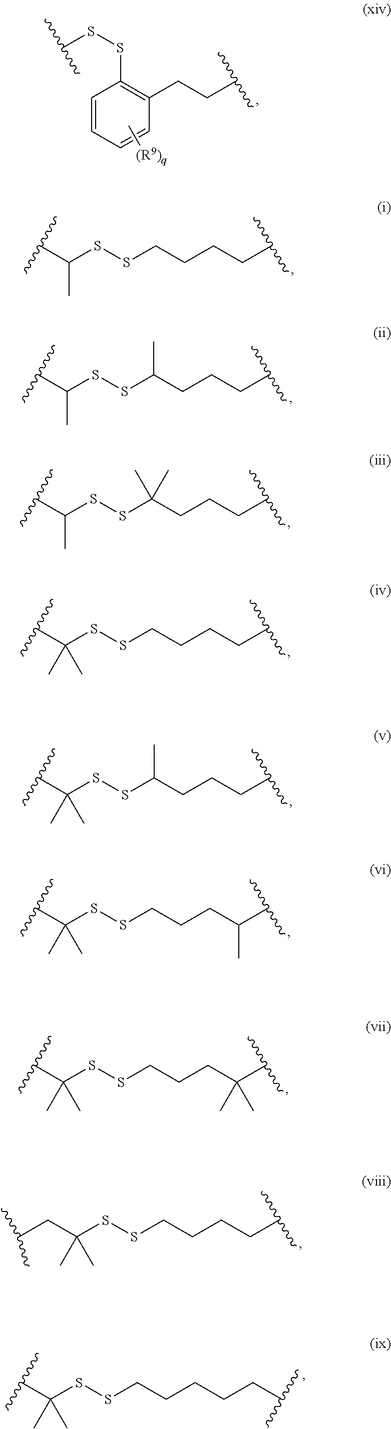 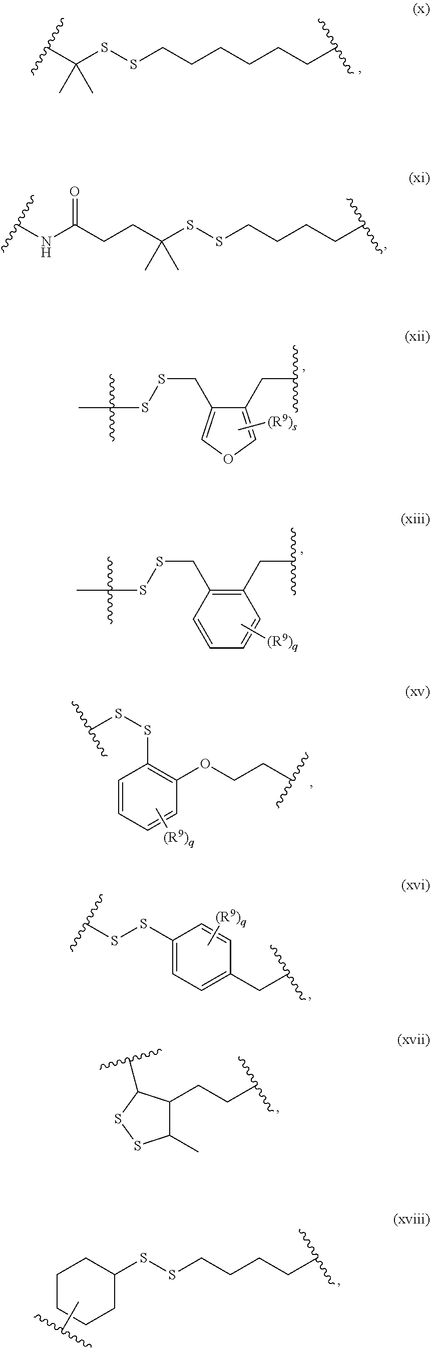 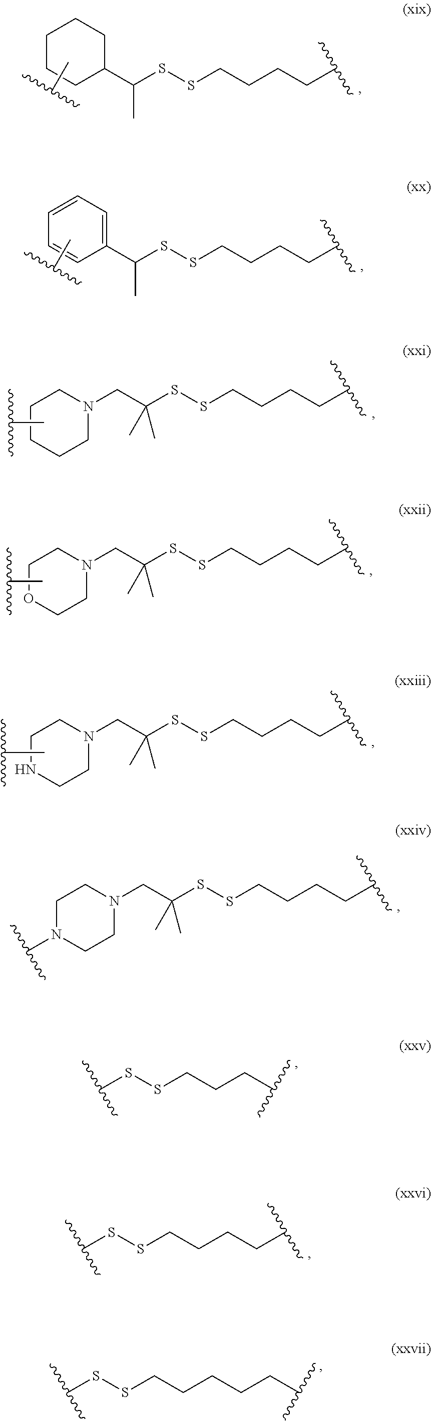 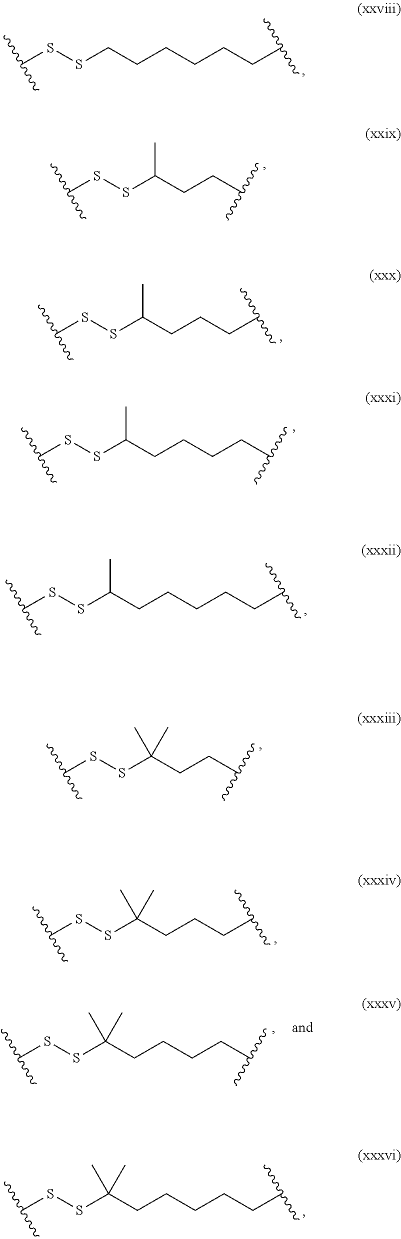
wherein,
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;
each q is independently 0, 1, 2, 3, or 4;
each s is independently 0, 1, or 2; and
wherein, when group of Formula (III) is attached at 5′ or 3′ end of said polynucleotide, A⁵ is not (i), (xviii), (xxv), (xxvi), (xxvii), or (xxviii).

158. The polynucleotide construct of claim 157, wherein R⁹ is halo or optionally substituted C₁₋₆ alkyl.

159. The polynucleotide construct of claim 157, wherein two adjacent R⁹ groups, together with the atoms to which each said R⁹ is attached, combine to form C₂₋₅ heteroaryl optionally substituted with 1, 2, or 3 C₁₋₆ alkyl groups.

160. The polynucleotide construct of claim 157, wherein A⁵ is: 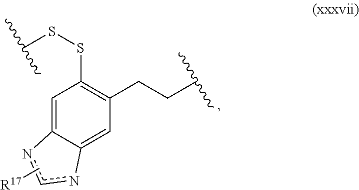
wherein the dotted lines represent one and only one double bond, and
R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

161. The polynucleotide construct of claim 160, wherein R¹⁷ is H or C₁₋₆ alkyl.

162. The polynucleotide construct of any one of claims 157 to 161, wherein s is 0 or 1.

163. The polynucleotide construct of claim 162, wherein s is 0.

164. The polynucleotide construct of any one of claims 157 to 163, wherein q is 0, 1, or 2.

165. The polynucleotide construct of claim 164, wherein q is 0 or 1.

166. A hybridized polynucleotide comprising the polynucleotide construct of any one of claims 1 to 165 hybridized to a complementary polynucleotide.

167. The hybridized polynucleotide of claim 166, wherein said complementary polynucleotide comprises one or more components (i), one or more groups of formula (II), or one or more groups of formula (V).

168. The hybridized polynucleotide of claim 166 or 167, wherein no more than 75% of the total number of nucleotides have the component (i), a group of formula (II), or a group of formula (V).

169. The hybridized polynucleotide of any one of claims 166 to 168, wherein said polynucleotide construct of any one of claims 1 to 165 and said complementary nucleotide each have between 10 and 32 nucleotides.

170. The hybridized polynucleotide of any one of claims 166 to 169, wherein said polynucleotide construct of any one of claims 1 to 165 and said complementary nucleotide each have between 19 and 25 nucleotides.

171. The hybridized polynucleotide of any one of claims 166 to 170, wherein said polynucleotide construct of any one of claims 1 to 165 is the guide strand, and the complementary polynucleotide is the passenger strand.

172. The hybridized polynucleotide of claim 171, wherein the passenger strand comprises one or more phosphotriesters having a moiety that is not cleavable by an intracellular enzyme.

173. The hybridized polynucleotide of claim 172, wherein said moiety that is not cleavable by the intracellular enzyme is optionally substituted C₁₋₆ alkyl.

174. A compound having a structure of Formula (VII): 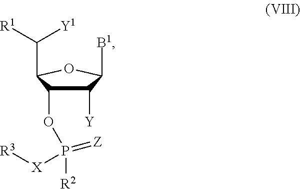
or a salt thereof,
wherein
B¹ is a nucleobase;
X is selected from the group consisting of O, S, and NR⁴;
Y is selected from the group consisting of hydrogen, hydroxyl, halo, optionally substituted C₁₋₆ alkoxy, and a protected hydroxyl group;
Y¹ is H or optionally substituted C₁₋₆ alkyl;
Z is absent, O, or S;
R¹ is selected from the group consisting of hydroxyl, optionally substituted C₁₋₆ alkoxy, a protected hydroxyl group, a monophosphate, a diphosphate, a triphosphate, a tetraphosphate, and a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and any combination thereof;
R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an amino, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and a combination thereof; and
R³ is a group having the structure of Formula (VIII): 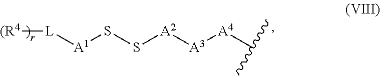
wherein A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with —S—S—, join to form an optionally substituted 5 to 16 membered ring;
A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;
A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;
L is a bond or a conjugating group comprising one or more conjugating moieties;
R⁴ is absent, hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and any combination thereof;
r is an integer from 1 to 10;
wherein A², A³, and A⁴ combine to form a group having at least three atoms in the shortest chain connecting —S—S— and X.

175. The compound of claim 174, wherein r is 1 to 7.

176. The compound of claim 174 or 175, wherein each X is O.

177. The compound of any one of claims 174 to 176, wherein each Z is O.

178. The compound of any one of claims 174 to 177, wherein Y is halo, optionally substituted C₁₋₆ alkoxy, or hydroxyl.

179. The compound of claim 178, wherein said Y is F.

180. The compound of claim 179, wherein said Y is OMe.

181. The compound of any one of claims 174 to 180, wherein R⁴ is bound to L, to A¹, or to disulfide via a bond formed by a reaction selected from the group consisting of a pericyclic reaction; an alkylation or arylation of a hydroxyl, thiol, or amino moiety; and a reaction of a hydroxyl, thiol, or amino nucleophile with an electrophile.

182. The compound of any one of claims 174 to 181, wherein R⁴ is bound to L, to A¹, or to disulfide via amide bond, a sulfonamide bond, a carboxylic ester, a thioester, an optionally substituted C₆₋₁₄ aryl or C₁₋₉ heteroaryl having 1 to 4 heteroatoms selected from N, O, and S; an imine; a hydrazone; an oxime; or a succinimide.

183. The compound of any one of claims 174 to 182, wherein at least one R⁴ is a targeting moiety.

184. The compound of any one of claims 174 to 183, wherein at least one R⁴ is a carbohydrate.

185. The compound of any one of claims 174 to 184, wherein at least one R⁴ is mannose.

186. The compound of any one of claims 174 to 185, wherein at least one R⁴ is N-acetyl galactosamine.

187. The compound of any one of claims 174 to 186, wherein at least one R⁴ comprises a folate ligand.

188. The compound of any one of claims 174 to 187, wherein at least one R⁴ comprises at least one protein transduction domain.

189. The compound of any one of claims 174 to 188, wherein at least one R⁴ is an endosomal escape moiety.

190. The compound of any one of claims 174 to 189, wherein L comprises 1 to 500 monomers, each of said monomers is, independently, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; carbonyl; thiocarbonyl; imino; optionally substituted N; O; or S(O)ₘ, wherein m is 0, 1, or 2.

191. The compound of any one of claims 174 to 190, wherein L comprises one or more C₁₋₆ alkyleneoxy groups.

192. The compound of claim 191, wherein L comprises less than 100 C₁₋₆ alkyleneoxy groups.

193. The compound of any one of claims 174 to 192, wherein L comprises one or more ethyleneoxy groups.

194. The compound of claim 174, wherein L comprises less than 100 ethyleneoxy groups.

195. The compound of any one of claims 174 to 193, wherein L comprises one or more poly(alkylene oxide).

196. The compound of claim 195, wherein said poly(alkylene oxide) is selected from polyethylene oxide, polypropylene oxide, poly(trimethylene oxide), polybutylene oxide, poly(tetramethylene oxide), and diblock or triblock co-polymers thereof.

197. The compound of claim 195 or 196, wherein said poly(alkylene oxide) is polyethylene oxide.

198. The compound of any one of claims 174 to 197, wherein L comprises one or more amino acid residues.

199. The compound of claim 198, wherein at least one of said amino acid residues is selected from the group consisting of Arg, Asn, Asp, Cys, Glu, Gin, His, Lys, Ser, Thr, Trp, and Tyr.

200. The compound of any one of claims 174 to 199, wherein L comprises a group having the structure of formula (III): 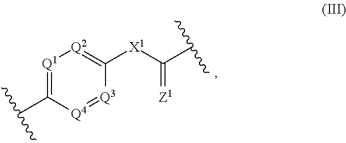
wherein each Q¹, Q², Q³, and Q⁴ is independently N or CR⁷;
X¹ is O or NR⁶;
Z¹ is O or S;
each R⁷ is independently selected from the group consisting of H; optionally substituted C₁₋₆ alkyl; optionally substituted C₂₋₆ alkenyl; optionally substituted C₂₋₆ alkynyl; halo; hydroxyl; —CHO; optionally substituted C₁₋₆ alkanoyl; carboxyl; cyano; nitro; amino; thiol; optionally substituted C₁₋₉ heterocyclyl having 1 to 4 heteroatoms selected from N, O, and S; optionally substituted C₁₋₉ heteroaryl having 1 to 4 heteroatoms selected from N, O, and S; optionally substituted C₆₋₁₄ aryl; optionally substituted C₃₋₈ cycloalkyl; and optionally substituted C₃₋₈ cycloalkenyl.

201. The compound of claim 200, wherein Q¹ is CR⁷.

202. The compound of claim 200 or 201, wherein Q² is CR⁷.

203. The compound of any one of claims 200 to 202, wherein Q³ is CR⁷.

204. The compound of any one of claims 200 to 203, wherein Q⁴ is CR⁷.

205. The compound of any one of claims 200 to 204, wherein each R⁷ is independently H, optionally substituted C₁₋₆ alkyl, or halo.

206. The compound of claim 205, wherein R⁷ is H.

207. The compound of any one of claims 200 to 206, wherein X¹ is NR⁶.

208. The compound of any one of claims 200 to 207, wherein Z¹ is S.

209. The compound of any one of claims 174 to 208, wherein L comprises one or more groups having the structure of formula (IV): 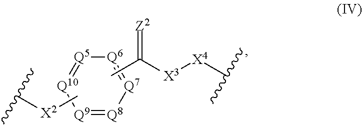
wherein each Q⁵, Q⁶, Q⁷, Q⁸, Q⁹, and Q¹⁰ is, independently, N, CR⁷, or C bonded to —X² or —C(Z²)X³X⁴, wherein one and only one of Q⁵, Q⁶, Q⁷, Q⁸, Q⁹, and Q¹⁰ is C bonded to —X², and one and only one of Q⁵, Q⁶, Q⁷, Q⁸, Q⁹, and Q¹⁰ is C bonded to —C(Z²)X³X⁴;
X² is optionally substituted C₁₋₆ cycloalkylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted diazaalkenylene; optionally substituted saturated diaza; unsaturated diaza; optionally substituted azacarbonyl; or oxacarbonyl;
X³ is a bond, O, NR⁷, or S;
X⁴ is absent, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O; optionally substituted C₁₋₉ heterocyclylene having 1 to 4 heteroatoms selected from N, O, and S; or optionally substituted (C₁₋₉ heterocyclyl)-C₁₋₄-alkylene having 1 to 4 heteroatoms selected from N, O, and S; and
Z² is O, S, or NR⁷; and
each R⁷ is independently selected from the group consisting of H, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; and optionally substituted C₁₋₆ alkoxy; and
wherein the two of Q⁵, Q⁶, Q⁷, Q⁸, Q⁹, and Q¹⁰ linked to X² and —C(Z¹)X³X⁴ are not N.

210. The compound of claim 209, wherein Q⁵ is N.

211. The compound of claim 209 or 210, wherein Q⁶ is CR⁷.

212. The compound of any one of claims 209 to 211, wherein Q⁷ is C bonded to —C(Z²)X³X⁴.

213. The compound of any one of claims 209 to 212, wherein Q⁸ is CR⁷.

214. The compound of any one of claims 209 to 213, wherein Q⁹ is CR⁷.

215. The compound of any one of claims 209 to 214, wherein Q¹⁰ is C bonded to X².

216. The compound of any one of claims 209 to 215, wherein each R⁷ is independently selected from the group consisting of H, halo, and optionally substituted C₁₋₆ alkyl.

217. The compound of claim 216, wherein R⁷ is H.

218. The compound of any one of claims 209 to 217, wherein X² is optionally substituted diazaalkenylene or optionally substituted saturated diaza.

219. The compound of any one of claims 209 to 218, wherein X³ is NR⁷.

220. The compound of any one of claims 209 to 219, wherein X⁴ is absent.

221. The compound of any one of claims 209 to 220, wherein Z² is O.

222. The compound of any one of claims 174 to 221, wherein L comprises one or more groups having the structure:

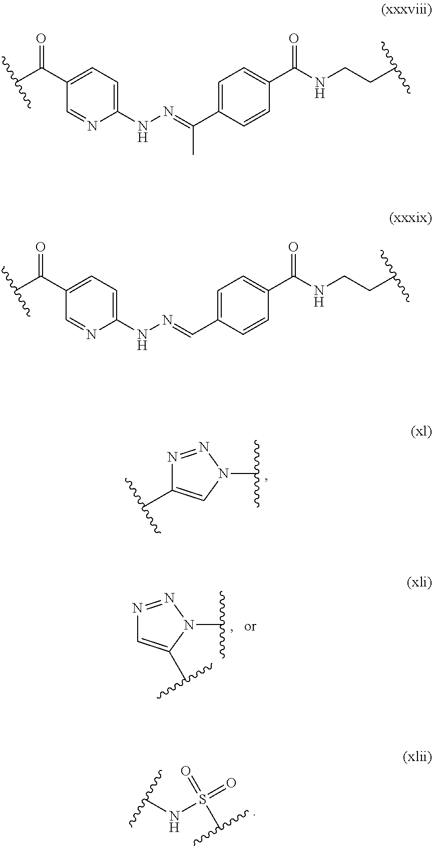 [SMILES: *CCNC(=O)c1ccc(/C(C)=N/Nc2ccc(C(*)=O)cn2)cc1 ; *CCNC(=O)c1ccc(/C=N/Nc2ccc(C(*)=O)cn2)cc1 ; *c1cn(*)nn1 ; *c1cnnn1* ; *NS(*)(=O)=O]

223. The compound of any one of claims 174 to 222, wherein L is a bond.

224. The compound of any one of claims 174 to 223, wherein A³ is selected from the group consisting of a bond, optionally substituted C₁₋₆ alkylene; optionally substituted C₆₋₁₄ arylene; O; optionally substituted N; and S.

225. The compound of any one of claims 174 to 224, wherein A³ is selected from the group consisting of a bond, optionally substituted C₁₋₆ alkylene; optionally substituted C₆₋₁₄ arylene; and O.

226. The compound of claim 225, wherein A³ has a structure of formula (VI): 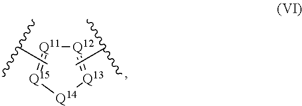
wherein
Q¹¹ is N, or C bonded to R¹⁰ or to A²;
Q¹² is N, or C bonded to R¹¹ or to A⁴;
Q¹³ is N or C bonded to R¹² or to A⁴;
Q¹⁴ is O, S, N bonded to R¹³ or to A⁴, or —C(R¹⁴ or bond to A⁴)=C(R¹⁵ or bond to A⁴)-;
Q¹⁵ is N, or C bonded to R¹⁶ or to A²;
each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and
wherein
one and only one of Q¹¹ and Q¹⁵ is bonded to A², and
one and only one of Q¹², Q¹³, and Q¹⁴ is bonded to A⁴.

227. The compound of claim 226, wherein Q¹¹ is C bonded to A².

228. The compound of claim 226 or 227, wherein Q¹² is C bonded to A⁴.

229. The compound of any one of claims 226 to 228, wherein Q¹³ is C bonded to R¹².

230. The compound of claim 229, wherein R¹² is H, halo, or C₁₋₆ alkyl.

231. The compound of any one of claims 226 to 230, wherein Q¹⁴ is O.

232. The compound of any one of claims 226 to 231, wherein Q¹⁴ is —C(R¹⁴)═C(R¹⁵)—.

233. The compound of claim 232, wherein R¹⁴ is H, halo, or C₁₋₆ alkyl.

234. The compound of claim 232 or 233, wherein R¹⁵ is H, halo, or C₁₋₆ alkyl.

235. The compound of any one of claims 226 to 234, wherein Q¹⁵ is C bonded to R¹⁶.

236. The compound of claim 235, wherein R¹⁶ is H, halo, or C₁₋₆ alkyl.

237. The compound of any one of claims 174 to 236, wherein A⁴ is optionally substituted C₁₋₆ alkylene.

238. The compound of any one of claims 174 to 237, wherein A comprises a group having the structure:

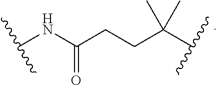 [SMILES: *NC(=O)CCC(*)(C)C]

239. The compound of any one of claims 174 to 237, wherein
A¹ is a bond or comprises one or more groups selected independently from the group consisting of optionally substituted C₁₋₆ alkylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; optionally substituted (C₁₋₉ heterocyclyl)-C₁₋₄-alkylene having 1 to 4 heteroatoms selected from N, O, and S; optionally substituted N; and O.

240. The compound of claim 239, wherein A¹ is a bond or comprises one or more groups selected independently from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

241. The compound of claim 240, wherein A¹ is a bond or comprises one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

242. The compound of claim 241, wherein A¹ is a bond or comprises one or more groups selected independently from the group consisting of optionally substituted C₁₋₆ alkylene; optionally substituted C₃₋₈ cycloalkylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; and O.

243. The compound of any one of claims 174 to 242, wherein A¹ is a bond.

244. The compound of any one of claims 174 to 243, wherein A² is optionally substituted C₁₋₆ alkylene, optionally substituted C₃₋₈ cycloalkylene; optionally substituted C₃₋₈ cycloalkenylene; optionally substituted C₆₋₁₄ arylene; or optionally substituted C₁₋₉ heteroarylene having 1 to 4 heteroatoms selected from N, O, and S.

245. The compound of claim 244, wherein A² is optionally substituted C₁₋₆ alkylene, optionally substituted C₃₋₈ cycloalkylene; optionally substituted C₆₋₁₄ arylene; or optionally substituted C₁₋₉ heteroarylene having 1 to 4 heteroatoms selected from N, O, and S.

246. The compound of claim 245, wherein A² is optionally substituted optionally substituted C₆₋₁₄ arylene or optionally substituted C₁₋₉ heteroarylene having 1 to 4 heteroatoms selected from N, O, and S.

247. The compound of claim 246, wherein A² has a structure of formula (VI): 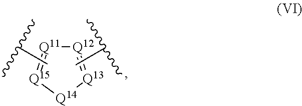
wherein
Q¹¹ is N, or C bonded to R¹⁰ or to the disulfide linkage;
Q¹² is N, or C bonded to R¹¹ or to A³;
Q¹³ is N or C bonded to R¹² or to A³;
Q¹⁴ is O, S, N bonded to R¹³ or to A³, or —C(R¹⁴ or bond to A³)=C(R¹⁵ or bond to A³)-;
Q¹⁵ is N, or C bonded to R¹⁶ or to the disulfide linkage;
each of R¹⁰, R¹¹, R¹², R¹³, R¹⁴, R¹⁵, and R¹⁶ is, independently, H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl; and
wherein
one and only one of Q¹¹ and Q¹⁵ is bonded to the disulfide linkage, and
one and only one of Q¹², Q¹³, and Q¹⁴ is bonded to A³.

248. The compound of claim 247, wherein Q¹¹ is C bonded to the disulfide linkage.

249. The compound of claim 247 or 248, wherein Q¹² is C bonded to A³.

250. The compound of any one of claims 247 to 249, wherein Q¹³ is C bonded to R¹².

251. The compound of claim 250, wherein R¹² is H, halo, or C₁₋₆ alkyl.

252. The compound of any one of claims 247 to 251, wherein Q¹⁴ is O.

253. The compound of any one of claims 247 to 252, wherein Q¹⁴ is —C(R¹⁴)═C(R¹⁵)—.

254. The compound of claim 253, wherein R¹⁴ is H, halo, or C₁₋₆ alkyl.

255. The compound of claim 253 or 254, wherein R¹⁵ is H, halo, or C₁₋₆ alkyl.

256. The compound of any one of claims 247 to 255, wherein Q¹⁵ is C bonded to R¹⁶.

257. The compound of claim 256, wherein R¹⁶ is H, halo, or C₁₋₆ alkyl.

258. The compound of any one of claims 174 to 257, wherein, when the carbon atom attached to the sulfur atom of —S—S-A²-A³-A⁴- is an alkylene carbon atom, said alkylene carbon atom is connected to at most one hydrogen atom.

259. The compound of any one of claims 174 to 258, wherein, when the carbon atom attached to the sulfur atom of —S—S-A²-A³-A⁴- is an alkylene carbon atom, said alkylene carbon atom is not connected to a hydrogen atom.

260. The compound of any one of claims 174 to 257, wherein, when the carbon atom attached to the sulfur atom of —S—S-A²-A³-A⁴- is an alkenylene carbon atom, said alkenylene carbon atom is not connected to a hydrogen atom.

261. The polynucleotide construct of any one of claims 174 to 260, wherein the carbon atom attached to the sulfur atom of —S—S-A²-A³-A⁴- is not an alkynylene carbon atom.

262. The compound of any one of claims 174 to 261, wherein, when the carbon atom attached to the sulfur atom of (R⁴)ᵣ-L-A¹-S—S— is an alkylene carbon atom, said carbon atom is connected to at most one hydrogen atom.

263. The compound of any one of claims 174 to 262, wherein, when the carbon atom attached to the sulfur atom of (R⁴)ᵣ-L-A¹-S—S— group is an alkylene carbon atom, said carbon atom is not connected to a hydrogen atom.

264. The compound of any one of claims 174 to 263, wherein A¹ and A², together with —S—S— to which they are attached, join to form an optionally substituted 5 to 16 membered ring.

265. The compound of claim 264, wherein A¹ and A², together with —S—S— to which they are attached, join to form an optionally substituted 5 to 7 membered ring.

266. The compound of claim 174, wherein A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₂₋₉ heteroarylene having 1 to 4 heteroatoms selected from N, O, and S; optionally substituted (C₂₋₉ heteroaryl)-C₁₋₄-alkylene having 1 to 4 heteroatoms selected from N, O; optionally substituted C₂₋₉ heterocyclylene having 1 to 4 heteroatoms selected from N, O, and S; and optionally substituted (C₂₋₉ heterocyclyl)-C₁₋₄-alkylene having 1 to 4 heteroatoms selected from N, O, and S; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₂₋₉ heteroarylene having 1 to 4 heteroatoms selected from N, O, and S; and optionally substituted C₂₋₉ heterocyclylene having 1 to 4 heteroatoms selected from N, O, and S; or A¹ and A², together with —S—S—, join to form an optionally substituted 5 to 16 membered ring.

267. The compound of any one of claims 174 to 266, wherein -A¹-S—S-A²-A³-A⁴- or —S—S-A²-A³-A⁴- is: 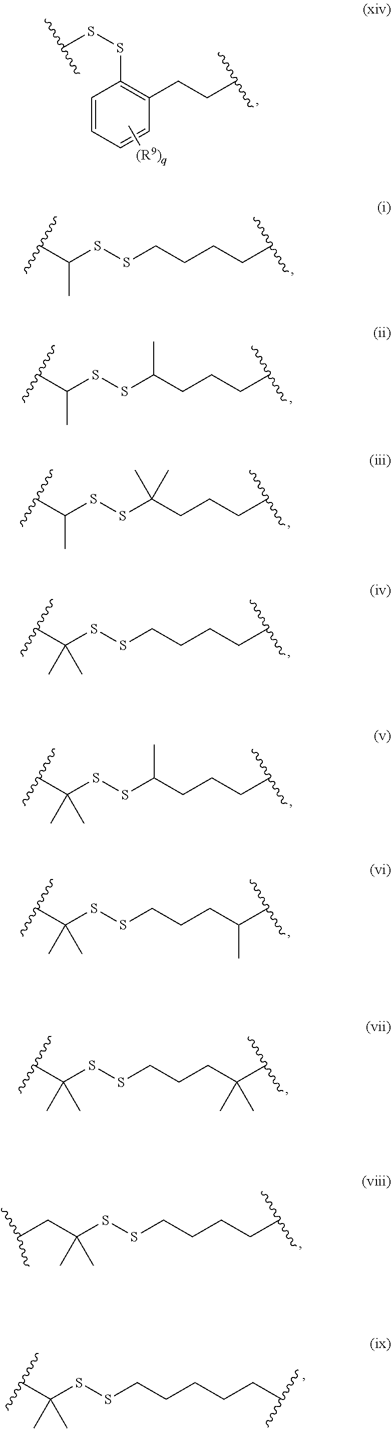 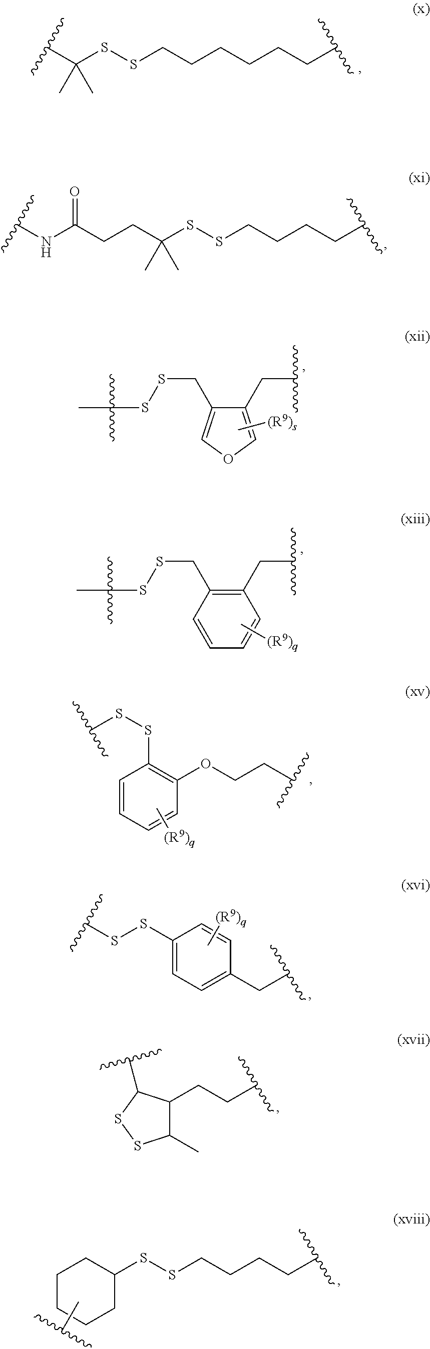 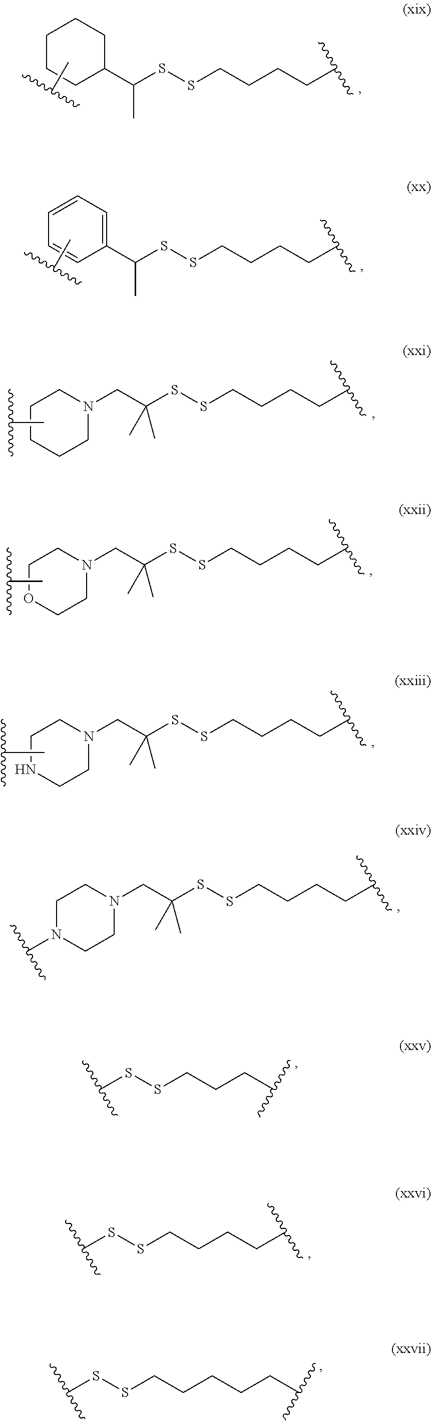 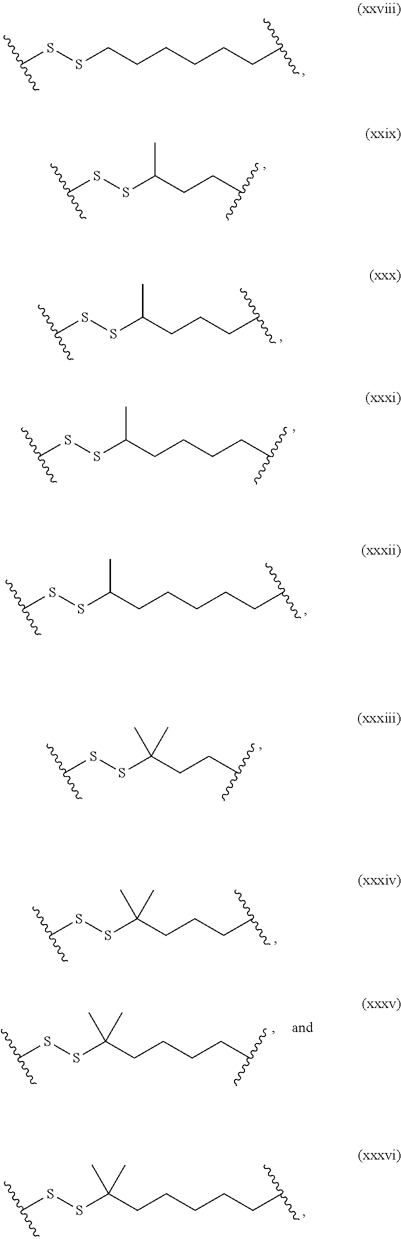
wherein,
each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S; optionally substituted (C₁₋₉ heterocyclyl)-C₁₋₄-alkyl having 1 to 4 heteroatoms selected from N, O, and S; amino; or optionally substituted C₁₋₆ alkoxy; or two adjacent R⁹ groups, together with the atoms to which each said R⁹ is attached, combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;
q is 0, 1, 2, 3, or 4; and
s is 0, 1, or 2.

268. The compound of claim 267, wherein two adjacent R⁹ groups, together with the atoms to which each said R⁹ is attached, combine to form C₂₋₅ heteroaryl optionally substituted with 1, 2, or 3 C₁₋₆ alkyl groups.

269. The compound of claim 267, wherein -A¹-S—S-A²-A³-A⁴- or —S—S-A²-A³-A⁴- is: 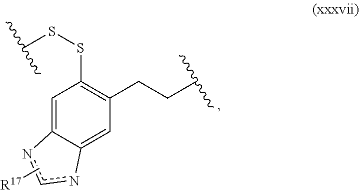
wherein the dotted lines represent one and only one double bond, and
R¹⁷ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen; C₁₋₆ alkyl; C₆₋₁₀ aryl; (C₆₋₁₀ aryl)-C₁₋₄-alkyl; thiol; C₆₋₁₀ aryloxy; C₃₋₈ cycloalkoxy; (C₆₋₁₀ aryl)-C₁₋₄-alkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

270. The compound of claim 269, wherein R¹⁷ is H or C₁₋₆ alkyl.

271. The compound of claim 267, wherein R⁹ is halo or optionally substituted C₁₋₆ alkyl.

272. The compound of any one of claims 267 to 271, wherein s is 0 or 1.

273. The compound of claim 272, wherein s is 0.

274. The compound of any one of claims 267 to 273, wherein q is 0, 1, or 2.

275. The compound of claim 274, wherein q is 0 or 1.

276. The compound of any one of claims 174-275, wherein Y¹ is H.

277. A method of delivering a polynucleotide construct to a cell comprising contacting said cell with the polynucleotide construct of any one of claims 1 to 165 or the hybridized polynucleotide of any one of claims 166 to 173.

278. A method of reducing the expression of a polypeptide in a cell comprising contacting said cell with the polynucleotide construct of any one of claims 1 to 165 or the hybridized polynucleotide of any one of claims 166 to 173.


SEQUENCE LISTING (100 sequences)

SEQ ID NO: 1 - protein, 9 aa, Artificial Sequence
  Synthetic Construct
RKKRRQRRR

SEQ ID NO: 2 - protein, 8 aa, Artificial Sequence
  Synthetic Construct
RRRRRRRR

SEQ ID NO: 3 - protein, 16 aa, Artificial Sequence
  Synthetic Construct
RQIKIWFQNRRMKWKK

SEQ ID NO: 4 - protein, 21 aa, Artificial Sequence
  Synthetic Construct
TRSSRAGLQFPVGRVHRLLRK

SEQ ID NO: 5 - protein, 26 aa, Artificial Sequence
  Synthetic Construct
GWTLNSAGYLLGKINKALAALAKKIL

SEQ ID NO: 6 - protein, 18 aa, Artificial Sequence
  Synthetic Construct
KLALKLALKALKAALKLA

SEQ ID NO: 7 - protein, 16 aa, Artificial Sequence
  Synthetic Construct
AAVALLPAVLLALLAP

SEQ ID NO: 8 - protein, 10 aa, Artificial Sequence
  Synthetic Construct
VPMLKPMLKE

SEQ ID NO: 9 - protein, 28 aa, Artificial Sequence
  Synthetic Construct
MANLGYWLLALFVTMWTDVGLCKKRPKP

SEQ ID NO: 10 - protein, 18 aa, Artificial Sequence
  Synthetic Construct
LLIILRRRIRKQAHAHSK

SEQ ID NO: 11 - protein, 21 aa, Artificial Sequence
  Synthetic Construct
KETWWETWWTEWSQPKKKRKV

SEQ ID NO: 12 - protein, 18 aa, Artificial Sequence
  Synthetic Construct
RGGRLSYSRRRFSTSTGR

SEQ ID NO: 13 - protein, 15 aa, Artificial Sequence
  Synthetic Construct
SDLWEMMMVSLACQY

SEQ ID NO: 14 - protein, 12 aa, Artificial Sequence
  Synthetic Construct
TSPLNIHNGQKL

SEQ ID NO: 15 - protein, 7 aa, Artificial Sequence
  Synthetic Construct
KKRPKPG

SEQ ID NO: 16 - protein, 86 aa, Human immunodeficiency virus type 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 17 - protein, 130 aa, Human immunodeficiency virus type 2
METPLKEPESSLESYNEPSSCTSERDVTAQERAKQGEELLAQLHRPLEACTNSCYCKQCSYHCQLCFLKKGLGIWYARQGRRRRTPRKTKTHPPPASDKSISTRTGDSQPTKKQKKTPETTVVSACGLGH

SEQ ID NO: 18 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYKRGSSMVLFSSPPVILLISFLIFLIVG

SEQ ID NO: 19 - protein, 17 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
GGRXRXRXRXRXRXRXR

SEQ ID NO: 20 - protein, 25 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXR

SEQ ID NO: 21 - protein, 33 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
  MOD_RES (26)..(26): Xaa is Boc-Lys(Fmoc)-OH
  MOD_RES (28)..(28): Xaa is Boc-Lys(Fmoc)-OH
  MOD_RES (30)..(30): Xaa is Boc-Lys(Fmoc)-OH
  MOD_RES (32)..(32): Xaa is Boc-Lys(Fmoc)-OH
GGRXRXRXRXRXRXRXRXRXRXRXRXRXRXRXR

SEQ ID NO: 22 - protein, 55 aa, Artificial Sequence
  Synthetic Construct
GGESDSELEIKRYKNRVASRKSRAKFKQLLQHYREVAAAKSSENDRLRLLLKQSS

SEQ ID NO: 23 - protein, 18 aa, Artificial Sequence
  Synthetic Construct
GGSRRHHSRSKAKRSRHH

SEQ ID NO: 24 - protein, 23 aa, Artificial Sequence
  Synthetic Construct
GGAYDLRRRERQSRLRRRERQSR

SEQ ID NO: 25 - protein, 31 aa, Artificial Sequence
  Synthetic Construct
GGMAPQRDTVGGRTTPPSWGPAKAQLRNSCA

SEQ ID NO: 26 - protein, 31 aa, Artificial Sequence
  Synthetic Construct
GGMAPQRDTVGGRTTPPSWGPAKAQLRNSSA

SEQ ID NO: 27 - protein, 8 aa, Artificial Sequence
  Synthetic Construct
GGFCIGRL

SEQ ID NO: 28 - protein, 8 aa, Artificial Sequence
  Synthetic Construct
GGGVIGRL

SEQ ID NO: 29 - protein, 17 aa, Artificial Sequence
  Synthetic Construct
GGRAWMRWYSPTTRRYG

SEQ ID NO: 30 - protein, 11 aa, Artificial Sequence
  Synthetic Construct
GGPLILLRLLR

SEQ ID NO: 31 - protein, 12 aa, Artificial Sequence
  Synthetic Construct
GGMIIYRDLISH

SEQ ID NO: 32 - protein, 13 aa, Artificial Sequence
  Synthetic Construct
GGACTGSTQHQCG

SEQ ID NO: 33 - protein, 25 aa, Artificial Sequence
  Synthetic Construct
GGALFLGWLGAAGSTMGAPKSKRKV

SEQ ID NO: 34 - protein, 25 aa, Artificial Sequence
  Synthetic Construct
GGLIRLWSHLIHIWFQNRRLKWKKK

SEQ ID NO: 35 - protein, 27 aa, Artificial Sequence
  Synthetic Construct
GGIGAVLKVLTTGLPALISWIKRKRQQ

SEQ ID NO: 36 - protein, 28 aa, Artificial Sequence
  Synthetic Construct
GGLHKLLHHLLHHLHKLLHHLHHLLHKL

SEQ ID NO: 37 - protein, 6 aa, Artificial Sequence
  Synthetic Construct
GGRKKR

SEQ ID NO: 38 - protein, 11 aa, Artificial Sequence
  Synthetic Construct
GGRKKRRQRRR

SEQ ID NO: 39 - protein, 17 aa, Artificial Sequence
  Synthetic Construct
GGRKKRRQRRRGGRKKR

SEQ ID NO: 40 - protein, 11 aa, Artificial Sequence
  Synthetic Construct
GGRKKRRQRRR

SEQ ID NO: 41 - protein, 11 aa, Artificial Sequence
  Synthetic Construct
GGRKKRRQRRR

SEQ ID NO: 42 - protein, 26 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXRC

SEQ ID NO: 43 - protein, 26 aa, Artificial Sequence
  Sythetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXRC

SEQ ID NO: 44 - protein, 11 aa, Artificial Sequence
  Synthetic Construct
GGRKKRRQRRR

SEQ ID NO: 45 - protein, 28 aa, Artificial Sequence
  Synthetic Construct
GGLHKLLHHLLHHLHKLLHHLHHLLHKL

SEQ ID NO: 46 - protein, 13 aa, Artificial Sequence
  Synthetic Construct
GGACTGSTQHQCG

SEQ ID NO: 47 - protein, 25 aa, Artificial Sequence
  Synthetic Construct
GGLIRLWSHLIHIWFQNRRLKWKKK

SEQ ID NO: 48 - protein, 25 aa, Artificial Sequence
  Synthetic Construct
GGALFLGWLGAAGSTMGAPKSKRKV

SEQ ID NO: 49 - protein, 27 aa, Artificial Sequence
  Synthetic Construct
GGIGAVLKVLTTGLPALISWIKRKRQQ

SEQ ID NO: 50 - protein, 24 aa, Artificial Sequence
  Synthetic Construct
GGLFGAIAGFIENGWEGMIDGWYG

SEQ ID NO: 51 - protein, 24 aa, Artificial Sequence
  Synthetic Construct
GGLFEAIEGFIENGWEGMIDGWYG

SEQ ID NO: 52 - protein, 33 aa, Artificial Sequence
  Synthetic Construct
GGLFEAIEGFIENGWEGMIDGWYGRKKRRQRRR

SEQ ID NO: 53 - protein, 24 aa, Artificial Sequence
  Synthetic Construct
GGLFEAIEGFIENGWEGLIEGWYG

SEQ ID NO: 54 - protein, 31 aa, Artificial Sequence
  Synthetic Construct
GGKWKLFKKIGAVLKVLTTGYGRKKRRQRRR

SEQ ID NO: 55 - protein, 18 aa, Artificial Sequence
  Synthetic Construct
ILSSLTVTQLLRRLHQWI

SEQ ID NO: 56 - protein, 19 aa, Artificial Sequence
  Synthetic Construct
MKWVTFISLLFLFFSSAYS

SEQ ID NO: 57 - protein, 16 aa, Artificial Sequence
  Synthetic Construct
MIRTLLLSTLVAGALS

SEQ ID NO: 58 - protein, 18 aa, Artificial Sequence
  Synthetic Construct
RLIEDICLPRWGCLWEDD

SEQ ID NO: 59 - protein, 4 aa, Artificial Sequence
  Synthetic Construct
KDEL

SEQ ID NO: 60 - protein, 26 aa, Artificial Sequence
  Synthetic Construct
LFEAIEGFIENGWEGMIDGWYGKDEL

SEQ ID NO: 61 - protein, 35 aa, Artificial Sequence
  Synthetic Construct
LFEAIEGFIENGWEGMIDGWYGRKKRRQRRRKDEL

SEQ ID NO: 62 - protein, 18 aa, Artificial Sequence
  Synthetic Construct
RLIEDICLPRWGCLWEDD

SEQ ID NO: 63 - protein, 18 aa, Artificial Sequence
  Synthetic Construct
MKLSLVAAMLLLLSAARA

SEQ ID NO: 64 - protein, 21 aa, Artificial Sequence
  Synthetic Construct
MKLAVTLTLVTLALSSSSASA

SEQ ID NO: 65 - protein, 26 aa, Artificial Sequence
  Synthetic Construct
FFKKLAHALHLLALLALHLAHALKKA

SEQ ID NO: 66 - protein, 35 aa, Artificial Sequence
  Synthetic Construct
PSQPTYPGDDAPVRDLIRFYRDLRRYLNVVTRHRY

SEQ ID NO: 67 - protein, 22 aa, Artificial Sequence
  Synthetic Construct
RLIEDICLPRWGCLWEDDKDEL

SEQ ID NO: 68 - protein, 21 aa, Artificial Sequence
  Synthetic Construct
LFEAIEGFIENGWGMIDGWYG

SEQ ID NO: 69 - protein, 31 aa, Artificial Sequence
  Synthetic Construct
LFEAIEGFIENGWEGMIDGWYGRKKRRQRRR

SEQ ID NO: 70 - protein, 20 aa, Artificial Sequence
  Synthetic Construct
MIRTLLLSTLVAGALSKDEL

SEQ ID NO: 71 - protein, 13 aa, Artificial Sequence
  Synthetic Construct
YEQDPWGVKWWYK

SEQ ID NO: 72 - protein, 17 aa, Artificial Sequence
  Synthetic Construct
MIRTLLLSTLVAGALSK

SEQ ID NO: 73 - protein, 13 aa, Artificial Sequence
  Synthetic Construct
YEQDPWGVKWWYK

SEQ ID NO: 74 - protein, 5 aa, Artificial Sequence
  Synthetic Construct
  MOD_RES (2)..(2): Xaa is Bis-phenylalanine
  MOD_RES (4)..(4): Xaa is Bis-phenylalanine
RXRXR

SEQ ID NO: 75 - skipped

SEQ ID NO: 76 - protein, 19 aa, Artificial Sequence
  Synthetic Construct
ILSSLTVTQLLRRLHQWIK

SEQ ID NO: 77 - protein, 20 aa, Artificial Sequence
  Synthetic Construct
MIRTLLLSTLVAGALSKDEL

SEQ ID NO: 78 - protein, 35 aa, Artificial Sequence; identical to SEQ ID NO: 61
  Synthetic Construct

SEQ ID NO: 79 - protein, 25 aa, Artificial Sequence
  Synthetic Construct
IGAVLKVLTTGLPALISWIKRKRQQ

SEQ ID NO: 80 - protein, 29 aa, Artificial Sequence
  Synthetic Construct
IGAVLKVLTTGLPALISWIKRKRQQKDEL

SEQ ID NO: 81 - protein, 6 aa, Artificial Sequence
  Synthetic Construct
  MOD_RES (3)..(3): Xaa is D-Trp
CFXKTC

SEQ ID NO: 82 - protein, 5 aa, Artificial Sequence
  Synthetic Construct
YIGSR

SEQ ID NO: 83 - protein, 4 aa, Artificial Sequence
  Synthetic Construct
MLFK

SEQ ID NO: 84 - skipped

SEQ ID NO: 85 - protein, 5 aa, Artificial Sequence
  Synthetic Construct
GGGGS

SEQ ID NO: 86 - protein, 12 aa, Artificial Sequence
  Synthetic Construct
GKSSGSGSESKS

SEQ ID NO: 87 - protein, 14 aa, Artificial Sequence
  Synthetic Construct
GSTSGSGKSSEGKG

SEQ ID NO: 88 - protein, 18 aa, Artificial Sequence
  Synthetic Construct
GSTSGSGKSSEGSGSTKG

SEQ ID NO: 89 - protein, 18 aa, Artificial Sequence
  Synthetic Construct
GSTSGSGKPGSGEGSTKG

SEQ ID NO: 90 - protein, 14 aa, Artificial Sequence
  Synthetic Construct
EGKSSGSGSESKEF

SEQ ID NO: 91 - DNA, 21 bp, Artificial Sequence
  Synthetic Construct
gcuacauucuggagacauaut

SEQ ID NO: 92 - DNA, 21 bp, Artificial Sequence
  Synthetic Construct
uaugucuccagaauguagcut

SEQ ID NO: 93 - DNA, 21 bp, Artificial Sequence
  Synthetic Construct
ucuacauguuccaguaugaut

SEQ ID NO: 94 - DNA, 21 bp, Artificial Sequence
  Synthetic Construct
ucauacuggaacauguagaut

SEQ ID NO: 95 - DNA, 21 bp, Artificial Sequence
  Synthetic Construct
ucaucacacugaauaccaaut

SEQ ID NO: 96 - DNA, 21 bp, Artificial Sequence
  Synthetic Construct
uugguauucagugugaugaut

SEQ ID NO: 97 - DNA, 21 bp, Artificial Sequence
  Synthetic Construct
aguacugcuuacgauacggut

SEQ ID NO: 98 - DNA, 21 bp, Artificial Sequence
  Synthetic Construct
ccguaucguaagcaguacuut

SEQ ID NO: 99 - DNA, 21 bp, Artificial Sequence
  Synthetic Construct
aggaucaucucaagucuuaut

SEQ ID NO: 100 - DNA, 21 bp, Artificial Sequence
  Synthetic Construct
uaagacuugagaugauccuut